SEQUENCE LISTING (35 sequences)

SEQ ID NO: 1 - DNA, 10715 bp, Sorghum propinquum
  misc_feature (6480)..(6480): n is a, c, g, or t
aaaagaaaagtgagcacaccacgacctgtcatcagctcatggtcagctctacaaacttatagattgcatcgagatctaagactcaggtacaaatcatgtcaacatctaatggtttagaaaatgaaaagttttgagtttcaaaatatgatacgtgatattaacatttgaacttttagcaagatctgaaataaaaaattcaactagatcatgttaacattgatataatcgcttccaatcgcctcccatcacttccgctagaaaactttttttctcgatttaattaatgaaagggtaataacatcattgtacaagattctttcaaacctcaacccctatcatcgacggtgacggctccctataacacgcactagtggacgccgggcgggtggaaccctaagaagatttaaaaaaacttaagaagaagatttttatctaactaactatagtacttatatcatacactatactattcaaaatattattttcacaattatgaatttacccttttactcttcattaaaaaaatacgaaaaaagaatcaccacgtctctatttagggtcctagtccccataatttaagaggcggtgagagacgatgtgacgtctatggaccaccgaccaaagacacacctatcgtctcccatcgccttgcttccatcgcctctcatcgcttttcatattctagatccagcggccatagacacaccaatcgtttctcatcgcctctccaaccattgtaaaaatatttataattttgatataaaatttgtcttcacttgagttcatgccaaaaaaattatacatattattttcgtgtgagaatttacagaagtggactcttaagatgtccaaatgtaaatgaccctatttattatgaggcgcggatctataggcctgactctgaaaatggattatggatttgagataataaatttaagggcctatcttcgcacataacatctatagttcctaaatttttttttattgtagtagtagaacttttctccctgtaaaccaagttgacgctgggctttattttgcgacacagaacaccaaattggtggctatgaactcttccacctgggcagggaaaacggtttattatgtttctctttaatttatctatcgtggcactataacacaacatggctttgccgacacttccaactatcggcaaagggtacctttaccgacacttaacgtctcacgaaaggttttgccgacaattttcaaacagtcgcggtagaagcagttggcgaaacttttgccgacagttaaaggcatcgccgacacattttctgtagtcaaatggcatacctacgccgacagttgaactttcaccgacagtgaaccctttgccgacagtttggacctacgccgacagtttggaccttttccgacagttggtatgttagcgaaaccgtttctagggtgtttcataaaccatgccttgtccaacagtagaagtgtcggcaaaactatattgctaggatgtagatacaatttaaatattttaataaatacacatcacattgattgagcaaaatcacatggtctgttttcactaaaactgtcagaggtacactccagtactaccagtacgtcgcccgcacagtggccaaggattttactgctactgttgattaacataagcacttgcgactttccctaaaatcttttataaaacaacggccgcaataatattgaactattttttttctagtaccaaaattagaatttgatccctcacctcattacatccatagtaacatgaccagatatatatggacaggatgggatcactcagcgagcagatacactgagcgattcataatcagattttttaatttcttctagtgaagtggggttttcctagtcttttaacattcaaaatttagtacaaactttccctagtaaatgccttctagtaaagatttcctagtattttgactagcgatagtgttttattactaattaaaaacattagaagaactccatttagtgattggttgtttggattagtcttctcacgttagacctatatatgcaggacaactcaagccagcataaatatatgaaatatcttggtgtttgtttgtctgacacaggcaaccgcgtttggtataaatgtgttttcttgtttacattttaccatctatagtcatctcaatgttatatagtagaggcttcatgtttgtagtagataaggtagagaattgagaatattttatttttgtgcgaccatcaattttatgtaatctgcattgtctaatgctttatttgacatttgaaactacttaatttgacagttatgcaggtccgcatgatcctatgaaagcaattaattagtacgggtaaactgcactacacaagtttgctagtactattctattaaccgacctgtcaatattaccttaagttactgatttcaattagaatctaacacattcaggaaaagaagtttcactagtacaaaaatcattttcgttggcacgttgttttttttttcacaggcagttcacaatatcatggtgctagtagaaaaatttcaacgggcccaacaagagaaccgccaggcggtcttcttaattcaaccgcctgtgtaaactttccatttacataggcggcttacgataaaaaccgtgtgtataaataccattaacacaggcagtcgagttacgacaaccgcctgtgtaaatgtgtctttttacacaggcggtttgtatagagggccgcctgtgctaatatatttacacaggctatgagccgcctgtgttaagtcttctataaatacccttcgtccacctccagacaagaacagttactcccatgagctctgcacactggcggaccagacgattccagtttccaaggggggaggttttgattttcatttctttggtgagaaacttccaaaaggttagttagtgccattgatgctattttttaagcgattctttggttcaattcttgtattggaggtgctctagatctagagttcatcatgcattcttgcttagggttagagttcatagggcaaaaagagagagatttagctaaatttttatgtaaattcatagtaaattgtaaaaattaaaaaaaataaaaaataaatactttttagaattcttgtgagtagatctatacaatagagtaatgatgaggatattttgaagtttataattttgattcagttttagcttttcttttttcagatgaattagactttataaactcaaacattaaaatgttgaaaatcataaaatggcaaataaatactttttcaaatctttgtgcataaatacttcatagaaatccttgaattattcctaaattttatacaattgtttcttataattatgaaaatgagtttaaacaattatttaaattccataaattgtaactccgtaaggtgtaggttttcatctctgtttaatagaaggaggttagtatcttagttaagtctgttttcgggggttatattagttttgtttttagattgacctacattaattgttcttaactaattacagctaaatatggagaggtcattatggatgtacaacttatcaagattggacctatcatatgtagtgcaggtccaaaaatttattgatgtcgcaaagatacatgctcgcagaacaaaggcgaagcacatatgttgtccatgcgcagactgcaaaaatattatggtatttgacaatgtagaagcaattacttcccatctggtttgaagaggatttatggaggactacttgatttggacaaaacatggtgagggtagttttgcaccttatatgcggacaactgacaacactgcaactaacatcaatgtggagggtccaatgccacctctcaatgaatttcatgctatgccagatgttaatgaaactcatacgtctgatgtcaatgaaactcagcatgctaacacagatgttgttgaagatgcagatttcttagaggcaataatgaaccgttgtgcggatccatcaatattcttcatgaagggaatgaaagcattgaagaaggcagcagaggacactttgtacgacgagtcaaaaggttgtaccaaacaatggtcgacattatgtgttgttcttcagtttttgacgatgaaggctagacatggttggtccgatgctagcttcaatgatttcttgcgtgtacttggagaccttcttcctaaggagaacaaagtgcctgctaacacatactatgcaaagaagctagtcagtccacttacgataggtgttgagaagatccacgcatgtagaaatcattgtattctatatcgaggtgatcaatataaagacttagacagttgtccaaactgtggtgccagtaggtacaagacaaacaaagattttcgggaggaagagaatctagcctctgtttctacagggaggaagcgaaagaagacccaaacaaagactcaacaagacaagcgctcaaagcctagtagcaatgaagaagtggactattatgcattgagaagagtctccctatgagccaaaaaaggggacagcagcaggcacaactctctttctgaaaggacttggaaagcagcggacggcacggctcattgagctcgaaccgtcacagaaaaaggaagccaccgcccagtcaatagaagccatgcccccatcaaaggaagccccaagtggcgatgtacatattgaacagccatcaagtcaaccattgaccctaaaggatatcagaaagccaacgattgatgattatgtcaatgtccctagtgactatgtgcccggaaggcctatgctccaatggacgctgctcgattagattcaatggctgataaaaaggtttcatgactggtacatgagagcagtgcatgctagcctccatggaatcagagttgatataccaacagacatgtttgctactggtaacaaaaaaagcaagacatttgttacctttgaggacatgcacttgttattgaactataggcggcttgacgtccaactcataacaatctggtgcctgtaagtatcactcatgcacacacaattattatatattaatatgtagtgtgaaactctaatatgtagatgttgtctgtagtttgcaagatcacgagcagatgtcattattatctgccggatcgatggtcggttatctgagccctatcaagttacaagaaaatatgaacaaattcgtattatcaaaggaagatagagcaaagatagaggaagacaaaacaccaggataattatgccatctatcttggtagatcaatgctgaggtataaatatagggattttatattggcaccatacaacattaggtaagcttgacttcatatacgtatttcaaattatcgtgtaaacaatatacatgtgtcgctcactcatttattcatgcagtgaccattggattgttttttatatttatcccttcgaagggaaggtgcttgtcctagactctttacatgttcctcccgagaagtatcaaccattcttggttcaattagaaaggtgagccaacatgaaaccacatgcgtacttatataaattagagtttcaaaataactttagtgatttaggttcgatatctacggggcatggcggttttataagaaacaaaagggacctgtcgacgctgcacgctcagatcctaggatcccattgatgatacaacaccactatccggtaagttttctgaacacatttcatcatataaataatacataaagcatggcaaatttagaataatccgttgctcattatatagtgccacaagcaaccacctggatcggtctattgtgggtactatgtctgtgagtttataaggcagcggggacgttacgtcaaggacaaaaatatggtaaataatatctatgtatgaaagttttctcattaaagctgcaaaattatatattgaacatgtgtcaatcatgcttttaaactttattttcagccgaaaaagcaaggaaaagacgtgccctttacaccaaagactctggaagatatagtagcatacttgtgtggttttattatgagagaaataatttcaagtgacagtgcatattttgatcatgagggcgatttagcaagtgataaatttagagtgctgacagacatagcaggtctaaatctgaagcgaaacgacatgtaaacattgtatggttgtgcggataacatgcattgacgtgtatatatataattttatggttgatgtttgatttgtttacaattctataatatatatatgtggtgtatgtatgatgttgtgtgtgtatatatatatatatatatatatatatatatatatatatatatatatatatatatatataatgtttagcactgtgtttggtgggaaaaattaaaatttgaaatatatataaaaaattatttacacagacagtgtacgtgtcgagcgtcgtcctgtgctatacaaatacattctaacaggcggctcgccttgtccaccggtcggttaaaaatacatttccacacnggcctggctgggagagccgcctgtgaaaacataattttcacaggcggctcgcacagccccgcctgtactgtggtccattttgtactgacccctggtacaggcggtgggcttggccgcctgtgaagatgcttttagcaccgcctgtaaaaatgttttttgtagcagtgtttttcttattagtagtatcttttatactaattaagattcaataaaaattcaccatgacatccccattgccaagagaatatttcgccgcccctcaaagcagccaataaggctttactaaaaagactatccacgcagtagagatttagtcaaaatattccaatagcaattgtttcctgcctgcttgaccttcgtcagccactcactgtataaatatcgcaccacgccctttgcaggcttacagagcttgtattacgtactaacaaggcacacacagtaccctgtgttcaccggccctgcacaaaactcaagcagttattactaacatggcggctaacgattccttggttactgctcatgtgataggagatgtcttggaccccttctatacaaccgttgacatgatgatcctattcgatggtactcctattatcagcggcatggagttgcgcgctccggcggtttctgacaggccaagggttgaaattggaggagatgattatcgagttgcatatactctggtaaactcatgccatgtcaattaactagtagttgaatttagatgctggtggtatcgtggatacatgtactatatgttatggttgatacatatttgtttaattgatcgcaacaccatttgcggtaacttcaaattacattctttcaatatataggtgatggtcgatcctgatgctcctaacccaagcaacccaaccttgagggagtacttgcactggtaagagaaacctatagacgacaattattgttgttggcatgttttgcccacatatactttgtgtgtgtatatttgtgcttatgcttctccataaaattttggtgtatgtctcaagagagataggtatagaggttagcagtcctttaaaaatggtttaatccagtagttttttttcggtcggactgctcgaattattgtatatatggagatcacatgctagtaactttttcaataatttcatgtttcgagcaggatggtgactgacatcccagcatcaactgataatacatacggtgagtacacccctattcccattttgaaacaagtagaatgtctatttttatgatttagtatgttcgtgacaataggctatagctattttgaaacttcgggagcataaaatagtactcgattttgtataaccataaacacacagctagccaatctctattcatatttattttagttttatttgccgaaccatcctcaacatcatagccacttgatcgatcatctcaatcagcgtttgtatccttgcccgcttgattatcatccatggcagttcatattttttttcatttctttcatgcttgttatagttttatctgatgaatccaagatgttattgatcaattagttcagatgagcagtaatgcatgttggaggtttggtagtatatatacgttcaaaatttcacgaaatcggtaattacggtgggagccaaaaaaaattccaaaatttcgtattacattaataatgcatgtgctgtagactcatattttctatgatttcgattctgtcaccatcctgctcgaatatttaaatcatgctaatattttgtttacatctaaatcttttataaaaattataatttatatttgggtttaacaatttcgggcgcgtttagtgagattgggtaatttcggagcgaggccaccggccacacgaaaaattnctatacacgnactatatgtgtacatgtacatgcatggcaccctgataggctaccccatggggaaaaaattggaaacggaccattcatacgcagtcgtggtgcagactgtgggccacaatagcagtgtaaacataattacggtaatcaaataccccatgggaccatatatatcatccacagatccgtacggtgcttccgtgtggatggtctacaccagatcttttccacaccataagggcagcaatgcagcatcatattcatatatgcactagtgatgtaccatttggcttatatcatattcaacctaactccttggaaacattatgatattctattgggttgaagatgtcactactacaaaaaaaaatcttatgagaggtgttttgaaaactgccggaggtgcttaaaggagacagacgagttaggacaaccgtctctattaatgtgtactaactgaggtagttaccgtaacgtgcctgacttgattaacagattcaaccgtctcagtaaaggccatgattaaccgaaacagattcgagagttttcttaagtagttaaactattttaatcttcaccgaacttatagaaaatgaaagagctaacaccaatatttataaaaataaattagtatcactaaatacatcacgaaatctatttggtgttgtagaagttatccttttctataaaattgatcaaatttatgataacttagttttaggaattcatttattttaggacaactgaggaagtacatattttttaagtcatccacaaagtagtggatccaatttattacattactctactacttcaaactgaacaaaagcctaatcctggttatttttagagtgattttttacaacatcagcagtagtccagaaaatgggaggacattaataaaagtgaaaaggagcagaagaaagattacggtattttatttgtgctatttgtttaactattggcagtttgggaccgaaataaataactgttcgtagctctatatttgtcgattcaaaaagtgtaacgatgatttttgtgtttcaaaagaaaaataaagaagtgcaccaatgattggatatcataggctatatatgttggattaattgcatccaacgtatatagtgaaaatgcttttcaatcaagtaatcttcgagcggttaccagttttaatagttgcgagtcgtcgttttttatgtaccctaggacatatatatccgcatgtagacgatgatgagactagcaagtttttttttttttttgagcaaatacataattattggatttgcaggccgtgagatgatgtgctacgagccccctgccccgtccacgggcatccaccgtatggtgctggtgctattccagcagcttggccgtgacacggtgttcgcggcgccgtccaggcgccacaacttcaacacccgtgccttcgcccgccgctacaacctcggcgcgcccgtcgccgccatgttcttcaactgccagcgccagaccggctccggtggccccaggttcaccgggccctacaccagccgacgtcgtgcgggctgatgacgacgatcgtcgttacgtcacgtgtaccgtacacatatatgtatagatatacatgcatgcatgttccatggtataggatcggtgacaaaacgtctaataatgtatacacacacatgcatggaatgcatgtaataagagaatatatgtataataagtaggggagagcatgcatatattgtgtacacgcgtccgatgcgtatagccctttacattattgtagttgtaatcagctgtttaagcattctgctgtgtcagaacatgatgcatatatagtttggtgtgagtattgatctagtggaactcttatcagccttcaactcttatcacaagtgtaagatatagcttttataccttcaggtgtcttcccagtgtacctagaaatgctacaacggttgtattttatctatgcgcttcactactggaaacctgaatacttctgtggatgtcgaatttttctgtgcgtttttttcgatacacacggaaaaattataattattctgtgggttttaaaatatcctcatagaaaaatacaaatacccacagaaaaattatatcatttttctgtgcgtgacaatacactcacagaaaaattacaatttttgtgtgtgtttatataaaacgcacagaaaaaataatcacacacagaaaaattataattattctgtaggtttctataaaacgcacataaaaaataaacacacactgaaaaatagaacaagcaccctcatactaaattcatataaacacccatatttttttctttttaatctctctgtaaaacttgtaactagtttttccctctcgtactaactccaaattggatgattt

SEQ ID NO: 2 - DNA, 2913 bp, Sorghum propinquum
  misc_feature (1365)..(1365): n is a, c, g, or t
atggcggctaacgattccttggttactgctcatgtgataggagatgtcttggaccccttctatacaaccgttgacatgatgatcctattcgatggtactcctattatcagcggcatggagttgcgcgctccggcggtttctgacaggccaagggttgaaattggaggagatgattatcgagttgcatatactctggtaaactcatgccatgtcaattaactagtagttgaatttagatgctggtggtatcgtggatacatgtactatatgttatggttgatacatatttgtttaattgatcgcaacaccatttgcggtaacttcaaattacattctttcaatatataggtgatggtcgatcctgatgctcctaacccaagcaacccaaccttgagggagtacttgcactggtaagagaaacctatagacgacaattattgttgttggcatgttttgcccacatatactttgtgtgtgtatatttgtgcttatgcttctccataaaattttggtgtatgtctcaagagagataggtatagaggttagcagtcctttaaaaatggtttaatccagtagttttttttcggtcggactgctcgaattattgtatatatggagatcacatgctagtaactttttcaataatttcatgtttcgagcaggatggtgactgacatcccagcatcaactgataatacatacggtgagtacacccctattcccattttgaaacaagtagaatgtctatttttatgatttagtatgttcgtgacaataggctatagctattttgaaacttcgggagcataaaatagtactcgattttgtataaccataaacacacagctagccaatctctattcatatttattttagttttatttgccgaaccatcctcaacatcatagccacttgatcgatcatctcaatcagcgtttgtatccttgcccgcttgattatcatccatggcagttcatattttttttcatttctttcatgcttgttatagttttatctgatgaatccaagatgttattgatcaattagttcagatgagcagtaatgcatgttggaggtttggtagtatatatacgttcaaaatttcacgaaatcggtaattacggtgggagccaaaaaaaattccaaaatttcgtattacattaataatgcatgtgctgtagactcatattttctatgatttcgattctgtcaccatcctgctcgaatatttaaatcatgctaatattttgtttacatctaaatcttttataaaaattataatttatatttgggtttaacaatttcgggcgcgtttagtgagattgggtaatttcggagcgaggccaccggccacacgaaaaattnctatacacgnactatatgtgtacatgtacatgcatggcaccctgataggctaccccatggggaaaaaattggaaacggaccattcatacgcagtcgtggtgcagactgtgggccacaatagcagtgtaaacataattacggtaatcaaataccccatgggaccatatatatcatccacagatccgtacggtgcttccgtgtggatggtctacaccagatcttttccacaccataagggcagcaatgcagcatcatattcatatatgcactagtgatgtaccatttggcttatatcatattcaacctaactccttggaaacattatgatattctattgggttgaagatgtcactactacaaaaaaaaatcttatgagaggtgttttgaaaactgccggaggtgcttaaaggagacagacgagttaggacaaccgtctctattaatgtgtactaactgaggtagttaccgtaacgtgcctgacttgattaacagattcaaccgtctcagtaaaggccatgattaaccgaaacagattcgagagttttcttaagtagttaaactattttaatcttcaccgaacttatagaaaatgaaagagctaacaccaatatttataaaaataaattagtatcactaaatacatcacgaaatctatttggtgttgtagaagttatccttttctataaaattgatcaaatttatgataacttagttttaggaattcatttattttaggacaactgaggaagtacatattttttaagtcatccacaaagtagtggatccaatttattacattactctactacttcaaactgaacaaaagcctaatcctggttatttttagagtgattttttacaacatcagcagtagtccagaaaatgggaggacattaataaaagtgaaaaggagcagaagaaagattacggtattttatttgtgctatttgtttaactattggcagtttgggaccgaaataaataactgttcgtagctctatatttgtcgattcaaaaagtgtaacgatgatttttgtgtttcaaaagaaaaataaagaagtgcaccaatgattggatatcataggctatatatgttggattaattgcatccaacgtatatagtgaaaatgcttttcaatcaagtaatcttcgagcggttaccagttttaatagttgcgagtcgtcgttttttatgtaccctaggacatatatatccgcatgtagacgatgatgagactagcaagtttttttttttttttgagcaaatacataattattggatttgcaggccgtgagatgatgtgctacgagccccctgccccgtccacgggcatccaccgtatggtgctggtgctattccagcagcttggccgtgacacggtgttcgcggcgccgtccaggcgccacaacttcaacacccgtgccttcgcccgccgctacaacctcggcgcgcccgtcgccgccatgttcttcaactgccagcgccagaccggctccggtggccccaggttcaccgggccctacaccagccgacgtcgtgcgggctga

SEQ ID NO: 3 - DNA, 10624 bp, Sorghum propinquum
  misc_feature (6906)..(6909): N=1, 2, 3, 4, or 5 nucleotides
ccctgacccttgttgggcaacatttagagtcgttagctttgcaattctttggttccaatggatggttatcatttagacatattggtcatgcttagtcaaaactttattgttcggctataaacttttcagtactttgtaataattggctcgatagatgaagccgggtataacatatcctttatctaaaaaaattagttaacatgaacttcatattcaattcttcatatctcactagcatctttattgtctagttagttttgtagcattgcaaaaagcatgcaactatatacaatgaaacggaataaaatttcagctctattaatttatatttcaaatataggccactatagccatatttcgtgctcaaggccacaaaatcttgcgtacttccctgttggtaccaaagagaagacgttatttaactttgtttgactcttcaatatggtttgaatcagaaaattagttaaaagaaaagtgagcacaccacgacctgtcatcagctcatggtcagctctacaaacttatagattgcatcgagatctaagactcaggtacaaatcatgtcaacatctaatggtttagaaaatgaaaagttttgagtttcaaaatatgatacgtgatattaacatttgaacttttagcaagatctgaaataaaaaattcaactagatcatgttaacattgatataatcgcttccaatcgcctcccatcacttccgctagaaaactttttttctcgatttaattaatgaaagggtaataacatcattgtacaagattctttcaaacctcaacccctatcatcgacggtgacggctccctataacacgcactagtggacgccgggcgggtggaaccctaagaagatttaaaaaaacttaagaagaagatttttatctaactaactatagtacttatatcatacactatactattcaaaatattattttcacaattatgaatttacccttttactcttcattaaaaaaatacgaaaaaagaatcaccacgtctctatttagggtcctagtccccataatttaagaggcggtgagagacgatgtgacgtctatggaccaccgaccaaagacacacctatcgtctcccatcgccttgcttccatcgcctctcatcgcttttcatattctagatccagcggccatagacacaccaatcgtttctcatcgcctctccaaccattgtaaaaatatttataattttgatataaaatttgtcttcacttgagttcatgccaaaaaaattatacatattattttcgtgtgagaatttacagaagtggactcttaagatgtccaaatgtaaatgaccctatttattatgaggcgcggatctataggcctgactctgaaaatggattatggatttgagataataaatttaagggcctatcttcgcacataacatctatagttcctaaatttttttttattgtagtagtagaacttttctccctgtaaaccaagttgacgctgggctttattttgcgacacagaacaccaaattggtggctatgaactcttccacctgggcagggaaaacggtttattatgtttctctttaatttatctatcgtggcactataacacaacatggctttgccgacacttccaactatcggcaaagggtacctttaccgacacttaacgtctcacgaaaggttttgccgacaattttcaaacagtcgcggtagaagcagttggcgaaacttttgccgacagttaaaggcatcgccgacacattttctgtagtcaaatggcatacctacgccgacagttgaactttcaccgacagtgaaccctttgccgacagtttggacctacgccgacagtttggaccttttccgacagttggtatgttagcgaaaccgtttctagggtgtttcataaaccatgccttgtccaacagtagaagtgtcggcaaaactatattgctaggatgtagatacaatttaaatattttaataaatacacatcacattgattgagcaaaatcacatggtctgttttcactaaaactgtcagaggtacactccagtactaccagtacgtcgcccgcacagtggccaaggattttactgctactgttgattaacataagcacttgcgactttccctaaaatcttttataaaacaacggccgcaataatattgaactattttttttctagtaccaaaattagaatttgatccctcacctcattacatccatagtaacatgaccagatatatatggacaggatgggatcactcagcgagcagatacactgagcgattcataatcagattttttaatttcttctagtgaagtggggttttcctagtcttttaacattcaaaatttagtacaaactttccctagtaaatgccttctagtaaagatttcctagtattttgactagcgatagtgttttattactaattaaaaacattagaagaactccatttagtgattggttgtttggattagtcttctcacgttagacctatatatgcaggacaactcaagccagcataaatatatgaaatatcttggtgtttgtttgtctgacacaggcaaccgcgtttggtataaatgtgttttcttgtttacattttaccatctatagtcatctcaatgttatatagtagaggcttcatgtttgtagtagataaggtagagaattgagaatattttatttttgtgcgaccatcaattttatgtaatctgcattgtctaatgctttatttgacatttgaaactacttaatttgacagttatgcaggtccgcatgatcctatgaaagcaattaattagtacgggtaaactgcactacacaagtttgctagtactattctattaaccgacctgtcaatattaccttaagttactgatttcaattagaatctaacacattcaggaaaagaagtttcactagtacaaaaatcattttcgttggcacgttgttttttttttcacaggcagttcacaatatcatggtgctagtagaaaaatttcaacgggcccaacaagagaaccgccaggcggtcttcttaattcaaccgcctgtgtaaactttccatttacataggcggcttacgataaaaaccgtgtgtataaataccattaacacaggcagtcgagttacgacaaccgcctgtgtaaatgtgtctttttacacaggcggtttgtatagagggccgcctgtgctaatatatttacacaggctatgagccgcctgtgttaagtcttctataaatacccttcgtccacctccagacaagaacagttactcccatgagctctgcacactggcggaccagacgattccagtttccaaggggggaggttttgattttcatttctttggtgagaaacttccaaaaggttagttagtgccattgatgctattttttaagcgattctttggttcaattcttgtattggaggtgctctagatctagagttcatcatgcattcttgcttagggttagagttcatagggcaaaaagagagagatttagctaaatttttatgtaaattcatagtaaattgtaaaaattaaaaaaaataaaaaataaatactttttagaattcttgtgagtagatctatacaatagagtaatgatgaggatattttgaagtttataattttgattcagttttagcttttcttttttcagatgaattagactttataaactcaaacattaaaatgttgaaaatcataaaatggcaaataaatactttttcaaatctttgtgcataaatacttcatagaaatccttgaattattcctaaattttatacaattgtttcttataattatgaaaatgagtttaaacaattatttaaattccataaattgtaactccgtaaggtgtaggttttcatctctgtttaatagaaggaggttagtatcttagttaagtctgttttcgggggttatattagttttgtttttagattgacctacattaattgttcttaactaattacagctaaatatggagaggtcattatggatgtacaacttatcaagattggacctatcatatgtagtgcaggtccaaaaatttattgatgtcgcaaagatacatgctcgcagaacaaaggcgaagcacatatgttgtccatgcgcagactgcaaaaatattatggtatttgacaatgtagaagcaattacttcccatctggtttgaagaggatttatggaggactacttgatttggacaaaacatggtgagggtagttttgcaccttatatgcggacaactgacaacactgcaactaacatcaatgtggagggtccaatgccacctctcaatgaatttcatgctatgccagatgttaatgaaactcatacgtctgatgtcaatgaaactcagcatgctaacacagatgttgttgaagatgcagatttcttagaggcaataatgaaccgttgtgcggatccatcaatattcttcatgaagggaatgaaagcattgaagaaggcagcagaggacactttgtacgacgagtcaaaaggttgtaccaaacaatggtcgacattatgtgttgttcttcagtttttgacgatgaaggctagacatggttggtccgatgctagcttcaatgatttcttgcgtgtacttggagaccttcttcctaaggagaacaaagtgcctgctaacacatactatgcaaagaagctagtcagtccacttacgataggtgttgagaagatccacgcatgtagaaatcattgtattctatatcgaggtgatcaatataaagacttagacagttgtccaaactgtggtgccagtaggtacaagacaaacaaagattttcgggaggaagagaatctagcctctgtttctacagggaggaagcgaaagaagacccaaacaaagactcaacaagacaagcgctcaaagcctagtagcaatgaagaagtggactattatgcattgagaagagtctccctatgagccaaaaaaggggacagcagcaggcacaactctctttctgaaaggacttggaaagcagcggacggcacggctcattgagctcgaaccgtcacagaaaaaggaagccaccgcccagtcaatagaagccatgcccccatcaaaggaagccccaagtggcgatgtacatattgaacagccatcaagtcaaccattgaccctaaaggatatcagaaagccaacgattgatgattatgtcaatgtccctagtgactatgtgcccggaaggcctatgctccaatggacgctgctcgattagattcaatggctgataaaaaggtttcatgactggtacatgagagcagtgcatgctagcctccatggaatcagagttgatataccaacagacatgtttgctactggtaacaaaaaaagcaagacatttgttacctttgaggacatgcacttgttattgaactataggcggcttgacgtccaactcataacaatctggtgcctgtaagtatcactcatgcacacacaattattatatattaatatgtagtgtgaaactctaatatgtagatgttgtctgtagtttgcaagatcacgagcagatgtcattattatctgccggatcgatggtcggttatctgagccctatcaagttacaagaaaatatgaacaaattcgtattatcaaaggaagatagagcaaagatagaggaagacaaaacaccaggataattatgccatctatcttggtagatcaatgctgaggtataaatatagggattttatattggcaccatacaacattaggtaagcttgacttcatatacgtatttcaaattatcgtgtaaacaatatacatgtgtcgctcactcatttattcatgcagtgaccattggattgttttttatatttatcccttcgaagggaaggtgcttgtcctagactctttacatgttcctcccgagaagtatcaaccattcttggttcaattagaaaggtgagccaacatgaaaccacatgcgtacttatataaattagagtttcaaaataactttagtgatttaggttcgatatctacggggcatggcggttttataagaaacaaaagggacctgtcgacgctgcacgctcagatcctaggatcccattgatgatacaacaccactatccggtaagttttctgaacacatttcatcatataaataatacataaagcatggcaaatttagaataatccgttgctcattatatagtgccacaagcaaccacctggatcggtctattgtgggtactatgtctgtgagtttataaggcagcggggacgttacgtcaaggacaaaaatatggtaaataatatctatgtatgaagttttctcattaaagctgcaaaattatatattgaacatgtgtcaatcatgcttttaaactttattttcagccgaaaaagcaaggaaaagacgtgccctttacaccaaagactctggaagatatagtagcatacttgtgtggttttattatgagagaaataatttcaagtgacagtgcatattttgatcatgagggcgatttagcaagtgataaatttagagtgctgacagacatagcaggtctaaatctgaagcgaaacgacatgtaaacattgtatggttgtgcggataacatgcattgacgtgtatatatataattttatggttgatgtttgatttgtttacaattctataatatatatatgtggtgtatgtatgatgttgtgtgtgtatatatatatatatatatatatatatatatatatatatatatatatatatatatatataatgtttagcactgtgtttggtgggaaaaattaaaatttgaaatatatataaaaaattatttacacagacagtgtagtgtgagctgcctgtgtaaaaatacatttatacaggcggctcaccttgtcnnnncaggcggtgctaaaagcatcttcacaggcggccaagcccaccgcctgtaccaggggtcagtacaaaatggaccacagtacaggcggggctgtgcgagccgcctgtgaaaacataattttcacaggcggctcgcacagccccgcctgtactgtggtccattttgtactgacccctggtacaggcggtgggcttggccgcctgtgaagatgcttttagcaccgcctgtaaaaatgttttttgtagcagtgtttttcttattagtagtatcttttatactaattaagattcaataaaaattcaccatgacatccccattgccaagagaatatttcgccgcccctcaaagcagccaataaggctttactaaaaagactatccacgcagtagagatttagtcaaaatattccaatagcaattgtttcctgcctgcttgaccttcgtcagccactcactgtataaatatcgcaccacgccctttgcaggcttacagagcttgtattacgtactaacaaggcacacacagtaccctgtgttcaccggccctgcacaaaactcaagcagttattactaacatggcggctaacgattccttggttactgctcatgtgataggagatgtcttggaccccttctatacaaccgttgacatgatgatcctattcgatggtactcctattatcagcggcatggagttgcgcgctccggcggtttctgacaggccaagggttgaaattggaggagatgattatcgagttgcatatactctggtaaactcatgccatgtcaattaactagtagttgaatttagatgctggtggtatcgtggatacatgtactatatgttatggttgatacatatttgtttaattgatcgcaacaccatttgcggtaacttcaaattacattctttcaatatataggtgatggtcgatcctgatgctcctaacccaagcaacccaaccttgagggagtacttgcactggtaagagaaacctatagacgacaattattgttgttggcatgttttgcccacatatactttgtgtgtgtatatttgtgcttatgcttctccataaaattttggtgtatgtctcaagagagataggtatagaggttagcagtcctttaaaaatggtttaatccagtagttttttttcggtcggactgctcgaattattgtatatatggagatcacatgctagtaactttttcaataatttcatgtttcgagcaggatggtgactgacatcccagcatcaactgataatacatacggtgagtacacccctattcccattttgaaacaagtagaatgtctatttttatgatttagtatgttcgtgacaataggctatagctattttgaaacttcgggagcataaaatagtactcgattttgtataaccataaacacacagctagccaatctctattcatatttattttagttttatttgccgaaccatcctcaacatcatagccacttgatcgatcatctcaatcagcgtttgtatccttgcccgcttgattatcatccatggcagttcatattttttttcatttctttcatgcttgttatagttttatctgatgaatccaagatgttattgatcaattagttcagatgagcagtaatgcatgttggaggtttggtagtatatatacgttcaaaatttcacgaaatcggtaattacggtgggagccaaaaaaaattccaaaatttcgtattacattaataatgcatgtgctgtagactcatattttctatgatttcgattctgtcaccatcctgctcgaatatttaaatcatgctaatattttgtttacatctaaatcttttataaaaattataatttatatttgggtttaacaatttcgggcgcgtttagtgagattgggtaatttcggagcgaggccaccggccacacgaaaaattctatacacgactatatgtgtacatgtacatgcatggcaccctgataggctaccccatggggaaaaaattggaaacggaccattcatacgcagtcgtggtgcagactgtgggccacaatagcagtgtaaacataattacggtaatcaaataccccatgggaccatatatatcatccacagatccgtacggtgcttccgtgtggatggtctacaccagatcttttccacaccataagggcagcaatgcagcatcatattcatatatgcactagtgatgtaccatttggcttatatcatattcaacctaactccttggaaacattatgatattctattgggttgaagatgtcactactacaaaaaaaaatcttatgagaggtgttttgaaaactgccggaggtgcttaaaggagacagacgagttaggacaaccgtctctattaatgtgtactaactgaggtagttaccgtaacgtgcctgacttgattaacagattcaaccgtctcagtaaaggccatgattaaccgaaacagattcgagagttttcttaagtagttaaactattttaatcttcaccgaacttatagaaaatgaaagagctaacaccaatatttataaaaataaattagtatcactaaatacatcacgaaatctatttggtgttgtagaagttatccttttctataaaattgatcaaatttatgataacttagttttaggaattcatttattttaggacaactgaggaagtacatattttttaagtcatccacaaagtagtggatccaatttattacattactctactacttcaaactgaacaaaagcctaatcctggttatttttagagtgattttttacaacatcagcagtagtccagaaaatgggaggacattaataaaagtgaaaaggagcagaagaaagattacggtattttatttgtgctatttgtttaactattggcagtttgggaccgaaataaataactgttcgtagctctatatttgtcgattcgaaagtgtaacgatgatttttgtgtttcaaaagaaaaataaagaagtgcaccaatgattggatatcataggctatatatgttggattaattgcatccaacgtatatagtgaaaatgcttttcaatcaagtaatcttcgagcggttaccagttttaatagttgcgagtcgtcgttttttatgtaccctaggacatatatatccgcatgtagacgatgatgagactagcaagtttttttttttttttgagcaaatacataattattggatttgcaggccgtgagatgatgtgctacgagccccctgccccgtccacgggcatccaccgtatggtgctggtgctattccagcagcttggccgtgacacggtgttcgcggcgccgtccaggcgccacaacttcaacacccgtgccttcgcccgccgctacaacctcggcgcgcccgtcgccgccatgttcttcaactgccagcgccagaccggctccggtggccccaggttcaccgggccctacaccagccgacgtcgtgcgggctgatgacgacgatcgtcgttacgtcacgtgtaccgtacacatatatgtatagatatacatgcatgcatgttccatggtataggatcggtgacaaaacgtctaataatgtatacacacacatgcatggaatgcatgtaataagagaatatatgtataataagtaggggagagcatgcatatattgtgtacacgcgtccgatgcgtatagccctttacattattgtagttgtaatcag

SEQ ID NO: 4 - DNA, 2910 bp, Sorghum propinquum
atggcggctaacgattccttggttactgctcatgtgataggagatgtcttggaccccttctatacaaccgttgacatgatgatcctattcgatggtactcctattatcagcggcatggagttgcgcgctccggcggtttctgacaggccaagggttgaaattggaggagatgattatcgagttgcatatactctggtaaactcatgccatgtcaattaactagtagttgaatttagatgctggtggtatcgtggatacatgtactatatgttatggttgatacatatttgtttaattgatcgcaacaccatttgcggtaacttcaaattacattctttcaatatataggtgatggtcgatcctgatgctcctaacccaagcaacccaaccttgagggagtacttgcactggtaagagaaacctatagacgacaattattgttgttggcatgttttgcccacatatactttgtgtgtgtatatttgtgcttatgcttctccataaaattttggtgtatgtctcaagagagataggtatagaggttagcagtcctttaaaaatggtttaatccagtagttttttttcggtcggactgctcgaattattgtatatatggagatcacatgctagtaactttttcaataatttcatgtttcgagcaggatggtgactgacatcccagcatcaactgataatacatacggtgagtacacccctattcccattttgaaacaagtagaatgtctatttttatgatttagtatgttcgtgacaataggctatagctattttgaaacttcgggagcataaaatagtactcgattttgtataaccataaacacacagctagccaatctctattcatatttattttagttttatttgccgaaccatcctcaacatcatagccacttgatcgatcatctcaatcagcgtttgtatccttgcccgcttgattatcatccatggcagttcatattttttttcatttctttcatgcttgttatagttttatctgatgaatccaagatgttattgatcaattagttcagatgagcagtaatgcatgttggaggtttggtagtatatatacgttcaaaatttcacgaaatcggtaattacggtgggagccaaaaaaaattccaaaatttcgtattacattaataatgcatgtgctgtagactcatattttctatgatttcgattctgtcaccatcctgctcgaatatttaaatcatgctaatattttgtttacatctaaatcttttataaaaattataatttatatttgggtttaacaatttcgggcgcgtttagtgagattgggtaatttcggagcgaggccaccggccacacgaaaaattctatacacgactatatgtgtacatgtacatgcatggcaccctgataggctaccccatggggaaaaaattggaaacggaccattcatacgcagtcgtggtgcagactgtgggccacaatagcagtgtaaacataattacggtaatcaaataccccatgggaccatatatatcatccacagatccgtacggtgcttccgtgtggatggtctacaccagatcttttccacaccataagggcagcaatgcagcatcatattcatatatgcactagtgatgtaccatttggcttatatcatattcaacctaactccttggaaacattatgatattctattgggttgaagatgtcactactacaaaaaaaaatcttatgagaggtgttttgaaaactgccggaggtgcttaaaggagacagacgagttaggacaaccgtctctattaatgtgtactaactgaggtagttaccgtaacgtgcctgacttgattaacagattcaaccgtctcagtaaaggccatgattaaccgaaacagattcgagagttttcttaagtagttaaactattttaatcttcaccgaacttatagaaaatgaaagagctaacaccaatatttataaaaataaattagtatcactaaatacatcacgaaatctatttggtgttgtagaagttatccttttctataaaattgatcaaatttatgataacttagttttaggaattcatttattttaggacaactgaggaagtacatattttttaagtcatccacaaagtagtggatccaatttattacattactctactacttcaaactgaacaaaagcctaatcctggttatttttagagtgattttttacaacatcagcagtagtccagaaaatgggaggacattaataaaagtgaaaaggagcagaagaaagattacggtattttatttgtgctatttgtttaactattggcagtttgggaccgaaataaataactgttcgtagctctatatttgtcgattcgaaagtgtaacgatgatttttgtgtttcaaaagaaaaataaagaagtgcaccaatgattggatatcataggctatatatgttggattaattgcatccaacgtatatagtgaaaatgcttttcaatcaagtaatcttcgagcggttaccagttttaatagttgcgagtcgtcgttttttatgtaccctaggacatatatatccgcatgtagacgatgatgagactagcaagtttttttttttttttgagcaaatacataattattggatttgcaggccgtgagatgatgtgctacgagccccctgccccgtccacgggcatccaccgtatggtgctggtgctattccagcagcttggccgtgacacggtgttcgcggcgccgtccaggcgccacaacttcaacacccgtgccttcgcccgccgctacaacctcggcgcgcccgtcgccgccatgttcttcaactgccagcgccagaccggctccggtggccccaggttcaccgggccctacaccagccgacgtcgtgcgggctga

SEQ ID NO: 5 - DNA, 5259 bp, Sorghum propinquum
  misc_feature (1666)..(1670): N=1, 2, 3, 4, or 5 nucleotides
ctatgctccaatggacgctgctcgattagattcaatggctgataaaaaggtttcatgactggtacatgagagcagtgcatgctagcctccatggaatcagagttgatataccaacagacatgtttgctactggtaacaaaaaaagcaagacatttgttacctttgaggacatgcacttgttattgaactataggcggcttgacgtccaactcataacaatctggtgcctgtaagtatcactcatgcacacacaattattatatattaatatgtagtgtgaaactctaatatgtagatgttgtctgtagtttgcaagatcacgagcagatgtcattattatctgccggatcgatggtcggttatctgagccctatcaagttacaagaaaatatgaacaaattcgtattatcaaaggaagatagagcaaagatagaggaagacaaaacaccaggataattatgccatctatcttggtagatcaatgctgaggtataaatatagggattttatattggcaccatacaacattaggtaagcttgacttcatatacgtatttcaaattatcgtgtaaacaatatacatgtgtcgctcactcatttattcatgcagtgaccattggattgttttttatatttatcccttcgaagggaaggtgcttgtcctagactctttacatgttcctcccgagaagtatcaaccattcttggttcaattagaaaggtgagccaacatgaaaccacatgcgtacttatataaattagagtttcaaaataactttagtgatttaggttcgatatctacggggcatggcggttttataagaaacaaaagggacctgtcgacgctgcacgctcagatcctaggatcccattgatgatacaacaccactatccggtaagttttctgaacacatttcatcatataaataatacataaagcatggcaaatttagaataatccgttgctcattatatagtgccacaagcaaccacctggatcggtctattgtgggtactatgtctgtgagtttataaggcagcggggacgttacgtcaaggacaaaaatatggtaaataatatctatgtatgaagttttctcattaaagctgcaaaattatatattgaacatgtgtcaatcatgcttttaaactttattttcagccgaaaaagcaaggaaaagacgtgccctttacaccaaagactctggaagatatagtagcatacttgtgtggttttattatgagagaaataatttcaagtgacagtgcatattttgatcatgagggcgatttagcaagtgataaatttagagtgctgacagacatagcaggtctaaatctgaagcgaaacgacatgtaaacattgtatggttgtgcggataacatgcattgacgtgtatatatataattttatggttgatgtttgatttgtttacaattctataatatatatatgtggtgtatgtatgatgttgtgtgtgtatatatatatatatatatatatatatatatatatatatatatatatatatatatatataatgtttagcactgtgtttggtgggaaaaattaaaatttgaaatatatataaaaaattatttacacagacagtgtagtgtgagctgcctgtgtaaaaatacatttatacaggcggctcaccttgtnnnnncaggcggtgctaaaagcatcttcacaggcggccaagcccaccgcctgtaccaggggtcagtacaaaatggaccacagtacaggcggggctgtgcgagccgcctgtgaaaacataattttcacaggcggctcgcacagccccgcctgtactgtggtccattttgtactgacccctggtacaggcggtgggcttggccgcctgtgaagatgcttttagcaccgcctgtaaaaatgttttttgtagcagtgtttttcttattagtagtatcttttatactaattaagattcaataaaaattcaccatgacatccccattgccaagagaatatttcgccgcccctcaaagcagccaataaggctttactaaaaagactatccacgcagtagagatttagtcaaaatattccaatagcaattgtttcctgcctgcttgaccttcgtcagccactcactgtataaatatcgcaccacgccctttgcaggcttacagagcttgtattacgtactaacaaggcacacacagtaccctgtgttcaccggccctgcacaaaactcaagcagttattactaacatggcggctaacgattccttggttactgctcatgtgataggagatgtcttggaccccttctatacaaccgttgacatgatgatcctattcgatggtactcctattatcagcggcatggagttgcgcgctccggcggtttctgacaggccaagggttgaaattggaggagatgattatcgagttgcatatactctggtaaactcatgccatgtcaattaactagtagttgaatttagatgctggtggtatcgtggatacatgtactatatgttatggttgatacatatttgtttaattgatcgcaacaccatttgcggtaacttcaaattacattctttcaatatataggtgatggtcgatcctgatgctcctaacccaagcaacccaaccttgagggagtacttgcactggtaagagaaacctatagacgacaattattgttgttggcatgttttgcccacatatactttgtgtgtgtatatttgtgcttatgcttctccataaaattttggtgtatgtctcaagagagataggtatagaggttagcagtcctttaaaaatggtttaatccagtagttttttttcggtcggactgctcgaattattgtatatatggagatcacatgctagtaactttttcaataatttcatgtttcgagcaggatggtgactgacatcccagcatcaactgataatacatacggtgagtacacccctattcccattttgaaacaagtagaatgtctatttttatgatttagtatgttcgtgacaataggctatagctattttgaaacttcgggagcataaaatagtactcgattttgtataaccataaacacacagctagccaatctctattcatatttattttagttttatttgccgaaccatcctcaacatcatagccacttgatcgatcatctcaatcagcgtttgtatccttgcccgcttgattatcatccatggcagttcatattttttttcatttctttcatgcttgttatagttttatctgatgaatccaagatgttattgatcaattagttcagatgagcagtaatgcatgttggaggtttggtagtatatatacgttcaaaatttcacgaaatcggtaattacggtgggagccaaaaaaaattccaaaatttcgtattacattaataatgcatgtgctgtagactcatattttctatgatttcgattctgtcaccatcctgctcgaatatttaaatcatgctaatattttgtttacatctaaatcttttataaaaattataatttatatttgggtttaacaatttcgggcgcgtttagtgagattgggtaatttcggagcgaggccaccggccacacgaaaaattctatacacgactatatgtgtacatgtacatgcatggcaccctgataggctaccccatggggaaaaaattggaaacggaccattcatacgcagtcgtggtgcagactgtgggccacaatagcagtgtaaacataattacggtaatcaaataccccatgggaccatatatatcatccacagatccgtacggtgcttccgtgtggatggtctacaccagatcttttccacaccataagggcagcaatgcagcatcatattcatatatgcactagtgatgtaccatttggcttatatcatattcaacctaactccttggaaacattatgatattctattgggttgaagatgtcactactacaaaaaaaaatcttatgagaggtgttttgaaaactgccggaggtgcttaaaggagacagacgagttaggacaaccgtctctattaatgtgtactaactgaggtagttaccgtaacgtgcctgacttgattaacagattcaaccgtctcagtaaaggccatgattaaccgaaacagattcgagagttttcttaagtagttaaactattttaatcttcaccgaacttatagaaaatgaaagagctaacaccaatatttataaaaataaattagtatcactaaatacatcacgaaatctatttggtgttgtagaagttatccttttctataaaattgatcaaatttatgataacttagttttaggaattcatttattttaggacaactgaggaagtacatattttttaagtcatccacaaagtagtggatccaatttattacattactctactacttcaaactgaacaaaagcctaatcctggttatttttagagtgattttttacaacatcagcagtagtccagaaaatgggaggacattaataaaagtgaaaaggagcagaagaaagattacggtattttatttgtgctatttgtttaactattggcagtttgggaccgaaataaataactgttcgtagctctatatttgtcgattcgaaagtgtaacgatgatttttgtgtttcaaaagaaaaataaagaagtgcaccaatgattggatatcataggctatatatgttggattaattgcatccaacgtatatagtgaaaatgcttttcaatcaagtaatcttcgagcggttaccagttttaatagttgcgagtcgtcgttttttatgtaccctaggacatatatatccgcatgtagacgatgatgagactagcaagtttttttttttttttgagcaaatacataattattggatttgcaggccgtgagatgatgtgctacgagccccctgccccgtccacgggcatccaccgtatggtgctggtgctattccagcagcttggccgtgacacggtgttcgcggcgccgtccaggcgccacaacttcaacacccgtgccttcgcccgccgctacaacctcggcgcgcccgtcgccgccatgttcttcaactgccagcgccagaccggctccggtggccccaggttcaccgggccctacaccagccgacgtcgtgcgggctgatgacgacgatcgtcgttacgtcacgtgtaccgtacacatatatgtatagatatacatgcatgcatgttccatggtataggatcggtgacaaaacgtctaataatgta

SEQ ID NO: 6 - DNA, 2910 bp, Sorghum propinquum; identical to SEQ ID NO: 4

SEQ ID NO: 7 - DNA, 558 bp, Sorghum propinquum
atggcggctaacgattccttggttactgctcatgtgataggagatgtcttggaccccttctatacaaccgttgacatgatgatcctattcgatggtactcctattatcagcggcatggagttgcgcgctccggcggtttctgacaggccaagggttgaaattggaggagatgattatcgagttgcatatactctggtgatggtcgatcctgatgctcctaacccaagcaacccaaccttgagggagtacttgcactggatggtgactgacatcccagcatcaactgataatacatacggccgtgagatgatgtgctacgagccccctgccccgtccacgggcatccaccgtatggtgctggtgctattccagcagcttggccgtgacacggtgttcgcggcgccgtccaggcgccacaacttcaacacccgtgccttcgcccgccgctacaacctcggcgcgcccgtcgccgccatgttcttcaactgccagcgccagaccggctccggtggccccaggttcaccgggccctacaccagccgacgtcgtgcgggctga

SEQ ID NO: 8 - protein, 185 aa, Sorghum propinquum
MAANDSLVTAHVIGDVLDPFYTTVDMMILFDGTPIISGMELRAPAVSDRPRVEIGGDDYRVAYTLVMVDPDAPNPSNPTLREYLHWMVTDIPASTDNTYGREMMCYEPPAPSTGIHRMVLVLFQQLGRDTVFAAPSRRHNFNTRAFARRYNLGAPVAAMFFNCQRQTGSGGPRFTGPYTSRRRAG

SEQ ID NO: 9 - DNA, 6080 bp, Sorghum bicolor
aaaagaaaagtgagcacaccacgacctatcatcagctcatggtcagctctacaaacttatagattgcatcgagatctaagactcaggtacaaatcatgtcaacatctaatggtttagaaaatgaaaaaagttttgagtttcaaaatatgatacttgaaattaacatttgaactttttagcaagatctgaaaataaaaaattcaactaaaaaatttatagatcatgttaacattgatataatcgcttccaatcgcctcccatcgcttcagctagaaaactttttttctcgatttaattaatgaaatagtaataacgtcattgtacaagattctttcaaaccccaacccctatcatcgacggtgagggctcctataatatgcactagtggacgccgggtgggtggaacctaagaagattttaaaaaaaaaattaagaagaagatttttatctaactaactatatatagtacttatatcatacactatactattcaaaatattattttcacaattatgaatttacccttttactctttattaaaaaaatatgaataaagaattatcacgcctctatttagggtcctaatccccataatttaagaggcgatgagaggcgatgtgacatctatggcccaccgaccaaagacacaactatcgcctcccatcaccttgcttctatcgcctctcatagcttttcatattctaggtccaccggccatagacacaccaatcgcttatcatcgccttttccaaccattgtaaaaatattcataattttgatataaaatttgtcttcacttgagtatgggaaaaaaattatacataatgttttcgtgtgagaatttacaggaatgaacccttaagatgtccaaatgtaaatgaccctatttattaagaggagcggatctataggcctggctctgaaaatggattatggattggagatactaaatttaagggcctatcttcgcacataacatctatagttcctaaataattttttattgtagtagtagaacttttctccctgtaaaccataaaccaagttgacgctgggctttattttgcgacacagaacaccaaattggtggctatgaactcttccacctgggcagggaaaacggtttattatgttcctctttaatttatctatcgtggtctgttttcactaaaactgtcatattgctacactccagtactaccagtacgtcgcccgcacatagtggccaaggattttactgctactgttgattaacataagcacttgcgactttccctaacatcttttataaaacaacggccgcaataatattgaactgtttttttctagtaccaaaaatagaatttgatccctcacctcattacatccatagtaacatgaccagatatatatggacaggccgggatcactcgccagcagataccctgagcgattcataaccagaatttttaattttttctagtgaagtggggttctcctagtcctttaacattcaaaatttagtacaaactttccttagtaaatgtcttctagtaaagatttcctagtgttttgatttggtagtgttttattactaattaaaaatattagaagaactccatcattttggtagtgattggttgtttggattagtcttctcacgttagacctatatatgcaggacaactcaagccagcataaatatatgaaatatcttggtgtttgtttgtctgacacaggcaaccgtgtttggtataaatgtgttttcttgtttacgttttaccatctatagtcatctcaatgtttatatagtagagacttcatgtttgtagtagataaggtagagaattgagaatattttatttttgtgcgaccatcaattttatgtaatctgcattgtctaatgctttatttgacatttgaaactacttaatttgaccgttatgcaggtccgcatgatcctatgaaagcaattaattagtacgggtactgcactacacaagtttgctagtactattctattaaccgacctgtcaatattaccttaagttactgatttcaattagaatctaacacattcaggaaaagaagttttccttattagtagtaactttttatactaattaagattcaataaaaattcaccatgacatccccattgccaagagaatatttcgccgcccctcaaagcagccaaggctttactaaaaagactatccacgcagtagagatttagtcaaaatattccaatagcaattgttttctgcctgcttgaccttcgtcagccactcactgtataaatatcgcaccacgccctttgcaggcttacagagcttgtactacgtactaacaaggcacacacaataccctgtgttcaccggccctgcacaaaactcaagcagttattactaacatggcggctaacgattccttggttactgctcatgtgataggagatgtcttggaccccttctatacaaccgttgatatgatgatcctattcgatggtactcctattatcagcggcatggagttgcgtgctccggcggtttctgacaggccaagggttgagattggaggagatgattatcgagttgcatatactctggtaaactcatgtcatgtcaattaactagtagttgaatttagatgctggtcgtatcgtggatacatgaactatatgttatggttgatacatatttgtttaattgatcgcaacaccatttgtggtaacttcaaataacattctttcaatatataggtgatggtcgatcctgatgctcctaacccaagcaacccaaccttgagggagtacttgcactggtaagagaaacctatagacgacaattattgttgttggcatgttctgcccacatatactttgctagtgtgtgtatatttgtgcttatgcttctccataaattttggtgtatgtcccaagagagataggtatagaggttagcagtcctttaaaaatggtttaatccagtagttttttttcggtcggccggactgctagtaactttcaatcatttcatgtttcgagcaggatggtgactgacatcccagcatcaactgataatacatacggtgagatcacccctattcccattttgagacaagtagaatgtctatttttatgatctagtatgttcgtgacaataggctagctattttgaaacttcgggagcataaaatagtactcgattttgtataaccataaacacagctagccaatctctattcatatttattttagttttatttgccgaaccatcctcaacatcatagccacttgatcgatcatctcaatcagcgtttgtatccttgcccgctttgattatcatccatgacagttcatattttttttcatttctttcatgcttgttatagttttatctgatgaatccgagatgttattgatcaattagttcagatgagcagtaatgtatgttggaggtttggtagtatatatacgttcaatatttcacgaaatcggtaattacgaaaatcccaaaattttgaattacattaataatgcatgtgactcatattttctatgatttctattctgttgcatattcttgtactcaatagatatttaaatcatgctaatattttgtttagatctaaatcttttagaaaaattataatttatatttgggtttaacaatttcgggcgcgtttagtgagattgggtaatttcggagcgaggcggccgccggccacgaaaaattctatacacgactatatgtgtacatgtacatgcatggcaccttgataggctaccccggcccgcatggggaaaaaattggaaacggaccattcatacgcagtcgtggtgccgactgtgggccacaatagcagtgtaaacataattacggtaatcaaataccccgtgggaccatatatatcatccacagatccgtacggtgcttccgtgtggatggtctaccccagatcttttccaccccataagggcagcaatgcagcatcatattcatatgcactagtgatgtaccatttggcttatatcatattcaacctaactccttggaaacattatgatgttctattggggtgaagatgtcactactaaaaaaagatcttatgagaggtgttttgaaaactgcccgaggtggttaaaggagacggacgagttaggacaactgcctctattaatgtgtattaaccgaggtagttaccgtaacgtgcctgacttgattaacagattcaaccgtctcagtaaagaccatgattaaccgaaacggaatcgagagttttctcaagtagttaaactattttaaactgcaccgaacttataaaaatggtagagctaacaccaatatttataaaaataaattagtatcactaaatacatcacgaaatctatttggtgttgtagaagttatccttttctataaaattgatcaaatttatgataacttagttttaggaattgatttattttaggacaactaaggaagtacattttttaaagtcatccacaaagtagtggatccaatttattacattactccactacttcaaactgaacaaaagcctaatcctggttattttgagagtgattttttacaacatcagcagtagtccagaaaatgggaggacattaataaaagtgaaaaggagcagaagaaagattacggtattttatttgtgctatttgtttaactattggcagtttgggaccgaaaataaataactgttcgtagctctatatttgtccattcgaaagtgtaacgatgattattgtgtttcaaaagataaataaagaagtgcaccaatgatttgatatcataggctatataatccaacatggtgaaaatgcttttcaatcaagtaatcttcgagcggttaccagttttaatagttgcgagtcgtcgttttttatgtaccctaggacatatatatatccgcatgtagacgatgagactagctagtttttttttttttgagcaaatacataattattggatttgcaggccgtgagatgatgtgctacgagccccctgccccgtccacgggcatccaccggatggtgctggtgctattccagcagcttggccgtgacacggtgttcgcggcgccgtccaggcgccacaacttcaacacccgtgccttcgcccgccgctacaacctcggcgcgcccgtcgccgccatgttcttcaactgccagcgccagaccggctccggtggccccaggttcaccgggccctacaccagccgccgtcgtgcgggctgatgacgacgatcgtcgttacgtcacgtgtaccgtacatatatatgtaagatatacatgcatgttccatggtaaggatcggtgacaaaacgtctaataatgtatacacacatatgcatggaatgcatgtaataagagaatatatgtataataagtaggggggagcatgcatatattgtacacgcgtccgatgcgtatatagccctatacattattgtagttgtaatcagctgtttaagcattctgctgtgtcagaacatgatgcatatatagtttggtgtcagtattgatgttgtggaactcttatcagccttcatctcatcacaagtgaaagatatagcttttatacctccaagtgtcttcccaatgtacgtacctagaacttttctaagaaatgctacaaatgttgtattttatctgtgcgcttcactactggaaacccgaatatttctgtggatgtcgaatttttctgtgcgtttttttcgatacgcacggaaaaattataattattttgtgagttttaaaataccctcacagaaaaatacaaatacccacagaacaattatatcatttttctgtgcgtgacaatacactcacaaaaattacaatttttgtgtgtgtttatataaaatgcacagaaaaaaataatcacacacagaaaaattatacttattctgtgggtttctataaaacgcacataaaaaaataaacacacagagaaaaatagaacaagcaccctcatactaacttcatatgaacacgcatattttttctttttaatctctctgtaaaacttgtaactagtttttcccactcgtactaactccaaattggatgattt

SEQ ID NO: 10 - DNA, 2850 bp, Sorghum bicolor
atggcggctaacgattccttggttactgctcatgtgataggagatgtcttggaccccttctatacaaccgttgatatgatgatcctattcgatggtactcctattatcagcggcatggagttgcgtgctccggcggtttctgacaggccaagggttgagattggaggagatgattatcgagttgcatatactctggtaaactcatgtcatgtcaattaactagtagttgaatttagatgctggtcgtatcgtggatacatgaactatatgttatggttgatacatatttgtttaattgatcgcaacaccatttgtggtaacttcaaataacattctttcaatatataggtgatggtcgatcctgatgctcctaacccaagcaacccaaccttgagggagtacttgcactggtaagagaaacctatagacgacaattattgttgttggcatgttctgcccacatatactttgctagtgtgtgtatatttgtgcttatgcttctccataaattttggtgtatgtcccaagagagataggtatagaggttagcagtcctttaaaaatggtttaatccagtagttttttttcggtcggccggactgctagtaactttcaatcatttcatgtttcgagcaggatggtgactgacatcccagcatcaactgataatacatacggtgagatcacccctattcccattttgagacaagtagaatgtctatttttatgatctagtatgttcgtgacaataggctagctattttgaaacttcgggagcataaaatagtactcgattttgtataaccataaacacagctagccaatctctattcatatttattttagttttatttgccgaaccatcctcaacatcatagccacttgatcgatcatctcaatcagcgtttgtatccttgcccgctttgattatcatccatgacagttcatattttttttcatttctttcatgcttgttatagttttatctgatgaatccgagatgttattgatcaattagttcagatgagcagtaatgtatgttggaggtttggtagtatatatacgttcaatatttcacgaaatcggtaattacgaaaatcccaaaattttgaattacattaataatgcatgtgactcatattttctatgatttctattctgttgcatattcttgtactcaatagatatttaaatcatgctaatattttgtttagatctaaatcttttagaaaaattataatttatatttgggtttaacaatttcgggcgcgtttagtgagattgggtaatttcggagcgaggcggccgccggccacgaaaaattctatacacgactatatgtgtacatgtacatgcatggcaccttgataggctaccccggcccgcatggggaaaaaattggaaacggaccattcatacgcagtcgtggtgccgactgtgggccacaatagcagtgtaaacataattacggtaatcaaataccccgtgggaccatatatatcatccacagatccgtacggtgcttccgtgtggatggtctaccccagatcttttccaccccataagggcagcaatgcagcatcatattcatatgcactagtgatgtaccatttggcttatatcatattcaacctaactccttggaaacattatgatgttctattggggtgaagatgtcactactaaaaaaagatcttatgagaggtgttttgaaaactgcccgaggtggttaaaggagacggacgagttaggacaactgcctctattaatgtgtattaaccgaggtagttaccgtaacgtgcctgacttgattaacagattcaaccgtctcagtaaagaccatgattaaccgaaacggaatcgagagttttctcaagtagttaaactattttaaactgcaccgaacttataaaaatggtagagctaacaccaatatttataaaaataaattagtatcactaaatacatcacgaaatctatttggtgttgtagaagttatccttttctataaaattgatcaaatttatgataacttagttttaggaattgatttattttaggacaactaaggaagtacattttttaaagtcatccacaaagtagtggatccaatttattacattactccactacttcaaactgaacaaaagcctaatcctggttattttgagagtgattttttacaacatcagcagtagtccagaaaatgggaggacattaataaaagtgaaaaggagcagaagaaagattacggtattttatttgtgctatttgtttaactattggcagtttgggaccgaaaataaataactgttcgtagctctatatttgtccattcgaaagtgtaacgatgattattgtgtttcaaaagataaataaagaagtgcaccaatgatttgatatcataggctatataatccaacatggtgaaaatgcttttcaatcaagtaatcttcgagcggttaccagttttaatagttgcgagtcgtcgttttttatgtaccctaggacatatatatatccgcatgtagacgatgagactagctagtttttttttttttgagcaaatacataattattggatttgcaggccgtgagatgatgtgctacgagccccctgccccgtccacgggcatccaccggatggtgctggtgctattccagcagcttggccgtgacacggtgttcgcggcgccgtccaggcgccacaacttcaacacccgtgccttcgcccgccgctacaacctcggcgcgcccgtcgccgccatgttcttcaactgccagcgccagaccggctccggtggccccaggttcaccgggccctacaccagccgccgtcgtgcgggctga

SEQ ID NO: 11 - DNA, 558 bp, Sorghum bicolor
atggcggctaacgattccttggttactgctcatgtgataggagatgtcttggaccccttctatacaaccgttgatatgatgatcctattcgatggtactcctattatcagcggcatggagttgcgtgctccggcggtttctgacaggccaagggttgagattggaggagatgattatcgagttgcatatactctggtgatggtcgatcctgatgctcctaacccaagcaacccaaccttgagggagtacttgcactggatggtgactgacatcccagcatcaactgataatacatacggccgtgagatgatgtgctacgagccccctgccccgtccacgggcatccaccggatggtgctggtgctattccagcagcttggccgtgacacggtgttcgcggcgccgtccaggcgccacaacttcaacacccgtgccttcgcccgccgctacaacctcggcgcgcccgtcgccgccatgttcttcaactgccagcgccagaccggctccggtggccccaggttcaccgggccctacaccagccgccgtcgtgcgggctga

SEQ ID NO: 12 - DNA, 4371 bp, Sorghum bicolor
ttccacctgggcagggaaaacggtttattatgttcctctttaatttatctatcgtggtctgttttcactaaaactgtcatattgctacactccagtactaccagtacgtcgcccgcacatagtggccaaggattttactgctactgttgattaacataagcacttgcgactttccctaacatcttttataaaacaacggccgcaataatattgaactgtttttttctagtaccaaaaatagaatttgatccctcacctcattacatccatagtaacatgaccagatatatatggacaggccgggatcactcgccagcagataccctgagcgattcataaccagaatttttaattttttctagtgaagtggggttctcctagtcctttaacattcaaaatttagtacaaactttccttagtaaatgtcttctagtaaagatttcctagtgttttgatttggtagtgttttattactaattaaaaatattagaagaactccatcattttggtagtgattggttgtttggattagtcttctcacgttagacctatatatgcaggacaactcaagccagcataaatatatgaaatatcttggtgtttgtttgtctgacacaggcaaccgtgtttggtataaatgtgttttcttgtttacgttttaccatctatagtcatctcaatgtttatatagtagagacttcatgtttgtagtagataaggtagagaattgagaatattttatttttgtgcgaccatcaattttatgtaatctgcattgtctaatgctttatttgacatttgaaactacttaatttgaccgttatgcaggtccgcatgatcctatgaaagcaattaattagtacgggtactgcactacacaagtttgctagtactattctattaaccgacctgtcaatattaccttaagttactgatttcaattagaatctaacacattcaggaaaagaagttttccttattagtagtaactttttatactaattaagattcaataaaaattcaccatgacatccccattgccaagagaatatttcgccgcccctcaaagcagccaaggctttactaaaaagactatccacgcagtagagatttagtcaaaatattccaatagcaattgttttctgcctgcttgaccttcgtcagccactcactgtataaatatcgcaccacgccctttgcaggcttacagagcttgtactacgtactaacaaggcacacacaataccctgtgttcaccggccctgcacaaaactcaagcagttattactaacatggcggctaacgattccttggttactgctcatgtgataggagatgtcttggaccccttctatacaaccgttgatatgatgatcctattcgatggtactcctattatcagcggcatggagttgcgtgctccggcggtttctgacaggccaagggttgagattggaggagatgattatcgagttgcatatactctggtaaactcatgtcatgtcaattaactagtagttgaatttagatgctggtcgtatcgtggatacatgaactatatgttatggttgatacatatttgtttaattgatcgcaacaccatttgtggtaacttcaaataacattctttcaatatataggtgatggtcgatcctgatgctcctaacccaagcaacccaaccttgagggagtacttgcactggtaagagaaacctatagacgacaattattgttgttggcatgttctgcccacatatactttgctagtgtgtgtatatttgtgcttatgcttctccataaattttggtgtatgtcccaagagagataggtatagaggttagcagtcctttaaaaatggtttaatccagtagttttttttcggtcggccggactgctagtaactttcaatcatttcatgtttcgagcaggatggtgactgacatcccagcatcaactgataatacatacggccgtgagatcacccctattcccattttgagacaagtagaatgtctatttttatgatctagtatgttcgtgacaataggctagctattttgaaacttcgggagcataaaatagtactcgattttgtataaccataaacacagctagccaatctctattcatatttattttagttttatttgccgaaccatcctcaacatcatagccacttgatcgatcatctcaatcagcgtttgtatccttgcccgctttgattatcatccatgacagttcatattttttttcatttctttcatgcttgttatagttttatctgatgaatccgagatgttattgatcaattagttcagatgagcagtaatgtatgttggaggtttggtagtatatatacgttcaatatttcacgaaatcggtaattacgaaaatcccaaaattttgaattacattaataatgcatgtgactcatattttctatgatttctattctgttgcatattcttgtactcaatagatatttaaatcatgctaatattttgtttagatctaaatcttttagaaaaattataatttatatttgggtttaacaatttcgggcgcgtttagtgagattgggtaatttcggagcgaggcggccgccggccacgaaaaattctatacacgactatatgtgtacatgtacatgcatggcaccttgataggctaccccggcccgcatggggaaaaaattggaaacggaccattcatacgcagtcgtggtgccgactgtgggccacaatagcagtgtaaacataattacggtaatcaaataccccgtgggaccatatatatcatccacagatccgtacggtgcttccgtgtggatggtctaccccagatcttttccaccccataagggcagcaatgcagcatcatattcatatgcactagtgatgtaccatttggcttatatcatattcaacctaactccttggaaacattatgatgttctattggggtgaagatgtcactactaaaaaaagatcttatgagaggtgttttgaaaactgcccgaggtggttaaaggagacggacgagttaggacaactgcctctattaatgtgtattaaccgaggtagttaccgtaacgtgcctgacttgattaacagattcaaccgtctcagtaaagaccatgattaaccgaaacggaatcgagagttttctcaagtagttaaactattttaaactgcaccgaacttataaaaatggtagagctaacaccaatatttataaaaataaattagtatcactaaatacatcacgaaatctatttggtgttgtagaagttatccttttctataaaattgatcaaatttatgataacttagttttaggaattgatttattttaggacaactaaggaagtacattttttaaagtcatccacaaagtagtggatccaatttattacattactccactacttcaaactgaacaaaagcctaatcctggttattttgagagtgattttttacaacatcagcagtagtccagaaaatgggaggacattaataaaagtgaaaaggagcagaagaaagattacggtattttatttgtgctatttgtttaactattggcagtttgggaccgaaaataaataactgttcgtagctctatatttgtccattcgaaagtgtaacgatgattattgtgtttcaaaagataaataaagaagtgcaccaatgatttgatatcataggctatataatccaacatggtgaaaatgcttttcaatcaagtaatcttcgagcggttaccagttttaatagttgcgagtcgtcgttttttatgtaccctaggacatatatatatccgcatgtagacgatgagactagctagtttttttttttttgagcaaatacataattattggatttgcaggccgtgagatgatgtgctacgagccccctgccccgtccacgggcatccaccggatggtgctggtgctattccagcagcttggccgtgacacggtgttcgcggcgccgtccaggcgccacaacttcaacacccgtgccttcgcccgccgctacaacctcggcgcgcccgtcgccgccatgttcttcaactgccagcgccagaccggctccggtggccccaggttcaccgggccctacaccagccgccgtcgtgcgggctgatgacgacgatcgtcgttacgtcacgtgtaccgtacatatatatgtaagatatacatgcatgttccatggtaaggatcggtgacaaaacgtctaataatgtatacacacatatgcatggaatgcatgtaataagagaatatatgtataataagtaggggggagcatgcatatattgtacacgcgtccgatgcgtatatagccctatacattattgtagttgtaatca

SEQ ID NO: 13 - DNA, 2853 bp, Sorghum bicolor
atggcggctaacgattccttggttactgctcatgtgataggagatgtcttggaccccttctatacaaccgttgatatgatgatcctattcgatggtactcctattatcagcggcatggagttgcgtgctccggcggtttctgacaggccaagggttgagattggaggagatgattatcgagttgcatatactctggtaaactcatgtcatgtcaattaactagtagttgaatttagatgctggtcgtatcgtggatacatgaactatatgttatggttgatacatatttgtttaattgatcgcaacaccatttgtggtaacttcaaataacattctttcaatatataggtgatggtcgatcctgatgctcctaacccaagcaacccaaccttgagggagtacttgcactggtaagagaaacctatagacgacaattattgttgttggcatgttctgcccacatatactttgctagtgtgtgtatatttgtgcttatgcttctccataaattttggtgtatgtcccaagagagataggtatagaggttagcagtcctttaaaaatggtttaatccagtagttttttttcggtcggccggactgctagtaactttcaatcatttcatgtttcgagcaggatggtgactgacatcccagcatcaactgataatacatacggccgtgagatcacccctattcccattttgagacaagtagaatgtctatttttatgatctagtatgttcgtgacaataggctagctattttgaaacttcgggagcataaaatagtactcgattttgtataaccataaacacagctagccaatctctattcatatttattttagttttatttgccgaaccatcctcaacatcatagccacttgatcgatcatctcaatcagcgtttgtatccttgcccgctttgattatcatccatgacagttcatattttttttcatttctttcatgcttgttatagttttatctgatgaatccgagatgttattgatcaattagttcagatgagcagtaatgtatgttggaggtttggtagtatatatacgttcaatatttcacgaaatcggtaattacgaaaatcccaaaattttgaattacattaataatgcatgtgactcatattttctatgatttctattctgttgcatattcttgtactcaatagatatttaaatcatgctaatattttgtttagatctaaatcttttagaaaaattataatttatatttgggtttaacaatttcgggcgcgtttagtgagattgggtaatttcggagcgaggcggccgccggccacgaaaaattctatacacgactatatgtgtacatgtacatgcatggcaccttgataggctaccccggcccgcatggggaaaaaattggaaacggaccattcatacgcagtcgtggtgccgactgtgggccacaatagcagtgtaaacataattacggtaatcaaataccccgtgggaccatatatatcatccacagatccgtacggtgcttccgtgtggatggtctaccccagatcttttccaccccataagggcagcaatgcagcatcatattcatatgcactagtgatgtaccatttggcttatatcatattcaacctaactccttggaaacattatgatgttctattggggtgaagatgtcactactaaaaaaagatcttatgagaggtgttttgaaaactgcccgaggtggttaaaggagacggacgagttaggacaactgcctctattaatgtgtattaaccgaggtagttaccgtaacgtgcctgacttgattaacagattcaaccgtctcagtaaagaccatgattaaccgaaacggaatcgagagttttctcaagtagttaaactattttaaactgcaccgaacttataaaaatggtagagctaacaccaatatttataaaaataaattagtatcactaaatacatcacgaaatctatttggtgttgtagaagttatccttttctataaaattgatcaaatttatgataacttagttttaggaattgatttattttaggacaactaaggaagtacattttttaaagtcatccacaaagtagtggatccaatttattacattactccactacttcaaactgaacaaaagcctaatcctggttattttgagagtgattttttacaacatcagcagtagtccagaaaatgggaggacattaataaaagtgaaaaggagcagaagaaagattacggtattttatttgtgctatttgtttaactattggcagtttgggaccgaaaataaataactgttcgtagctctatatttgtccattcgaaagtgtaacgatgattattgtgtttcaaaagataaataaagaagtgcaccaatgatttgatatcataggctatataatccaacatggtgaaaatgcttttcaatcaagtaatcttcgagcggttaccagttttaatagttgcgagtcgtcgttttttatgtaccctaggacatatatatatccgcatgtagacgatgagactagctagtttttttttttttgagcaaatacataattattggatttgcaggccgtgagatgatgtgctacgagccccctgccccgtccacgggcatccaccggatggtgctggtgctattccagcagcttggccgtgacacggtgttcgcggcgccgtccaggcgccacaacttcaacacccgtgccttcgcccgccgctacaacctcggcgcgcccgtcgccgccatgttcttcaactgccagcgccagaccggctccggtggccccaggttcaccgggccctacaccagccgccgtcgtgcgggctga

SEQ ID NO: 14 - DNA, 6971 bp, Sorghum propinquum
  misc_feature (6480)..(6480): n is a, c, g, or t
aaaagaaaagtgagcacaccacgacctgtcatcagctcatggtcagctctacaaacttatagattgcatcgagatctaagactcaggtacaaatcatgtcaacatctaatggtttagaaaatgaaaagttttgagtttcaaaatatgatacgtgatattaacatttgaacttttagcaagatctgaaataaaaaattcaactagatcatgttaacattgatataatcgcttccaatcgcctcccatcacttccgctagaaaactttttttctcgatttaattaatgaaagggtaataacatcattgtacaagattctttcaaacctcaacccctatcatcgacggtgacggctccctataacacgcactagtggacgccgggcgggtggaaccctaagaagatttaaaaaaacttaagaagaagatttttatctaactaactatagtacttatatcatacactatactattcaaaatattattttcacaattatgaatttacccttttactcttcattaaaaaaatacgaaaaaagaatcaccacgtctctatttagggtcctagtccccataatttaagaggcggtgagagacgatgtgacgtctatggaccaccgaccaaagacacacctatcgtctcccatcgccttgcttccatcgcctctcatcgcttttcatattctagatccagcggccatagacacaccaatcgtttctcatcgcctctccaaccattgtaaaaatatttataattttgatataaaatttgtcttcacttgagttcatgccaaaaaaattatacatattattttcgtgtgagaatttacagaagtggactcttaagatgtccaaatgtaaatgaccctatttattatgaggcgcggatctataggcctgactctgaaaatggattatggatttgagataataaatttaagggcctatcttcgcacataacatctatagttcctaaatttttttttattgtagtagtagaacttttctccctgtaaaccaagttgacgctgggctttattttgcgacacagaacaccaaattggtggctatgaactcttccacctgggcagggaaaacggtttattatgtttctctttaatttatctatcgtggcactataacacaacatggctttgccgacacttccaactatcggcaaagggtacctttaccgacacttaacgtctcacgaaaggttttgccgacaattttcaaacagtcgcggtagaagcagttggcgaaacttttgccgacagttaaaggcatcgccgacacattttctgtagtcaaatggcatacctacgccgacagttgaactttcaccgacagtgaaccctttgccgacagtttggacctacgccgacagtttggaccttttccgacagttggtatgttagcgaaaccgtttctagggtgtttcataaaccatgccttgtccaacagtagaagtgtcggcaaaactatattgctaggatgtagatacaatttaaatattttaataaatacacatcacattgattgagcaaaatcacatggtctgttttcactaaaactgtcagaggtacactccagtactaccagtacgtcgcccgcacagtggccaaggattttactgctactgttgattaacataagcacttgcgactttccctaaaatcttttataaaacaacggccgcaataatattgaactattttttttctagtaccaaaattagaatttgatccctcacctcattacatccatagtaacatgaccagatatatatggacaggatgggatcactcagcgagcagatacactgagcgattcataatcagattttttaatttcttctagtgaagtggggttttcctagtcttttaacattcaaaatttagtacaaactttccctagtaaatgccttctagtaaagatttcctagtattttgactagcgatagtgttttattactaattaaaaacattagaagaactccatttagtgattggttgtttggattagtcttctcacgttagacctatatatgcaggacaactcaagccagcataaatatatgaaatatcttggtgtttgtttgtctgacacaggcaaccgcgtttggtataaatgtgttttcttgtttacattttaccatctatagtcatctcaatgttatatagtagaggcttcatgtttgtagtagataaggtagagaattgagaatattttatttttgtgcgaccatcaattttatgtaatctgcattgtctaatgctttatttgacatttgaaactacttaatttgacagttatgcaggtccgcatgatcctatgaaagcaattaattagtacgggtaaactgcactacacaagtttgctagtactattctattaaccgacctgtcaatattaccttaagttactgatttcaattagaatctaacacattcaggaaaagaagtttcactagtacaaaaatcattttcgttggcacgttgttttttttttcacaggcagttcacaatatcatggtgctagtagaaaaatttcaacgggcccaacaagagaaccgccaggcggtcttcttaattcaaccgcctgtgtaaactttccatttacataggcggcttacgataaaaaccgtgtgtataaataccattaacacaggcagtcgagttacgacaaccgcctgtgtaaatgtgtctttttacacaggcggtttgtatagagggccgcctgtgctaatatatttacacaggctatgagccgcctgtgttaagtcttctataaatacccttcgtccacctccagacaagaacagttactcccatgagctctgcacactggcggaccagacgattccagtttccaaggggggaggttttgattttcatttctttggtgagaaacttccaaaaggttagttagtgccattgatgctattttttaagcgattctttggttcaattcttgtattggaggtgctctagatctagagttcatcatgcattcttgcttagggttagagttcatagggcaaaaagagagagatttagctaaatttttatgtaaattcatagtaaattgtaaaaattaaaaaaaataaaaaataaatactttttagaattcttgtgagtagatctatacaatagagtaatgatgaggatattttgaagtttataattttgattcagttttagcttttcttttttcagatgaattagactttataaactcaaacattaaaatgttgaaaatcataaaatggcaaataaatactttttcaaatctttgtgcataaatacttcatagaaatccttgaattattcctaaattttatacaattgtttcttataattatgaaaatgagtttaaacaattatttaaattccataaattgtaactccgtaaggtgtaggttttcatctctgtttaatagaaggaggttagtatcttagttaagtctgttttcgggggttatattagttttgtttttagattgacctacattaattgttcttaactaattacagctaaatatggagaggtcattatggatgtacaacttatcaagattggacctatcatatgtagtgcaggtccaaaaatttattgatgtcgcaaagatacatgctcgcagaacaaaggcgaagcacatatgttgtccatgcgcagactgcaaaaatattatggtatttgacaatgtagaagcaattacttcccatctggtttgaagaggatttatggaggactacttgatttggacaaaacatggtgagggtagttttgcaccttatatgcggacaactgacaacactgcaactaacatcaatgtggagggtccaatgccacctctcaatgaatttcatgctatgccagatgttaatgaaactcatacgtctgatgtcaatgaaactcagcatgctaacacagatgttgttgaagatgcagatttcttagaggcaataatgaaccgttgtgcggatccatcaatattcttcatgaagggaatgaaagcattgaagaaggcagcagaggacactttgtacgacgagtcaaaaggttgtaccaaacaatggtcgacattatgtgttgttcttcagtttttgacgatgaaggctagacatggttggtccgatgctagcttcaatgatttcttgcgtgtacttggagaccttcttcctaaggagaacaaagtgcctgctaacacatactatgcaaagaagctagtcagtccacttacgataggtgttgagaagatccacgcatgtagaaatcattgtattctatatcgaggtgatcaatataaagacttagacagttgtccaaactgtggtgccagtaggtacaagacaaacaaagattttcgggaggaagagaatctagcctctgtttctacagggaggaagcgaaagaagacccaaacaaagactcaacaagacaagcgctcaaagcctagtagcaatgaagaagtggactattatgcattgagaagagtctccctatgagccaaaaaaggggacagcagcaggcacaactctctttctgaaaggacttggaaagcagcggacggcacggctcattgagctcgaaccgtcacagaaaaaggaagccaccgcccagtcaatagaagccatgcccccatcaaaggaagccccaagtggcgatgtacatattgaacagccatcaagtcaaccattgaccctaaaggatatcagaaagccaacgattgatgattatgtcaatgtccctagtgactatgtgcccggaaggcctatgctccaatggacgctgctcgattagattcaatggctgataaaaaggtttcatgactggtacatgagagcagtgcatgctagcctccatggaatcagagttgatataccaacagacatgtttgctactggtaacaaaaaaagcaagacatttgttacctttgaggacatgcacttgttattgaactataggcggcttgacgtccaactcataacaatctggtgcctgtaagtatcactcatgcacacacaattattatatattaatatgtagtgtgaaactctaatatgtagatgttgtctgtagtttgcaagatcacgagcagatgtcattattatctgccggatcgatggtcggttatctgagccctatcaagttacaagaaaatatgaacaaattcgtattatcaaaggaagatagagcaaagatagaggaagacaaaacaccaggataattatgccatctatcttggtagatcaatgctgaggtataaatatagggattttatattggcaccatacaacattaggtaagcttgacttcatatacgtatttcaaattatcgtgtaaacaatatacatgtgtcgctcactcatttattcatgcagtgaccattggattgttttttatatttatcccttcgaagggaaggtgcttgtcctagactctttacatgttcctcccgagaagtatcaaccattcttggttcaattagaaaggtgagccaacatgaaaccacatgcgtacttatataaattagagtttcaaaataactttagtgatttaggttcgatatctacggggcatggcggttttataagaaacaaaagggacctgtcgacgctgcacgctcagatcctaggatcccattgatgatacaacaccactatccggtaagttttctgaacacatttcatcatataaataatacataaagcatggcaaatttagaataatccgttgctcattatatagtgccacaagcaaccacctggatcggtctattgtgggtactatgtctgtgagtttataaggcagcggggacgttacgtcaaggacaaaaatatggtaaataatatctatgtatgaaagttttctcattaaagctgcaaaattatatattgaacatgtgtcaatcatgcttttaaactttattttcagccgaaaaagcaaggaaaagacgtgccctttacaccaaagactctggaagatatagtagcatacttgtgtggttttattatgagagaaataatttcaagtgacagtgcatattttgatcatgagggcgatttagcaagtgataaatttagagtgctgacagacatagcaggtctaaatctgaagcgaaacgacatgtaaacattgtatggttgtgcggataacatgcattgacgtgtatatatataattttatggttgatgtttgatttgtttacaattctataatatatatatgtggtgtatgtatgatgttgtgtgtgtatatatatatatatatatatatatatatatatatatatatatatatatatatatatataatgtttagcactgtgtttggtgggaaaaattaaaatttgaaatatatataaaaaattatttacacagacagtgtacgtgtcgagcgtcgtcctgtgctatacaaatacattctaacaggcggctcgccttgtccaccggtcggttaaaaatacatttccacacnggcctggctgggagagccgcctgtgaaaacataattttcacaggcggctcgcacagccccgcctgtactgtggtccattttgtactgacccctggtacaggcggtgggcttggccgcctgtgaagatgcttttagcaccgcctgtaaaaatgttttttgtagcagtgtttttcttattagtagtatcttttatactaattaagattcaataaaaattcaccatgacatccccattgccaagagaatatttcgccgcccctcaaagcagccaataaggctttactaaaaagactatccacgcagtagagatttagtcaaaatattccaatagcaattgtttcctgcctgcttgaccttcgtcagccactcactgtataaatatcgcaccacgccctttgcaggcttacagagcttgtattacgtactaacaaggcacacacagtaccctgtgttcaccggccctgcacaaaactcaagcagttattactaac

SEQ ID NO: 15 - DNA, 7481 bp, Sorghum propinquum
  misc_feature (6906)..(6909): N=1, 2, 3, 4, or 5 nucleotides in length
ccctgacccttgttgggcaacatttagagtcgttagctttgcaattctttggttccaatggatggttatcatttagacatattggtcatgcttagtcaaaactttattgttcggctataaacttttcagtactttgtaataattggctcgatagatgaagccgggtataacatatcctttatctaaaaaaattagttaacatgaacttcatattcaattcttcatatctcactagcatctttattgtctagttagttttgtagcattgcaaaaagcatgcaactatatacaatgaaacggaataaaatttcagctctattaatttatatttcaaatataggccactatagccatatttcgtgctcaaggccacaaaatcttgcgtacttccctgttggtaccaaagagaagacgttatttaactttgtttgactcttcaatatggtttgaatcagaaaattagttaaaagaaaagtgagcacaccacgacctgtcatcagctcatggtcagctctacaaacttatagattgcatcgagatctaagactcaggtacaaatcatgtcaacatctaatggtttagaaaatgaaaagttttgagtttcaaaatatgatacgtgatattaacatttgaacttttagcaagatctgaaataaaaaattcaactagatcatgttaacattgatataatcgcttccaatcgcctcccatcacttccgctagaaaactttttttctcgatttaattaatgaaagggtaataacatcattgtacaagattctttcaaacctcaacccctatcatcgacggtgacggctccctataacacgcactagtggacgccgggcgggtggaaccctaagaagatttaaaaaaacttaagaagaagatttttatctaactaactatagtacttatatcatacactatactattcaaaatattattttcacaattatgaatttacccttttactcttcattaaaaaaatacgaaaaaagaatcaccacgtctctatttagggtcctagtccccataatttaagaggcggtgagagacgatgtgacgtctatggaccaccgaccaaagacacacctatcgtctcccatcgccttgcttccatcgcctctcatcgcttttcatattctagatccagcggccatagacacaccaatcgtttctcatcgcctctccaaccattgtaaaaatatttataattttgatataaaatttgtcttcacttgagttcatgccaaaaaaattatacatattattttcgtgtgagaatttacagaagtggactcttaagatgtccaaatgtaaatgaccctatttattatgaggcgcggatctataggcctgactctgaaaatggattatggatttgagataataaatttaagggcctatcttcgcacataacatctatagttcctaaatttttttttattgtagtagtagaacttttctccctgtaaaccaagttgacgctgggctttattttgcgacacagaacaccaaattggtggctatgaactcttccacctgggcagggaaaacggtttattatgtttctctttaatttatctatcgtggcactataacacaacatggctttgccgacacttccaactatcggcaaagggtacctttaccgacacttaacgtctcacgaaaggttttgccgacaattttcaaacagtcgcggtagaagcagttggcgaaacttttgccgacagttaaaggcatcgccgacacattttctgtagtcaaatggcatacctacgccgacagttgaactttcaccgacagtgaaccctttgccgacagtttggacctacgccgacagtttggaccttttccgacagttggtatgttagcgaaaccgtttctagggtgtttcataaaccatgccttgtccaacagtagaagtgtcggcaaaactatattgctaggatgtagatacaatttaaatattttaataaatacacatcacattgattgagcaaaatcacatggtctgttttcactaaaactgtcagaggtacactccagtactaccagtacgtcgcccgcacagtggccaaggattttactgctactgttgattaacataagcacttgcgactttccctaaaatcttttataaaacaacggccgcaataatattgaactattttttttctagtaccaaaattagaatttgatccctcacctcattacatccatagtaacatgaccagatatatatggacaggatgggatcactcagcgagcagatacactgagcgattcataatcagattttttaatttcttctagtgaagtggggttttcctagtcttttaacattcaaaatttagtacaaactttccctagtaaatgccttctagtaaagatttcctagtattttgactagcgatagtgttttattactaattaaaaacattagaagaactccatttagtgattggttgtttggattagtcttctcacgttagacctatatatgcaggacaactcaagccagcataaatatatgaaatatcttggtgtttgtttgtctgacacaggcaaccgcgtttggtataaatgtgttttcttgtttacattttaccatctatagtcatctcaatgttatatagtagaggcttcatgtttgtagtagataaggtagagaattgagaatattttatttttgtgcgaccatcaattttatgtaatctgcattgtctaatgctttatttgacatttgaaactacttaatttgacagttatgcaggtccgcatgatcctatgaaagcaattaattagtacgggtaaactgcactacacaagtttgctagtactattctattaaccgacctgtcaatattaccttaagttactgatttcaattagaatctaacacattcaggaaaagaagtttcactagtacaaaaatcattttcgttggcacgttgttttttttttcacaggcagttcacaatatcatggtgctagtagaaaaatttcaacgggcccaacaagagaaccgccaggcggtcttcttaattcaaccgcctgtgtaaactttccatttacataggcggcttacgataaaaaccgtgtgtataaataccattaacacaggcagtcgagttacgacaaccgcctgtgtaaatgtgtctttttacacaggcggtttgtatagagggccgcctgtgctaatatatttacacaggctatgagccgcctgtgttaagtcttctataaatacccttcgtccacctccagacaagaacagttactcccatgagctctgcacactggcggaccagacgattccagtttccaaggggggaggttttgattttcatttctttggtgagaaacttccaaaaggttagttagtgccattgatgctattttttaagcgattctttggttcaattcttgtattggaggtgctctagatctagagttcatcatgcattcttgcttagggttagagttcatagggcaaaaagagagagatttagctaaatttttatgtaaattcatagtaaattgtaaaaattaaaaaaaataaaaaataaatactttttagaattcttgtgagtagatctatacaatagagtaatgatgaggatattttgaagtttataattttgattcagttttagcttttcttttttcagatgaattagactttataaactcaaacattaaaatgttgaaaatcataaaatggcaaataaatactttttcaaatctttgtgcataaatacttcatagaaatccttgaattattcctaaattttatacaattgtttcttataattatgaaaatgagtttaaacaattatttaaattccataaattgtaactccgtaaggtgtaggttttcatctctgtttaatagaaggaggttagtatcttagttaagtctgttttcgggggttatattagttttgtttttagattgacctacattaattgttcttaactaattacagctaaatatggagaggtcattatggatgtacaacttatcaagattggacctatcatatgtagtgcaggtccaaaaatttattgatgtcgcaaagatacatgctcgcagaacaaaggcgaagcacatatgttgtccatgcgcagactgcaaaaatattatggtatttgacaatgtagaagcaattacttcccatctggtttgaagaggatttatggaggactacttgatttggacaaaacatggtgagggtagttttgcaccttatatgcggacaactgacaacactgcaactaacatcaatgtggagggtccaatgccacctctcaatgaatttcatgctatgccagatgttaatgaaactcatacgtctgatgtcaatgaaactcagcatgctaacacagatgttgttgaagatgcagatttcttagaggcaataatgaaccgttgtgcggatccatcaatattcttcatgaagggaatgaaagcattgaagaaggcagcagaggacactttgtacgacgagtcaaaaggttgtaccaaacaatggtcgacattatgtgttgttcttcagtttttgacgatgaaggctagacatggttggtccgatgctagcttcaatgatttcttgcgtgtacttggagaccttcttcctaaggagaacaaagtgcctgctaacacatactatgcaaagaagctagtcagtccacttacgataggtgttgagaagatccacgcatgtagaaatcattgtattctatatcgaggtgatcaatataaagacttagacagttgtccaaactgtggtgccagtaggtacaagacaaacaaagattttcgggaggaagagaatctagcctctgtttctacagggaggaagcgaaagaagacccaaacaaagactcaacaagacaagcgctcaaagcctagtagcaatgaagaagtggactattatgcattgagaagagtctccctatgagccaaaaaaggggacagcagcaggcacaactctctttctgaaaggacttggaaagcagcggacggcacggctcattgagctcgaaccgtcacagaaaaaggaagccaccgcccagtcaatagaagccatgcccccatcaaaggaagccccaagtggcgatgtacatattgaacagccatcaagtcaaccattgaccctaaaggatatcagaaagccaacgattgatgattatgtcaatgtccctagtgactatgtgcccggaaggcctatgctccaatggacgctgctcgattagattcaatggctgataaaaaggtttcatgactggtacatgagagcagtgcatgctagcctccatggaatcagagttgatataccaacagacatgtttgctactggtaacaaaaaaagcaagacatttgttacctttgaggacatgcacttgttattgaactataggcggcttgacgtccaactcataacaatctggtgcctgtaagtatcactcatgcacacacaattattatatattaatatgtagtgtgaaactctaatatgtagatgttgtctgtagtttgcaagatcacgagcagatgtcattattatctgccggatcgatggtcggttatctgagccctatcaagttacaagaaaatatgaacaaattcgtattatcaaaggaagatagagcaaagatagaggaagacaaaacaccaggataattatgccatctatcttggtagatcaatgctgaggtataaatatagggattttatattggcaccatacaacattaggtaagcttgacttcatatacgtatttcaaattatcgtgtaaacaatatacatgtgtcgctcactcatttattcatgcagtgaccattggattgttttttatatttatcccttcgaagggaaggtgcttgtcctagactctttacatgttcctcccgagaagtatcaaccattcttggttcaattagaaaggtgagccaacatgaaaccacatgcgtacttatataaattagagtttcaaaataactttagtgatttaggttcgatatctacggggcatggcggttttataagaaacaaaagggacctgtcgacgctgcacgctcagatcctaggatcccattgatgatacaacaccactatccggtaagttttctgaacacatttcatcatataaataatacataaagcatggcaaatttagaataatccgttgctcattatatagtgccacaagcaaccacctggatcggtctattgtgggtactatgtctgtgagtttataaggcagcggggacgttacgtcaaggacaaaaatatggtaaataatatctatgtatgaagttttctcattaaagctgcaaaattatatattgaacatgtgtcaatcatgcttttaaactttattttcagccgaaaaagcaaggaaaagacgtgccctttacaccaaagactctggaagatatagtagcatacttgtgtggttttattatgagagaaataatttcaagtgacagtgcatattttgatcatgagggcgatttagcaagtgataaatttagagtgctgacagacatagcaggtctaaatctgaagcgaaacgacatgtaaacattgtatggttgtgcggataacatgcattgacgtgtatatatataattttatggttgatgtttgatttgtttacaattctataatatatatatgtggtgtatgtatgatgttgtgtgtgtatatatatatatatatatatatatatatatatatatatatatatatatatatatatataatgtttagcactgtgtttggtgggaaaaattaaaatttgaaatatatataaaaaattatttacacagacagtgtagtgtgagctgcctgtgtaaaaatacatttatacaggcggctcaccttgtcnnnncaggcggtgctaaaagcatcttcacaggcggccaagcccaccgcctgtaccaggggtcagtacaaaatggaccacagtacaggcggggctgtgcgagccgcctgtgaaaacataattttcacaggcggctcgcacagccccgcctgtactgtggtccattttgtactgacccctggtacaggcggtgggcttggccgcctgtgaagatgcttttagcaccgcctgtaaaaatgttttttgtagcagtgtttttcttattagtagtatcttttatactaattaagattcaataaaaattcaccatgacatccccattgccaagagaatatttcgccgcccctcaaagcagccaataaggctttactaaaaagactatccacgcagtagagatttagtcaaaatattccaatagcaattgtttcctgcctgcttgaccttcgtcagccactcactgtataaatatcgcaccacgccctttgcaggcttacagagcttgtattacgtactaacaaggcacacacagtaccctgtgttcaccggccctgcacaaaactcaagcagttattactaac

SEQ ID NO: 16 - DNA, 2242 bp, Sorghum propinquum
  misc_feature (1666)..(1670): N=1, 2, 3, 4, or 5 nucleotides in length
ctatgctccaatggacgctgctcgattagattcaatggctgataaaaaggtttcatgactggtacatgagagcagtgcatgctagcctccatggaatcagagttgatataccaacagacatgtttgctactggtaacaaaaaaagcaagacatttgttacctttgaggacatgcacttgttattgaactataggcggcttgacgtccaactcataacaatctggtgcctgtaagtatcactcatgcacacacaattattatatattaatatgtagtgtgaaactctaatatgtagatgttgtctgtagtttgcaagatcacgagcagatgtcattattatctgccggatcgatggtcggttatctgagccctatcaagttacaagaaaatatgaacaaattcgtattatcaaaggaagatagagcaaagatagaggaagacaaaacaccaggataattatgccatctatcttggtagatcaatgctgaggtataaatatagggattttatattggcaccatacaacattaggtaagcttgacttcatatacgtatttcaaattatcgtgtaaacaatatacatgtgtcgctcactcatttattcatgcagtgaccattggattgttttttatatttatcccttcgaagggaaggtgcttgtcctagactctttacatgttcctcccgagaagtatcaaccattcttggttcaattagaaaggtgagccaacatgaaaccacatgcgtacttatataaattagagtttcaaaataactttagtgatttaggttcgatatctacggggcatggcggttttataagaaacaaaagggacctgtcgacgctgcacgctcagatcctaggatcccattgatgatacaacaccactatccggtaagttttctgaacacatttcatcatataaataatacataaagcatggcaaatttagaataatccgttgctcattatatagtgccacaagcaaccacctggatcggtctattgtgggtactatgtctgtgagtttataaggcagcggggacgttacgtcaaggacaaaaatatggtaaataatatctatgtatgaagttttctcattaaagctgcaaaattatatattgaacatgtgtcaatcatgcttttaaactttattttcagccgaaaaagcaaggaaaagacgtgccctttacaccaaagactctggaagatatagtagcatacttgtgtggttttattatgagagaaataatttcaagtgacagtgcatattttgatcatgagggcgatttagcaagtgataaatttagagtgctgacagacatagcaggtctaaatctgaagcgaaacgacatgtaaacattgtatggttgtgcggataacatgcattgacgtgtatatatataattttatggttgatgtttgatttgtttacaattctataatatatatatgtggtgtatgtatgatgttgtgtgtgtatatatatatatatatatatatatatatatatatatatatatatatatatatatatataatgtttagcactgtgtttggtgggaaaaattaaaatttgaaatatatataaaaaattatttacacagacagtgtagtgtgagctgcctgtgtaaaaatacatttatacaggcggctcaccttgtnnnnncaggcggtgctaaaagcatcttcacaggcggccaagcccaccgcctgtaccaggggtcagtacaaaatggaccacagtacaggcggggctgtgcgagccgcctgtgaaaacataattttcacaggcggctcgcacagccccgcctgtactgtggtccattttgtactgacccctggtacaggcggtgggcttggccgcctgtgaagatgcttttagcaccgcctgtaaaaatgttttttgtagcagtgtttttcttattagtagtatcttttatactaattaagattcaataaaaattcaccatgacatccccattgccaagagaatatttcgccgcccctcaaagcagccaataaggctttactaaaaagactatccacgcagtagagatttagtcaaaatattccaatagcaattgtttcctgcctgcttgaccttcgtcagccactcactgtataaatatcgcaccacgccctttgcaggcttacagagcttgtattacgtactaacaaggcacacacagtaccctgtgttcaccggccctgcacaaaactcaagcagttattactaac

SEQ ID NO: 17 - DNA, 5622 bp, Sorghum propinquum
  misc_feature (5349)..(5349): n is a, c, g, or t
cactataacacaacatggctttgccgacacttccaactatcggcaaagggtacctttaccgacacttaacgtctcacgaaaggttttgccgacaattttcaaacagtcgcggtagaagcagttggcgaaacttttgccgacagttaaaggcatcgccgacacattttctgtagtcaaatggcatacctacgccgacagttgaactttcaccgacagtgaaccctttgccgacagtttggacctacgccgacagtttggaccttttccgacagttggtatgttagcgaaaccgtttctagggtgtttcataaaccatgccttgtccaacagtagaagtgtcggcaaaactatattgctaggatgtagatacaatttaaatattttaataaatacacatcacattgattgagcaaaatcacatggtctgttttcactaaaactgtcagaggtacactccagtactaccagtacgtcgcccgcacagtggccaaggattttactgctactgttgattaacataagcacttgcgactttccctaaaatcttttataaaacaacggccgcaataatattgaactattttttttctagtaccaaaattagaatttgatccctcacctcattacatccatagtaacatgaccagatatatatggacaggatgggatcactcagcgagcagatacactgagcgattcataatcagattttttaatttcttctagtgaagtggggttttcctagtcttttaacattcaaaatttagtacaaactttccctagtaaatgccttctagtaaagatttcctagtattttgactagcgatagtgttttattactaattaaaaacattagaagaactccatttagtgattggttgtttggattagtcttctcacgttagacctatatatgcaggacaactcaagccagcataaatatatgaaatatcttggtgtttgtttgtctgacacaggcaaccgcgtttggtataaatgtgttttcttgtttacattttaccatctatagtcatctcaatgttatatagtagaggcttcatgtttgtagtagataaggtagagaattgagaatattttatttttgtgcgaccatcaattttatgtaatctgcattgtctaatgctttatttgacatttgaaactacttaatttgacagttatgcaggtccgcatgatcctatgaaagcaattaattagtacgggtaaactgcactacacaagtttgctagtactattctattaaccgacctgtcaatattaccttaagttactgatttcaattagaatctaacacattcaggaaaagaagtttcactagtacaaaaatcattttcgttggcacgttgttttttttttcacaggcagttcacaatatcatggtgctagtagaaaaatttcaacgggcccaacaagagaaccgccaggcggtcttcttaattcaaccgcctgtgtaaactttccatttacataggcggcttacgataaaaaccgtgtgtataaataccattaacacaggcagtcgagttacgacaaccgcctgtgtaaatgtgtctttttacacaggcggtttgtatagagggccgcctgtgctaatatatttacacaggctatgagccgcctgtgttaagtcttctataaatacccttcgtccacctccagacaagaacagttactcccatgagctctgcacactggcggaccagacgattccagtttccaaggggggaggttttgattttcatttctttggtgagaaacttccaaaaggttagttagtgccattgatgctattttttaagcgattctttggttcaattcttgtattggaggtgctctagatctagagttcatcatgcattcttgcttagggttagagttcatagggcaaaaagagagagatttagctaaatttttatgtaaattcatagtaaattgtaaaaattaaaaaaaataaaaaataaatactttttagaattcttgtgagtagatctatacaatagagtaatgatgaggatattttgaagtttataattttgattcagttttagcttttcttttttcagatgaattagactttataaactcaaacattaaaatgttgaaaatcataaaatggcaaataaatactttttcaaatctttgtgcataaatacttcatagaaatccttgaattattcctaaattttatacaattgtttcttataattatgaaaatgagtttaaacaattatttaaattccataaattgtaactccgtaaggtgtaggttttcatctctgtttaatagaaggaggttagtatcttagttaagtctgttttcgggggttatattagttttgtttttagattgacctacattaattgttcttaactaattacagctaaatatggagaggtcattatggatgtacaacttatcaagattggacctatcatatgtagtgcaggtccaaaaatttattgatgtcgcaaagatacatgctcgcagaacaaaggcgaagcacatatgttgtccatgcgcagactgcaaaaatattatggtatttgacaatgtagaagcaattacttcccatctggtttgaagaggatttatggaggactacttgatttggacaaaacatggtgagggtagttttgcaccttatatgcggacaactgacaacactgcaactaacatcaatgtggagggtccaatgccacctctcaatgaatttcatgctatgccagatgttaatgaaactcatacgtctgatgtcaatgaaactcagcatgctaacacagatgttgttgaagatgcagatttcttagaggcaataatgaaccgttgtgcggatccatcaatattcttcatgaagggaatgaaagcattgaagaaggcagcagaggacactttgtacgacgagtcaaaaggttgtaccaaacaatggtcgacattatgtgttgttcttcagtttttgacgatgaaggctagacatggttggtccgatgctagcttcaatgatttcttgcgtgtacttggagaccttcttcctaaggagaacaaagtgcctgctaacacatactatgcaaagaagctagtcagtccacttacgataggtgttgagaagatccacgcatgtagaaatcattgtattctatatcgaggtgatcaatataaagacttagacagttgtccaaactgtggtgccagtaggtacaagacaaacaaagattttcgggaggaagagaatctagcctctgtttctacagggaggaagcgaaagaagacccaaacaaagactcaacaagacaagcgctcaaagcctagtagcaatgaagaagtggactattatgcattgagaagagtctccctatgagccaaaaaaggggacagcagcaggcacaactctctttctgaaaggacttggaaagcagcggacggcacggctcattgagctcgaaccgtcacagaaaaaggaagccaccgcccagtcaatagaagccatgcccccatcaaaggaagccccaagtggcgatgtacatattgaacagccatcaagtcaaccattgaccctaaaggatatcagaaagccaacgattgatgattatgtcaatgtccctagtgactatgtgcccggaaggcctatgctccaatggacgctgctcgattagattcaatggctgataaaaaggtttcatgactggtacatgagagcagtgcatgctagcctccatggaatcagagttgatataccaacagacatgtttgctactggtaacaaaaaaagcaagacatttgttacctttgaggacatgcacttgttattgaactataggcggcttgacgtccaactcataacaatctggtgcctgtaagtatcactcatgcacacacaattattatatattaatatgtagtgtgaaactctaatatgtagatgttgtctgtagtttgcaagatcacgagcagatgtcattattatctgccggatcgatggtcggttatctgagccctatcaagttacaagaaaatatgaacaaattcgtattatcaaaggaagatagagcaaagatagaggaagacaaaacaccaggataattatgccatctatcttggtagatcaatgctgaggtataaatatagggattttatattggcaccatacaacattaggtaagcttgacttcatatacgtatttcaaattatcgtgtaaacaatatacatgtgtcgctcactcatttattcatgcagtgaccattggattgttttttatatttatcccttcgaagggaaggtgcttgtcctagactctttacatgttcctcccgagaagtatcaaccattcttggttcaattagaaaggtgagccaacatgaaaccacatgcgtacttatataaattagagtttcaaaataactttagtgatttaggttcgatatctacggggcatggcggttttataagaaacaaaagggacctgtcgacgctgcacgctcagatcctaggatcccattgatgatacaacaccactatccggtaagttttctgaacacatttcatcatataaataatacataaagcatggcaaatttagaataatccgttgctcattatatagtgccacaagcaaccacctggatcggtctattgtgggtactatgtctgtgagtttataaggcagcggggacgttacgtcaaggacaaaaatatggtaaataatatctatgtatgaaagttttctcattaaagctgcaaaattatatattgaacatgtgtcaatcatgcttttaaactttattttcagccgaaaaagcaaggaaaagacgtgccctttacaccaaagactctggaagatatagtagcatacttgtgtggttttattatgagagaaataatttcaagtgacagtgcatattttgatcatgagggcgatttagcaagtgataaatttagagtgctgacagacatagcaggtctaaatctgaagcgaaacgacatgtaaacattgtatggttgtgcggataacatgcattgacgtgtatatatataattttatggttgatgtttgatttgtttacaattctataatatatatatgtggtgtatgtatgatgttgtgtgtgtatatatatatatatatatatatatatatatatatatatatatatatatatatatatataatgtttagcactgtgtttggtgggaaaaattaaaatttgaaatatatataaaaaattatttacacagacagtgtacgtgtcgagcgtcgtcctgtgctatacaaatacattctaacaggcggctcgccttgtccaccggtcggttaaaaatacatttccacacnggcctggctgggagagccgcctgtgaaaacataattttcacaggcggctcgcacagccccgcctgtactgtggtccattttgtactgacccctggtacaggcggtgggcttggccgcctgtgaagatgcttttagcaccgcctgtaaaaatgttttttgtagcagtgtttttcttattagtagtatcttttatactaattaagattcaataaaaattcaccatgacatccccattgccaagagaatatttcgccgcccctcaaagcagccaat

SEQ ID NO: 18 - DNA, 5667 bp, Sorghum propinquum
  misc_feature (5310)..(5313): N=1, 2, 3, 4, or 5 nucleotides in length
cactataacacaacatggctttgccgacacttccaactatcggcaaagggtacctttaccgacacttaacgtctcacgaaaggttttgccgacaattttcaaacagtcgcggtagaagcagttggcgaaacttttgccgacagttaaaggcatcgccgacacattttctgtagtcaaatggcatacctacgccgacagttgaactttcaccgacagtgaaccctttgccgacagtttggacctacgccgacagtttggaccttttccgacagttggtatgttagcgaaaccgtttctagggtgtttcataaaccatgccttgtccaacagtagaagtgtcggcaaaactatattgctaggatgtagatacaatttaaatattttaataaatacacatcacattgattgagcaaaatcacatggtctgttttcactaaaactgtcagaggtacactccagtactaccagtacgtcgcccgcacagtggccaaggattttactgctactgttgattaacataagcacttgcgactttccctaaaatcttttataaaacaacggccgcaataatattgaactattttttttctagtaccaaaattagaatttgatccctcacctcattacatccatagtaacatgaccagatatatatggacaggatgggatcactcagcgagcagatacactgagcgattcataatcagattttttaatttcttctagtgaagtggggttttcctagtcttttaacattcaaaatttagtacaaactttccctagtaaatgccttctagtaaagatttcctagtattttgactagcgatagtgttttattactaattaaaaacattagaagaactccatttagtgattggttgtttggattagtcttctcacgttagacctatatatgcaggacaactcaagccagcataaatatatgaaatatcttggtgtttgtttgtctgacacaggcaaccgcgtttggtataaatgtgttttcttgtttacattttaccatctatagtcatctcaatgttatatagtagaggcttcatgtttgtagtagataaggtagagaattgagaatattttatttttgtgcgaccatcaattttatgtaatctgcattgtctaatgctttatttgacatttgaaactacttaatttgacagttatgcaggtccgcatgatcctatgaaagcaattaattagtacgggtaaactgcactacacaagtttgctagtactattctattaaccgacctgtcaatattaccttaagttactgatttcaattagaatctaacacattcaggaaaagaagtttcactagtacaaaaatcattttcgttggcacgttgttttttttttcacaggcagttcacaatatcatggtgctagtagaaaaatttcaacgggcccaacaagagaaccgccaggcggtcttcttaattcaaccgcctgtgtaaactttccatttacataggcggcttacgataaaaaccgtgtgtataaataccattaacacaggcagtcgagttacgacaaccgcctgtgtaaatgtgtctttttacacaggcggtttgtatagagggccgcctgtgctaatatatttacacaggctatgagccgcctgtgttaagtcttctataaatacccttcgtccacctccagacaagaacagttactcccatgagctctgcacactggcggaccagacgattccagtttccaaggggggaggttttgattttcatttctttggtgagaaacttccaaaaggttagttagtgccattgatgctattttttaagcgattctttggttcaattcttgtattggaggtgctctagatctagagttcatcatgcattcttgcttagggttagagttcatagggcaaaaagagagagatttagctaaatttttatgtaaattcatagtaaattgtaaaaattaaaaaaaataaaaaataaatactttttagaattcttgtgagtagatctatacaatagagtaatgatgaggatattttgaagtttataattttgattcagttttagcttttcttttttcagatgaattagactttataaactcaaacattaaaatgttgaaaatcataaaatggcaaataaatactttttcaaatctttgtgcataaatacttcatagaaatccttgaattattcctaaattttatacaattgtttcttataattatgaaaatgagtttaaacaattatttaaattccataaattgtaactccgtaaggtgtaggttttcatctctgtttaatagaaggaggttagtatcttagttaagtctgttttcgggggttatattagttttgtttttagattgacctacattaattgttcttaactaattacagctaaatatggagaggtcattatggatgtacaacttatcaagattggacctatcatatgtagtgcaggtccaaaaatttattgatgtcgcaaagatacatgctcgcagaacaaaggcgaagcacatatgttgtccatgcgcagactgcaaaaatattatggtatttgacaatgtagaagcaattacttcccatctggtttgaagaggatttatggaggactacttgatttggacaaaacatggtgagggtagttttgcaccttatatgcggacaactgacaacactgcaactaacatcaatgtggagggtccaatgccacctctcaatgaatttcatgctatgccagatgttaatgaaactcatacgtctgatgtcaatgaaactcagcatgctaacacagatgttgttgaagatgcagatttcttagaggcaataatgaaccgttgtgcggatccatcaatattcttcatgaagggaatgaaagcattgaagaaggcagcagaggacactttgtacgacgagtcaaaaggttgtaccaaacaatggtcgacattatgtgttgttcttcagtttttgacgatgaaggctagacatggttggtccgatgctagcttcaatgatttcttgcgtgtacttggagaccttcttcctaaggagaacaaagtgcctgctaacacatactatgcaaagaagctagtcagtccacttacgataggtgttgagaagatccacgcatgtagaaatcattgtattctatatcgaggtgatcaatataaagacttagacagttgtccaaactgtggtgccagtaggtacaagacaaacaaagattttcgggaggaagagaatctagcctctgtttctacagggaggaagcgaaagaagacccaaacaaagactcaacaagacaagcgctcaaagcctagtagcaatgaagaagtggactattatgcattgagaagagtctccctatgagccaaaaaaggggacagcagcaggcacaactctctttctgaaaggacttggaaagcagcggacggcacggctcattgagctcgaaccgtcacagaaaaaggaagccaccgcccagtcaatagaagccatgcccccatcaaaggaagccccaagtggcgatgtacatattgaacagccatcaagtcaaccattgaccctaaaggatatcagaaagccaacgattgatgattatgtcaatgtccctagtgactatgtgcccggaaggcctatgctccaatggacgctgctcgattagattcaatggctgataaaaaggtttcatgactggtacatgagagcagtgcatgctagcctccatggaatcagagttgatataccaacagacatgtttgctactggtaacaaaaaaagcaagacatttgttacctttgaggacatgcacttgttattgaactataggcggcttgacgtccaactcataacaatctggtgcctgtaagtatcactcatgcacacacaattattatatattaatatgtagtgtgaaactctaatatgtagatgttgtctgtagtttgcaagatcacgagcagatgtcattattatctgccggatcgatggtcggttatctgagccctatcaagttacaagaaaatatgaacaaattcgtattatcaaaggaagatagagcaaagatagaggaagacaaaacaccaggataattatgccatctatcttggtagatcaatgctgaggtataaatatagggattttatattggcaccatacaacattaggtaagcttgacttcatatacgtatttcaaattatcgtgtaaacaatatacatgtgtcgctcactcatttattcatgcagtgaccattggattgttttttatatttatcccttcgaagggaaggtgcttgtcctagactctttacatgttcctcccgagaagtatcaaccattcttggttcaattagaaaggtgagccaacatgaaaccacatgcgtacttatataaattagagtttcaaaataactttagtgatttaggttcgatatctacggggcatggcggttttataagaaacaaaagggacctgtcgacgctgcacgctcagatcctaggatcccattgatgatacaacaccactatccggtaagttttctgaacacatttcatcatataaataatacataaagcatggcaaatttagaataatccgttgctcattatatagtgccacaagcaaccacctggatcggtctattgtgggtactatgtctgtgagtttataaggcagcggggacgttacgtcaaggacaaaaatatggtaaataatatctatgtatgaagttttctcattaaagctgcaaaattatatattgaacatgtgtcaatcatgcttttaaactttattttcagccgaaaaagcaaggaaaagacgtgccctttacaccaaagactctggaagatatagtagcatacttgtgtggttttattatgagagaaataatttcaagtgacagtgcatattttgatcatgagggcgatttagcaagtgataaatttagagtgctgacagacatagcaggtctaaatctgaagcgaaacgacatgtaaacattgtatggttgtgcggataacatgcattgacgtgtatatatataattttatggttgatgtttgatttgtttacaattctataatatatatatgtggtgtatgtatgatgttgtgtgtgtatatatatatatatatatatatatatatatatatatatatatatatatatatatatataatgtttagcactgtgtttggtgggaaaaattaaaatttgaaatatatataaaaaattatttacacagacagtgtagtgtgagctgcctgtgtaaaaatacatttatacaggcggctcaccttgtcnnnncaggcggtgctaaaagcatcttcacaggcggccaagcccaccgcctgtaccaggggtcagtacaaaatggaccacagtacaggcggggctgtgcgagccgcctgtgaaaacataattttcacaggcggctcgcacagccccgcctgtactgtggtccattttgtactgacccctggtacaggcggtgggcttggccgcctgtgaagatgcttttagcaccgcctgtaaaaatgttttttgtagcagtgtttttcttattagtagtatcttttatactaattaagattcaataaaaattcaccatgacatccccattgccaagagaatatttcgccgcccctcaaagcagccaat

SEQ ID NO: 19 - DNA, 4186 bp, Sorghum propinquum
  misc_feature (4016)..(4016): n is a, c, g, or t
cactagtacaaaaatcattttcgttggcacgttgttttttttttcacaggcagttcacaatatcatggtgctagtagaaaaatttcaacgggcccaacaagagaaccgccaggcggtcttcttaattcaaccgcctgtgtaaactttccatttacataggcggcttacgataaaaaccgtgtgtataaataccattaacacaggcagtcgagttacgacaaccgcctgtgtaaatgtgtctttttacacaggcggtttgtatagagggccgcctgtgctaatatatttacacaggctatgagccgcctgtgttaagtcttctataaatacccttcgtccacctccagacaagaacagttactcccatgagctctgcacactggcggaccagacgattccagtttccaaggggggaggttttgattttcatttctttggtgagaaacttccaaaaggttagttagtgccattgatgctattttttaagcgattctttggttcaattcttgtattggaggtgctctagatctagagttcatcatgcattcttgcttagggttagagttcatagggcaaaaagagagagatttagctaaatttttatgtaaattcatagtaaattgtaaaaattaaaaaaaataaaaaataaatactttttagaattcttgtgagtagatctatacaatagagtaatgatgaggatattttgaagtttataattttgattcagttttagcttttcttttttcagatgaattagactttataaactcaaacattaaaatgttgaaaatcataaaatggcaaataaatactttttcaaatctttgtgcataaatacttcatagaaatccttgaattattcctaaattttatacaattgtttcttataattatgaaaatgagtttaaacaattatttaaattccataaattgtaactccgtaaggtgtaggttttcatctctgtttaatagaaggaggttagtatcttagttaagtctgttttcgggggttatattagttttgtttttagattgacctacattaattgttcttaactaattacagctaaatatggagaggtcattatggatgtacaacttatcaagattggacctatcatatgtagtgcaggtccaaaaatttattgatgtcgcaaagatacatgctcgcagaacaaaggcgaagcacatatgttgtccatgcgcagactgcaaaaatattatggtatttgacaatgtagaagcaattacttcccatctggtttgaagaggatttatggaggactacttgatttggacaaaacatggtgagggtagttttgcaccttatatgcggacaactgacaacactgcaactaacatcaatgtggagggtccaatgccacctctcaatgaatttcatgctatgccagatgttaatgaaactcatacgtctgatgtcaatgaaactcagcatgctaacacagatgttgttgaagatgcagatttcttagaggcaataatgaaccgttgtgcggatccatcaatattcttcatgaagggaatgaaagcattgaagaaggcagcagaggacactttgtacgacgagtcaaaaggttgtaccaaacaatggtcgacattatgtgttgttcttcagtttttgacgatgaaggctagacatggttggtccgatgctagcttcaatgatttcttgcgtgtacttggagaccttcttcctaaggagaacaaagtgcctgctaacacatactatgcaaagaagctagtcagtccacttacgataggtgttgagaagatccacgcatgtagaaatcattgtattctatatcgaggtgatcaatataaagacttagacagttgtccaaactgtggtgccagtaggtacaagacaaacaaagattttcgggaggaagagaatctagcctctgtttctacagggaggaagcgaaagaagacccaaacaaagactcaacaagacaagcgctcaaagcctagtagcaatgaagaagtggactattatgcattgagaagagtctccctatgagccaaaaaaggggacagcagcaggcacaactctctttctgaaaggacttggaaagcagcggacggcacggctcattgagctcgaaccgtcacagaaaaaggaagccaccgcccagtcaatagaagccatgcccccatcaaaggaagccccaagtggcgatgtacatattgaacagccatcaagtcaaccattgaccctaaaggatatcagaaagccaacgattgatgattatgtcaatgtccctagtgactatgtgcccggaaggcctatgctccaatggacgctgctcgattagattcaatggctgataaaaaggtttcatgactggtacatgagagcagtgcatgctagcctccatggaatcagagttgatataccaacagacatgtttgctactggtaacaaaaaaagcaagacatttgttacctttgaggacatgcacttgttattgaactataggcggcttgacgtccaactcataacaatctggtgcctgtaagtatcactcatgcacacacaattattatatattaatatgtagtgtgaaactctaatatgtagatgttgtctgtagtttgcaagatcacgagcagatgtcattattatctgccggatcgatggtcggttatctgagccctatcaagttacaagaaaatatgaacaaattcgtattatcaaaggaagatagagcaaagatagaggaagacaaaacaccaggataattatgccatctatcttggtagatcaatgctgaggtataaatatagggattttatattggcaccatacaacattaggtaagcttgacttcatatacgtatttcaaattatcgtgtaaacaatatacatgtgtcgctcactcatttattcatgcagtgaccattggattgttttttatatttatcccttcgaagggaaggtgcttgtcctagactctttacatgttcctcccgagaagtatcaaccattcttggttcaattagaaaggtgagccaacatgaaaccacatgcgtacttatataaattagagtttcaaaataactttagtgatttaggttcgatatctacggggcatggcggttttataagaaacaaaagggacctgtcgacgctgcacgctcagatcctaggatcccattgatgatacaacaccactatccggtaagttttctgaacacatttcatcatataaataatacataaagcatggcaaatttagaataatccgttgctcattatatagtgccacaagcaaccacctggatcggtctattgtgggtactatgtctgtgagtttataaggcagcggggacgttacgtcaaggacaaaaatatggtaaataatatctatgtatgaaagttttctcattaaagctgcaaaattatatattgaacatgtgtcaatcatgcttttaaactttattttcagccgaaaaagcaaggaaaagacgtgccctttacaccaaagactctggaagatatagtagcatacttgtgtggttttattatgagagaaataatttcaagtgacagtgcatattttgatcatgagggcgatttagcaagtgataaatttagagtgctgacagacatagcaggtctaaatctgaagcgaaacgacatgtaaacattgtatggttgtgcggataacatgcattgacgtgtatatatataattttatggttgatgtttgatttgtttacaattctataatatatatatgtggtgtatgtatgatgttgtgtgtgtatatatatatatatatatatatatatatatatatatatatatatatatatatatatataatgtttagcactgtgtttggtgggaaaaattaaaatttgaaatatatataaaaaattatttacacagacagtgtacgtgtcgagcgtcgtcctgtgctatacaaatacattctaacaggcggctcgccttgtccaccggtcggttaaaaatacatttccacacnggcctggctgggagagccgcctgtgaaaacataattttcacaggcggctcgcacagccccgcctgtactgtggtccattttgtactgacccctggtacaggcggtgggcttggccgcctgtgaagatgcttttagcaccgcctgtaaaaatgttttttgtagcagtgttt

SEQ ID NO: 20 - DNA, 4231 bp, Sorghum propinquum
  misc_feature (3977)..(3980): N=1, 2, 3, 4, or 5 nucleotides in length
cactagtacaaaaatcattttcgttggcacgttgttttttttttcacaggcagttcacaatatcatggtgctagtagaaaaatttcaacgggcccaacaagagaaccgccaggcggtcttcttaattcaaccgcctgtgtaaactttccatttacataggcggcttacgataaaaaccgtgtgtataaataccattaacacaggcagtcgagttacgacaaccgcctgtgtaaatgtgtctttttacacaggcggtttgtatagagggccgcctgtgctaatatatttacacaggctatgagccgcctgtgttaagtcttctataaatacccttcgtccacctccagacaagaacagttactcccatgagctctgcacactggcggaccagacgattccagtttccaaggggggaggttttgattttcatttctttggtgagaaacttccaaaaggttagttagtgccattgatgctattttttaagcgattctttggttcaattcttgtattggaggtgctctagatctagagttcatcatgcattcttgcttagggttagagttcatagggcaaaaagagagagatttagctaaatttttatgtaaattcatagtaaattgtaaaaattaaaaaaaataaaaaataaatactttttagaattcttgtgagtagatctatacaatagagtaatgatgaggatattttgaagtttataattttgattcagttttagcttttcttttttcagatgaattagactttataaactcaaacattaaaatgttgaaaatcataaaatggcaaataaatactttttcaaatctttgtgcataaatacttcatagaaatccttgaattattcctaaattttatacaattgtttcttataattatgaaaatgagtttaaacaattatttaaattccataaattgtaactccgtaaggtgtaggttttcatctctgtttaatagaaggaggttagtatcttagttaagtctgttttcgggggttatattagttttgtttttagattgacctacattaattgttcttaactaattacagctaaatatggagaggtcattatggatgtacaacttatcaagattggacctatcatatgtagtgcaggtccaaaaatttattgatgtcgcaaagatacatgctcgcagaacaaaggcgaagcacatatgttgtccatgcgcagactgcaaaaatattatggtatttgacaatgtagaagcaattacttcccatctggtttgaagaggatttatggaggactacttgatttggacaaaacatggtgagggtagttttgcaccttatatgcggacaactgacaacactgcaactaacatcaatgtggagggtccaatgccacctctcaatgaatttcatgctatgccagatgttaatgaaactcatacgtctgatgtcaatgaaactcagcatgctaacacagatgttgttgaagatgcagatttcttagaggcaataatgaaccgttgtgcggatccatcaatattcttcatgaagggaatgaaagcattgaagaaggcagcagaggacactttgtacgacgagtcaaaaggttgtaccaaacaatggtcgacattatgtgttgttcttcagtttttgacgatgaaggctagacatggttggtccgatgctagcttcaatgatttcttgcgtgtacttggagaccttcttcctaaggagaacaaagtgcctgctaacacatactatgcaaagaagctagtcagtccacttacgataggtgttgagaagatccacgcatgtagaaatcattgtattctatatcgaggtgatcaatataaagacttagacagttgtccaaactgtggtgccagtaggtacaagacaaacaaagattttcgggaggaagagaatctagcctctgtttctacagggaggaagcgaaagaagacccaaacaaagactcaacaagacaagcgctcaaagcctagtagcaatgaagaagtggactattatgcattgagaagagtctccctatgagccaaaaaaggggacagcagcaggcacaactctctttctgaaaggacttggaaagcagcggacggcacggctcattgagctcgaaccgtcacagaaaaaggaagccaccgcccagtcaatagaagccatgcccccatcaaaggaagccccaagtggcgatgtacatattgaacagccatcaagtcaaccattgaccctaaaggatatcagaaagccaacgattgatgattatgtcaatgtccctagtgactatgtgcccggaaggcctatgctccaatggacgctgctcgattagattcaatggctgataaaaaggtttcatgactggtacatgagagcagtgcatgctagcctccatggaatcagagttgatataccaacagacatgtttgctactggtaacaaaaaaagcaagacatttgttacctttgaggacatgcacttgttattgaactataggcggcttgacgtccaactcataacaatctggtgcctgtaagtatcactcatgcacacacaattattatatattaatatgtagtgtgaaactctaatatgtagatgttgtctgtagtttgcaagatcacgagcagatgtcattattatctgccggatcgatggtcggttatctgagccctatcaagttacaagaaaatatgaacaaattcgtattatcaaaggaagatagagcaaagatagaggaagacaaaacaccaggataattatgccatctatcttggtagatcaatgctgaggtataaatatagggattttatattggcaccatacaacattaggtaagcttgacttcatatacgtatttcaaattatcgtgtaaacaatatacatgtgtcgctcactcatttattcatgcagtgaccattggattgttttttatatttatcccttcgaagggaaggtgcttgtcctagactctttacatgttcctcccgagaagtatcaaccattcttggttcaattagaaaggtgagccaacatgaaaccacatgcgtacttatataaattagagtttcaaaataactttagtgatttaggttcgatatctacggggcatggcggttttataagaaacaaaagggacctgtcgacgctgcacgctcagatcctaggatcccattgatgatacaacaccactatccggtaagttttctgaacacatttcatcatataaataatacataaagcatggcaaatttagaataatccgttgctcattatatagtgccacaagcaaccacctggatcggtctattgtgggtactatgtctgtgagtttataaggcagcggggacgttacgtcaaggacaaaaatatggtaaataatatctatgtatgaagttttctcattaaagctgcaaaattatatattgaacatgtgtcaatcatgcttttaaactttattttcagccgaaaaagcaaggaaaagacgtgccctttacaccaaagactctggaagatatagtagcatacttgtgtggttttattatgagagaaataatttcaagtgacagtgcatattttgatcatgagggcgatttagcaagtgataaatttagagtgctgacagacatagcaggtctaaatctgaagcgaaacgacatgtaaacattgtatggttgtgcggataacatgcattgacgtgtatatatataattttatggttgatgtttgatttgtttacaattctataatatatatatgtggtgtatgtatgatgttgtgtgtgtatatatatatatatatatatatatatatatatatatatatatatatatatatatatataatgtttagcactgtgtttggtgggaaaaattaaaatttgaaatatatataaaaaattatttacacagacagtgtagtgtgagctgcctgtgtaaaaatacatttatacaggcggctcaccttgtcnnnncaggcggtgctaaaagcatcttcacaggcggccaagcccaccgcctgtaccaggggtcagtacaaaatggaccacagtacaggcggggctgtgcgagccgcctgtgaaaacataattttcacaggcggctcgcacagccccgcctgtactgtggtccattttgtactgacccctggtacaggcggtgggcttggccgcctgtgaagatgcttttagcaccgcctgtaaaaatgttttttgtagcagtgttt

SEQ ID NO: 21 - DNA, 423 bp, Sorghum propinquum
  misc_feature (3977)..(3980): N=1, 2, 3, 4, or 5 nucleotides in length
cactataacacaacatggctttgccgacacttccaactatcggcaaagggtacctttaccgacacttaacgtctcacgaaaggttttgccgacaattttcaaacagtcgcggtagaagcagttggcgaaacttttgccgacagttaaaggcatcgccgacacattttctgtagtcaaatggcatacctacgccgacagttgaactttcaccgacagtgaaccctttgccgacagtttggacctacgccgacagtttggaccttttccgacagttggtatgttagcgaaaccgtttctagggtgtttcataaaccatgccttgtccaacagtagaagtgtcggcaaaactatattgctaggatgtagatacaatttaaatattttaataaatacacatcacattgattgagcaaaatcacatgg

SEQ ID NO: 22 - DNA, 691 bp, Sorgum propinquum
ttccacctgggcagggaaaacggtttattatgttcctctttaatttatctatcgtggcactataacacaacatggctttgccgacacttccaactatcggcaaagggtacctttgccgacacttaacgtctcacgaaaggttttgccgacaattttcaaacagtcgcggtagaagcagtcggcgaaacttttgccgacagttaaaggaggacacattttctgtagtcaaatgggcatgcctcccgcgttgactttcaccgacagtgaaccctttgccgacagtttggacctacgccgacagtttggatcttttccgacagttggtatgttagcgaaaccgtttctagggtgtttcataaaccatgccttgtccaacagtagaagtgtcggcaaaactatattgcagatagtagggtgtagatacaatttaaatattttaataaatacacatcacattgatcgagcaaaatcacatggtctgttttcactaaaactgtcataggtacactccagtactaccagtacgtcgcccgcacatagtggccaaggattttactgctactgttgattaacataagcacttgcgactttccctaaaatcttttataaaacaacggccgcaataatattgaactatttttgttctagtaccaaaattagaatttgatccctcacctcattacatccatag

SEQ ID NO: 23 - DNA, 423 bp, Sorghum propinquum
tggcactataacacaacatggctttgccgacacttccaactatcggcaaagggtacctttgccgacacttaacgtctcacgaaaggttttgccgacaattttcaaacagtcgcggtagaagcagtcggcgaaacttttgccgacagttaaaggaggacacattttctgtagtcaaatgggcatgcctcccgcgttgactttcaccgacagtgaaccctttgccgacagtttggacctacgccgacagtttggatcttttccgacagttggtatgttagcgaaaccgtttctagggtgtttcataaaccatgccttgtccaacagtagaagtgtcggcaaaactatattgcagatagtagggtgtagatacaatttaaatattttaataaatacacatcacattgatcgagcaaaatcacatgg

SEQ ID NO: 24 - DNA, 6 bp, Sorghum propinquum
gccaat

SEQ ID NO: 25 - DNA, 9 bp, Artificial Sequence
  synthetic consensus CAAT Box sequence
ggccaatct

SEQ ID NO: 26 - DNA, 321 bp, Sorgum propinquum
ttcttattagtagtatcttttatactaattaagattcaataaaaattcaccatgacatccccattgccaagagaatatttcgccgcccctcaaagcagccaataaggctttactaaaaagactatccacgcagtagagatttagtcaaaatattccaatagcaattgtttcctgcctgcttgaccttcgtcagccactcactgtataaatatcgcaccacgccctttgcaggcttacagagcttgtattacgtactaacaaggcacacacagtaccctgtgttcaccggccctgcacaaaactcaagcagttattactaac

SEQ ID NO: 27 - DNA, 2392 bp, Sorghum bicolor
aaaagaaaagtgagcacaccacgacctatcatcagctcatggtcagctctacaaacttatagattgcatcgagatctaagactcaggtacaaatcatgtcaacatctaatggtttagaaaatgaaaaaagttttgagtttcaaaatatgatacttgaaattaacatttgaactttttagcaagatctgaaaataaaaaattcaactaaaaaatttatagatcatgttaacattgatataatcgcttccaatcgcctcccatcgcttcagctagaaaactttttttctcgatttaattaatgaaatagtaataacgtcattgtacaagattctttcaaaccccaacccctatcatcgacggtgagggctcctataatatgcactagtggacgccgggtgggtggaacctaagaagattttaaaaaaaaaattaagaagaagatttttatctaactaactatatatagtacttatatcatacactatactattcaaaatattattttcacaattatgaatttacccttttactctttattaaaaaaatatgaataaagaattatcacgcctctatttagggtcctaatccccataatttaagaggcgatgagaggcgatgtgacatctatggcccaccgaccaaagacacaactatcgcctcccatcaccttgcttctatcgcctctcatagcttttcatattctaggtccaccggccatagacacaccaatcgcttatcatcgccttttccaaccattgtaaaaatattcataattttgatataaaatttgtcttcacttgagtatgggaaaaaaattatacataatgttttcgtgtgagaatttacaggaatgaacccttaagatgtccaaatgtaaatgaccctatttattaagaggagcggatctataggcctggctctgaaaatggattatggattggagatactaaatttaagggcctatcttcgcacataacatctatagttcctaaataattttttattgtagtagtagaacttttctccctgtaaaccataaaccaagttgacgctgggctttattttgcgacacagaacaccaaattggtggctatgaactcttccacctgggcagggaaaacggtttattatgttcctctttaatttatctatcgtggtctgttttcactaaaactgtcatattgctacactccagtactaccagtacgtcgcccgcacatagtggccaaggattttactgctactgttgattaacataagcacttgcgactttccctaacatcttttataaaacaacggccgcaataatattgaactgtttttttctagtaccaaaaatagaatttgatccctcacctcattacatccatagtaacatgaccagatatatatggacaggccgggatcactcgccagcagataccctgagcgattcataaccagaatttttaattttttctagtgaagtggggttctcctagtcctttaacattcaaaatttagtacaaactttccttagtaaatgtcttctagtaaagatttcctagtgttttgatttggtagtgttttattactaattaaaaatattagaagaactccatcattttggtagtgattggttgtttggattagtcttctcacgttagacctatatatgcaggacaactcaagccagcataaatatatgaaatatcttggtgtttgtttgtctgacacaggcaaccgtgtttggtataaatgtgttttcttgtttacgttttaccatctatagtcatctcaatgtttatatagtagagacttcatgtttgtagtagataaggtagagaattgagaatattttatttttgtgcgaccatcaattttatgtaatctgcattgtctaatgctttatttgacatttgaaactacttaatttgaccgttatgcaggtccgcatgatcctatgaaagcaattaattagtacgggtactgcactacacaagtttgctagtactattctattaaccgacctgtcaatattaccttaagttactgatttcaattagaatctaacacattcaggaaaagaagttttccttattagtagtaactttttatactaattaagattcaataaaaattcaccatgacatccccattgccaagagaatatttcgccgcccctcaaagcagccaaggctttactaaaaagactatccacgcagtagagatttagtcaaaatattccaatagcaattgttttctgcctgcttgaccttcgtcagccactcactgtataaatatcgcaccacgccctttgcaggcttacagagcttgtactacgtactaacaaggcacacacaataccctgtgttcaccggccctgcacaaaactcaagcagttattactaac

SEQ ID NO: 28 - DNA, 1500 bp, Sorgum propinquum
atgcccccatcaaaggaagccccaagtggcgatgtacatattgaacagccatcaagtcaaccattgaccctaaaggatatcagaaagccaacgattgatgattatgtcaatgtccctagtgactatgtgcccggaaggcctatgctccaatggacgctgctcgattagattcaatggctgataaaaaggtttcatgactggtacatgagagcagtgcatgctagcctccatggaatcagagttgatataccaacagacatgtttgctactggtaacaaaaaaagcaagacatttgttacctttgaggacatgcacttgttattgaactataggcggcttgacgtccaactcataacaatctggtgcctgtaagtatcactcatgcacacacaattattatatattaatatgtagtgtgaaactctaatatgtagatgttgtctgtagtttgcaagatcacgagcagatgtcattattatctgccggatcgatggtcggttatctgagccctatcaagttacaagaaaatatgaacaaattcgtattatcaaaggaagatagagcaaagatagaggaagacaaaacaccaggataattatgccatctatcttggtagatcaatgctgaggtataaatatagggattttatattggcaccatacaacattaggtaagcttgacttcatatacgtatttcaaattatcgtgtaaacaatatacatgtgtcgctcactcatttattcatgcagtgaccattggattgttttttatatttatcccttcgaagggaaggtgcttgtcctagactctttacatgttcctcccgagaagtatcaaccattcttggttcaattagaaaggtgagccaacatgaaaccacatgcgtacttatataaattagagtttcaaaataactttagtgatttaggttcgatatctacggggcatggcggttttataagaaacaaaagggacctgtcgacgctgcacgctcagatcctaggatcccattgatgatacaacaccactatccggtaagttttctgaacacatttcatcatataaataatacataaagcatggcaaatttagaataatccgttgctcattatatagtgccacaagcaaccacctggatcggtctattgtgggtactatgtctgtgagtttataaggcagcggggacgttacgtcaaggacaaaaatatggtaaataatatctatgtatgaaagttttctcattaaagctgcaaaattatatattgaacatgtgtcaatcatgcttttaaactttattttcagccgaaaaagcaaggaaaagacgtgccctttacaccaaagactctggaagatatagtagcatacttgtgtggttttattatgagagaaataatttcaagtgacagtgcatattttgatcatgagggcgatttagcaagtgataaatttagagtgctgacagacatagcaggtctaaatctgaagcgaaacgacatgtaa

SEQ ID NO: 29 - DNA, 861 bp, Sorghum propinquum
atgcccccatcaaaggaagccccaagtggcgatgtacatattgaacagccatcaagtcaaccattgaccctaaaggatatcagaaagccaacgattgatgattatgtcaatgtccctagtgactatgtgcccggaaggcctatgctccaatggacgctgctcgattagattcaatggctgataaaaaggtttcatgactggtacatgagagcagtgcatgctagcctccatggaatcagagttgatataccaacagacatgtttgctactggtaacaaaaaaagcaagacatttgttacctttgaggacatgcacttgttattgaactataggcggcttgacgtccaactcataacaatctggtgcctggaccattggattgttttttatatttatcccttcgaagggaaggtgcttgtcctagactctttacatgttcctcccgagaagtatcaaccattcttggttcaattagaaagggcatggcggttttataagaaacaaaagggacctgtcgacgctgcacgctcagatcctaggatcccattgatgatacaacaccactatccgtgccacaagcaaccacctggatcggtctattgtgggtactatgtctgtgagtttataaggcagcggggacgttacgtcaaggacaaaaatatgccgaaaaagcaaggaaaagacgtgccctttacaccaaagactctggaagatatagtagcatacttgtgtggttttattatgagagaaataatttcaagtgacagtgcatattttgatcatgagggcgatttagcaagtgataaatttagagtgctgacagacatagcaggtctaaatctgaagcgaaacgacatgtaa

SEQ ID NO: 30 - DNA, 1499 bp, Sorghum propinquum
atgcccccatcaaaggaagccccaagtggcgatgtacatattgaacagccatcaagtcaaccattgaccctaaaggatatcagaaagccaacgattgatgattatgtcaatgtccctagtgactatgtgcccggaaggcctatgctccaatggacgctgctcgattagattcaatggctgataaaaaggtttcatgactggtacatgagagcagtgcatgctagcctccatggaatcagagttgatataccaacagacatgtttgctactggtaacaaaaaaagcaagacatttgttacctttgaggacatgcacttgttattgaactataggcggcttgacgtccaactcataacaatctggtgcctgtaagtatcactcatgcacacacaattattatatattaatatgtagtgtgaaactctaatatgtagatgttgtctgtagtttgcaagatcacgagcagatgtcattattatctgccggatcgatggtcggttatctgagccctatcaagttacaagaaaatatgaacaaattcgtattatcaaaggaagatagagcaaagatagaggaagacaaaacaccaggataattatgccatctatcttggtagatcaatgctgaggtataaatatagggattttatattggcaccatacaacattaggtaagcttgacttcatatacgtatttcaaattatcgtgtaaacaatatacatgtgtcgctcactcatttattcatgcagtgaccattggattgttttttatatttatcccttcgaagggaaggtgcttgtcctagactctttacatgttcctcccgagaagtatcaaccattcttggttcaattagaaaggtgagccaacatgaaaccacatgcgtacttatataaattagagtttcaaaataactttagtgatttaggttcgatatctacggggcatggcggttttataagaaacaaaagggacctgtcgacgctgcacgctcagatcctaggatcccattgatgatacaacaccactatccggtaagttttctgaacacatttcatcatataaataatacataaagcatggcaaatttagaataatccgttgctcattatatagtgccacaagcaaccacctggatcggtctattgtgggtactatgtctgtgagtttataaggcagcggggacgttacgtcaaggacaaaaatatggtaaataatatctatgtatgaagttttctcattaaagctgcaaaattatatattgaacatgtgtcaatcatgcttttaaactttattttcagccgaaaaagcaaggaaaagacgtgccctttacaccaaagactctggaagatatagtagcatacttgtgtggttttattatgagagaaataatttcaagtgacagtgcatattttgatcatgagggcgatttagcaagtgataaatttagagtgctgacagacatagcaggtctaaatctgaagcgaaacgacatgtaa

SEQ ID NO: 31 - DNA, 861 bp, Sorghum propinquum; identical to SEQ ID NO: 29

SEQ ID NO: 32 - DNA, 1539 bp, Sorghum bicolor
atgcccccatcaaaggaagccccaagtggcgatgtacatgtcaaacagccatcaagtcaaccattgaccctaaaggatatcagaaagccaacgattgatgattatgtcaatgtccccagtgactatgtgcccggaaggcctatgctccaatggacgctgcttgataagattcaatggccgataaaaaggtttcatgactggtacatgagagcagtgcatgctggcctccatgcaatcagagttgatataccagcaaacgtgtttgctactggtaacgaaaaaagcaaggcatttgttatctttgaggacatgcacttgttattgaactataggcggcttgacgtccaactcataacaatctggtgtctgtaagtaccactcatgcacacacaattattattaatatgtagtgtgaaactctaatatgtagatgttgtctgtagtttgcaagatcacgagtagaggtcattattatctaccggatcaatggtcggttatctgagccctatcaagttacaagaaaatatgcacaaatttgtattatcaaaggaagatagagcaaagatagaggaagacaaaacaccagaaaaagttgcagaagctataaaagagttgcaaagaaaatacgaggataattatgccctctaccttggtagatcaatgctgaggtataagtatagggattttatattggcaccttacaactttaggtaagcttgacttcatatacgtacttcaaataattatcgtgtaaacaatatacatgtgtcgctcactcatttattcatgcagtgaccattggattgttttttatatttatcccttcgaaaggaaggtgcttgtcctagactctttacatgttcctcccgagaagtatcaaccattcttggttcaattagaaaggtgagccaacatgaaaccacatgcgtacttatataaattagagtttcaaaacaactttagtgatttatattcgatatctacagggcatggcggttttataagaaacaaaagggaccggtcgacgccgcacgctcagatcctagggtgccattgatgatacaacaccactatccggtaagttgtccgaacacatttcatcatataaataatacataaagcatggcaaatttagaataatccgttgctcattatatagtgccacaagcaaccatctggatcggtctattgtgggtactatgtctgtgagtttataaggcagcggggacgttacgtcacggacaaaaatatggtaaataatatctatgtatgaagttttctcattaaagttgcaaaattatatattgaacatgtgtcaatcatgcttttaaactttgtttccagccaaaaaagcaaaaaaaggacgtgccctttacaccaaagactctggaagatatagtagcagacttgtgtggttttattatgagagaaataattccaagtgacggtgcatattttgatcatgagggcgatttagcaagtgataaatttagagtgctgacagacatagcaggtctaaatctgaagcgaaatgacatg

SEQ ID NO: 33 - DNA, 861 bp, Sorghum bicolor
atgcccccatcaaaggaagccccaagtggcgatgtacatgtcaaacagccatcaagtcaaccattgaccctaaaggatatcagaaagccaacgattgatgattatgtcaatgtccccagtgactatgtgcccggaaggcctatgctccaatggacgctgcttgataagattcaatggccgataaaaaggtttcatgactggtacatgagagcagtgcatgctggcctccatgcaatcagagttgatataccagcaaacgtgtttgctactggtaacgaaaaaagcaaggcatttgttatctttgaggacatgcacttgttattgaactataggcggcttgacgtccaactcataacaatctggtgtctggaccattggattgttttttatatttatcccttcgaaaggaaggtgcttgtcctagactctttacatgttcctcccgagaagtatcaaccattcttggttcaattagaaagggcatggcggttttataagaaacaaaagggaccggtcgacgccgcacgctcagatcctagggtgccattgatgatacaacaccactatccgtgccacaagcaaccatctggatcggtctattgtgggtactatgtctgtgagtttataaggcagcggggacgttacgtcacggacaaaaatatgccaaaaaagcaaaaaaaggacgtgccctttacaccaaagactctggaagatatagtagcagacttgtgtggttttattatgagagaaataattccaagtgacggtgcatattttgatcatgagggcgatttagcaagtgataaatttagagtgctgacagacatagcaggtctaaatctgaagcgaaatgacatgtaa

SEQ ID NO: 34 - protein, 286 aa, Sorghum bicolor
MPPSKEAPSGDVHVKQPSSQPLTLKDIRKPTIDDYVNVPSDYVPGRPMLQWTLLDKIQWPIKRFHDWYMRAVHAGLHAIRVDIPANVFATGNEKSKAFVIFEDMHLLLNYRRLDVQLITIWCLDHWIVFYIYPFERKVLVLDSLHVPPEKYQPFLVQLERAWRFYKKQKGPVDAARSDPRVPLMIQHHYPCHKQPSGSVYCGYYVCEFIRQRGRYVTDKNMPKKQKKDVPFTPKTLEDIVADLCGFIMREIIPSDGAYFDHEGDLASDKFRVLTDIAGLNLKRNDM

SEQ ID NO: 35 - DNA, 319 bp, Sorghum bicolor
tccttattagtagtaactttttatactaattaagattcaataaaaattcaccatgacatccccattgccaagagaatatttcgccgcccctcaaagcagccaaggctttactaaaaagactatccacgcagtagagatttagtcaaaatattccaatagcaattgttttctgcctgcttgaccttcgtcagccactcactgtataaatatcgcaccacgccctttgcaggcttacagagcttgtactacgtactaacaaggcacacacaataccctgtgttcaccggccctgcacaaaactcaagcagttattactaac

(19) United States
(12) Patent Application Publication
(10) Pub. No.: US 2014/0068815 A1
(43) Pub. Date: Mar. 6, 2014

(54) Sorghum Maturity Gene and Uses Thereof in Modulating Photoperiod Sensitivity

(71) Applicant: University of Georgia Research Foundation, Inc., Athens, GA (US)

(72) Inventors: Andrew H. Paterson, Arnoldsville, GA (US); Haibao Tang, Germantown, MD (US); Hugo E. Cuevas, Lawrenceville, GA (US)

(73) Assignee: University of Georgia Research Foundation, Inc., Athens, GA (US)

(21) Appl. No.: 14/075,844

(22) Filed: Nov. 8, 2013

Related U.S. Application Data
(63) Continuation-in-part of application No. PCT/US2012/037809, filed on May 14, 2012.
(60) Provisional application No. 61/486,024, filed on May 13, 2011.

(51) Int. Cl. C12N 15/82

(52) U.S. Cl. CPC C12N 15/827
USPC 800/285; 800/278

(57) ABSTRACT

Compositions relating to the sorghum maturity gene 1 (Ma1) and expression control sequences and methods of use thereof are provided. The compositions can be used to modulate flowering and photoperiod sensitivity in a plant. For example, methods are provided for developing genetically modified plant varieties in which flowering is accelerated, delayed or prevented. Methods are provided for treating a plant in order to delay flowering in the plant. Methods of placing a polynucleotide of interest, such a gene, under photoperiod sensitive control or photoperiod insensitive control are also provided. Screening methods are for identifying chemical agents that can modify photoperiod sensitivity are also disclosed.

### CROSS REFERENCE TO RELATED APPLICATIONS

This application is a continuation-in-part of International Application No. PCT/US2012/037809, entitled “Sorghum Maturity Gene and Uses Thereof in Modulating Photoperiod Sensitivity” by Andrew H. Paterson, Haibao Tang, and Hugo E. Cuevas, filed in the United States Receiving Office for the PCT on May 14, 2012, which claims benefit of and priority to U.S. Provisional Application No. 61/486,024, filed May 13, 2011, which is hereby incorporated herein by reference in its entirety.

### STATEMENT REGARDING FEDERALLY SPONSORED RESEARCH OR DEVELOPMENT

This invention was made with government Support under Agreement 00-35300-9215 awarded by the US Department of Agriculture. The government has certain rights in the invention.

### REFERENCE TO SEQUENCE LISTING

The Sequence Listing submitted Nov. 8, 2013 as a text file named “UGA_{—}1540_ST25.txt,” created on Nov. 8, 2013, and having a size of 140,800 bytes is hereby incorporated by reference pursuant to 37 C.F.R. §1.52(e)(5).

### FIELD OF THE INVENTION

The invention is generally related to the field of plant genetics and molecular biology, more particularly to genes involved in plant photoperiod sensitivity, and methods for modifying photoperiod sensitivity in plants.

### BACKGROUND OF THE INVENTION

Biomass yield is one of the most important attributes of a biomass or bioenergy crop designed for ligno-cellulosic conversion to biofuels or bioenergy. To maximize yield, it is essential to tailor the plants' life cycle to the agro-environments in which they are grown. The transition from vegetative to reproductive growth is a critical developmental switch and a key adaptive trait that ensures that plants set their flowers at an optimum time for pollination, seed development, and dispersal. For example, temperate environments with a long growing season allow cereal crops to exploit an extended vegetative period for resource storage. Conversely, early flowering has evolved as an adaptation to short growing seasons.

For example, once grain sorghum initiates flowering, growth of the vegetative plant (stem, leaves) decreases so that carbon and nitrogen compounds can be used for grain production. As a consequence, biomass accumulation overall decreases to some extent during the reproductive phase and largely ceases once grain filling has been completed.

In contrast, a late or non-flowering bioenergy sorghum crop grown for biomass production will continue to accumulate biomass by building larger vegetative plants until frost or adverse environmental conditions inhibit photosynthesis. It is estimated that late/non-flowering biomass sorghum will generate more than two times the biomass accumulated by grain sorghum per acre assuming reasonable growth conditions throughout the growing season.

Flowering is generally controlled by environmental factors, such as daylength. Daylength regulates flowering by a phenomenon known as photoperiod sensitivity, which allows plants to coordinate their reproduction with the environment or with other members of their species. Photoperiod sensitivity refers to the fact that some plants will not flower until they are exposed to day lengths that are less than a critical photoperiod (short day plants) or greater than a critical photoperiod (long day plants). Long day (LD) and short day (SD) plant designations refer to the day length required to induce flowering. Facultative LD or SD plants are those that show accelerated flowering in LD or SD but will eventually flower regardless of photoperiod.

Therefore, it is an object of the invention to provide a gene in sorghum responsible for genetic control of photoperiod sensitivity.

It is another object of the invention to provide late or non-flowering recombinant sorghum plants.

It is yet another object of the invention to provide methods for modifying photoperiod sensitivity in plants.

It is a further object of the invention to provide methods for imposing photoperiod sensitivity on a plant process.

### SUMMARY OF THE INVENTION

Compositions including the nucleic acid sequence of the sorghum Maturity gene 1 (Ma1), and expression control sequences thereof are disclosed. The expression control sequence can be photoperiod sensitive or photoperiod insensitive. The compositions and methods can be used to modulating flowering in plants, particularly sorghum.

Methods of using the compositions for modulating photoperiod sensitivity for flowering and other plant processes in a plant are provided. For example, methods are provided for developing genetically modified plant varieties in which flowering is accelerated, or delayed or prevented. Methods are also provided for treating a plant in order to accelerate or delay flowering in the plant.

Methods and compositions for placing a polynucleotide of interest under photoperiod sensitive or photoperiod insensitive control are also disclosed. The compositions and methods and can be used, for example, to make photoperiod sensitive a gene that is normally or naturally photoperiod insensitive. In other embodiments, compositions and methods and can be used to make photoperiod insensitive a gene that is normally or naturally photoperiod sensitive.

Screening methods are also provided for identifying plants for photoperiod sensitivity and chemical agents that can modify photoperiod sensitivity.

### BRIEF DESCRIPTION OF THE DRAWINGS

FIG. 1 is a bar graph showing frequency distribution of F₂ population of S. bicolor×S. propinquum as a function of flowering time. Also shown is a boxed line indicating average day length (hrs) over the time period. Also shown are two lines indicating the high (solid line) and low (dashed) temperature during the time period. S. propinquum and most F₂s flowered when photoperiod was less than 12.5 hours. Segregation of the S. bicolor and S. propinquum alleles at the Ma1 locus imparts dichotomous phenotype when grown in a temperate environment.

FIG. 2A is a diagram mapping the 1.1 centiMorgan (cM) interval delineated by progeny testing of recombinants. FIG. 2B is a diagram showing the % of conversion at the DNA marker loci plotted along the sorghum genome sequence (on base pair, bp, scale). The diagram also maps the relative locations of the FT gene (Sb06g012260) and SbPRR37 (Sb06g012570). The dark line at the top of the diagram indicates the span of converted regions with approximate locations of genes in the sequence shown as cross-hatches along the axis. While the terminal regions that these data exclude from consideration are physically small, they contain the majority of genes.

FIG. 3A is a diagram illustrating two major S. bicolor haplotypes (each with two rare variants) for the gene Sb06g012260 identified from analysis of re-sequencing data. One of the haplotypes (haplotype 1) closely resembled the allele found in the short-day flowering accession of Sorghum propinquum. FIG. 3B is a physical map showing the positions of four insertion-deletion events relative to the coding region of Sb06g012260. FIG. 3C is a diagram comparing the PRR37 alleles in S. bicolor (top) and S. propinquum (bottom). The S. propinquum allele has an “AT” insertion between 97 and 98 nucleotides after the translation starting site. This insertion causes frameshift shortly before the beginning of the PRR domain (arrowhead), leading to numerous nonsense mutations (arrows) and resulting in premature protein termination near the end of the PRR domain. Coding regions are shown as boxes, introns as solid horizontal lines, vertical bars indicate nucleotide substitutions between the two alleles.

FIG. 4 is a series of pie graphs showing haplotype frequencies for the gene Sb06g012260 in sub-populations from West Africa, South Africa, Central/East Africa, and Asia/India.

FIG. 5A-5C are bar graphs showing flowering (days) for individuals having haplotype 1 of FIG. 3A (empty bars) or haplotype 2 of FIG. 3A (shaded bars) for the gene Sb06g012260 in West Africa (FIG. 5A, 2008 p=0.005; R²=0.13) and South Africa (FIG. 5B (2008), p=3.84 E-08; R²=0.33) and FIG. 5C (2007), p=0.0346; R²=0.08). These data show a statistically-significant association of the haplotypes with flowering in subpopulations in which the two haplotypes each occur at similar frequencies.

FIG. 6 is a line graph of log p value versus Ma1 region (Mbp) showing the association analysis of Ma1 region markers and photoperiod sensitive in Sorghum bicolor based on routine application of the software TASSEL (Bradbury, et al., Bioinformatics, 23:2633-2635 (2007)), as detailed below. (♦) single marker analysis; (▪) analysis considering population structure.

FIG. 7 is a diagram showing homologs identified by BLAST of a candidate Ma1 gene (Sb06g012260) in sorghum, rice, and Arabidopsis genomes; and maize and sugarcane ESTs.

### DETAILED DESCRIPTION OF THE INVENTION

### I. Definitions

Before describing the various embodiments, it is to be understood that the invention is not limited in its application to the details of construction and the arrangement of the components set forth in the following description. Other embodiments can be practiced or carried out in various ways. Also, it is to be understood that the phraseology and terminology employed herein is for the purpose of description and should not be regarded as limiting.

Unless otherwise indicated, the disclosure encompasses conventional techniques of plant breeding, microbiology, cell biology and recombinant DNA, which are within the skill of the art. See, e.g., Sambrook and Russell, Molecular Cloning: A Laboratory Manual, 3rd edition (2001); Current Protocols In Molecular Biology [(F. M. Ausubel, et al. eds., (1987)]; Plant Breeding: Principles and Prospects (Plant Breeding, Vol 1) M. D. Hayward, N. O. Bosemark, I. Romagosa; Chapman & Hall, (1993); Coligan, Dunn, Ploegh, Speicher and Wingfeld, eds. (1995) Current Protocols in Protein Science (John Wiley & Sons, Inc.); the series Methods in Enzymology (Academic Press, Inc.): PCR 2: A Practical Approach (M. J. MacPherson, B. D. Hames and G. R. Taylor eds. (1995)].

Unless otherwise noted, technical terms are used according to conventional usage. Definitions of common terms in molecular biology may be found in Lewin, Genes VII, published by Oxford University Press, 2000; Kendrew et al. (eds.), The Encyclopedia of Molecular Biology, published by Wiley-Interscience., 1999; and Robert A. Meyers (ed.), Molecular Biology and Biotechnology, a Comprehensive Desk Reference, published by VCH Publishers, Inc., 1995; Sambrook and Russell. (2001) Molecular Cloning: A Laboratory Manual 3rd. edition, Cold Spring Harbor Laboratory Press.

To facilitate understanding of the disclosure, the following definitions are provided:

The term “plant” is used in it broadest sense. It includes, but is not limited to, any species of woody, ornamental or decorative crop or cereal, and fruit or vegetable plant. It also refers to a plurality of plant cells that are largely differentiated into a structure that is present at any stage of a plant's development. Such structures include, but are not limited to, a fruit, shoot, stem, leaf, flower petal, etc.

The term “photoperiod” refers to the period of a plant's exposure to daylight every 24 hours.

The term “photoperiod sensitivity” refers to the photoperiod that is required to induce a specific response, such as flowering. Some plants will not flower until they are exposed to day lengths that are less than a critical photoperiod (short day plants) or greater than a critical photoperiod (long day plants). In some plant species, photoperiodic control enforces long-day flowering. Therefore, a photoperiod sensitive plant can have either short-day or long-day flowering, but in both cases, the flowering is controlled by day length.

A plant is “photoperiod insensitive” or “day neutral” if the day length does not impact when flowering occurs. In order to modulate flowering based on day length, photoperiod sensitivity can be increased.

A “non-flowering” plant does not flower under the agronomic conditions, regardless of the photoperiod.

“Delayed flowering” refers to a plant that flowers on average at least 1 day later, including at least 1, 2, 3, 4, 5, 6, 7, 8, 9, 10 days later, than a wild-type plant of the same species.

The term “non-naturally occurring plant” refers to a plant that does not occur in nature without human intervention. Non-naturally occurring plants include transgenic plants and plants produced by non-transgenic means such as plant breeding.

The term “plant tissue” includes differentiated and undifferentiated tissues of plants including those present in roots, shoots, leaves, pollen, seeds and tumors, as well as cells in culture (e.g., single cells, protoplasts, embryos, callus, etc.). Plant tissue may be in planta, in organ culture, tissue culture, or cell culture. The term “plant part” as used herein refers to a plant structure, a plant organ, or a plant tissue.

The term “plant material” refers to leaves, stems, roots, flowers or flower parts, fruits, pollen, egg cells, zygotes, seeds, cuttings, cell or tissue cultures, or any other part or product of a plant.

The term “plant organ” refers to a distinct and visibly structured and differentiated part of a plant such as a root, stem, leaf, flower bud, or embryo.

The term “plant cell” refers to a structural and physiological unit of a plant, comprising a protoplast and a cell wall. The plant cell may be in form of an isolated single cell or a cultured cell, or as a part of higher organized unit such as, for example, a plant tissue, a plant organ, or a whole plant.

The term “plant cell culture” refers to cultures of plant units such as, for example, protoplasts, cell culture cells, cells in plant tissues, pollen, pollen tubes, ovules, embryo sacs, zygotes and embryos at various stages of development.

The term “transgenic plant” refers to a plant or tree that contains recombinant genetic material not normally found in plants or trees of this type and which has been introduced into the plant in question (or into progenitors of the plant) by human manipulation. Thus, a plant that is grown from a plant cell into which recombinant DNA is introduced by transformation is a transgenic plant, as are all offspring of that plant that contain the introduced transgene (whether produced sexually or asexually). It is understood that the term transgenic plant encompasses the entire plant or tree and parts of the plant or tree, for instance grains, seeds, flowers, leaves, roots, fruit, pollen, stems etc.

The term “construct” refers to a recombinant genetic molecule having one or more isolated polynucleotide sequences. Genetic constructs used for transgene expression in a host organism include in the 5′-3′ direction, a promoter sequence; a sequence encoding a gene of interest; and a termination sequence. The construct may also include selectable marker gene(s) and other regulatory elements for expression.

The term “gene” refers to a DNA sequence that encodes through its template or messenger RNA a sequence of amino acids characteristic of a specific peptide, polypeptide, or protein. The term “gene” also refers to a DNA sequence that encodes an RNA product. The term gene as used herein with reference to genomic DNA includes intervening, non-coding regions as well as regulatory regions and can include 5′ and 3′ ends.

The term “orthologous genes” or “orthologs” refer to genes that have a similar nucleic acid sequence because they were separated by a speciation event.

As used herein, “polypeptide” refers generally to peptides and proteins having more than about ten amino acids. The polypeptides can be “exogenous,” meaning that they are “heterologous,” i.e., foreign to the host cell being utilized, such as human polypeptide produced by a bacterial cell.

The term “isolated” is meant to describe a compound of interest (e.g., nucleic acids) that is in an environment different from that in which the compound naturally occurs, e.g., separated from its natural milieu such as by concentrating a peptide to a concentration at which it is not found in nature. “Isolated” is meant to include compounds that are within samples that are substantially enriched for the compound of interest and/or in which the compound of interest is partially or substantially purified. Isolated nucleic acids are at least 60% free, preferably 75% free, and most preferably 90% free from other associated components. An “isolated” nucleic acid molecule or polynucleotide is a nucleic acid molecule that is identified and separated from at least one contaminant nucleic acid molecule with which it is ordinarily associated in the natural source. The isolated nucleic can be, for example, free of association with all components with which it is naturally associated. An isolated nucleic acid molecule is other than in the form or setting in which it is found in nature.

As used herein, the term “linkage disequilibrium” or “LD” refers to the situation in which the alleles for two or more loci do not occur together in individuals sampled from a population at frequencies predicted by the product of their individual allele frequencies. Markers that are in LD do not follow Mendel's second law of independent random segregation. LD can be caused by any of several demographic or population artifacts as well as by the presence of genetic linkage between markers. However, when these artifacts are controlled and eliminated as sources of LD, then LD results directly from the fact that the loci involved are located close to each other on the same chromosome so that specific combinations of alleles for different markers (haplotypes) are inherited together. Markers that are in high LD can be assumed to be located near each other and a marker or haplotype that is in high LD with a genetic trait can be assumed to be located near the gene that affects that trait.

As used herein, the term “locus” refers to a specific position along a chromosome or DNA sequence. Depending upon context, a locus could be a gene, a marker, a chromosomal band or a specific sequence of one or more nucleotides.

The term “vector” refers to a replicon, such as a plasmid, phage, or cosmid, into which another DNA segment may be inserted so as to bring about the replication of the inserted segment. The vectors can be expression vectors.

The term “expression vector” refers to a vector that includes one or more expression control sequences

The term “expression control sequence” refers to a DNA sequence that controls and regulates the transcription and/or translation of another DNA sequence. Control sequences that are suitable for prokaryotes, for example, include a promoter, optionally an operator sequence, a ribosome binding site, and the like. Eukaryotic cells are known to utilize promoters, polyadenylation signals, and enhancers.

The term “promoter” refers to a regulatory nucleic acid sequence, typically located upstream (5′) of a gene or protein coding sequence that, in conjunction with various elements, is responsible for regulating the expression of the gene or protein coding sequence. The promoters suitable for use in the constructs of this disclosure are functional in plants and in host organisms used for expressing the disclosed polynucleotides. Many plant promoters are publicly known. These include constitutive promoters, inducible promoters, tissue- and cell-specific promoters and developmentally-regulated promoters. Exemplary promoters and fusion promoters are described, e.g., in U.S. Pat. No. 6,717,034, which is herein incorporated by reference in its entirety.

A nucleic acid sequence or polynucleotide is “operably linked” when it is placed into a functional relationship with another nucleic acid sequence. For example, DNA for a presequence or secretory leader is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are contiguous and, in the case of a secretory leader, contiguous and in reading frame. Linking can be accomplished by ligation at convenient restriction sites. If such sites do not exist, synthetic oligonucleotide adaptors or linkers are used in accordance with conventional practice.

“Transformed,” “transgenic,” “transfected” and “recombinant” refer to a host organism such as a bacterium or a plant into which a heterologous nucleic acid molecule has been introduced. The nucleic acid molecule can be stably integrated into the genome of the host or the nucleic acid molecule can also be present as an extrachromosomal molecule. Such an extrachromosomal molecule can be auto-replicating. Transformed cells, tissues, or plants are understood to encompass not only the end product of a transformation process, but also transgenic progeny thereof. A “non-transformed,” “non-transgenic,” or “non-recombinant” host refers to a wild-type organism, e.g., a bacterium or plant, which does not contain the heterologous nucleic acid molecule.

The term “endogenous” with regard to a nucleic acid refers to nucleic acids normally present in the host.

The term “heterologous” refers to elements occurring where they are not normally found. For example, a promoter may be linked to a heterologous nucleic acid sequence, e.g., a sequence that is not normally found operably linked to the promoter. When used herein to describe a promoter element, heterologous means a promoter element that differs from that normally found in the native promoter, either in sequence, species, or number. For example, a heterologous control element in a promoter sequence may be a control/regulatory element of a different promoter added to enhance promoter control, or an additional control element of the same promoter. The term “heterologous” thus can also encompasses “exogenous” and “non-native” elements.

The term “percent (%) sequence identity” is defined as the percentage of nucleotides or amino acids in a candidate sequence that are identical with the nucleotides or amino acids in a reference nucleic acid sequence, after aligning the sequences and introducing gaps, if necessary, to achieve the maximum percent sequence identity. Alignment for purposes of determining percent sequence identity can be achieved in various ways that are within the skill in the art, for instance, using publicly available computer software such as BLAST, BLAST-2, ALIGN, ALIGN-2 or Megalign (DNASTAR) software. Appropriate parameters for measuring alignment, including any algorithms needed to achieve maximal alignment over the full-length of the sequences being compared can be determined by known methods.

For purposes herein, the % sequence identity of a given nucleotides or amino acids sequence C to, with, or against a given nucleic acid sequence D (which can alternatively be phrased as a given sequence C that has or comprises a certain % sequence identity to, with, or against a given sequence D) is calculated as follows:

100 times the fraction W/Z,

where W is the number of nucleotides or amino acids scored as identical matches by the sequence alignment program in that program's alignment of C and D, and where Z is the total number of nucleotides or amino acids in D. It will be appreciated that where the length of sequence C is not equal to the length of sequence D, the % sequence identity of C to D will not equal the % sequence identity of D to C.

As used herein, “polypeptide” refers generally to peptides and proteins having more than about ten amino acids. The polypeptides can be “exogenous,” meaning that they are “heterologous,” i.e., foreign to the host cell being utilized, such as human polypeptide produced by a bacterial cell.

The term “stringent hybridization conditions” as used herein mean that hybridization will generally occur if there is at least 95% and preferably at least 97% sequence identity between the probe and the target sequence. Examples of stringent hybridization conditions are overnight incubation in a solution comprising 50% formamide, 5×SSC (150 mM NaCl, 15 mM trisodium citrate), 50 mM sodium phosphate (pH 7.6), 5×Denhardt's solution, 10% dextran sulfate, and 20 μg/ml denatured, sheared carrier DNA such as salmon sperm DNA, followed by washing the hybridization support in 0.1×SSC at approximately 65° C. Other hybridization and wash conditions are well known and are exemplified in Sambrook et al, Molecular Cloning: A Laboratory Manual, Third Edition, Cold Spring Harbor, N.Y. (2000).

### II. Compositions

Photoperiod sensitivity refers to the fact that some plants will not flower until they are exposed to day lengths that are less than a critical photoperiod (short day plants) or greater than a critical photoperiod (long day plants). Long day (LD) and short day (SD) plant designations refer to the day length required to induce flowering. Facultative LD or SD plants are those that show accelerated flowering in LD or SD but will eventually flower regardless of photoperiod. Most plants including sorghum must pass through a juvenile stage (lasting about 14-21 days for sorghum) before they become sensitive to photoperiod.

In general, Sorghum is a facultative SD plant where long days inhibit flowering and short days accelerate flowering. The degree of flowering photoperiod sensitivity in sorghum refers to the length of the short days that are required to induce flowering. Different sorghum genotypes vary in their degree of photoperiod sensitivity. For example, Sorghum inbreds have been identified with photoperiod sensitivity ranging from ˜10.5 to ˜14 hours and still others that are nearly completely insensitive to photoperiod.

Flowering depends on when seeds are planted and on the latitude in which they are planted. Therefore, in some embodiments, a photoperiod insensitive sorghum planted in Georgia in April can flower in approximately 48-55 days; whereas a highly photoperiod sensitive sorghum planted in Georgia in April can flower in ˜175-180 days, or may even fail to flower at all.

The maturity gene (Ma1) contains one or more mutation or deletions in some S. bicolor genotypes such that sorghum plants containing this mutant gene are photoperiod insensitive (day-neutral). Identification of this gene allows for identification of orthologous genes in related plants. Moreover, based on this identification, methods of modulating photoperiod sensitivity in plants by modulating the expression control sequences of maturity gene in that plant are disclosed. Methods are also disclosed for modulating photoperiod sensitivity involving modulating the activity of the protein encoded by the Maturity (Ma1) gene in the plant.

A. Ma1

Compositions and methods for modifying photoperiod sensitivity in plants are provided. The methods can involve modulating the activity of the endogenous gene or gene(s) responsible for photoperiod sensitivity in the plant.

For example, the methods can involve promoting the expression of one or more endogenous gene orthologous to sorghum grain maturity gene 1 (Ma1). Thus, the methods can involve introducing to the plant a composition that promotes maturity gene 1 (Ma1) activity in a Sorghum plant.

The term “Maturity gene” refers to the Ma1 gene found in Sorghum as well as orthologous genes serving the same function in related plants.

Sorghum

Sorghum has been an excellent biomass source with its high yield potential, high water use efficiency, and established production systems and is a representative plant that can be used with the disclosed methods and compositions. Sorghum is a genus of numerous species of grasses, some of which are raised for grain and some of which are used as fodder plants either cultivated or as part of pasture. The plants are cultivated in warmer climates worldwide. Sorghum is in the subfamily Panicoideae and the tribe Andropogoneae.

Sorghum is well adapted to growth in hot, arid or semi-arid areas. The many subspecies are divided into four groups—grain sorghums (such as milo), grass sorghums (for pasture and hay), sweet sorghums (used to produce sorghum syrups), and broom corn (for brooms and brushes).

Sorghum species include, but are not limited to Sorghum almum, Sorghum amplum, Sorghum angustum, Sorghum arundinaceum, Sorghum bicolor, Sorghum brachypodum, Sorghum bulbosum, Sorghum burmahicum, Sorghum controversum, Sorghum drummondii, Sorghum ecarinatum, Sorghum exstans, Sorghum grande, Sorghum halepense, Sorghum interjectum, Sorghum intrans, Sorghum laxiflorum, Sorghum leiocladum, Sorghum macrospermum, Sorghum matarankense, Sorghum miliaceum, Sorghum nigrum, Sorghum nitidum, Sorghum plumosum, Sorghum propinquum, Sorghum purpureosericeum, Sorghum stipoideum, Sorghum timorense, Sorghum trichocladum, Sorghum versicolor, Sorghum virgatum, and Sorghum vulgare.

Sorghum Maturity Gene 1

There are six classic maturity genes in sorghum that control flowering time termed Ma1-Ma6. Therefore, in general, sorghum plants with recessive Ma1-Ma6 genes (with low or no activity) flower earlier than plants with dominant or active Ma1-Ma6 genes that repress flowering.

Nucleic acid sequences for Ma1 genes in Sorghum bicolor and Sorghum propinquum are provided. It is understood that the skilled artisan can identify orthologous sequences in other Sorghum species for use in the present compositions and methods. For example, Ma1 genes from Sorghum almum, Sorghum amplum, Sorghum angustum, Sorghum arundinaceum, Sorghum brachypodum, Sorghum bulbosum, Sorghum burmahicum, Sorghum controversum, Sorghum drummondii, Sorghum ecarinatum, Sorghum exstans, Sorghum grande, Sorghum halepense, Sorghum interjectum, Sorghum intrans, Sorghum laxiflorum, Sorghum leiocladum, Sorghum macrospermum, Sorghum matarankense, Sorghum miliaceum, Sorghum nigrum, Sorghum nitidum, Sorghum plumosum, Sorghum purpureosericeum, Sorghum stipoideum, Sorghum timorense, Sorghum trichocladum, Sorghum versicolor, Sorghum virgatum, and Sorghum vulgare can be identified and used in the disclosed methods.

Within the species Sorghum bicolor, there are both day-neutral (photoperiod insensitive) and short-day flowering forms. The vast majority of wild members of the species are short-day, as are forms cultivated in the tropics. Forms cultivated in temperate latitudes (such as most of the USA) for seed/grain have been selected for day-neutral mutations. Therefore, the skilled artisan can use the guidance provided by the sequence comparisons to identify variants of Ma1 genes that can generate a photoperiod sensitive or insensitive phenotype.

Also disclosed is a transgenic plant having a nucleic acid molecule, or antisense constructs thereof, encoding a Ma1 gene product, or variant, such as a codon optimized variant thereof, optionally operatively linked to an heterologous regulatory element. For example, disclosed is a transgenic plant characterized by high photoperiod sensitivity, low photoperiod sensitivity, or photoperiod insensitivity, wherein the cells of the plant express a nucleic acid molecule encoding an Ma1 gene product, or antisense construct thereof, that is operatively linked to an expression control sequence. In some embodiments, the construct encodes an inhibitory nucleic acid such as siRNA or RNAi that when express down regulates the expression of Ma1.

Nucleic Acids

Ma1 Gene

Disclosed are polynucleotides containing a maturity gene from a sorghum plant. It is understood that where coding sequences for a maturity gene are provided, also provided are the non-coding sequences that are known or can be identified to correspond to the coding sequences that are provided. For example, where a maturity gene is provided, also provided for use in the disclosed compositions and methods is the 5′ untranslated region (UTR), which contains the endogenous promoter for the maturity gene. It is understood that the skilled artisan can identify these sequences with routine skill and experimentation based on the sequences that are provided.

1. Sequences for Short Day Flowering

The S. propinquum cultivar from which the sequences described below are derived is a short-day cultivar, that has a dominant (functional) Ma1 allele. Sequences for a dominant Ma1 gene are therefore provided.

In some embodiments, the maturity Ma1 gene (including non-coding sequence) as it is found in short day S. propinquum includes the nucleic acid sequence:

| | |
|---|---|
| 1 | AAAAGAAAAG TGAGCACACC ACGACCTGTC ATCAGCTCAT GGTCAGCTCT ACAAACTTAT |
| 61 | AGATTGCATC GAGATCTAAG ACTCAGGTAC AAATCATGTC AACATCTAAT GGTTTAGAAA |
| 121 | ATGAAAAGTT TTGAGTTTCA AAATATGATA CGTGATATTA ACATTTGAAC TTTTAGCAAG |
| 181 | ATCTGAAATA AAAAATTCAA CTAGATCATG TTAACATTGA TATAATCGCT TCCAATCGCC |
| 241 | TCCCATCACT TCCGCTAGAA AACTTTTTTT CTCGATTTAA TTAATGAAAG GGTAATAACA |
| 301 | TCATTGTACA AGATTCTTTC AAACCTCAAC CCCTATCATC GACGGTGACG GCTCCCTATA |
| 361 | ACACGCACTA GTGGACGCCG GGCGGGTGGA ACCCTAAGAA GATTTAAAAA AACTTAAGAA |
| 421 | GAAGATTTTT ATCTAACTAA CTATAGTACT TATATCATAC ACTATACTAT TCAAAATATT |
| 481 | ATTTTCACAA TTATGAATTT ACCCTTTTAC TCTTCATTAA AAAAATACGA AAAAAGAATC |
| 541 | ACCACGTCTC TATTTAGGGT CCTAGTCCCC ATAATTTAAG AGGCGGTGAG AGACGATGTG |
| 601 | ACGTCTATGG ACCACCGACC AAAGACACAC CTATCGTCTC CCATCGCCTT GCTTCCATCG |
| 661 | CCTCTCATCG CTTTTCATAT TCTAGATCCA GCGGCCATAG ACACACCAAT CGTTTCTCAT |
| 721 | CGCCTCTCCA ACCATTGTAA AAATATTTAT AATTTTGATA TAAAATTTGT CTTCACTTGA |
| 781 | GTTCATGCCA AAAAAATTAT ACATATTATT TTCGTGTGAG AATTTACAGA AGTGGACTCT |
| 841 | TAAGATGTCC AAATGTAAAT GACCCTATTT ATTATGAGGC GCGGATCTAT AGGCCTGACT |
| 901 | CTGAAAATGG ATTATGGATT TGAGATAATA AATTTAAGGG CCTATCTTCG CACATAACAT |
| 961 | CTATAGTTCC TAAATTTTTT TTTATTGTAG TAGTAGAACT TTTCTCCCTG TAAACCAAGT |
| 1021 | TGACGCTGGG CTTTATTTTG CGACACAGAA CACCAAATTG GTGGCTATGA ACTCTTCCAC |
| 1081 | CTGGGCAGGG AAAACGGTTT ATTATGTTTC TCTTTAATTT ATCTATCGTG GCACTATAAC |
| 1141 | ACAACATGGC TTTGCCGACA CTTCCAACTA TCGGCAAAGG GTACCTTTAC CGACACTTAA |
| 1201 | CGTCTCACGA AAGGTTTTGC CGACAATTTT CAAACAGTCG CGGTAGAAGC AGTTGGCGAA |
| 1261 | ACTTTTGCCG ACAGTTAAAG GCATCGCCGA CACATTTTCT GTAGTCAAAT GGCATACCTA |
| 1321 | CGCCGACAGT TGAACTTTCA CCGACAGTGA ACCCTTTGCC GACAGTTTGG ACCTACGCCG |
| 1381 | ACAGTTTGGA CCTTTTCCGA CAGTTGGTAT GTTAGCGAAA CCGTTTCTAG GGTGTTTCAT |
| 1441 | AAACCATGCC TTGTCCAACA GTAGAAGTGT CGGCAAAACT ATATTGCTAG GATGTAGATA |
| 1501 | CAATTTAAAT ATTTTAATAA ATACACATCA CATTGATTGA GCAAAATCAC ATGGTCTGTT |
| 1561 | TTCACTAAAA CTGTCAGAGG TACACTCCAG TACTACCAGT ACGTCGCCCG CACAGTGGCC |
| 1621 | AAGGATTTTA CTGCTACTGT TGATTAACAT AAGCACTTGC GACTTTCCCT AAAATCTTTT |
| 1681 | ATAAAACAAC GGCCGCAATA ATATTGAACT ATTTTTTTTC TAGTACCAAA ATTAGAATTT |
| 1741 | GATCCCTCAC CTCATTACAT CCATAGTAAC ATGACCAGAT ATATATGGAC AGGATGGGAT |
| 1801 | CACTCAGCGA GCAGATACAC TGAGCGATTC ATAATCAGAT TTTTTAATTT CTTCTAGTGA |
| 1861 | AGTGGGGTTT TCCTAGTCTT TTAACATTCA AAATTTAGTA CAAACTTTCC CTAGTAAATG |
| 1921 | CCTTCTAGTA AAGATTTCCT AGTATTTTGA CTAGCGATAG TGTTTTATTA CTAATTAAAA |
| 1981 | ACATTAGAAG AACTCCATTT AGTGATTGGT TGTTTGGATT AGTCTTCTCA CGTTAGACCT |
| 2041 | ATATATGCAG GACAACTCAA GCCAGCATAA ATATATGAAA TATCTTGGTG TTTGTTTGTC |
| 2101 | TGACACAGGC AACCGCGTTT GGTATAAATG TGTTTTCTTG TTTACATTTT ACCATCTATA |
| 2161 | GTCATCTCAA TGTTATATAG TAGAGGCTTC ATGTTTGTAG TAGATAAGGT AGAGAATTGA |
| 2221 | GAATATTTTA TTTTTGTGCG ACCATCAATT TTATGTAATC TGCATTGTCT AATGCTTTAT |
| 2281 | TTGACATTTG AAACTACTTA ATTTGACAGT TATGCAGGTC CGCATGATCC TATGAAAGCA |
| 2341 | ATTAATTAGT ACGGGTAAAC TGCACTACAC AAGTTTGCTA GTACTATTCT ATTAACCGAC |
| 2401 | CTGTCAATAT TACCTTAAGT TACTGATTTC AATTAGAATC TAACACATTC AGGAAAAGAA |
| 2461 | GTTTCACTAG TACAAAAATC ATTTTCGTTG GCACGTTGTT TTTTTTTTCA CAGGCAGTTC |
| 2521 | ACAATATCAT GGTGCTAGTA GAAAAATTTC AACGGGCCCA ACAAGAGAAC CGCCAGGCGG |
| 2581 | TCTTCTTAAT TCAACCGCCT GTGTAAACTT TCCATTTACA TAGGCGGCTT ACGATAAAAA |
| 2641 | CCGTGTGTAT AAATACCATT AACACAGGCA GTCGAGTTAC GACAACCGCC TGTGTAAATG |
| 2701 | TGTCTTTTTA CACAGGCGGT TTGTATAGAG GGCCGCCTGT GCTAATATAT TTACACAGGC |
| 2761 | TATGAGCCGC CTGTGTTAAG TCTTCTATAA ATACCCTTCG TCCACCTCCA GACAAGAACA |
| 2821 | GTTACTCCCA TGAGCTCTGC ACACTGGCGG ACCAGACGAT TCCAGTTTCC AAGGGGGGAG |
| 2881 | GTTTTGATTT TCATTTCTTT GGTGAGAAAC TTCCAAAAGG TTAGTTAGTG CCATTGATGC |
| 2941 | TATTTTTTAA GCGATTCTTT GGTTCAATTC TTGTATTGGA GGTGCTCTAG ATCTAGAGTT |
| 3001 | CATCATGCAT TCTTGCTTAG GGTTAGAGTT CATAGGGCAA AAAGAGAGAG ATTTAGCTAA |
| 3061 | ATTTTTATGT AAATTCATAG TAAATTGTAA AAATTAAAAA AAATAAAAAA TAAATACTTT |
| 3121 | TTAGAATTCT TGTGAGTAGA TCTATACAAT AGAGTAATGA TGAGGATATT TTGAAGTTTA |
| 3181 | TAATTTTGAT TCAGTTTTAG CTTTTCTTTT TTCAGATGAA TTAGACTTTA TAAACTCAAA |
| 3241 | CATTAAAATG TTGAAAATCA TAAAATGGCA AATAAATACT TTTTCAAATC TTTGTGCATA |
| 3301 | AATACTTCAT AGAAATCCTT GAATTATTCC TAAATTTTAT ACAATTGTTT CTTATAATTA |
| 3361 | TGAAAATGAG TTTAAACAAT TATTTAAATT CCATAAATTG TAACTCCGTA AGGTGTAGGT |
| 3421 | TTTCATCTCT GTTTAATAGA AGGAGGTTAG TATCTTAGTT AAGTCTGTTT TCGGGGGTTA |
| 3481 | TATTAGTTTT GTTTTTAGAT TGACCTACAT TAATTGTTCT TAACTAATTA CAGCTAAATA |
| 3541 | TGGAGAGGTC ATTATGGATG TACAACTTAT CAAGATTGGA CCTATCATAT GTAGTGCAGG |
| 3601 | TCCAAAAATT TATTGATGTC GCAAAGATAC ATGCTCGCAG AACAAAGGCG AAGCACATAT |
| 3661 | GTTGTCCATG CGCAGACTGC AAAAATATTA TGGTATTTGA CAATGTAGAA GCAATTACTT |
| 3721 | CCCATCTGGT TTGAAGAGGA TTTATGGAGG ACTACTTGAT TTGGACAAAA CATGGTGAGG |
| 3781 | GTAGTTTTGC ACCTTATATG CGGACAACTG ACAACACTGC AACTAACATC AATGTGGAGG |
| 3841 | GTCCAATGCC ACCTCTCAAT GAATTTCATG CTATGCCAGA TGTTAATGAA ACTCATACGT |
| 3901 | CTGATGTCAA TGAAACTCAG CATGCTAACA CAGATGTTGT TGAAGATGCA GATTTCTTAG |
| 3961 | AGGCAATAAT GAACCGTTGT GCGGATCCAT CAATATTCTT CATGAAGGGA ATGAAAGCAT |
| 4021 | TGAAGAAGGC AGCAGAGGAC ACTTTGTACG ACGAGTCAAA AGGTTGTACC AAACAATGGT |
| 4081 | CGACATTATG TGTTGTTCTT CAGTTTTTGA CGATGAAGGC TAGACATGGT TGGTCCGATG |
| 4141 | CTAGCTTCAA TGATTTCTTG CGTGTACTTG GAGACCTTCT TCCTAAGGAG AACAAAGTGC |
| 4201 | CTGCTAACAC ATACTATGCA AAGAAGCTAG TCAGTCCACT TACGATAGGT GTTGAGAAGA |
| 4261 | TCCACGCATG TAGAAATCAT TGTATTCTAT ATCGAGGTGA TCAATATAAA GACTTAGACA |
| 4321 | GTTGTCCAAA CTGTGGTGCC AGTAGGTACA AGACAAACAA AGATTTTCGG GAGGAAGAGA |
| 4381 | ATCTAGCCTC TGTTTCTACA GGGAGGAAGC GAAAGAAGAC CCAAACAAAG ACTCAACAAG |
| 4441 | ACAAGCGCTC AAAGCCTAGT AGCAATGAAG AAGTGGACTA TTATGCATTG AGAAGAGTCT |
| 4501 | CCCTATGAGC CAAAAAAGGG GACAGCAGCA GGCACAACTC TCTTTCTGAA AGGACTTGGA |
| 4561 | AAGCAGCGGA CGGCACGGCT CATTGAGCTC GAACCGTCAC AGAAAAAGGA AGCCACCGCC |
| 4621 | CAGTCAATAG AAGCCATGCC CCCATCAAAG GAAGCCCCAA GTGGCGATGT ACATATTGAA |
| 4681 | CAGCCATCAA GTCAACCATT GACCCTAAAG GATATCAGAA AGCCAACGAT TGATGATTAT |
| 4741 | GTCAATGTCC CTAGTGACTA TGTGCCCGGA AGGCCTATGC TCCAATGGAC GCTGCTCGAT |
| 4801 | TAGATTCAAT GGCTGATAAA AAGGTTTCAT GACTGGTACA TGAGAGCAGT GCATGCTAGC |
| 4861 | CTCCATGGAA TCAGAGTTGA TATACCAACA GACATGTTTG CTACTGGTAA CAAAAAAAGC |
| 4921 | AAGACATTTG TTACCTTTGA GGACATGCAC TTGTTATTGA ACTATAGGCG GCTTGACGTC |
| 4981 | CAACTCATAA CAATCTGGTG CCTGTAAGTA TCACTCATGC ACACACAATT ATTATATATT |
| 5041 | AATATGTAGT GTGAAACTCT AATATGTAGA TGTTGTCTGT AGTTTGCAAG ATCACGAGCA |
| 5101 | GATGTCATTA TTATCTGCCG GATCGATGGT CGGTTATCTG AGCCCTATCA AGTTACAAGA |
| 5161 | AAATATGAAC AAATTCGTAT TATCAAAGGA AGATAGAGCA AAGATAGAGG AAGACAAAAC |
| 5221 | ACCAGGATAA TTATGCCATC TATCTTGGTA GATCAATGCT GAGGTATAAA TATAGGGATT |
| 5281 | TTATATTGGC ACCATACAAC ATTAGGTAAG CTTGACTTCA TATACGTATT TCAAATTATC |
| 5341 | GTGTAAACAA TATACATGTG TCGCTCACTC ATTTATTCAT GCAGTGACCA TTGGATTGTT |
| 5401 | TTTTATATTT ATCCCTTCGA AGGGAAGGTG CTTGTCCTAG ACTCTTTACA TGTTCCTCCC |
| 5461 | GAGAAGTATC AACCATTCTT GGTTCAATTA GAAAGGTGAG CCAACATGAA ACCACATGCG |
| 5521 | TACTTATATA AATTAGAGTT TCAAAATAAC TTTAGTGATT TAGGTTCGAT ATCTACGGGG |
| 5581 | CATGGCGGTT TTATAAGAAA CAAAAGGGAC CTGTCGACGC TGCACGCTCA GATCCTAGGA |
| 5641 | TCCCATTGAT GATACAACAC CACTATCCGG TAAGTTTTCT GAACACATTT CATCATATAA |
| 5701 | ATAATACATA AAGCATGGCA AATTTAGAAT AATCCGTTGC TCATTATATA GTGCCACAAG |
| 5761 | CAACCACCTG GATCGGTCTA TTGTGGGTAC TATGTCTGTG AGTTTATAAG GCAGCGGGGA |
| 5821 | CGTTACGTCA AGGACAAAAA TATGGTAAAT AATATCTATG TATGAAAGTT TTCTCATTAA |
| 5881 | AGCTGCAAAA TTATATATTG AACATGTGTC AATCATGCTT TTAAACTTTA TTTTCAGCCG |
| 5941 | AAAAAGCAAG GAAAAGACGT GCCCTTTACA CCAAAGACTC TGGAAGATAT AGTAGCATAC |
| 6001 | TTGTGTGGTT TTATTATGAG AGAAATAATT TCAAGTGACA GTGCATATTT TGATCATGAG |
| 6061 | GGCGATTTAG CAAGTGATAA ATTTAGAGTG CTGACAGACA TAGCAGGTCT AAATCTGAAG |
| 6121 | CGAAACGACA TGTAAACATT GTATGGTTGT GCGGATAACA TGCATTGACG TGTATATATA |
| 6181 | TAATTTTATG GTTGATGTTT GATTTGTTTA CAATTCTATA ATATATATAT GTGGTGTATG |
| 6241 | TATGATGTTG TGTGTGTATA TATATATATA TATATATATA TATATATATA TATATATATA |
| 6301 | TATATATATA TATATAATGT TTAGCACTGT GTTTGGTGGG AAAAATTAAA ATTTGAAATA |
| 6361 | TATATAAAAA ATTATTTACA CAGACAGTGT ACGTGTCGAG CGTCGTCCTG TGCTATACAA |
| 6421 | ATACATTCTA ACAGGCGGCT CGCCTTGTCC ACCGGTCGGT TAAAAATACA TTTCCACACN |
| 6481 | GGCCTGGCTG GGAGAGCCGC CTGTGAAAAC ATAATTTTCA CAGGCGGCTC GCACAGCCCC |
| 6541 | GCCTGTACTG TGGTCCATTT TGTACTGACC CCTGGTACAG GCGGTGGGCT TGGCCGCCTG |
| 6601 | TGAAGATGCT TTTAGCACCG CCTGTAAAAA TGTTTTTTGT AGCAGTGTTT TTCTTATTAG |
| 6661 | TAGTATCTTT TATACTAATT AAGATTCAAT AAAAATTCAC CATGACATCC CCATTGCCAA |
| 6721 | GAGAATATTT CGCCGCCCCT CAAAGCAGCC AATAAGGCTT TACTAAAAAG ACTATCCACG |
| 6781 | CAGTAGAGAT TTAGTCAAAA TATTCCAATA GCAATTGTTT CCTGCCTGCT TGACCTTCGT |
| 6841 | CAGCCACTCA CTGTATAAAT ATCGCACCAC GCCCTTTGCA GGCTTACAGA GCTTGTATTA |
| 6901 | CGTACTAACA AGGCACACAC AGTACCCTGT GTTCACCGGC CCTGCACAAA ACTCAAGCAG |
| 6961 | TTATTACTAA CATGGCGGCT AACGATTCCT TGGTTACTGC TCATGTGATA GGAGATGTCT |
| 7021 | TGGACCCCTT CTATACAACC GTTGACATGA TGATCCTATT CGATGGTACT CCTATTATCA |
| 7081 | GCGGCATGGA GTTGCGCGCT CCGGCGGTTT CTGACAGGCC AAGGGTTGAA ATTGGAGGAG |
| 7141 | ATGATTATCG AGTTGCATAT ACTCTGGTAA ACTCATGCCA TGTCAATTAA CTAGTAGTTG |
| 7201 | AATTTAGATG CTGGTGGTAT CGTGGATACA TGTACTATAT GTTATGGTTG ATACATATTT |
| 7261 | GTTTAATTGA TCGCAACACC ATTTGCGGTA ACTTCAAATT ACATTCTTTC AATATATAGG |
| 7321 | TGATGGTCGA TCCTGATGCT CCTAACCCAA GCAACCCAAC CTTGAGGGAG TACTTGCACT |
| 7381 | GGTAAGAGAA ACCTATAGAC GACAATTATT GTTGTTGGCA TGTTTTGCCC ACATATACTT |
| 7441 | TGTGTGTGTA TATTTGTGCT TATGCTTCTC CATAAAATTT TGGTGTATGT CTCAAGAGAG |
| 7501 | ATAGGTATAG AGGTTAGCAG TCCTTTAAAA ATGGTTTAAT CCAGTAGTTT TTTTTCGGTC |
| 7561 | GGACTGCTCG AATTATTGTA TATATGGAGA TCACATGCTA GTAACTTTTT CAATAATTTC |
| 7621 | ATGTTTCGAG CAGGATGGTG ACTGACATCC CAGCATCAAC TGATAATACA TACGGTGAGT |
| 7681 | ACACCCCTAT TCCCATTTTG AAACAAGTAG AATGTCTATT TTTATGATTT AGTATGTTCG |
| 7741 | TGACAATAGG CTATAGCTAT TTTGAAACTT CGGGAGCATA AAATAGTACT CGATTTTGTA |
| 7801 | TAACCATAAA CACACAGCTA GCCAATCTCT ATTCATATTT ATTTTAGTTT TATTTGCCGA |
| 7861 | ACCATCCTCA ACATCATAGC CACTTGATCG ATCATCTCAA TCAGCGTTTG TATCCTTGCC |
| 7921 | CGCTTGATTA TCATCCATGG CAGTTCATAT TTTTTTTCAT TTCTTTCATG CTTGTTATAG |
| 7981 | TTTTATCTGA TGAATCCAAG ATGTTATTGA TCAATTAGTT CAGATGAGCA GTAATGCATG |
| 8041 | TTGGAGGTTT GGTAGTATAT ATACGTTCAA AATTTCACGA AATCGGTAAT TACGGTGGGA |
| 8101 | GCCAAAAAAA ATTCCAAAAT TTCGTATTAC ATTAATAATG CATGTGCTGT AGACTCATAT |
| 8161 | TTTCTATGAT TTCGATTCTG TCACCATCCT GCTCGAATAT TTAAATCATG CTAATATTTT |
| 8221 | GTTTACATCT AAATCTTTTA TAAAAATTAT AATTTATATT TGGGTTTAAC AATTTCGGGC |
| 8281 | GCGTTTAGTG AGATTGGGTA ATTTCGGAGC GAGGCCACCG GCCACACGAA AAATTNCTAT |
| 8341 | ACACGNACTA TATGTGTACA TGTACATGCA TGGCACCCTG ATAGGCTACC CCATGGGGAA |
| 8401 | AAAATTGGAA ACGGACCATT CATACGCAGT CGTGGTGCAG ACTGTGGGCC ACAATAGCAG |
| 8461 | TGTAAACATA ATTACGGTAA TCAAATACCC CATGGGACCA TATATATCAT CCACAGATCC |
| 8521 | GTACGGTGCT TCCGTGTGGA TGGTCTACAC CAGATCTTTT CCACACCATA AGGGCAGCAA |
| 8581 | TGCAGCATCA TATTCATATA TGCACTAGTG ATGTACCATT TGGCTTATAT CATATTCAAC |
| 8641 | CTAACTCCTT GGAAACATTA TGATATTCTA TTGGGTTGAA GATGTCACTA CTACAAAAAA |
| 8701 | AAATCTTATG AGAGGTGTTT TGAAAACTGC CGGAGGTGCT TAAAGGAGAC AGACGAGTTA |
| 8761 | GGACAACCGT CTCTATTAAT GTGTACTAAC TGAGGTAGTT ACCGTAACGT GCCTGACTTG |
| 8821 | ATTAACAGAT TCAACCGTCT CAGTAAAGGC CATGATTAAC CGAAACAGAT TCGAGAGTTT |
| 8881 | TCTTAAGTAG TTAAACTATT TTAATCTTCA CCGAACTTAT AGAAAATGAA AGAGCTAACA |
| 8941 | CCAATATTTA TAAAAATAAA TTAGTATCAC TAAATACATC ACGAAATCTA TTTGGTGTTG |
| 9001 | TAGAAGTTAT CCTTTTCTAT AAAATTGATC AAATTTATGA TAACTTAGTT TTAGGAATTC |
| 9061 | ATTTATTTTA GGACAACTGA GGAAGTACAT ATTTTTTAAG TCATCCACAA AGTAGTGGAT |
| 9121 | CCAATTTATT ACATTACTCT ACTACTTCAA ACTGAACAAA AGCCTAATCC TGGTTATTTT |
| 9181 | TAGAGTGATT TTTTACAACA TCAGCAGTAG TCCAGAAAAT GGGAGGACAT TAATAAAAGT |
| 9241 | GAAAAGGAGC AGAAGAAAGA TTACGGTATT TTATTTGTGC TATTTGTTTA ACTATTGGCA |
| 9301 | GTTTGGGACC GAAATAAATA ACTGTTCGTA GCTCTATATT TGTCGATTCA AAAAGTGTAA |
| 9361 | CGATGATTTT TGTGTTTCAA AAGAAAAATA AAGAAGTGCA CCAATGATTG GATATCATAG |
| 9421 | GCTATATATG TTGGATTAAT TGCATCCAAC GTATATAGTG AAAATGCTTT TCAATCAAGT |
| 9481 | AATCTTCGAG CGGTTACCAG TTTTAATAGT TGCGAGTCGT CGTTTTTTAT GTACCCTAGG |
| 9541 | ACATATATAT CCGCATGTAG ACGATGATGA GACTAGCAAG TTTTTTTTTT TTTTTGAGCA |
| 9601 | AATACATAAT TATTGGATTT GCAGGCCGTG AGATGATGTG CTACGAGCCC CCTGCCCCGT |
| 9661 | CCACGGGCAT CCACCGTATG GTGCTGGTGC TATTCCAGCA GCTTGGCCGT GACACGGTGT |
| 9721 | TCGCGGCGCC GTCCAGGCGC CACAACTTCA ACACCCGTGC CTTCGCCCGC CGCTACAACC |
| 9781 | TCGGCGCGCC CGTCGCCGCC ATGTTCTTCA ACTGCCAGCG CCAGACCGGC TCCGGTGGCC |
| 9841 | CCAGGTTCAC CGGGCCCTAC ACCAGCCGAC GTCGTGCGGG CTGATGACGA CGATCGTCGT |
| 9901 | TACGTCACGT GTACCGTACA CATATATGTA TAGATATACA TGCATGCATG TTCCATGGTA |
| 9961 | TAGGATCGGT GACAAAACGT CTAATAATGT ATACACACAC ATGCATGGAA TGCATGTAAT |
| 10021 | AAGAGAATAT ATGTATAATA AGTAGGGGAG AGCATGCATA TATTGTGTAC ACGCGTCCGA |
| 10081 | TGCGTATAGC CCTTTACATT ATTGTAGTTG TAATCAGCTG TTTAAGCATT CTGCTGTGTC |
| 10141 | AGAACATGAT GCATATATAG TTTGGTGTGA GTATTGATCT AGTGGAACTC TTATCAGCCT |
| 10201 | TCAACTCTTA TCACAAGTGT AAGATATAGC TTTTATACCT TCAGGTGTCT TCCCAGTGTA |
| 10261 | CCTAGAAATG CTACAACGGT TGTATTTTAT CTATGCGCTT CACTACTGGA AACCTGAATA |
| 10321 | CTTCTGTGGA TGTCGAATTT TTCTGTGCGT TTTTTTCGAT ACACACGGAA AAATTATAAT |
| 10381 | TATTCTGTGG GTTTTAAAAT ATCCTCATAG AAAAATACAA ATACCCACAG AAAAATTATA |
| 10441 | TCATTTTTCT GTGCGTGACA ATACACTCAC AGAAAAATTA CAATTTTTGT GTGTGTTTAT |
| 10501 | ATAAAACGCA CAGAAAAAAT AATCACACAC AGAAAAATTA TAATTATTCT GTAGGTTTCT |
| 10561 | ATAAAACGCA CATAAAAAAT AAACACACAC TGAAAAATAG AACAAGCACC CTCATACTAA |
| 10621 | ATTCATATAA ACACCCATAT TTTTTTCTTT TTAATCTCTC TGTAAAACTT GTAACTAGTT |
| 10681 | TTTCCCTCTC GTACTAACTC CAAATTGGAT GATTT |

(SEQ ID NO:1 Sb06g012260—S. propinquum) or functional fragment, or variant, such as a codon optimized variant, thereof having at least 75%, 80%, 85%, 90%, 95%, 96%, 97%, 98%, or 99% sequence identity to SEQ ID NO:1.

The coding sequence of the maturity Ma1 gene of SEQ ID NO:1, including introns, can be:

| | |
|---|---|
| 1 | ATGGCGGCTA ACGATTCCTT GGTTACTGCT CATGTGATAG GAGATGTCTT GGACCCCTTC |
| 61 | TATACAACCG TTGACATGAT GATCCTATTC GATGGTACTC CTATTATCAG CGGCATGGAG |
| 121 | TTGCGCGCTC CGGCGGTTTC TGACAGGCCA AGGGTTGAAA TTGGAGGAGA TGATTATCGA |
| 181 | GTTGCATATA CTCTGGTAAA CTCATGCCAT GTCAATTAAC TAGTAGTTGA ATTTAGATGC |
| 241 | TGGTGGTATC GTGGATACAT GTACTATATG TTATGGTTGA TACATATTTG TTTAATTGAT |
| 301 | CGCAACACCA TTTGCGGTAA CTTCAAATTA CATTCTTTCA ATATATAGGT GATGGTCGAT |
| 361 | CCTGATGCTC CTAACCCAAG CAACCCAACC TTGAGGGAGT ACTTGCACTG GTAAGAGAAA |
| 421 | CCTATAGACG ACAATTATTG TTGTTGGCAT GTTTTGCCCA CATATACTTT GTGTGTGTAT |
| 481 | ATTTGTGCTT ATGCTTCTCC ATAAAATTTT GGTGTATGTC TCAAGAGAGA TAGGTATAGA |
| 541 | GGTTAGCAGT CCTTTAAAAA TGGTTTAATC CAGTAGTTTT TTTTCGGTCG GACTGCTCGA |
| 601 | ATTATTGTAT ATATGGAGAT CACATGCTAG TAACTTTTTC AATAATTTCA TGTTTCGAGC |
| 661 | AGGATGGTGA CTGACATCCC AGCATCAACT GATAATACAT ACGGTGAGTA CACCCCTATT |
| 721 | CCCATTTTGA AACAAGTAGA ATGTCTATTT TTATGATTTA GTATGTTCGT GACAATAGGC |
| 781 | TATAGCTATT TTGAAACTTC GGGAGCATAA AATAGTACTC GATTTTGTAT AACCATAAAC |
| 841 | ACACAGCTAG CCAATCTCTA TTCATATTTA TTTTAGTTTT ATTTGCCGAA CCATCCTCAA |
| 901 | CATCATAGCC ACTTGATCGA TCATCTCAAT CAGCGTTTGT ATCCTTGCCC GCTTGATTAT |
| 961 | CATCCATGGC AGTTCATATT TTTTTTCATT TCTTTCATGC TTGTTATAGT TTTATCTGAT |
| 1021 | GAATCCAAGA TGTTATTGAT CAATTAGTTC AGATGAGCAG TAATGCATGT TGGAGGTTTG |
| 1081 | GTAGTATATA TACGTTCAAA ATTTCACGAA ATCGGTAATT ACGGTGGGAG CCAAAAAAAA |
| 1141 | TTCCAAAATT TCGTATTACA TTAATAATGC ATGTGCTGTA GACTCATATT TTCTATGATT |
| 1201 | TCGATTCTGT CACCATCCTG CTCGAATATT TAAATCATGC TAATATTTTG TTTACATCTA |
| 1261 | AATCTTTTAT AAAAATTATA ATTTATATTT GGGTTTAACA ATTTCGGGCG CGTTTAGTGA |
| 1321 | GATTGGGTAA TTTCGGAGCG AGGCCACCGG CCACACGAAA AATTNCTATA CACGNACTAT |
| 1381 | ATGTGTACAT GTACATGCAT GGCACCCTGA TAGGCTACCC CATGGGGAAA AAATTGGAAA |
| 1441 | CGGACCATTC ATACGCAGTC GTGGTGCAGA CTGTGGGCCA CAATAGCAGT GTAAACATAA |
| 1501 | TTACGGTAAT CAAATACCCC ATGGGACCAT ATATATCATC CACAGATCCG TACGGTGCTT |
| 1561 | CCGTGTGGAT GGTCTACACC AGATCTTTTC CACACCATAA GGGCAGCAAT GCAGCATCAT |
| 1621 | ATTCATATAT GCACTAGTGA TGTACCATTT GGCTTATATC ATATTCAACC TAACTCCTTG |
| 1681 | GAAACATTAT GATATTCTAT TGGGTTGAAG ATGTCACTAC TACAAAAAAA AATCTTATGA |
| 1741 | GAGGTGTTTT GAAAACTGCC GGAGGTGCTT AAAGGAGACA GACGAGTTAG GACAACCGTC |
| 1801 | TCTATTAATG TGTACTAACT GAGGTAGTTA CCGTAACGTG CCTGACTTGA TTAACAGATT |
| 1861 | CAACCGTCTC AGTAAAGGCC ATGATTAACC GAAACAGATT CGAGAGTTTT CTTAAGTAGT |
| 1921 | TAAACTATTT TAATCTTCAC CGAACTTATA GAAAATGAAA GAGCTAACAC CAATATTTAT |
| 1981 | AAAAATAAAT TAGTATCACT AAATACATCA CGAAATCTAT TTGGTGTTGT AGAAGTTATC |
| 2041 | CTTTTCTATA AAATTGATCA AATTTATGAT AACTTAGTTT TAGGAATTCA TTTATTTTAG |
| 2101 | GACAACTGAG GAAGTACATA TTTTTTAAGT CATCCACAAA GTAGTGGATC CAATTTATTA |
| 2161 | CATTACTCTA CTACTTCAAA CTGAACAAAA GCCTAATCCT GGTTATTTTT AGAGTGATTT |
| 2221 | TTTACAACAT CAGCAGTAGT CCAGAAAATG GGAGGACATT AATAAAAGTG AAAAGGAGCA |
| 2281 | GAAGAAAGAT TACGGTATTT TATTTGTGCT ATTTGTTTAA CTATTGGCAG TTTGGGACCG |
| 2341 | AAATAAATAA CTGTTCGTAG CTCTATATTT GTCGATTCAA AAAGTGTAAC GATGATTTTT |
| 2401 | GTGTTTCAAA AGAAAAATAA AGAAGTGCAC CAATGATTGG ATATCATAGG CTATATATGT |
| 2461 | TGGATTAATT GCATCCAACG TATATAGTGA AAATGCTTTTCAATCAAGTA ATCTTCGAGC |
| 2521 | GGTTACCAGT TTTAATAGTT GCGAGTCGTC GTTTTTTATG TACCCTAGGA CATATATATC |
| 2581 | CGCATGTAGA CGATGATGAG ACTAGCAAGT TTTTTTTTTT TTTTGAGCAA ATACATAATT |
| 2641 | ATTGGATTTG CAGGCCGTGA GATGATGTGC TACGAGCCCC CTGCCCCGTC CACGGGCATC |
| 2701 | CACCGTATGG TGCTGGTGCT ATTCCAGCAG CTTGGCCGTG ACACGGTGTT CGCGGCGCCG |
| 2761 | TCCAGGCGCC ACAACTTCAA CACCCGTGCC TTCGCCCGCC GCTACAACCT CGGCGCGCCC |
| 2821 | GTCGCCGCCA TGTTCTTCAA CTGCCAGCGC CAGACCGGCT CCGGTGGCCC CAGGTTCACC |
| 2881 | GGGCCCTACA CCAGCCGACG TCGTGCGGGC TGA |

(SEQ ID NO:2 Sb06g012260—S. propinquum), or functional fragment, or variant, such as a codon optimized variant, thereof having at least 75%, 80%, 85%, 90%, 95%, 96%, 97%, 98%, or 99% sequence identity to SEQ ID NO:2.

In some embodiments, the maturity Ma1 gene (including non-coding sequence) as it is found in short day S. propinquum includes the nucleic acid sequence:

| | |
|---|---|
| 1 | CCCTGACCCT TGTTGGGCAA CATTTAGAGT CGTTAGCTTT GCAATTCTTT GGTTCCAATG |
| 61 | GATGGTTATC ATTTAGACAT ATTGGTCATG CTTAGTCAAA ACTTTATTGT TCGGCTATAA |
| 121 | ACTTTTCAGT ACTTTGTAAT AATTGGCTCG ATAGATGAAG CCGGGTATAA CATATCCTTT |
| 181 | ATCTAAAAAA ATTAGTTAAC ATGAACTTCA TATTCAATTC TTCATATCTC ACTAGCATCT |
| 241 | TTATTGTCTA GTTAGTTTTG TAGCATTGCA AAAAGCATGC AACTATATAC AATGAAACGG |
| 301 | AATAAAATTT CAGCTCTATT AATTTATATT TCAAATATAG GCCACTATAG CCATATTTCG |
| 361 | TGCTCAAGGC CACAAAATCT TGCGTACTTC CCTGTTGGTA CCAAAGAGAA GACGTTATTT |
| 421 | AACTTTGTTT GACTCTTCAA TATGGTTTGA ATCAGAAAAT TAGTTAAAAG AAAAGTGAGC |
| 481 | ACACCACGAC CTGTCATCAG CTCATGGTCA GCTCTACAAA CTTATAGATT GCATCGAGAT |
| 541 | CTAAGACTCA GGTACAAATC ATGTCAACAT CTAATGGTTT AGAAAATGAA AAGTTTTGAG |
| 601 | TTTCAAAATA TGATACGTGA TATTAACATT TGAACTTTTA GCAAGATCTG AAATAAAAAA |
| 661 | TTCAACTAGA TCATGTTAAC ATTGATATAA TCGCTTCCAA TCGCCTCCCA TCACTTCCGC |
| 721 | TAGAAAACTT TTTTTCTCGA TTTAATTAAT GAAAGGGTAA TAACATCATT GTACAAGATT |
| 781 | CTTTCAAACC TCAACCCCTA TCATCGACGG TGACGGCTCC CTATAACACG CACTAGTGGA |
| 841 | CGCCGGGCGG GTGGAACCCT AAGAAGATTT AAAAAAACTT AAGAAGAAGA TTTTTATCTA |
| 901 | ACTAACTATA GTACTTATAT CATACACTAT ACTATTCAAA ATATTATTTT CACAATTATG |
| 961 | AATTTACCCT TTTACTCTTC ATTAAAAAAA TACGAAAAAA GAATCACCAC GTCTCTATTT |
| 1021 | AGGGTCCTAG TCCCCATAAT TTAAGAGGCG GTGAGAGACG ATGTGACGTC TATGGACCAC |
| 1081 | CGACCAAAGA CACACCTATC GTCTCCCATC GCCTTGCTTC CATCGCCTCT CATCGCTTTT |
| 1141 | CATATTCTAG ATCCAGCGGC CATAGACACA CCAATCGTTT CTCATCGCCT CTCCAACCAT |
| 1201 | TGTAAAAATA TTTATAATTT TGATATAAAA TTTGTCTTCA CTTGAGTTCA TGCCAAAAAA |
| 1261 | ATTATACATA TTATTTTCGT GTGAGAATTT ACAGAAGTGG ACTCTTAAGA TGTCCAAATG |
| 1321 | TAAATGACCC TATTTATTAT GAGGCGCGGA TCTATAGGCC TGACTCTGAA AATGGATTAT |
| 1381 | GGATTTGAGA TAATAAATTT AAGGGCCTAT CTTCGCACAT AACATCTATA GTTCCTAAAT |
| 1441 | TTTTTTTTAT TGTAGTAGTA GAACTTTTCT CCCTGTAAAC CAAGTTGACG CTGGGCTTTA |
| 1501 | TTTTGCGACA CAGAACACCA AATTGGTGGC TATGAACTCT TCCACCTGGG CAGGGAAAAC |
| 1561 | GGTTTATTAT GTTTCTCTTT AATTTATCTA TCGTGGCACT ATAACACAAC ATGGCTTTGC |
| 1621 | CGACACTTCC AACTATCGGC AAAGGGTACC TTTACCGACA CTTAACGTCT CACGAAAGGT |
| 1681 | TTTGCCGACA ATTTTCAAAC AGTCGCGGTA GAAGCAGTTG GCGAAACTTT TGCCGACAGT |
| 1741 | TAAAGGCATC GCCGACACAT TTTCTGTAGT CAAATGGCAT ACCTACGCCG ACAGTTGAAC |
| 1801 | TTTCACCGAC AGTGAACCCT TTGCCGACAG TTTGGACCTA CGCCGACAGT TTGGACCTTT |
| 1861 | TCCGACAGTT GGTATGTTAG CGAAACCGTT TCTAGGGTGT TTCATAAACC ATGCCTTGTC |
| 1921 | CAACAGTAGA AGTGTCGGCA AAACTATATT GCTAGGATGT AGATACAATT TAAATATTTT |
| 1981 | AATAAATACA CATCACATTG ATTGAGCAAA ATCACATGGT CTGTTTTCAC TAAAACTGTC |
| 2041 | AGAGGTACAC TCCAGTACTA CCAGTACGTC GCCCGCACAG TGGCCAAGGA TTTTACTGCT |
| 2101 | ACTGTTGATT AACATAAGCA CTTGCGACTT TCCCTAAAAT CTTTTATAAA ACAACGGCCG |
| 2161 | CAATAATATT GAACTATTTT TTTTCTAGTA CCAAAATTAG AATTTGATCC CTCACCTCAT |
| 2221 | TACATCCATA GTAACATGAC CAGATATATA TGGACAGGAT GGGATCACTC AGCGAGCAGA |
| 2281 | TACACTGAGC GATTCATAAT CAGATTTTTT AATTTCTTCT AGTGAAGTGG GGTTTTCCTA |
| 2341 | GTCTTTTAAC ATTCAAAATT TAGTACAAAC TTTCCCTAGT AAATGCCTTC TAGTAAAGAT |
| 2401 | TTCCTAGTAT TTTGACTAGC GATAGTGTTT TATTACTAAT TAAAAACATT AGAAGAACTC |
| 2461 | CATTTAGTGA TTGGTTGTTT GGATTAGTCT TCTCACGTTA GACCTATATA TGCAGGACAA |
| 2521 | CTCAAGCCAG CATAAATATA TGAAATATCT TGGTGTTTGT TTGTCTGACA CAGGCAACCG |
| 2581 | CGTTTGGTAT AAATGTGTTT TCTTGTTTAC ATTTTACCAT CTATAGTCAT CTCAATGTTA |
| 2641 | TATAGTAGAG GCTTCATGTT TGTAGTAGAT AAGGTAGAGA ATTGAGAATA TTTTATTTTT |
| 2701 | GTGCGACCAT CAATTTTATG TAATCTGCAT TGTCTAATGC TTTATTTGAC ATTTGAAACT |
| 2761 | ACTTAATTTG ACAGTTATGC AGGTCCGCAT GATCCTATGA AAGCAATTAA TTAGTACGGG |
| 2821 | TAAACTGCAC TACACAAGTT TGCTAGTACT ATTCTATTAA CCGACCTGTC AATATTACCT |
| 2881 | TAAGTTACTG ATTTCAATTA GAATCTAACA CATTCAGGAA AAGAAGTTTC ACTAGTACAA |
| 2941 | AAATCATTTT CGTTGGCACG TTGTTTTTTT TTTCACAGGC AGTTCACAAT ATCATGGTGC |
| 3001 | TAGTAGAAAA ATTTCAACGG GCCCAACAAG AGAACCGCCA GGCGGTCTTC TTAATTCAAC |
| 3061 | CGCCTGTGTA AACTTTCCAT TTACATAGGC GGCTTACGAT AAAAACCGTG TGTATAAATA |
| 3121 | CCATTAACAC AGGCAGTCGA GTTACGACAA CCGCCTGTGT AAATGTGTCT TTTTACACAG |
| 3181 | GCGGTTTGTA TAGAGGGCCG CCTGTGCTAA TATATTTACA CAGGCTATGA GCCGCCTGTG |
| 3241 | TTAAGTCTTC TATAAATACC CTTCGTCCAC CTCCAGACAA GAACAGTTAC TCCCATGAGC |
| 3301 | TCTGCACACT GGCGGACCAG ACGATTCCAG TTTCCAAGGG GGGAGGTTTT GATTTTCATT |
| 3361 | TCTTTGGTGA GAAACTTCCA AAAGGTTAGT TAGTGCCATT GATGCTATTT TTTAAGCGAT |
| 3421 | TCTTTGGTTC AATTCTTGTA TTGGAGGTGC TCTAGATCTA GAGTTCATCA TGCATTCTTG |
| 3481 | CTTAGGGTTA GAGTTCATAG GGCAAAAAGA GAGAGATTTA GCTAAATTTT TATGTAAATT |
| 3541 | CATAGTAAAT TGTAAAAATT AAAAAAAATA AAAAATAAAT ACTTTTTAGA ATTCTTGTGA |
| 3601 | GTAGATCTAT ACAATAGAGT AATGATGAGG ATATTTTGAA GTTTATAATT TTGATTCAGT |
| 3661 | TTTAGCTTTT CTTTTTTCAG ATGAATTAGA CTTTATAAAC TCAAACATTA AAATGTTGAA |
| 3721 | AATCATAAAA TGGCAAATAA ATACTTTTTC AAATCTTTGT GCATAAATAC TTCATAGAAA |
| 3781 | TCCTTGAATT ATTCCTAAAT TTTATACAAT TGTTTCTTAT AATTATGAAA ATGAGTTTAA |
| 3841 | ACAATTATTT AAATTCCATA AATTGTAACT CCGTAAGGTG TAGGTTTTCA TCTCTGTTTA |
| 3901 | ATAGAAGGAG GTTAGTATCT TAGTTAAGTC TGTTTTCGGG GGTTATATTA GTTTTGTTTT |
| 3961 | TAGATTGACC TACATTAATT GTTCTTAACT AATTACAGCT AAATATGGAG AGGTCATTAT |
| 4021 | GGATGTACAA CTTATCAAGA TTGGACCTAT CATATGTAGT GCAGGTCCAA AAATTTATTG |
| 4081 | ATGTCGCAAA GATACATGCT CGCAGAACAA AGGCGAAGCA CATATGTTGT CCATGCGCAG |
| 4141 | ACTGCAAAAA TATTATGGTA TTTGACAATG TAGAAGCAAT TACTTCCCAT CTGGTTTGAA |
| 4201 | GAGGATTTAT GGAGGACTAC TTGATTTGGA CAAAACATGG TGAGGGTAGT TTTGCACCTT |
| 4261 | ATATGCGGAC AACTGACAAC ACTGCAACTA ACATCAATGT GGAGGGTCCA ATGCCACCTC |
| 4321 | TCAATGAATT TCATGCTATG CCAGATGTTA ATGAAACTCA TACGTCTGAT GTCAATGAAA |
| 4381 | CTCAGCATGC TAACACAGAT GTTGTTGAAG ATGCAGATTT CTTAGAGGCA ATAATGAACC |
| 4441 | GTTGTGCGGA TCCATCAATA TTCTTCATGA AGGGAATGAA AGCATTGAAG AAGGCAGCAG |
| 4501 | AGGACACTTT GTACGACGAG TCAAAAGGTT GTACCAAACA ATGGTCGACA TTATGTGTTG |
| 4561 | TTCTTCAGTT TTTGACGATG AAGGCTAGAC ATGGTTGGTC CGATGCTAGC TTCAATGATT |
| 4621 | TCTTGCGTGT ACTTGGAGAC CTTCTTCCTA AGGAGAACAA AGTGCCTGCT AACACATACT |
| 4681 | ATGCAAAGAA GCTAGTCAGT CCACTTACGA TAGGTGTTGA GAAGATCCAC GCATGTAGAA |
| 4741 | ATCATTGTAT TCTATATCGA GGTGATCAAT ATAAAGACTT AGACAGTTGT CCAAACTGTG |
| 4801 | GTGCCAGTAG GTACAAGACA AACAAAGATT TTCGGGAGGA AGAGAATCTA GCCTCTGTTT |
| 4861 | CTACAGGGAG GAAGCGAAAG AAGACCCAAA CAAAGACTCA ACAAGACAAG CGCTCAAAGC |
| 4921 | CTAGTAGCAA TGAAGAAGTG GACTATTATG CATTGAGAAG AGTCTCCCTA TGAGCCAAAA |
| 4981 | AAGGGGACAG CAGCAGGCAC AACTCTCTTT CTGAAAGGAC TTGGAAAGCA GCGGACGGCA |
| 5041 | CGGCTCATTG AGCTCGAACC GTCACAGAAA AAGGAAGCCA CCGCCCAGTC AATAGAAGCC |
| 5101 | ATGCCCCCAT CAAAGGAAGC CCCAAGTGGC GATGTACATA TTGAACAGCC ATCAAGTCAA |
| 5161 | CCATTGACCC TAAAGGATAT CAGAAAGCCA ACGATTGATG ATTATGTCAA TGTCCCTAGT |
| 5221 | GACTATGTGC CCGGAAGGCC TATGCTCCAA TGGACGCTGC TCGATTAGAT TCAATGGCTG |
| 5281 | ATAAAAAGGT TTCATGACTG GTACATGAGA GCAGTGCATG CTAGCCTCCA TGGAATCAGA |
| 5341 | GTTGATATAC CAACAGACAT GTTTGCTACT GGTAACAAAA AAAGCAAGAC ATTTGTTACC |
| 5401 | TTTGAGGACA TGCACTTGTT ATTGAACTAT AGGCGGCTTG ACGTCCAACT CATAACAATC |
| 5461 | TGGTGCCTGT AAGTATCACT CATGCACACA CAATTATTAT ATATTAATAT GTAGTGTGAA |
| 5521 | ACTCTAATAT GTAGATGTTG TCTGTAGTTT GCAAGATCAC GAGCAGATGT CATTATTATC |
| 5581 | TGCCGGATCG ATGGTCGGTT ATCTGAGCCC TATCAAGTTA CAAGAAAATA TGAACAAATT |
| 5641 | CGTATTATCA AAGGAAGATA GAGCAAAGAT AGAGGAAGAC AAAACACCAG GATAATTATG |
| 5701 | CCATCTATCT TGGTAGATCA ATGCTGAGGT ATAAATATAG GGATTTTATA TTGGCACCAT |
| 5761 | ACAACATTAG GTAAGCTTGA CTTCATATAC GTATTTCAAA TTATCGTGTA AACAATATAC |
| 5821 | ATGTGTCGCT CACTCATTTA TTCATGCAGT GACCATTGGA TTGTTTTTTA TATTTATCCC |
| 5881 | TTCGAAGGGA AGGTGCTTGT CCTAGACTCT TTACATGTTC CTCCCGAGAA GTATCAACCA |
| 5941 | TTCTTGGTTC AATTAGAAAG GTGAGCCAAC ATGAAACCAC ATGCGTACTT ATATAAATTA |
| 6001 | GAGTTTCAAA ATAACTTTAG TGATTTAGGT TCGATATCTA CGGGGCATGG CGGTTTTATA |
| 6061 | AGAAACAAAA GGGACCTGTC GACGCTGCAC GCTCAGATCC TAGGATCCCA TTGATGATAC |
| 6121 | AACACCACTA TCCGGTAAGT TTTCTGAACA CATTTCATCA TATAAATAAT ACATAAAGCA |
| 6181 | TGGCAAATTT AGAATAATCC GTTGCTCATT ATATAGTGCC ACAAGCAACC ACCTGGATCG |
| 6241 | GTCTATTGTG GGTACTATGT CTGTGAGTTT ATAAGGCAGC GGGGACGTTA CGTCAAGGAC |
| 6301 | AAAAATATGG TAAATAATAT CTATGTATGA AGTTTTCTCA TTAAAGCTGC AAAATTATAT |
| 6361 | ATTGAACATG TGTCAATCAT GCTTTTAAAC TTTATTTTCA GCCGAAAAAG CAAGGAAAAG |
| 6421 | ACGTGCCCTT TACACCAAAG ACTCTGGAAG ATATAGTAGC ATACTTGTGT GGTTTTATTA |
| 6481 | TGAGAGAAAT AATTTCAAGT GACAGTGCAT ATTTTGATCA TGAGGGCGAT TTAGCAAGTG |
| 6541 | ATAAATTTAG AGTGCTGACA GACATAGCAG GTCTAAATCT GAAGCGAAAC GACATGTAAA |
| 6601 | CATTGTATGG TTGTGCGGAT AACATGCATT GACGTGTATA TATATAATTT TATGGTTGAT |
| 6661 | GTTTGATTTG TTTACAATTC TATAATATAT ATATGTGGTG TATGTATGAT GTTGTGTGTG |
| 6721 | TATATATATA TATATATATA TATATATATA TATATATATA TATATATATA TATATATATA |
| 6781 | ATGTTTAGCA CTGTGTTTGG TGGGAAAAAT TAAAATTTGA AATATATATA AAAAATTATT |
| 6841 | TACACAGACA GTGTAGTGTG AGCTGCCTGT GTAAAAATAC ATTTATACAG GCGGCTCACC |
| 6901 | TTGTCNNNNC AGGCGGTGCT AAAAGCATCT TCACAGGCGG CCAAGCCCAC CGCCTGTACC |
| 6961 | AGGGGTCAGT ACAAAATGGA CCACAGTACA GGCGGGGCTG TGCGAGCCGC CTGTGAAAAC |
| 7021 | ATAATTTTCA CAGGCGGCTC GCACAGCCCC GCCTGTACTG TGGTCCATTT TGTACTGACC |
| 7081 | CCTGGTACAG GCGGTGGGCT TGGCCGCCTG TGAAGATGCT TTTAGCACCG CCTGTAAAAA |
| 7141 | TGTTTTTTGT AGCAGTGTTT TTCTTATTAG TAGTATCTTT TATACTAATT AAGATTCAAT |
| 7201 | AAAAATTCAC CATGACATCC CCATTGCCAA GAGAATATTT CGCCGCCCCT CAAAGCAGCC |
| 7261 | AATAAGGCTT TACTAAAAAG ACTATCCACG CAGTAGAGAT TTAGTCAAAA TATTCCAATA |
| 7321 | GCAATTGTTT CCTGCCTGCT TGACCTTCGT CAGCCACTCA CTGTATAAAT ATCGCACCAC |
| 7381 | GCCCTTTGCA GGCTTACAGA GCTTGTATTA CGTACTAACA AGGCACACAC AGTACCCTGT |
| 7441 | GTTCACCGGC CCTGCACAAA ACTCAAGCAG TTATTACTAA CATGGCGGCT AACGATTCCT |
| 7501 | TGGTTACTGC TCATGTGATA GGAGATGTCT TGGACCCCTT CTATACAACC GTTGACATGA |
| 7561 | TGATCCTATT CGATGGTACT CCTATTATCA GCGGCATGGA GTTGCGCGCT CCGGCGGTTT |
| 7621 | CTGACAGGCC AAGGGTTGAA ATTGGAGGAG ATGATTATCG AGTTGCATAT ACTCTGGTAA |
| 7681 | ACTCATGCCA TGTCAATTAA CTAGTAGTTG AATTTAGATG CTGGTGGTAT CGTGGATACA |
| 7741 | TGTACTATAT GTTATGGTTG ATACATATTT GTTTAATTGA TCGCAACACC ATTTGCGGTA |
| 7801 | ACTTCAAATT ACATTCTTTC AATATATAGG TGATGGTCGA TCCTGATGCT CCTAACCCAA |
| 7861 | GCAACCCAAC CTTGAGGGAG TACTTGCACT GGTAAGAGAA ACCTATAGAC GACAATTATT |
| 7921 | GTTGTTGGCA TGTTTTGCCC ACATATACTT TGTGTGTGTA TATTTGTGCT TATGCTTCTC |
| 7981 | CATAAAATTT TGGTGTATGT CTCAAGAGAG ATAGGTATAG AGGTTAGCAG TCCTTTAAAA |
| 8041 | ATGGTTTAAT CCAGTAGTTT TTTTTCGGTC GGACTGCTCG AATTATTGTA TATATGGAGA |
| 8101 | TCACATGCTA GTAACTTTTT CAATAATTTC ATGTTTCGAG CAGGATGGTG ACTGACATCC |
| 8161 | CAGCATCAAC TGATAATACA TACGGTGAGT ACACCCCTAT TCCCATTTTG AAACAAGTAG |
| 8221 | AATGTCTATT TTTATGATTT AGTATGTTCG TGACAATAGG CTATAGCTAT TTTGAAACTT |
| 8281 | CGGGAGCATA AAATAGTACT CGATTTTGTA TAACCATAAA CACACAGCTA GCCAATCTCT |
| 8341 | ATTCATATTT ATTTTAGTTT TATTTGCCGA ACCATCCTCA ACATCATAGC CACTTGATCG |
| 8401 | ATCATCTCAA TCAGCGTTTG TATCCTTGCC CGCTTGATTA TCATCCATGG CAGTTCATAT |
| 8461 | TTTTTTTCAT TTCTTTCATG CTTGTTATAG TTTTATCTGA TGAATCCAAG ATGTTATTGA |
| 8521 | TCAATTAGTT CAGATGAGCA GTAATGCATG TTGGAGGTTT GGTAGTATAT ATACGTTCAA |
| 8581 | AATTTCACGA AATCGGTAAT TACGGTGGGA GCCAAAAAAA ATTCCAAAAT TTCGTATTAC |
| 8641 | ATTAATAATG CATGTGCTGT AGACTCATAT TTTCTATGAT TTCGATTCTG TCACCATCCT |
| 8701 | GCTCGAATAT TTAAATCATG CTAATATTTT GTTTACATCT AAATCTTTTA TAAAAATTAT |
| 8761 | AATTTATATT TGGGTTTAAC AATTTCGGGC GCGTTTAGTG AGATTGGGTA ATTTCGGAGC |
| 8821 | GAGGCCACCG GCCACACGAA AAATTCTATA CACGACTATA TGTGTACATG TACATGCATG |
| 8881 | GCACCCTGAT AGGCTACCCC ATGGGGAAAA AATTGGAAAC GGACCATTCA TACGCAGTCG |
| 8941 | TGGTGCAGAC TGTGGGCCAC AATAGCAGTG TAAACATAAT TACGGTAATC AAATACCCCA |
| 9001 | TGGGACCATA TATATCATCC ACAGATCCGT ACGGTGCTTC CGTGTGGATG GTCTACACCA |
| 9061 | GATCTTTTCC ACACCATAAG GGCAGCAATG CAGCATCATA TTCATATATG CACTAGTGAT |
| 9121 | GTACCATTTG GCTTATATCA TATTCAACCT AACTCCTTGG AAACATTATG ATATTCTATT |
| 9181 | GGGTTGAAGA TGTCACTACT ACAAAAAAAA ATCTTATGAG AGGTGTTTTG AAAACTGCCG |
| 9241 | GAGGTGCTTA AAGGAGACAG ACGAGTTAGG ACAACCGTCT CTATTAATGT GTACTAACTG |
| 9301 | AGGTAGTTAC CGTAACGTGC CTGACTTGAT TAACAGATTC AACCGTCTCA GTAAAGGCCA |
| 9361 | TGATTAACCG AAACAGATTC GAGAGTTTTC TTAAGTAGTT AAACTATTTT AATCTTCACC |
| 9421 | GAACTTATAG AAAATGAAAG AGCTAACACC AATATTTATA AAAATAAATT AGTATCACTA |
| 9481 | AATACATCAC GAAATCTATT TGGTGTTGTA GAAGTTATCC TTTTCTATAA AATTGATCAA |
| 9541 | ATTTATGATA ACTTAGTTTT AGGAATTCAT TTATTTTAGG ACAACTGAGG AAGTACATAT |
| 9601 | TTTTTAAGTC ATCCACAAAG TAGTGGATCC AATTTATTAC ATTACTCTAC TACTTCAAAC |
| 9661 | TGAACAAAAG CCTAATCCTG GTTATTTTTA GAGTGATTTT TTACAACATC AGCAGTAGTC |
| 9721 | CAGAAAATGG GAGGACATTA ATAAAAGTGA AAAGGAGCAG AAGAAAGATT ACGGTATTTT |
| 9781 | ATTTGTGCTA TTTGTTTAAC TATTGGCAGT TTGGGACCGA AATAAATAAC TGTTCGTAGC |
| 9841 | TCTATATTTG TCGATTCGAA AGTGTAACGA TGATTTTTGT GTTTCAAAAG AAAAATAAAG |
| 9901 | AAGTGCACCA ATGATTGGAT ATCATAGGCT ATATATGTTG GATTAATTGC ATCCAACGTA |
| 9961 | TATAGTGAAA ATGCTTTTCA ATCAAGTAAT CTTCGAGCGG TTACCAGTTT TAATAGTTGC |
| 10021 | GAGTCGTCGT TTTTTATGTA CCCTAGGACA TATATATCCG CATGTAGACG ATGATGAGAC |
| 10081 | TAGCAAGTTT TTTTTTTTTT TTGAGCAAAT ACATAATTAT TGGATTTGCA GGCCGTGAGA |
| 10141 | TGATGTGCTA CGAGCCCCCT GCCCCGTCCA CGGGCATCCA CCGTATGGTG CTGGTGCTAT |
| 10201 | TCCAGCAGCT TGGCCGTGAC ACGGTGTTCG CGGCGCCGTC CAGGCGCCAC AACTTCAACA |
| 10261 | CCCGTGCCTT CGCCCGCCGC TACAACCTCG GCGCGCCCGT CGCCGCCATG TTCTTCAACT |
| 10321 | GCCAGCGCCA GACCGGCTCC GGTGGCCCCA GGTTCACCGG GCCCTACACC AGCCGACGTC |
| 10381 | GTGCGGGCTG ATGACGACGA TCGTCGTTAC GTCACGTGTA CCGTACACAT ATATGTATAG |
| 10441 | ATATACATGC ATGCATGTTC CATGGTATAG GATCGGTGAC AAAACGTCTA ATAATGTATA |
| 10501 | CACACACATG CATGGAATGC ATGTAATAAG AGAATATATG TATAATAAGT AGGGGAGAGC |
| 10561 | ATGCATATAT TGTGTACACG CGTCCGATGC GTATAGCCCT TTACATTATT GTAGTTGTAA |
| 10621 | TCAG |

(SEQ ID NO:3 Sb06g012260 (10.6 KB)—S. propinquum), or functional fragment, or variant, such as a codon optimized variant, thereof having at least 75%, 80%, 85%, 90%, 95%, 96%, 97%, 98%, or 99% sequence identity to SEQ ID NO:3. Each N=can be any nucleotide or combination of any 2, 3, 4, or 5 nucleotides.

The coding sequence of the maturity Ma1 gene of SEQ ID NO:3, including introns, can be:

| | |
|---|---|
| 1 | ATGGCGGCTA ACGATTCCTT GGTTACTGCT CATGTGATAG GAGATGTCTT GGACCCCTTC |
| 61 | TATACAACCG TTGACATGAT GATCCTATTC GATGGTACTC CTATTATCAG CGGCATGGAG |
| 121 | TTGCGCGCTC CGGCGGTTTC TGACAGGCCA AGGGTTGAAA TTGGAGGAGA TGATTATCGA |
| 181 | GTTGCATATA CTCTGGTAAA CTCATGCCAT GTCAATTAAC TAGTAGTTGA ATTTAGATGC |
| 241 | TGGTGGTATC GTGGATACAT GTACTATATG TTATGGTTGA TACATATTTG TTTAATTGAT |
| 301 | CGCAACACCA TTTGCGGTAA CTTCAAATTA CATTCTTTCA ATATATAGGT GATGGTCGAT |
| 361 | CCTGATGCTC CTAACCCAAG CAACCCAACC TTGAGGGAGT ACTTGCACTG GTAAGAGAAA |
| 421 | CCTATAGACG ACAATTATTG TTGTTGGCAT GTTTTGCCCA CATATACTTT GTGTGTGTAT |
| 481 | ATTTGTGCTT ATGCTTCTCC ATAAAATTTT GGTGTATGTC TCAAGAGAGA TAGGTATAGA |
| 541 | GGTTAGCAGT CCTTTAAAAA TGGTTTAATC CAGTAGTTTT TTTTCGGTCG GACTGCTCGA |
| 601 | ATTATTGTAT ATATGGAGAT CACATGCTAG TAACTTTTTC AATAATTTCA TGTTTCGAGC |
| 661 | AGGATGGTGA CTGACATCCC AGCATCAACT GATAATACAT ACGGTGAGTA CACCCCTATT |
| 721 | CCCATTTTGA AACAAGTAGA ATGTCTATTT TTATGATTTA GTATGTTCGT GACAATAGGC |
| 781 | TATAGCTATT TTGAAACTTC GGGAGCATAA AATAGTACTC GATTTTGTAT AACCATAAAC |
| 841 | ACACAGCTAG CCAATCTCTA TTCATATTTA TTTTAGTTTT ATTTGCCGAA CCATCCTCAA |
| 901 | CATCATAGCC ACTTGATCGA TCATCTCAAT CAGCGTTTGT ATCCTTGCCC GCTTGATTAT |
| 961 | CATCCATGGC AGTTCATATT TTTTTTCATT TCTTTCATGC TTGTTATAGT TTTATCTGAT |
| 1021 | GAATCCAAGA TGTTATTGAT CAATTAGTTC AGATGAGCAG TAATGCATGT TGGAGGTTTG |
| 1081 | GTAGTATATA TACGTTCAAA ATTTCACGAA ATCGGTAATT ACGGTGGGAG CCAAAAAAAA |
| 1141 | TTCCAAAATT TCGTATTACA TTAATAATGC ATGTGCTGTA GACTCATATT TTCTATGATT |
| 1201 | TCGATTCTGT CACCATCCTG CTCGAATATT TAAATCATGC TAATATTTTG TTTACATCTA |
| 1261 | AATCTTTTAT AAAAATTATA ATTTATATTT GGGTTTAACA ATTTCGGGCG CGTTTAGTGA |
| 1321 | GATTGGGTAA TTTCGGAGCG AGGCCACCGG CCACACGAAA AATTCTATAC ACGACTATAT |
| 1381 | GTGTACATGT ACATGCATGG CACCCTGATA GGCTACCCCA TGGGGAAAAA ATTGGAAACG |
| 1441 | GACCATTCAT ACGCAGTCGT GGTGCAGACT GTGGGCCACA ATAGCAGTGT AAACATAATT |
| 1501 | ACGGTAATCA AATACCCCAT GGGACCATAT ATATCATCCA CAGATCCGTA CGGTGCTTCC |
| 1561 | GTGTGGATGG TCTACACCAG ATCTTTTCCA CACCATAAGG GCAGCAATGC AGCATCATAT |
| 1621 | TCATATATGC ACTAGTGATG TACCATTTGG CTTATATCAT ATTCAACCTA ACTCCTTGGA |
| 1681 | AACATTATGA TATTCTATTG GGTTGAAGAT GTCACTACTA CAAAAAAAAA TCTTATGAGA |
| 1741 | GGTGTTTTGA AAACTGCCGG AGGTGCTTAA AGGAGACAGA CGAGTTAGGA CAACCGTCTC |
| 1801 | TATTAATGTG TACTAACTGA GGTAGTTACC GTAACGTGCC TGACTTGATT AACAGATTCA |
| 1861 | ACCGTCTCAG TAAAGGCCAT GATTAACCGA AACAGATTCG AGAGTTTTCT TAAGTAGTTA |
| 1921 | AACTATTTTA ATCTTCACCG AACTTATAGA AAATGAAAGA GCTAACACCA ATATTTATAA |
| 1981 | AAATAAATTA GTATCACTAA ATACATCACG AAATCTATTT GGTGTTGTAG AAGTTATCCT |
| 2041 | TTTCTATAAA ATTGATCAAA TTTATGATAA CTTAGTTTTA GGAATTCATT TATTTTAGGA |
| 2101 | CAACTGAGGA AGTACATATT TTTTAAGTCA TCCACAAAGT AGTGGATCCA ATTTATTACA |
| 2161 | TTACTCTACT ACTTCAAACT GAACAAAAGC CTAATCCTGG TTATTTTTAG AGTGATTTTT |
| 2221 | TACAACATCA GCAGTAGTCC AGAAAATGGG AGGACATTAA TAAAAGTGAA AAGGAGCAGA |
| 2281 | AGAAAGATTA CGGTATTTTA TTTGTGCTAT TTGTTTAACT ATTGGCAGTT TGGGACCGAA |
| 2341 | ATAAATAACT GTTCGTAGCT CTATATTTGT CGATTCGAAA GTGTAACGAT GATTTTTGTG |
| 2401 | TTTCAAAAGA AAAATAAAGA AGTGCACCAA TGATTGGATA TCATAGGCTA TATATGTTGG |
| 2461 | ATTAATTGCA TCCAACGTAT ATAGTGAAAA TGCTTTTCAA TCAAGTAATC TTCGAGCGGT |
| 2521 | TACCAGTTTT AATAGTTGCG AGTCGTCGTT TTTTATGTAC CCTAGGACAT ATATATCCGC |
| 2581 | ATGTAGACGA TGATGAGACT AGCAAGTTTT TTTTTTTTTT TGAGCAAATA CATAATTATT |
| 2641 | GGATTTGCAG GCCGTGAGAT GATGTGCTAC GAGCCCCCTG CCCCGTCCAC GGGCATCCAC |
| 2701 | CGTATGGTGC TGGTGCTATT CCAGCAGCTT GGCCGTGACA CGGTGTTCGC GGCGCCGTCC |
| 2761 | AGGCGCCACA ACTTCAACAC CCGTGCCTTC GCCCGCCGCT ACAACCTCGG CGCGCCCGTC |
| 2821 | GCCGCCATGT TCTTCAACTG CCAGCGCCAG ACCGGCTCCG GTGGCCCCAG GTTCACCGGG |
| 2881 | CCCTACACCA GCCGACGTCG TGCGGGCTGA |

(SEQ ID NO:4 Sb06g012260 (10.6 kb)—S. propinquum) or functional fragment or variant, such as a codon optimized variant, thereof having at least 75%, 80%, 85%, 90%, 95%, 96%, 97%, 98%, or 99% sequence identity to SEQ ID NO:4.

In some embodiments, the maturity Ma1 gene (including non-coding sequence) as it is found in short day S. propinquum includes the nucleic acid sequence:

| | |
|---|---|
| 1 | CTATGCTCCA ATGGACGCTG CTCGATTAGA TTCAATGGCT GATAAAAAGG TTTCATGACT |
| 61 | GGTACATGAG AGCAGTGCAT GCTAGCCTCC ATGGAATCAG AGTTGATATA CCAACAGACA |
| 121 | TGTTTGCTAC TGGTAACAAA AAAAGCAAGA CATTTGTTAC CTTTGAGGAC ATGCACTTGT |
| 181 | TATTGAACTA TAGGCGGCTT GACGTCCAAC TCATAACAAT CTGGTGCCTG TAAGTATCAC |
| 241 | TCATGCACAC ACAATTATTA TATATTAATA TGTAGTGTGA AACTCTAATA TGTAGATGTT |
| 301 | GTCTGTAGTT TGCAAGATCA CGAGCAGATG TCATTATTAT CTGCCGGATC GATGGTCGGT |
| 361 | TATCTGAGCC CTATCAAGTT ACAAGAAAAT ATGAACAAAT TCGTATTATC AAAGGAAGAT |
| 421 | AGAGCAAAGA TAGAGGAAGA CAAAACACCA GGATAATTAT GCCATCTATC TTGGTAGATC |
| 481 | AATGCTGAGG TATAAATATA GGGATTTTAT ATTGGCACCA TACAACATTA GGTAAGCTTG |
| 541 | ACTTCATATA CGTATTTCAA ATTATCGTGT AAACAATATA CATGTGTCGC TCACTCATTT |
| 601 | ATTCATGCAG TGACCATTGG ATTGTTTTTT ATATTTATCC CTTCGAAGGG AAGGTGCTTG |
| 661 | TCCTAGACTC TTTACATGTT CCTCCCGAGA AGTATCAACC ATTCTTGGTT CAATTAGAAA |
| 721 | GGTGAGCCAA CATGAAACCA CATGCGTACT TATATAAATT AGAGTTTCAA AATAACTTTA |
| 781 | GTGATTTAGG TTCGATATCT ACGGGGCATG GCGGTTTTAT AAGAAACAAA AGGGACCTGT |
| 841 | CGACGCTGCA CGCTCAGATC CTAGGATCCC ATTGATGATA CAACACCACT ATCCGGTAAG |
| 901 | TTTTCTGAAC ACATTTCATC ATATAAATAA TACATAAAGC ATGGCAAATT TAGAATAATC |
| 961 | CGTTGCTCAT TATATAGTGC CACAAGCAAC CACCTGGATC GGTCTATTGT GGGTACTATG |
| 1021 | TCTGTGAGTT TATAAGGCAG CGGGGACGTT ACGTCAAGGA CAAAAATATG GTAAATAATA |
| 1081 | TCTATGTATG AAGTTTTCTC ATTAAAGCTG CAAAATTATA TATTGAACAT GTGTCAATCA |
| 1141 | TGCTTTTAAA CTTTATTTTC AGCCGAAAAA GCAAGGAAAA GACGTGCCCT TTACACCAAA |
| 1201 | GACTCTGGAA GATATAGTAG CATACTTGTG TGGTTTTATT ATGAGAGAAA TAATTTCAAG |
| 1261 | TGACAGTGCA TATTTTGATC ATGAGGGCGA TTTAGCAAGT GATAAATTTA GAGTGCTGAC |
| 1321 | AGACATAGCA GGTCTAAATC TGAAGCGAAA CGACATGTAA ACATTGTATG GTTGTGCGGA |
| 1381 | TAACATGCAT TGACGTGTAT ATATATAATT TTATGGTTGA TGTTTGATTT GTTTACAATT |
| 1441 | CTATAATATA TATATGTGGT GTATGTATGA TGTTGTGTGT GTATATATAT ATATATATAT |
| 1501 | ATATATATAT ATATATATAT ATATATATAT ATATATATAT AATGTTTAGC ACTGTGTTTG |
| 1561 | GTGGGAAAAA TTAAAATTTG AAATATATAT AAAAAATTAT TTACACAGAC AGTGTAGTGT |
| 1621 | GAGCTGCCTG TGTAAAAATA CATTTATACA GGCGGCTCAC CTTGTNNNNN CAGGCGGTGC |
| 1681 | TAAAAGCATC TTCACAGGCG GCCAAGCCCA CCGCCTGTAC CAGGGGTCAG TACAAAATGG |
| 1741 | ACCACAGTAC AGGCGGGGCT GTGCGAGCCG CCTGTGAAAA CATAATTTTC ACAGGCGGCT |
| 1801 | CGCACAGCCC CGCCTGTACT GTGGTCCATT TTGTACTGAC CCCTGGTACA GGCGGTGGGC |
| 1861 | TTGGCCGCCT GTGAAGATGC TTTTAGCACC GCCTGTAAAA ATGTTTTTTG TAGCAGTGTT |
| 1921 | TTTCTTATTA GTAGTATCTT TTATACTAAT TAAGATTCAA TAAAAATTCA CCATGACATC |
| 1981 | CCCATTGCCA AGAGAATATT TCGCCGCCCC TCAAAGCAGC CAATAAGGCT TTACTAAAAA |
| 2041 | GACTATCCAC GCAGTAGAGA TTTAGTCAAA ATATTCCAAT AGCAATTGTT TCCTGCCTGC |
| 2101 | TTGACCTTCG TCAGCCACTC ACTGTATAAA TATCGCACCA CGCCCTTTGC AGGCTTACAG |
| 2161 | AGCTTGTATT ACGTACTAAC AAGGCACACA CAGTACCCTG TGTTCACCGG CCCTGCACAA |
| 2221 | AACTCAAGCA GTTATTACTA ACATGGCGGC TAACGATTCC TTGGTTACTG CTCATGTGAT |
| 2281 | AGGAGATGTC TTGGACCCCT TCTATACAAC CGTTGACATG ATGATCCTAT TCGATGGTAC |
| 2341 | TCCTATTATC AGCGGCATGG AGTTGCGCGC TCCGGCGGTT TCTGACAGGC CAAGGGTTGA |
| 2401 | AATTGGAGGA GATGATTATC GAGTTGCATA TACTCTGGTA AACTCATGCC ATGTCAATTA |
| 2461 | ACTAGTAGTT GAATTTAGAT GCTGGTGGTA TCGTGGATAC ATGTACTATA TGTTATGGTT |
| 2521 | GATACATATT TGTTTAATTG ATCGCAACAC CATTTGCGGT AACTTCAAAT TACATTCTTT |
| 2581 | CAATATATAG GTGATGGTCG ATCCTGATGC TCCTAACCCA AGCAACCCAA CCTTGAGGGA |
| 2641 | GTACTTGCAC TGGTAAGAGA AACCTATAGA CGACAATTAT TGTTGTTGGC ATGTTTTGCC |
| 2701 | CACATATACT TTGTGTGTGT ATATTTGTGC TTATGCTTCT CCATAAAATT TTGGTGTATG |
| 2761 | TCTCAAGAGA GATAGGTATA GAGGTTAGCA GTCCTTTAAA AATGGTTTAA TCCAGTAGTT |
| 2821 | TTTTTTCGGT CGGACTGCTC GAATTATTGT ATATATGGAG ATCACATGCT AGTAACTTTT |
| 2881 | TCAATAATTT CATGTTTCGA GCAGGATGGT GACTGACATC CCAGCATCAA CTGATAATAC |
| 2941 | ATACGGTGAG TACACCCCTA TTCCCATTTT GAAACAAGTA GAATGTCTAT TTTTATGATT |
| 3001 | TAGTATGTTC GTGACAATAG GCTATAGCTA TTTTGAAACT TCGGGAGCAT AAAATAGTAC |
| 3061 | TCGATTTTGT ATAACCATAA ACACACAGCT AGCCAATCTC TATTCATATT TATTTTAGTT |
| 3121 | TTATTTGCCG AACCATCCTC AACATCATAG CCACTTGATC GATCATCTCA ATCAGCGTTT |
| 3181 | GTATCCTTGC CCGCTTGATT ATCATCCATG GCAGTTCATA TTTTTTTTCA TTTCTTTCAT |
| 3241 | GCTTGTTATA GTTTTATCTG ATGAATCCAA GATGTTATTG ATCAATTAGT TCAGATGAGC |
| 3301 | AGTAATGCAT GTTGGAGGTT TGGTAGTATA TATACGTTCA AAATTTCACG AAATCGGTAA |
| 3361 | TTACGGTGGG AGCCAAAAAA AATTCCAAAA TTTCGTATTA CATTAATAAT GCATGTGCTG |
| 3421 | TAGACTCATA TTTTCTATGA TTTCGATTCT GTCACCATCC TGCTCGAATA TTTAAATCAT |
| 3481 | GCTAATATTT TGTTTACATC TAAATCTTTT ATAAAAATTA TAATTTATAT TTGGGTTTAA |
| 3541 | CAATTTCGGG CGCGTTTAGT GAGATTGGGT AATTTCGGAG CGAGGCCACC GGCCACACGA |
| 3601 | AAAATTCTAT ACACGACTAT ATGTGTACAT GTACATGCAT GGCACCCTGA TAGGCTACCC |
| 3661 | CATGGGGAAA AAATTGGAAA CGGACCATTC ATACGCAGTC GTGGTGCAGA CTGTGGGCCA |
| 3721 | CAATAGCAGT GTAAACATAA TTACGGTAAT CAAATACCCC ATGGGACCAT ATATATCATC |
| 3781 | CACAGATCCG TACGGTGCTT CCGTGTGGAT GGTCTACACC AGATCTTTTC CACACCATAA |
| 3841 | GGGCAGCAAT GCAGCATCAT ATTCATATAT GCACTAGTGA TGTACCATTT GGCTTATATC |
| 3901 | ATATTCAACC TAACTCCTTG GAAACATTAT GATATTCTAT TGGGTTGAAG ATGTCACTAC |
| 3961 | TACAAAAAAA AATCTTATGA GAGGTGTTTT GAAAACTGCC GGAGGTGCTT AAAGGAGACA |
| 4021 | GACGAGTTAG GACAACCGTC TCTATTAATG TGTACTAACT GAGGTAGTTA CCGTAACGTG |
| 4081 | CCTGACTTGA TTAACAGATT CAACCGTCTC AGTAAAGGCC ATGATTAACC GAAACAGATT |
| 4141 | CGAGAGTTTT CTTAAGTAGT TAAACTATTT TAATCTTCAC CGAACTTATA GAAAATGAAA |
| 4201 | GAGCTAACAC CAATATTTAT AAAAATAAAT TAGTATCACT AAATACATCA CGAAATCTAT |
| 4261 | TTGGTGTTGT AGAAGTTATC CTTTTCTATA AAATTGATCA AATTTATGAT AACTTAGTTT |
| 4321 | TAGGAATTCA TTTATTTTAG GACAACTGAG GAAGTACATA TTTTTTAAGT CATCCACAAA |
| 4381 | GTAGTGGATC CAATTTATTA CATTACTCTA CTACTTCAAA CTGAACAAAA GCCTAATCCT |
| 4441 | GGTTATTTTT AGAGTGATTT TTTACAACAT CAGCAGTAGT CCAGAAAATG GGAGGACATT |
| 4501 | AATAAAAGTG AAAAGGAGCA GAAGAAAGAT TACGGTATTT TATTTGTGCT ATTTGTTTAA |
| 4561 | CTATTGGCAG TTTGGGACCG AAATAAATAA CTGTTCGTAG CTCTATATTT GTCGATTCGA |
| 4621 | AAGTGTAACG ATGATTTTTG TGTTTCAAAA GAAAAATAAA GAAGTGCACC AATGATTGGA |
| 4681 | TATCATAGGC TATATATGTT GGATTAATTG CATCCAACGT ATATAGTGAA AATGCTTTTC |
| 4741 | AATCAAGTAA TCTTCGAGCG GTTACCAGTT TTAATAGTTG CGAGTCGTCG TTTTTTATGT |
| 4801 | ACCCTAGGAC ATATATATCC GCATGTAGAC GATGATGAGA CTAGCAAGTT TTTTTTTTTT |
| 4861 | TTTGAGCAAA TACATAATTA TTGGATTTGC AGGCCGTGAG ATGATGTGCT ACGAGCCCCC |
| 4921 | TGCCCCGTCC ACGGGCATCC ACCGTATGGT GCTGGTGCTA TTCCAGCAGC TTGGCCGTGA |
| 4981 | CACGGTGTTC GCGGCGCCGT CCAGGCGCCA CAACTTCAAC ACCCGTGCCT TCGCCCGCCG |
| 5041 | CTACAACCTC GGCGCGCCCG TCGCCGCCAT GTTCTTCAAC TGCCAGCGCC AGACCGGCTC |
| 5101 | CGGTGGCCCC AGGTTCACCG GGCCCTACAC CAGCCGACGT CGTGCGGGCT GATGACGACG |
| 5161 | ATCGTCGTTA CGTCACGTGT ACCGTACACA TATATGTATA GATATACATG CATGCATGTT |
| 5221 | CCATGGTATA GGATCGGTGA CAAAACGTCT AATAATGTA |

(SEQ ID NO:5 Sb06g012260 (5.2 kb)—S. propinquum) or functional fragment or variant, such as a codon optimized variant, thereof having at least 75%, 80%, 85%, 90%, 95%, 96%, 97%, 98%, or 99% sequence identity to SEQ ID NO:5. N=1, 2, 3, 4, or 5 nucleotides in length.

The coding sequence of the maturity Ma1 gene of SEQ ID NO:5, including introns, can be:

| | |
|---|---|
| 1 | ATGGCGGCTA ACGATTCCTT GGTTACTGCT CATGTGATAG GAGATGTCTT GGACCCCTTC |
| 61 | TATACAACCG TTGACATGAT GATCCTATTC GATGGTACTC CTATTATCAG CGGCATGGAG |
| 121 | TTGCGCGCTC CGGCGGTTTC TGACAGGCCA AGGGTTGAAA TTGGAGGAGA TGATTATCGA |
| 181 | GTTGCATATA CTCTGGTAAA CTCATGCCAT GTCAATTAAC TAGTAGTTGA ATTTAGATGC |
| 241 | TGGTGGTATC GTGGATACAT GTACTATATG TTATGGTTGA TACATATTTG TTTAATTGAT |
| 301 | CGCAACACCA TTTGCGGTAA CTTCAAATTA CATTCTTTCA ATATATAGGT GATGGTCGAT |
| 361 | CCTGATGCTC CTAACCCAAG CAACCCAACC TTGAGGGAGT ACTTGCACTG GTAAGAGAAA |
| 421 | CCTATAGACG ACAATTATTG TTGTTGGCAT GTTTTGCCCA CATATACTTT GTGTGTGTAT |
| 481 | ATTTGTGCTT ATGCTTCTCC ATAAAATTTT GGTGTATGTC TCAAGAGAGA TAGGTATAGA |
| 541 | GGTTAGCAGT CCTTTAAAAA TGGTTTAATC CAGTAGTTTT TTTTCGGTCG GACTGCTCGA |
| 601 | ATTATTGTAT ATATGGAGAT CACATGCTAG TAACTTTTTC AATAATTTCA TGTTTCGAGC |
| 661 | AGGATGGTGA CTGACATCCC AGCATCAACT GATAATACAT ACGGTGAGTA CACCCCTATT |
| 721 | CCCATTTTGA AACAAGTAGA ATGTCTATTT TTATGATTTA GTATGTTCGT GACAATAGGC |
| 781 | TATAGCTATT TTGAAACTTC GGGAGCATAA AATAGTACTC GATTTTGTAT AACCATAAAC |
| 841 | ACACAGCTAG CCAATCTCTA TTCATATTTA TTTTAGTTTT ATTTGCCGAA CCATCCTCAA |
| 901 | CATCATAGCC ACTTGATCGA TCATCTCAAT CAGCGTTTGT ATCCTTGCCC GCTTGATTAT |
| 961 | CATCCATGGC AGTTCATATT TTTTTTCATT TCTTTCATGC TTGTTATAGT TTTATCTGAT |
| 1021 | GAATCCAAGA TGTTATTGAT CAATTAGTTC AGATGAGCAG TAATGCATGT TGGAGGTTTG |
| 1081 | GTAGTATATA TACGTTCAAA ATTTCACGAA ATCGGTAATT ACGGTGGGAG CCAAAAAAAA |
| 1141 | TTCCAAAATT TCGTATTACA TTAATAATGC ATGTGCTGTA GACTCATATT TTCTATGATT |
| 1201 | TCGATTCTGT CACCATCCTG CTCGAATATT TAAATCATGC TAATATTTTG TTTACATCTA |
| 1261 | AATCTTTTAT AAAAATTATA ATTTATATTT GGGTTTAACA ATTTCGGGCG CGTTTAGTGA |
| 1321 | GATTGGGTAA TTTCGGAGCG AGGCCACCGG CCACACGAAA AATTCTATAC ACGACTATAT |
| 1381 | GTGTACATGT ACATGCATGG CACCCTGATA GGCTACCCCA TGGGGAAAAA ATTGGAAACG |
| 1441 | GACCATTCAT ACGCAGTCGT GGTGCAGACT GTGGGCCACA ATAGCAGTGT AAACATAATT |
| 1501 | ACGGTAATCA AATACCCCAT GGGACCATAT ATATCATCCA CAGATCCGTA CGGTGCTTCC |
| 1561 | GTGTGGATGG TCTACACCAG ATCTTTTCCA CACCATAAGG GCAGCAATGC AGCATCATAT |
| 1621 | TCATATATGC ACTAGTGATG TACCATTTGG CTTATATCAT ATTCAACCTA ACTCCTTGGA |
| 1681 | AACATTATGA TATTCTATTG GGTTGAAGAT GTCACTACTA CAAAAAAAAA TCTTATGAGA |
| 1741 | GGTGTTTTGA AAACTGCCGG AGGTGCTTAA AGGAGACAGA CGAGTTAGGA CAACCGTCTC |
| 1801 | TATTAATGTG TACTAACTGA GGTAGTTACC GTAACGTGCC TGACTTGATT AACAGATTCA |
| 1861 | ACCGTCTCAG TAAAGGCCAT GATTAACCGA AACAGATTCG AGAGTTTTCT TAAGTAGTTA |
| 1921 | AACTATTTTA ATCTTCACCG AACTTATAGA AAATGAAAGA GCTAACACCA ATATTTATAA |
| 1981 | AAATAAATTA GTATCACTAA ATACATCACG AAATCTATTT GGTGTTGTAG AAGTTATCCT |
| 2041 | TTTCTATAAA ATTGATCAAA TTTATGATAA CTTAGTTTTA GGAATTCATT TATTTTAGGA |
| 2101 | CAACTGAGGA AGTACATATT TTTTAAGTCA TCCACAAAGT AGTGGATCCA ATTTATTACA |
| 2161 | TTACTCTACT ACTTCAAACT GAACAAAAGC CTAATCCTGG TTATTTTTAG AGTGATTTTT |
| 2221 | TACAACATCA GCAGTAGTCC AGAAAATGGG AGGACATTAA TAAAAGTGAA AAGGAGCAGA |
| 2281 | AGAAAGATTA CGGTATTTTA TTTGTGCTAT TTGTTTAACT ATTGGCAGTT TGGGACCGAA |
| 2341 | ATAAATAACT GTTCGTAGCT CTATATTTGT CGATTCGAAA GTGTAACGAT GATTTTTGTG |
| 2401 | TTTCAAAAGA AAAATAAAGA AGTGCACCAA TGATTGGATA TCATAGGCTA TATATGTTGG |
| 2461 | ATTAATTGCA TCCAACGTAT ATAGTGAAAA TGCTTTTCAA TCAAGTAATC TTCGAGCGGT |
| 2521 | TACCAGTTTT AATAGTTGCG AGTCGTCGTT TTTTATGTAC CCTAGGACAT ATATATCCGC |
| 2581 | ATGTAGACGA TGATGAGACT AGCAAGTTTT TTTTTTTTTT TGAGCAAATA CATAATTATT |
| 2641 | GGATTTGCAG GCCGTGAGAT GATGTGCTAC GAGCCCCCTG CCCCGTCCAC GGGCATCCAC |
| 2701 | CGTATGGTGC TGGTGCTATT CCAGCAGCTT GGCCGTGACA CGGTGTTCGC GGCGCCGTCC |
| 2761 | AGGCGCCACA ACTTCAACAC CCGTGCCTTC GCCCGCCGCT ACAACCTCGG CGCGCCCGTC |
| 2821 | GCCGCCATGT TCTTCAACTG CCAGCGCCAG ACCGGCTCCG GTGGCCCCAG GTTCACCGGG |
| 2881 | CCCTACACCA GCCGACGTCG TGCGGGCTGA |

(SEQ ID NO:6 Sb06g012260 (5.2 kb)—S. propinquum) or functional fragment or variant, such as a codon optimized variant, thereof having at least 75%, 80%, 85%, 90%, 95%, 96%, 97%, 98%, or 99% sequence identity to SEQ ID NO:6.

The coding sequence of the maturity Ma1 gene, without introns, as it is found in short-day S. propinquum can include the nucleic acid sequence:

| | |
|---|---|
| 1 | ATGGCGGCTA ACGATTCCTT GGTTACTGCT CATGTGATAG GAGATGTCTT GGACCCCTTC |
| 61 | TATACAACCG TTGACATGAT GATCCTATTC GATGGTACTC CTATTATCAG CGGCATGGAG |
| 121 | TTGCGCGCTC CGGCGGTTTC TGACAGGCCA AGGGTTGAAA TTGGAGGAGA TGATTATCGA |
| 181 | GTTGCATATA CTCTGGTGAT GGTCGATCCT GATGCTCCTA ACCCAAGCAA CCCAACCTTG |
| 241 | AGGGAGTACT TGCACTGGAT GGTGACTGAC ATCCCAGCAT CAACTGATAA TACATACGGC |
| 301 | CGTGAGATGA TGTGCTACGA GCCCCCTGCC CCGTCCACGG GCATCCACCG TATGGTGCTG |
| 361 | GTGCTATTCC AGCAGCTTGG CCGTGACACG GTGTTCGCGG CGCCGTCCAG GCGCCACAAC |
| 421 | TTCAACACCC GTGCCTTCGC CCGCCGCTAC AACCTCGGCG CGCCCGTCGC CGCCATGTTC |
| 481 | TTCAACTGCC AGCGCCAGAC CGGCTCCGGT GGCCCCAGGT TCACCGGGCC CTACACCAGC |
| 541 | CGACGTCGTG CGGGCTGA |

(SEQ ID NO:7, Sb06g012260—S. propinquum, or fragment, or a variant, such as a codon optimized variant, thereof having at least 75%, 80%, 85%, 90%, 95%, 96%, 97%, 98%, or 99% sequence identity to SEQ ID NO:7.

A maturity Ma1 protein as it is found in short-day S. propinquum can include the amino acid sequence:

MAANDSLVTAHVIGDVLDPFYTTVDMMILFDGTPIISGMELRAPAVSDRP
RVEIGGDDYRVAYTLVMVDPDAPNPSNPTLREYLHWMVTDIPASTDNTYG
REMMCYEPPAPSTGIHRMVLVLFQQLGRDTVFAAPSRRHNFNTRAFARRY
NLGAPVAAMFFNCQRQTGSGGPRFTGPYTSRRRAG*

(SEQ ID NO:8, Sb06g012260) or functional fragment, or variant thereof having at least 75%, 80%, 85%, 90%, 95%, 96%, 97%, 98%, or 99% sequence identity to SEQ ID NO:8.

In some embodiments, the maturity Ma1 gene (including non-coding sequence) as it is found in short day S. propinquum includes the nucleic acid sequence:

| | |
|---|---|
| 1 | CACTAGTACA AAAATCATTT TCGTTGGCAC GTTGTTTTTT TTTTCACAGG CAGTTCACAA |
| 61 | TATCATGGTG CTAGTAGAAA AATTTCAACG GGCCCAACAA GAGAACCGCC AGGCGGTCTT |
| 121 | CTTAATTCAA CCGCCTGTGT AAACTTTCCA TTTACATAGG CGGCTTACGA TAAAAACCGT |
| 181 | GTGTATAAAT ACCATTAACA CAGGCAGTCG AGTTACGACA ACCGCCTGTG TAAATGTGTC |
| 241 | TTTTTACACA GGCGGTTTGT ATAGAGGGCC GCCTGTGCTA ATATATTTAC ACAGGCTATG |
| 301 | AGCCGCCTGT GTTAAGTCTT CTATAAATAC CCTTCGTCCA CCTCCAGACA AGAACAGTTA |
| 361 | CTCCCATGAG CTCTGCACAC TGGCGGACCA GACGATTCCA GTTTCCAAGG GGGGAGGTTT |
| 421 | TGATTTTCAT TTCTTTGGTG AGAAACTTCC AAAAGGTTAG TTAGTGCCAT TGATGCTATT |
| 481 | TTTTAAGCGA TTCTTTGGTT CAATTCTTGT ATTGGAGGTG CTCTAGATCT AGAGTTCATC |
| 541 | ATGCATTCTT GCTTAGGGTT AGAGTTCATA GGGCAAAAAG AGAGAGATTT AGCTAAATTT |
| 601 | TTATGTAAAT TCATAGTAAA TTGTAAAAAT TAAAAAAAAT AAAAAATAAA TACTTTTTAG |
| 661 | AATTCTTGTG AGTAGATCTA TACAATAGAG TAATGATGAG GATATTTTGA AGTTTATAAT |
| 721 | TTTGATTCAG TTTTAGCTTT TCTTTTTTCA GATGAATTAG ACTTTATAAA CTCAAACATT |
| 781 | AAAATGTTGA AAATCATAAA ATGGCAAATA AATACTTTTT CAAATCTTTG TGCATAAATA |
| 841 | CTTCATAGAA ATCCTTGAAT TATTCCTAAA TTTTATACAA TTGTTTCTTA TAATTATGAA |
| 901 | AATGAGTTTA AACAATTATT TAAATTCCAT AAATTGTAAC TCCGTAAGGT GTAGGTTTTC |
| 961 | ATCTCTGTTT AATAGAAGGA GGTTAGTATC TTAGTTAAGT CTGTTTTCGG GGGTTATATT |
| 1021 | AGTTTTGTTT TTAGATTGAC CTACATTAAT TGTTCTTAAC TAATTACAGC TAAATATGGA |
| 1081 | GAGGTCATTA TGGATGTACA ACTTATCAAG ATTGGACCTA TCATATGTAG TGCAGGTCCA |
| 1141 | AAAATTTATT GATGTCGCAA AGATACATGC TCGCAGAACA AAGGCGAAGC ACATATGTTG |
| 1201 | TCCATGCGCA GACTGCAAAA ATATTATGGT ATTTGACAAT GTAGAAGCAA TTACTTCCCA |
| 1261 | TCTGGTTTGA AGAGGATTTA TGGAGGACTA CTTGATTTGG ACAAAACATG GTGAGGGTAG |
| 1321 | TTTTGCACCT TATATGCGGA CAACTGACAA CACTGCAACT AACATCAATG TGGAGGGTCC |
| 1381 | AATGCCACCT CTCAATGAAT TTCATGCTAT GCCAGATGTT AATGAAACTC ATACGTCTGA |
| 1441 | TGTCAATGAA ACTCAGCATG CTAACACAGA TGTTGTTGAA GATGCAGATT TCTTAGAGGC |
| 1501 | AATAATGAAC CGTTGTGCGG ATCCATCAAT ATTCTTCATG AAGGGAATGA AAGCATTGAA |
| 1561 | GAAGGCAGCA GAGGACACTT TGTACGACGA GTCAAAAGGT TGTACCAAAC AATGGTCGAC |
| 1621 | ATTATGTGTT GTTCTTCAGT TTTTGACGAT GAAGGCTAGA CATGGTTGGT CCGATGCTAG |
| 1681 | CTTCAATGAT TTCTTGCGTG TACTTGGAGA CCTTCTTCCT AAGGAGAACA AAGTGCCTGC |
| 1741 | TAACACATAC TATGCAAAGA AGCTAGTCAG TCCACTTACG ATAGGTGTTG AGAAGATCCA |
| 1801 | CGCATGTAGA AATCATTGTA TTCTATATCG AGGTGATCAA TATAAAGACT TAGACAGTTG |
| 1861 | TCCAAACTGT GGTGCCAGTA GGTACAAGAC AAACAAAGAT TTTCGGGAGG AAGAGAATCT |
| 1921 | AGCCTCTGTT TCTACAGGGA GGAAGCGAAA GAAGACCCAA ACAAAGACTC AACAAGACAA |
| 1981 | GCGCTCAAAG CCTAGTAGCA ATGAAGAAGT GGACTATTAT GCATTGAGAA GAGTCTCCCT |
| 2041 | ATGAGCCAAA AAAGGGGACA GCAGCAGGCA CAACTCTCTT TCTGAAAGGA CTTGGAAAGC |
| 2101 | AGCGGACGGC ACGGCTCATT GAGCTCGAAC CGTCACAGAA AAAGGAAGCC ACCGCCCAGT |
| 2161 | CAATAGAAGC CATGCCCCCA TCAAAGGAAG CCCCAAGTGG CGATGTACAT ATTGAACAGC |
| 2221 | CATCAAGTCA ACCATTGACC CTAAAGGATA TCAGAAAGCC AACGATTGAT GATTATGTCA |
| 2281 | ATGTCCCTAG TGACTATGTG CCCGGAAGGC CTATGCTCCA ATGGACGCTG CTCGATTAGA |
| 2341 | TTCAATGGCT GATAAAAAGG TTTCATGACT GGTACATGAG AGCAGTGCAT GCTAGCCTCC |
| 2401 | ATGGAATCAG AGTTGATATA CCAACAGACA TGTTTGCTAC TGGTAACAAA AAAAGCAAGA |
| 2461 | CATTTGTTAC CTTTGAGGAC ATGCACTTGT TATTGAACTA TAGGCGGCTT GACGTCCAAC |
| 2521 | TCATAACAAT CTGGTGCCTG TAAGTATCAC TCATGCACAC ACAATTATTA TATATTAATA |
| 2581 | TGTAGTGTGA AACTCTAATA TGTAGATGTT GTCTGTAGTT TGCAAGATCA CGAGCAGATG |
| 2641 | TCATTATTAT CTGCCGGATC GATGGTCGGT TATCTGAGCC CTATCAAGTT ACAAGAAAAT |
| 2701 | ATGAACAAAT TCGTATTATC AAAGGAAGAT AGAGCAAAGA TAGAGGAAGA CAAAACACCA |
| 2761 | GGATAATTAT GCCATCTATC TTGGTAGATC AATGCTGAGG TATAAATATA GGGATTTTAT |
| 2821 | ATTGGCACCA TACAACATTA GGTAAGCTTG ACTTCATATA CGTATTTCAA ATTATCGTGT |
| 2881 | AAACAATATA CATGTGTCGC TCACTCATTT ATTCATGCAG TGACCATTGG ATTGTTTTTT |
| 2941 | ATATTTATCC CTTCGAAGGG AAGGTGCTTG TCCTAGACTC TTTACATGTT CCTCCCGAGA |
| 3001 | AGTATCAACC ATTCTTGGTT CAATTAGAAA GGTGAGCCAA CATGAAACCA CATGCGTACT |
| 3061 | TATATAAATT AGAGTTTCAA AATAACTTTA GTGATTTAGG TTCGATATCT ACGGGGCATG |
| 3121 | GCGGTTTTAT AAGAAACAAA AGGGACCTGT CGACGCTGCA CGCTCAGATC CTAGGATCCC |
| 3181 | ATTGATGATA CAACACCACT ATCCGGTAAG TTTTCTGAAC ACATTTCATC ATATAAATAA |
| 3241 | TACATAAAGC ATGGCAAATT TAGAATAATC CGTTGCTCAT TATATAGTGC CACAAGCAAC |
| 3301 | CACCTGGATC GGTCTATTGT GGGTACTATG TCTGTGAGTT TATAAGGCAG CGGGGACGTT |
| 3361 | ACGTCAAGGA CAAAAATATG GTAAATAATA TCTATGTATG AAAGTTTTCT CATTAAAGCT |
| 3421 | GCAAAATTAT ATATTGAACA TGTGTCAATC ATGCTTTTAA ACTTTATTTT CAGCCGAAAA |
| 3481 | AGCAAGGAAA AGACGTGCCC TTTACACCAA AGACTCTGGA AGATATAGTA GCATACTTGT |
| 3541 | GTGGTTTTAT TATGAGAGAA ATAATTTCAA GTGACAGTGC ATATTTTGAT CATGAGGGCG |
| 3601 | ATTTAGCAAG TGATAAATTT AGAGTGCTGA CAGACATAGC AGGTCTAAAT CTGAAGCGAA |
| 3661 | ACGACATGTA AACATTGTAT GGTTGTGCGG ATAACATGCA TTGACGTGTA TATATATAAT |
| 3721 | TTTATGGTTG ATGTTTGATT TGTTTACAAT TCTATAATAT ATATATGTGG TGTATGTATG |
| 3781 | ATGTTGTGTG TGTATATATA TATATATATA TATATATATA TATATATATA TATATATATA |
| 3841 | TATATATATA TAATGTTTAG CACTGTGTTT GGTGGGAAAA ATTAAAATTT GAAATATATA |
| 3901 | TAAAAAATTA TTTACACAGA CAGTGTACGT GTCGAGCGTC GTCCTGTGCT ATACAAATAC |
| 3961 | ATTCTAACAG GCGGCTCGCC TTGTCCACCG GTCGGTTAAA AATACATTTC CACACNGGCC |
| 4021 | TGGCTGGGAG AGCCGCCTGT GAAAACATAA TTTTCACAGG CGGCTCGCAC AGCCCCGCCT |
| 4081 | GTACTGTGGT CCATTTTGTA CTGACCCCTG GTACAGGCGG TGGGCTTGGC CGCCTGTGAA |
| 4141 | GATGCTTTTA GCACCGCCTG TAAAAATGTT TTTTGTAGCA GTGTTT |

(SEQ ID NO:19—Sb07g008600—S. propinquum) or a functional fragment or variant thereof having 75%, 80%, 85%, 90%, 95%, 96%, 97%, 98%, or 99% sequence identity to SEQ ID NO:19.

The coding sequence of the maturity Ma1 gene of SEQ ID NO:19, including introns, can be:

| | |
|---|---|
| 1 | ATGCCCCCAT CAAAGGAAGC CCCAAGTGGC GATGTACATA TTGAACAGCC ATCAAGTCAA |
| 61 | CCATTGACCC TAAAGGATAT CAGAAAGCCA ACGATTGATG ATTATGTCAA TGTCCCTAGT |
| 121 | GACTATGTGC CCGGAAGGCC TATGCTCCAA TGGACGCTGC TCGATTAGAT TCAATGGCTG |
| 181 | ATAAAAAGGT TTCATGACTG GTACATGAGA GCAGTGCATG CTAGCCTCCA TGGAATCAGA |
| 241 | GTTGATATAC CAACAGACAT GTTTGCTACT GGTAACAAAA AAAGCAAGAC ATTTGTTACC |
| 301 | TTTGAGGACA TGCACTTGTT ATTGAACTAT AGGCGGCTTG ACGTCCAACT CATAACAATC |
| 361 | TGGTGCCTGT AAGTATCACT CATGCACACA CAATTATTAT ATATTAATAT GTAGTGTGAA |
| 421 | ACTCTAATAT GTAGATGTTG TCTGTAGTTT GCAAGATCAC GAGCAGATGT CATTATTATC |
| 481 | TGCCGGATCG ATGGTCGGTT ATCTGAGCCC TATCAAGTTA CAAGAAAATA TGAACAAATT |
| 541 | CGTATTATCA AAGGAAGATA GAGCAAAGAT AGAGGAAGAC AAAACACCAG GATAATTATG |
| 601 | CCATCTATCT TGGTAGATCA ATGCTGAGGT ATAAATATAG GGATTTTATA TTGGCACCAT |
| 661 | ACAACATTAG GTAAGCTTGA CTTCATATAC GTATTTCAAA TTATCGTGTA AACAATATAC |
| 721 | ATGTGTCGCT CACTCATTTA TTCATGCAGT GACCATTGGA TTGTTTTTTA TATTTATCCC |
| 781 | TTCGAAGGGA AGGTGCTTGT CCTAGACTCT TTACATGTTC CTCCCGAGAA GTATCAACCA |
| 841 | TTCTTGGTTC AATTAGAAAG GTGAGCCAAC ATGAAACCAC ATGCGTACTT ATATAAATTA |
| 901 | GAGTTTCAAA ATAACTTTAG TGATTTAGGT TCGATATCTA CGGGGCATGG CGGTTTTATA |
| 961 | AGAAACAAAA GGGACCTGTC GACGCTGCAC GCTCAGATCC TAGGATCCCA TTGATGATAC |
| 1021 | AACACCACTA TCCGGTAAGT TTTCTGAACA CATTTCATCA TATAAATAAT ACATAAAGCA |
| 1081 | TGGCAAATTT AGAATAATCC GTTGCTCATT ATATAGTGCC ACAAGCAACC ACCTGGATCG |
| 1141 | GTCTATTGTG GGTACTATGT CTGTGAGTTT ATAAGGCAGC GGGGACGTTA CGTCAAGGAC |
| 1201 | AAAAATATGG TAAATAATAT CTATGTATGA AAGTTTTCTC ATTAAAGCTG CAAAATTATA |
| 1261 | TATTGAACAT GTGTCAATCA TGCTTTTAAA CTTTATTTTC AGCCGAAAAA GCAAGGAAAA |
| 1321 | GACGTGCCCT TTACACCAAA GACTCTGGAA GATATAGTAG CATACTTGTG TGGTTTTATT |
| 1381 | ATGAGAGAAA TAATTTCAAG TGACAGTGCA TATTTTGATC ATGAGGGCGA TTTAGCAAGT |
| 1441 | GATAAATTTA GAGTGCTGAC AGACATAGCA GGTCTAAATC TGAAGCGAAA CGACATGTAA |

(SEQ ID NO:28—Sb07g008600—S. propinquum) or functional fragment or variant, such as a codon optimized variant, thereof having at least 75%, 80%, 85%, 90%, 95%, 96%, 97%, 98%, or 99% sequence identity to SEQ ID NO:28.

The coding sequence of the maturity Ma1 gene of SEQ ID NO:28, without introns, can be:

| | |
|---|---|
| 1 | ATGCCCCCAT CAAAGGAAGC CCCAAGTGGC GATGTACATA TTGAACAGCC ATCAAGTCAA |
| 61 | CCATTGACCC TAAAGGATAT CAGAAAGCCA ACGATTGATG ATTATGTCAA TGTCCCTAGT |
| 121 | GACTATGTGC CCGGAAGGCC TATGCTCCAA TGGACGCTGC TCGATTAGAT TCAATGGCTG |
| 181 | ATAAAAAGGT TTCATGACTG GTACATGAGA GCAGTGCATG CTAGCCTCCA TGGAATCAGA |
| 241 | GTTGATATAC CAACAGACAT GTTTGCTACT GGTAACAAAA AAAGCAAGAC ATTTGTTACC |
| 301 | TTTGAGGACA TGCACTTGTT ATTGAACTAT AGGCGGCTTG ACGTCCAACT CATAACAATC |
| 361 | TGGTGCCTGG ACCATTGGAT TGTTTTTTAT ATTTATCCCT TCGAAGGGAA GGTGCTTGTC |
| 421 | CTAGACTCTT TACATGTTCC TCCCGAGAAG TATCAACCAT TCTTGGTTCA ATTAGAAAGG |
| 481 | GCATGGCGGT TTTATAAGAA ACAAAAGGGA CCTGTCGACG CTGCACGCTC AGATCCTAGG |
| 541 | ATCCCATTGA TGATACAACA CCACTATCCG TGCCACAAGC AACCACCTGG ATCGGTCTAT |
| 601 | TGTGGGTACT ATGTCTGTGA GTTTATAAGG CAGCGGGGAC GTTACGTCAA GGACAAAAAT |
| 661 | ATGCCGAAAA AGCAAGGAAA AGACGTGCCC TTTACACCAA AGACTCTGGA AGATATAGTA |
| 721 | GCATACTTGT GTGGTTTTAT TATGAGAGAA ATAATTTCAA GTGACAGTGC ATATTTTGAT |
| 781 | CATGAGGGCG ATTTAGCAAG TGATAAATTTAGAGTGCTGACAGACATAGC AGGTCTAAAT |
| 841 | CTGAAGCGAA ACGACATGTA A |

(SEQ ID NO:29—Sb07g008600—S. propinquum) or functional fragment or variant, such as a codon optimized variant, thereof having at least 75%, 80%, 85%, 90%, 95%, 96%, 97%, 98%, or 99% sequence identity to SEQ ID NO:29.

In some embodiments, the maturity Ma1 gene (including non-coding sequence) as it is found in short day S. propinquum includes the nucleic acid sequence:

| | |
|---|---|
| 1 | CACTAGTACA AAAATCATTT TCGTTGGCAC GTTGTTTTTT TTTTCACAGG CAGTTCACAA |
| 61 | TATCATGGTG CTAGTAGAAA AATTTCAACG GGCCCAACAA GAGAACCGCC AGGCGGTCTT |
| 121 | CTTAATTCAA CCGCCTGTGT AAACTTTCCA TTTACATAGG CGGCTTACGA TAAAAACCGT |
| 181 | GTGTATAAAT ACCATTAACA CAGGCAGTCG AGTTACGACA ACCGCCTGTG TAAATGTGTC |
| 241 | TTTTTACACA GGCGGTTTGT ATAGAGGGCC GCCTGTGCTA ATATATTTAC ACAGGCTATG |
| 301 | AGCCGCCTGT GTTAAGTCTT CTATAAATAC CCTTCGTCCA CCTCCAGACA AGAACAGTTA |
| 361 | CTCCCATGAG CTCTGCACAC TGGCGGACCA GACGATTCCA GTTTCCAAGG GGGGAGGTTT |
| 421 | TGATTTTCAT TTCTTTGGTG AGAAACTTCC AAAAGGTTAG TTAGTGCCAT TGATGCTATT |
| 481 | TTTTAAGCGA TTCTTTGGTT CAATTCTTGT ATTGGAGGTG CTCTAGATCT AGAGTTCATC |
| 541 | ATGCATTCTT GCTTAGGGTT AGAGTTCATA GGGCAAAAAG AGAGAGATTT AGCTAAATTT |
| 601 | TTATGTAAAT TCATAGTAAA TTGTAAAAAT TAAAAAAAAT AAAAAATAAA TACTTTTTAG |
| 661 | AATTCTTGTG AGTAGATCTA TACAATAGAG TAATGATGAG GATATTTTGA AGTTTATAAT |
| 721 | TTTGATTCAG TTTTAGCTTT TCTTTTTTCA GATGAATTAG ACTTTATAAA CTCAAACATT |
| 781 | AAAATGTTGA AAATCATAAA ATGGCAAATA AATACTTTTT CAAATCTTTG TGCATAAATA |
| 841 | CTTCATAGAA ATCCTTGAAT TATTCCTAAA TTTTATACAA TTGTTTCTTA TAATTATGAA |
| 901 | AATGAGTTTA AACAATTATT TAAATTCCAT AAATTGTAAC TCCGTAAGGT GTAGGTTTTC |
| 961 | ATCTCTGTTT AATAGAAGGA GGTTAGTATC TTAGTTAAGT CTGTTTTCGG GGGTTATATT |
| 1021 | AGTTTTGTTT TTAGATTGAC CTACATTAAT TGTTCTTAAC TAATTACAGC TAAATATGGA |
| 1081 | GAGGTCATTA TGGATGTACA ACTTATCAAG ATTGGACCTA TCATATGTAG TGCAGGTCCA |
| 1141 | AAAATTTATT GATGTCGCAA AGATACATGC TCGCAGAACA AAGGCGAAGC ACATATGTTG |
| 1201 | TCCATGCGCA GACTGCAAAA ATATTATGGT ATTTGACAAT GTAGAAGCAA TTACTTCCCA |
| 1261 | TCTGGTTTGA AGAGGATTTA TGGAGGACTA CTTGATTTGG ACAAAACATG GTGAGGGTAG |
| 1321 | TTTTGCACCT TATATGCGGA CAACTGACAA CACTGCAACT AACATCAATG TGGAGGGTCC |
| 1381 | AATGCCACCT CTCAATGAAT TTCATGCTAT GCCAGATGTT AATGAAACTC ATACGTCTGA |
| 1441 | TGTCAATGAA ACTCAGCATG CTAACACAGA TGTTGTTGAA GATGCAGATT TCTTAGAGGC |
| 1501 | AATAATGAAC CGTTGTGCGG ATCCATCAAT ATTCTTCATG AAGGGAATGA AAGCATTGAA |
| 1561 | GAAGGCAGCA GAGGACACTT TGTACGACGA GTCAAAAGGT TGTACCAAAC AATGGTCGAC |
| 1621 | ATTATGTGTT GTTCTTCAGT TTTTGACGAT GAAGGCTAGA CATGGTTGGT CCGATGCTAG |
| 1681 | CTTCAATGAT TTCTTGCGTG TACTTGGAGA CCTTCTTCCT AAGGAGAACA AAGTGCCTGC |
| 1741 | TAACACATAC TATGCAAAGA AGCTAGTCAG TCCACTTACG ATAGGTGTTG AGAAGATCCA |
| 1801 | CGCATGTAGA AATCATTGTA TTCTATATCG AGGTGATCAA TATAAAGACT TAGACAGTTG |
| 1861 | TCCAAACTGT GGTGCCAGTA GGTACAAGAC AAACAAAGAT TTTCGGGAGG AAGAGAATCT |
| 1921 | AGCCTCTGTT TCTACAGGGA GGAAGCGAAA GAAGACCCAA ACAAAGACTC AACAAGACAA |
| 1981 | GCGCTCAAAG CCTAGTAGCA ATGAAGAAGT GGACTATTAT GCATTGAGAA GAGTCTCCCT |
| 2041 | ATGAGCCAAA AAAGGGGACA GCAGCAGGCA CAACTCTCTT TCTGAAAGGA CTTGGAAAGC |
| 2101 | AGCGGACGGC ACGGCTCATT GAGCTCGAAC CGTCACAGAA AAAGGAAGCC ACCGCCCAGT |
| 2161 | CAATAGAAGC CATGCCCCCA TCAAAGGAAG CCCCAAGTGG CGATGTACAT ATTGAACAGC |
| 2221 | CATCAAGTCA ACCATTGACC CTAAAGGATA TCAGAAAGCC AACGATTGAT GATTATGTCA |
| 2281 | ATGTCCCTAG TGACTATGTG CCCGGAAGGC CTATGCTCCA ATGGACGCTG CTCGATTAGA |
| 2341 | TTCAATGGCT GATAAAAAGG TTTCATGACT GGTACATGAG AGCAGTGCAT GCTAGCCTCC |
| 2401 | ATGGAATCAG AGTTGATATA CCAACAGACA TGTTTGCTAC TGGTAACAAA AAAAGCAAGA |
| 2461 | CATTTGTTAC CTTTGAGGAC ATGCACTTGT TATTGAACTA TAGGCGGCTT GACGTCCAAC |
| 2521 | TCATAACAAT CTGGTGCCTG TAAGTATCAC TCATGCACAC ACAATTATTA TATATTAATA |
| 2581 | TGTAGTGTGA AACTCTAATA TGTAGATGTT GTCTGTAGTT TGCAAGATCA CGAGCAGATG |
| 2641 | TCATTATTAT CTGCCGGATC GATGGTCGGT TATCTGAGCC CTATCAAGTT ACAAGAAAAT |
| 2701 | ATGAACAAAT TCGTATTATC AAAGGAAGAT AGAGCAAAGA TAGAGGAAGA CAAAACACCA |
| 2761 | GGATAATTAT GCCATCTATC TTGGTAGATC AATGCTGAGG TATAAATATA GGGATTTTAT |
| 2821 | ATTGGCACCA TACAACATTA GGTAAGCTTG ACTTCATATA CGTATTTCAA ATTATCGTGT |
| 2881 | AAACAATATA CATGTGTCGC TCACTCATTT ATTCATGCAG TGACCATTGG ATTGTTTTTT |
| 2941 | ATATTTATCC CTTCGAAGGG AAGGTGCTTG TCCTAGACTC TTTACATGTT CCTCCCGAGA |
| 3001 | AGTATCAACC ATTCTTGGTT CAATTAGAAA GGTGAGCCAA CATGAAACCA CATGCGTACT |
| 3061 | TATATAAATT AGAGTTTCAA AATAACTTTA GTGATTTAGG TTCGATATCT ACGGGGCATG |
| 3121 | GCGGTTTTAT AAGAAACAAA AGGGACCTGT CGACGCTGCA CGCTCAGATC CTAGGATCCC |
| 3181 | ATTGATGATA CAACACCACT ATCCGGTAAG TTTTCTGAAC ACATTTCATC ATATAAATAA |
| 3241 | TACATAAAGC ATGGCAAATT TAGAATAATC CGTTGCTCAT TATATAGTGC CACAAGCAAC |
| 3301 | CACCTGGATC GGTCTATTGT GGGTACTATG TCTGTGAGTT TATAAGGCAG CGGGGACGTT |
| 3361 | ACGTCAAGGA CAAAAATATG GTAAATAATA TCTATGTATG AAGTTTTCTC ATTAAAGCTG |
| 3421 | CAAAATTATA TATTGAACAT GTGTCAATCA TGCTTTTAAA CTTTATTTTC AGCCGAAAAA |
| 3481 | GCAAGGAAAA GACGTGCCCT TTACACCAAA GACTCTGGAA GATATAGTAG CATACTTGTG |
| 3541 | TGGTTTTATT ATGAGAGAAA TAATTTCAAG TGACAGTGCA TATTTTGATC ATGAGGGCGA |
| 3601 | TTTAGCAAGT GATAAATTTA GAGTGCTGAC AGACATAGCA GGTCTAAATC TGAAGCGAAA |
| 3661 | CGACATGTAA ACATTGTATG GTTGTGCGGA TAACATGCAT TGACGTGTAT ATATATAATT |
| 3721 | TTATGGTTGA TGTTTGATTT GTTTACAATT CTATAATATA TATATGTGGT GTATGTATGA |
| 3781 | TGTTGTGTGT GTATATATAT ATATATATAT ATATATATAT ATATATATAT ATATATATAT |
| 3841 | ATATATATAT AATGTTTAGC ACTGTGTTTG GTGGGAAAAA TTAAAATTTG AAATATATAT |
| 3901 | AAAAAATTAT TTACACAGAC AGTGTAGTGT GAGCTGCCTG TGTAAAAATA CATTTATACA |
| 3961 | GGCGGCTCAC CTTGTCNNNN CAGGCGGTGC TAAAAGCATC TTCACAGGCG GCCAAGCCCA |
| 4021 | CCGCCTGTAC CAGGGGTCAG TACAAAATGG ACCACAGTAC AGGCGGGGCT GTGCGAGCCG |
| 4081 | CCTGTGAAAA CATAATTTTC ACAGGCGGCT CGCACAGCCC CGCCTGTACT GTGGTCCATT |
| 4141 | TTGTACTGAC CCCTGGTACA GGCGGTGGGC TTGGCCGCCT GTGAAGATGC TTTTAGCACC |
| 4201 | GCCTGTAAAA ATGTTTTTTG TAGCAGTGTT T |

(SEQ ID NO:20) or a functional fragment or variant thereof having 75%, 80%, 85%, 90%, 95%, 96%, 97%, 98%, or 99% sequence identity to SEQ ID NO:20 (Sb07g008600—S. propinquum). Each N=can be any nucleotide or combination of any 2, 3, 4, or 5 nucleotides.

The coding sequence of the maturity Ma1 gene of SEQ ID NO:20, including introns, can be:

| | |
|---|---|
| 1 | ATGCCCCCAT CAAAGGAAGC CCCAAGTGGC GATGTACATA TTGAACAGCC ATCAAGTCAA |
| 61 | CCATTGACCC TAAAGGATAT CAGAAAGCCA ACGATTGATG ATTATGTCAA TGTCCCTAGT |
| 121 | GACTATGTGC CCGGAAGGCC TATGCTCCAA TGGACGCTGC TCGATTAGAT TCAATGGCTG |
| 181 | ATAAAAAGGT TTCATGACTG GTACATGAGA GCAGTGCATG CTAGCCTCCA TGGAATCAGA |
| 241 | GTTGATATAC CAACAGACAT GTTTGCTACT GGTAACAAAA AAAGCAAGAC ATTTGTTACC |
| 301 | TTTGAGGACA TGCACTTGTT ATTGAACTAT AGGCGGCTTG ACGTCCAACT CATAACAATC |
| 361 | TGGTGCCTGT AAGTATCACT CATGCACACA CAATTATTAT ATATTAATAT GTAGTGTGAA |
| 421 | ACTCTAATAT GTAGATGTTG TCTGTAGTTT GCAAGATCAC GAGCAGATGT CATTATTATC |
| 481 | TGCCGGATCG ATGGTCGGTT ATCTGAGCCC TATCAAGTTA CAAGAAAATA TGAACAAATT |
| 541 | CGTATTATCA AAGGAAGATA GAGCAAAGAT AGAGGAAGAC AAAACACCAG GATAATTATG |
| 601 | CCATCTATCT TGGTAGATCA ATGCTGAGGT ATAAATATAG GGATTTTATA TTGGCACCAT |
| 661 | ACAACATTAG GTAAGCTTGA CTTCATATAC GTATTTCAAA TTATCGTGTA AACAATATAC |
| 721 | ATGTGTCGCT CACTCATTTA TTCATGCAGT GACCATTGGA TTGTTTTTTA TATTTATCCC |
| 781 | TTCGAAGGGA AGGTGCTTGT CCTAGACTCT TTACATGTTC CTCCCGAGAA GTATCAACCA |
| 841 | TTCTTGGTTC AATTAGAAAG GTGAGCCAAC ATGAAACCAC ATGCGTACTT ATATAAATTA |
| 901 | GAGTTTCAAA ATAACTTTAG TGATTTAGGT TCGATATCTA CGGGGCATGG CGGTTTTATA |
| 961 | AGAAACAAAA GGGACCTGTC GACGCTGCAC GCTCAGATCC TAGGATCCCA TTGATGATAC |
| 1021 | AACACCACTA TCCGGTAAGT TTTCTGAACA CATTTCATCA TATAAATAAT ACATAAAGCA |
| 1081 | TGGCAAATTT AGAATAATCC GTTGCTCATT ATATAGTGCC ACAAGCAACC ACCTGGATCG |
| 1141 | GTCTATTGTG GGTACTATGT CTGTGAGTTT ATAAGGCAGC GGGGACGTTA CGTCAAGGAC |
| 1201 | AAAAATATGG TAAATAATAT CTATGTATGA AGTTTTCTCA TTAAAGCTGC AAAATTATAT |
| 1261 | ATTGAACATG TGTCAATCAT GCTTTTAAAC TTTATTTTCA GCCGAAAAAG CAAGGAAAAG |
| 1321 | ACGTGCCCTT TACACCAAAG ACTCTGGAAG ATATAGTAGC ATACTTGTGT GGTTTTATTA |
| 1381 | TGAGAGAAAT AATTTCAAGT GACAGTGCAT ATTTTGATCA TGAGGGCGAT TTAGCAAGTG |
| 1441 | ATAAATTTAG AGTGCTGACA GACATAGCAG GTCTAAATCT GAAGCGAAAC GACATGTAA |

(SEQ ID NO:30—Sb07g008600 (10.6 kb)—S. propinquum) or functional fragment or variant, such as a codon optimized variant, thereof having at least 75%, 80%, 85%, 90%, 95%, 96%, 97%, 98%, or 99% sequence identity to SEQ ID NO:30.

The coding sequence of the maturity Ma1 gene of SEQ ID NO:30, without introns, can be:

| | |
|---|---|
| 1 | ATGCCCCCAT CAAAGGAAGC CCCAAGTGGC GATGTACATA TTGAACAGCC ATCAAGTCAA |
| 61 | CCATTGACCC TAAAGGATAT CAGAAAGCCA ACGATTGATG ATTATGTCAA TGTCCCTAGT |
| 121 | GACTATGTGC CCGGAAGGCC TATGCTCCAA TGGACGCTGC TCGATTAGAT TCAATGGCTG |
| 181 | ATAAAAAGGT TTCATGACTG GTACATGAGA GCAGTGCATG CTAGCCTCCA TGGAATCAGA |
| 241 | GTTGATATAC CAACAGACAT GTTTGCTACT GGTAACAAAA AAAGCAAGAC ATTTGTTACC |
| 301 | TTTGAGGACA TGCACTTGTT ATTGAACTAT AGGCGGCTTG ACGTCCAACT CATAACAATC |
| 361 | TGGTGCCTGG ACCATTGGAT TGTTTTTTAT ATTTATCCCT TCGAAGGGAA GGTGCTTGTC |
| 421 | CTAGACTCTT TACATGTTCC TCCCGAGAAG TATCAACCAT TCTTGGTTCA ATTAGAAAGG |
| 481 | GCATGGCGGT TTTATAAGAA ACAAAAGGGA CCTGTCGACG CTGCACGCTC AGATCCTAGG |
| 541 | ATCCCATTGA TGATACAACA CCACTATCCG TGCCACAAGC AACCACCTGG ATCGGTCTAT |
| 601 | TGTGGGTACT ATGTCTGTGA GTTTATAAGG CAGCGGGGAC GTTACGTCAA GGACAAAAAT |
| 661 | ATGCCGAAAA AGCAAGGAAA AGACGTGCCC TTTACACCAA AGACTCTGGA AGATATAGTA |
| 721 | GCATACTTGT GTGGTTTTAT TATGAGAGAA ATAATTTCAA GTGACAGTGC ATATTTTGAT |
| 781 | CATGAGGGCG ATTTAGCAAG TGATAAATTT AGAGTGCTGA CAGACATAGC AGGTCTAAAT |
| 841 | CTGAAGCGAA ACGACATGTA A |

(SEQ ID NO:31—Sb07g008600—S. propinquum) or functional fragment or variant, such as a codon optimized variant, thereof having at least 75%, 80%, 85%, 90%, 95%, 96%, 97%, 98%, or 99% sequence identity to SEQ ID NO:31.

2. Sequences for Day-Neutral Flowing

The S. bicolor cultivar from which the sequences described below are derived are day-neutral, and have the recessive (loss of function) Ma1 allele. Sequences for a recessive Ma1 gene are therefore provided.

In some embodiments, the maturity Ma1 gene (including non-coding sequence) as it is found in day-neutral S. bicolor can include the nucleic acid sequence:

| | |
|---|---|
| 1 | AAAAGAAAAG TGAGCACACC ACGACCTATC ATCAGCTCAT GGTCAGCTCT ACAAACTTAT |
| 61 | AGATTGCATC GAGATCTAAG ACTCAGGTAC AAATCATGTC AACATCTAAT GGTTTAGAAA |
| 121 | ATGAAAAAAG TTTTGAGTTT CAAAATATGA TACTTGAAAT TAACATTTGA ACTTTTTAGC |
| 181 | AAGATCTGAA AATAAAAAAT TCAACTAAAA AATTTATAGA TCATGTTAAC ATTGATATAA |
| 241 | TCGCTTCCAA TCGCCTCCCA TCGCTTCAGC TAGAAAACTT TTTTTCTCGA TTTAATTAAT |
| 301 | GAAATAGTAA TAACGTCATT GTACAAGATT CTTTCAAACC CCAACCCCTA TCATCGACGG |
| 361 | TGAGGGCTCC TATAATATGC ACTAGTGGAC GCCGGGTGGG TGGAACCTAA GAAGATTTTA |
| 421 | AAAAAAAAAT TAAGAAGAAG ATTTTTATCT AACTAACTAT ATATAGTACT TATATCATAC |
| 481 | ACTATACTAT TCAAAATATT ATTTTCACAA TTATGAATTT ACCCTTTTAC TCTTTATTAA |
| 541 | AAAAATATGA ATAAAGAATT ATCACGCCTC TATTTAGGGT CCTAATCCCC ATAATTTAAG |
| 601 | AGGCGATGAG AGGCGATGTG ACATCTATGG CCCACCGACC AAAGACACAA CTATCGCCTC |
| 661 | CCATCACCTT GCTTCTATCG CCTCTCATAG CTTTTCATAT TCTAGGTCCA CCGGCCATAG |
| 721 | ACACACCAAT CGCTTATCAT CGCCTTTTCC AACCATTGTA AAAATATTCA TAATTTTGAT |
| 781 | ATAAAATTTG TCTTCACTTG AGTATGGGAA AAAAATTATA CATAATGTTT TCGTGTGAGA |
| 841 | ATTTACAGGA ATGAACCCTT AAGATGTCCA AATGTAAATG ACCCTATTTA TTAAGAGGAG |
| 901 | CGGATCTATA GGCCTGGCTC TGAAAATGGA TTATGGATTG GAGATACTAA ATTTAAGGGC |
| 961 | CTATCTTCGC ACATAACATC TATAGTTCCT AAATAATTTT TTATTGTAGT AGTAGAACTT |
| 1021 | TTCTCCCTGT AAACCATAAA CCAAGTTGAC GCTGGGCTTT ATTTTGCGAC ACAGAACACC |
| 1081 | AAATTGGTGG CTATGAACTC TTCCACCTGG GCAGGGAAAA CGGTTTATTA TGTTCCTCTT |
| 1141 | TAATTTATCT ATCGTGGTCT GTTTTCACTA AAACTGTCAT ATTGCTACAC TCCAGTACTA |
| 1201 | CCAGTACGTC GCCCGCACAT AGTGGCCAAG GATTTTACTG CTACTGTTGA TTAACATAAG |
| 1261 | CACTTGCGAC TTTCCCTAAC ATCTTTTATA AAACAACGGC CGCAATAATA TTGAACTGTT |
| 1321 | TTTTTCTAGT ACCAAAAATA GAATTTGATC CCTCACCTCA TTACATCCAT AGTAACATGA |
| 1381 | CCAGATATAT ATGGACAGGC CGGGATCACT CGCCAGCAGA TACCCTGAGC GATTCATAAC |
| 1441 | CAGAATTTTT AATTTTTTCT AGTGAAGTGG GGTTCTCCTA GTCCTTTAAC ATTCAAAATT |
| 1501 | TAGTACAAAC TTTCCTTAGT AAATGTCTTC TAGTAAAGAT TTCCTAGTGT TTTGATTTGG |
| 1561 | TAGTGTTTTA TTACTAATTA AAAATATTAG AAGAACTCCA TCATTTTGGT AGTGATTGGT |
| 1621 | TGTTTGGATT AGTCTTCTCA CGTTAGACCT ATATATGCAG GACAACTCAA GCCAGCATAA |
| 1681 | ATATATGAAA TATCTTGGTG TTTGTTTGTC TGACACAGGC AACCGTGTTT GGTATAAATG |
| 1741 | TGTTTTCTTG TTTACGTTTT ACCATCTATA GTCATCTCAA TGTTTATATA GTAGAGACTT |
| 1801 | CATGTTTGTA GTAGATAAGG TAGAGAATTG AGAATATTTT ATTTTTGTGC GACCATCAAT |
| 1861 | TTTATGTAAT CTGCATTGTC TAATGCTTTA TTTGACATTT GAAACTACTT AATTTGACCG |
| 1921 | TTATGCAGGT CCGCATGATC CTATGAAAGC AATTAATTAG TACGGGTACT GCACTACACA |
| 1981 | AGTTTGCTAG TACTATTCTA TTAACCGACC TGTCAATATT ACCTTAAGTT ACTGATTTCA |
| 2041 | ATTAGAATCT AACACATTCA GGAAAAGAAG TTTTCCTTAT TAGTAGTAAC TTTTTATACT |
| 2101 | AATTAAGATT CAATAAAAAT TCACCATGAC ATCCCCATTG CCAAGAGAAT ATTTCGCCGC |
| 2161 | CCCTCAAAGC AGCCAAGGCT TTACTAAAAA GACTATCCAC GCAGTAGAGA TTTAGTCAAA |
| 2221 | ATATTCCAAT AGCAATTGTT TTCTGCCTGC TTGACCTTCG TCAGCCACTC ACTGTATAAA |
| 2281 | TATCGCACCA CGCCCTTTGC AGGCTTACAG AGCTTGTACT ACGTACTAAC AAGGCACACA |
| 2341 | CAATACCCTG TGTTCACCGG CCCTGCACAA AACTCAAGCA GTTATTACTA ACATGGCGGC |
| 2401 | TAACGATTCC TTGGTTACTG CTCATGTGAT AGGAGATGTC TTGGACCCCT TCTATACAAC |
| 2461 | CGTTGATATG ATGATCCTAT TCGATGGTAC TCCTATTATC AGCGGCATGG AGTTGCGTGC |
| 2521 | TCCGGCGGTT TCTGACAGGC CAAGGGTTGA GATTGGAGGA GATGATTATC GAGTTGCATA |
| 2581 | TACTCTGGTA AACTCATGTC ATGTCAATTA ACTAGTAGTT GAATTTAGAT GCTGGTCGTA |
| 2641 | TCGTGGATAC ATGAACTATA TGTTATGGTT GATACATATT TGTTTAATTG ATCGCAACAC |
| 2701 | CATTTGTGGT AACTTCAAAT AACATTCTTT CAATATATAG GTGATGGTCG ATCCTGATGC |
| 2761 | TCCTAACCCA AGCAACCCAA CCTTGAGGGA GTACTTGCAC TGGTAAGAGA AACCTATAGA |
| 2821 | CGACAATTAT TGTTGTTGGC ATGTTCTGCC CACATATACT TTGCTAGTGT GTGTATATTT |
| 2881 | GTGCTTATGC TTCTCCATAA ATTTTGGTGT ATGTCCCAAG AGAGATAGGT ATAGAGGTTA |
| 2941 | GCAGTCCTTT AAAAATGGTT TAATCCAGTA GTTTTTTTTC GGTCGGCCGG ACTGCTAGTA |
| 3001 | ACTTTCAATC ATTTCATGTT TCGAGCAGGA TGGTGACTGA CATCCCAGCA TCAACTGATA |
| 3061 | ATACATACGG TGAGATCACC CCTATTCCCA TTTTGAGACA AGTAGAATGT CTATTTTTAT |
| 3121 | GATCTAGTAT GTTCGTGACA ATAGGCTAGC TATTTTGAAA CTTCGGGAGC ATAAAATAGT |
| 3181 | ACTCGATTTT GTATAACCAT AAACACAGCT AGCCAATCTC TATTCATATT TATTTTAGTT |
| 3241 | TTATTTGCCG AACCATCCTC AACATCATAG CCACTTGATC GATCATCTCA ATCAGCGTTT |
| 3301 | GTATCCTTGC CCGCTTTGAT TATCATCCAT GACAGTTCAT ATTTTTTTTC ATTTCTTTCA |
| 3361 | TGCTTGTTAT AGTTTTATCT GATGAATCCG AGATGTTATT GATCAATTAG TTCAGATGAG |
| 3421 | CAGTAATGTA TGTTGGAGGT TTGGTAGTAT ATATACGTTC AATATTTCAC GAAATCGGTA |
| 3481 | ATTACGAAAA TCCCAAAATT TTGAATTACA TTAATAATGC ATGTGACTCA TATTTTCTAT |
| 3541 | GATTTCTATT CTGTTGCATA TTCTTGTACT CAATAGATAT TTAAATCATG CTAATATTTT |
| 3601 | GTTTAGATCT AAATCTTTTA GAAAAATTAT AATTTATATT TGGGTTTAAC AATTTCGGGC |
| 3661 | GCGTTTAGTG AGATTGGGTA ATTTCGGAGC GAGGCGGCCG CCGGCCACGA AAAATTCTAT |
| 3721 | ACACGACTAT ATGTGTACAT GTACATGCAT GGCACCTTGA TAGGCTACCC CGGCCCGCAT |
| 3781 | GGGGAAAAAA TTGGAAACGG ACCATTCATA CGCAGTCGTG GTGCCGACTG TGGGCCACAA |
| 3841 | TAGCAGTGTA AACATAATTA CGGTAATCAA ATACCCCGTG GGACCATATA TATCATCCAC |
| 3901 | AGATCCGTAC GGTGCTTCCG TGTGGATGGT CTACCCCAGA TCTTTTCCAC CCCATAAGGG |
| 3961 | CAGCAATGCA GCATCATATT CATATGCACT AGTGATGTAC CATTTGGCTT ATATCATATT |
| 4021 | CAACCTAACT CCTTGGAAAC ATTATGATGT TCTATTGGGG TGAAGATGTC ACTACTAAAA |
| 4081 | AAAGATCTTA TGAGAGGTGT TTTGAAAACT GCCCGAGGTG GTTAAAGGAG ACGGACGAGT |
| 4141 | TAGGACAACT GCCTCTATTA ATGTGTATTA ACCGAGGTAG TTACCGTAAC GTGCCTGACT |
| 4201 | TGATTAACAG ATTCAACCGT CTCAGTAAAG ACCATGATTA ACCGAAACGG AATCGAGAGT |
| 4261 | TTTCTCAAGT AGTTAAACTA TTTTAAACTG CACCGAACTT ATAAAAATGG TAGAGCTAAC |
| 4321 | ACCAATATTT ATAAAAATAA ATTAGTATCA CTAAATACAT CACGAAATCT ATTTGGTGTT |
| 4381 | GTAGAAGTTA TCCTTTTCTA TAAAATTGAT CAAATTTATG ATAACTTAGT TTTAGGAATT |
| 4441 | GATTTATTTT AGGACAACTA AGGAAGTACA TTTTTTAAAG TCATCCACAA AGTAGTGGAT |
| 4501 | CCAATTTATT ACATTACTCC ACTACTTCAA ACTGAACAAA AGCCTAATCC TGGTTATTTT |
| 4561 | GAGAGTGATT TTTTACAACA TCAGCAGTAG TCCAGAAAAT GGGAGGACAT TAATAAAAGT |
| 4621 | GAAAAGGAGC AGAAGAAAGA TTACGGTATT TTATTTGTGC TATTTGTTTA ACTATTGGCA |
| 4681 | GTTTGGGACC GAAAATAAAT AACTGTTCGT AGCTCTATAT TTGTCCATTC GAAAGTGTAA |
| 4741 | CGATGATTAT TGTGTTTCAA AAGATAAATA AAGAAGTGCA CCAATGATTT GATATCATAG |
| 4801 | GCTATATAAT CCAACATGGT GAAAATGCTT TTCAATCAAG TAATCTTCGA GCGGTTACCA |
| 4861 | GTTTTAATAG TTGCGAGTCG TCGTTTTTTA TGTACCCTAG GACATATATA TATCCGCATG |
| 4921 | TAGACGATGA GACTAGCTAG TTTTTTTTTT TTTGAGCAAA TACATAATTA TTGGATTTGC |
| 4981 | AGGCCGTGAG ATGATGTGCT ACGAGCCCCC TGCCCCGTCC ACGGGCATCC ACCGGATGGT |
| 5041 | GCTGGTGCTA TTCCAGCAGC TTGGCCGTGA CACGGTGTTC GCGGCGCCGT CCAGGCGCCA |
| 5101 | CAACTTCAAC ACCCGTGCCT TCGCCCGCCG CTACAACCTC GGCGCGCCCG TCGCCGCCAT |
| 5161 | GTTCTTCAAC TGCCAGCGCC AGACCGGCTC CGGTGGCCCC AGGTTCACCG GGCCCTACAC |
| 5221 | CAGCCGCCGT CGTGCGGGCT GATGACGACG ATCGTCGTTA CGTCACGTGT ACCGTACATA |
| 5281 | TATATGTAAG ATATACATGC ATGTTCCATG GTAAGGATCG GTGACAAAAC GTCTAATAAT |
| 5341 | GTATACACAC ATATGCATGG AATGCATGTA ATAAGAGAAT ATATGTATAA TAAGTAGGGG |
| 5401 | GGAGCATGCA TATATTGTAC ACGCGTCCGA TGCGTATATA GCCCTATACA TTATTGTAGT |
| 5461 | TGTAATCAGC TGTTTAAGCA TTCTGCTGTG TCAGAACATG ATGCATATAT AGTTTGGTGT |
| 5521 | CAGTATTGAT GTTGTGGAAC TCTTATCAGC CTTCATCTCA TCACAAGTGA AAGATATAGC |
| 5581 | TTTTATACCT CCAAGTGTCT TCCCAATGTA CGTACCTAGA ACTTTTCTAA GAAATGCTAC |
| 5641 | AAATGTTGTA TTTTATCTGT GCGCTTCACT ACTGGAAACC CGAATATTTC TGTGGATGTC |
| 5701 | GAATTTTTCT GTGCGTTTTT TTCGATACGC ACGGAAAAAT TATAATTATT TTGTGAGTTT |
| 5761 | TAAAATACCC TCACAGAAAA ATACAAATAC CCACAGAACA ATTATATCAT TTTTCTGTGC |
| 5821 | GTGACAATAC ACTCACAAAA ATTACAATTT TTGTGTGTGT TTATATAAAA TGCACAGAAA |
| 5881 | AAAATAATCA CACACAGAAA AATTATACTT ATTCTGTGGG TTTCTATAAA ACGCACATAA |
| 5941 | AAAAATAAAC ACACAGAGAA AAATAGAACA AGCACCCTCA TACTAACTTC ATATGAACAC |
| 6001 | GCATATTTTT TCTTTTTAAT CTCTCTGTAA AACTTGTAAC TAGTTTTTCC CACTCGTACT |
| 6061 | AACTCCAAAT TGGATGATTT |

(SEQ ID NO:9, Sb06g012260—S. bicolor), or a variant, such as a codon optimized variant, thereof having at least 75%, 80%, 85%, 90%, 95%, 96%, 97%, 98%, or 99% sequence identity to SEQ ID NO:9.

The coding sequence of the maturity Ma1 gene of SEQ ID NO:10, including introns, can be:

| | |
|---|---|
| 1 | ATGGCGGCTA ACGATTCCTT GGTTACTGCT CATGTGATAG GAGATGTCTT GGACCCCTTC |
| 61 | TATACAACCG TTGATATGAT GATCCTATTC GATGGTACTC CTATTATCAG CGGCATGGAG |
| 121 | TTGCGTGCTC CGGCGGTTTC TGACAGGCCA AGGGTTGAGA TTGGAGGAGA TGATTATCGA |
| 181 | GTTGCATATA CTCTGGTAAA CTCATGTCAT GTCAATTAAC TAGTAGTTGA ATTTAGATGC |
| 241 | TGGTCGTATC GTGGATACAT GAACTATATG TTATGGTTGA TACATATTTG TTTAATTGAT |
| 301 | CGCAACACCA TTTGTGGTAA CTTCAAATAA CATTCTTTCA ATATATAGGT GATGGTCGAT |
| 361 | CCTGATGCTC CTAACCCAAG CAACCCAACC TTGAGGGAGT ACTTGCACTG GTAAGAGAAA |
| 421 | CCTATAGACG ACAATTATTG TTGTTGGCAT GTTCTGCCCA CATATACTTT GCTAGTGTGT |
| 481 | GTATATTTGT GCTTATGCTT CTCCATAAAT TTTGGTGTAT GTCCCAAGAG AGATAGGTAT |
| 541 | AGAGGTTAGC AGTCCTTTAA AAATGGTTTA ATCCAGTAGT TTTTTTTCGG TCGGCCGGAC |
| 601 | TGCTAGTAAC TTTCAATCAT TTCATGTTTC GAGCAGGATG GTGACTGACA TCCCAGCATC |
| 661 | AACTGATAAT ACATACGGTG AGATCACCCC TATTCCCATT TTGAGACAAG TAGAATGTCT |
| 721 | ATTTTTATGA TCTAGTATGT TCGTGACAAT AGGCTAGCTA TTTTGAAACT TCGGGAGCAT |
| 781 | AAAATAGTAC TCGATTTTGT ATAACCATAA ACACAGCTAG CCAATCTCTA TTCATATTTA |
| 841 | TTTTAGTTTT ATTTGCCGAA CCATCCTCAA CATCATAGCC ACTTGATCGA TCATCTCAAT |
| 901 | CAGCGTTTGT ATCCTTGCCC GCTTTGATTA TCATCCATGA CAGTTCATAT TTTTTTTCAT |
| 961 | TTCTTTCATG CTTGTTATAG TTTTATCTGA TGAATCCGAG ATGTTATTGA TCAATTAGTT |
| 1021 | CAGATGAGCA GTAATGTATG TTGGAGGTTT GGTAGTATAT ATACGTTCAA TATTTCACGA |
| 1081 | AATCGGTAAT TACGAAAATC CCAAAATTTT GAATTACATT AATAATGCAT GTGACTCATA |
| 1141 | TTTTCTATGA TTTCTATTCT GTTGCATATT CTTGTACTCA ATAGATATTT AAATCATGCT |
| 1201 | AATATTTTGT TTAGATCTAA ATCTTTTAGA AAAATTATAA TTTATATTTG GGTTTAACAA |
| 1261 | TTTCGGGCGC GTTTAGTGAG ATTGGGTAAT TTCGGAGCGA GGCGGCCGCC GGCCACGAAA |
| 1321 | AATTCTATAC ACGACTATAT GTGTACATGT ACATGCATGG CACCTTGATA GGCTACCCCG |
| 1381 | GCCCGCATGG GGAAAAAATT GGAAACGGAC CATTCATACG CAGTCGTGGT GCCGACTGTG |
| 1441 | GGCCACAATA GCAGTGTAAA CATAATTACG GTAATCAAAT ACCCCGTGGG ACCATATATA |
| 1501 | TCATCCACAG ATCCGTACGG TGCTTCCGTG TGGATGGTCT ACCCCAGATC TTTTCCACCC |
| 1561 | CATAAGGGCA GCAATGCAGC ATCATATTCA TATGCACTAG TGATGTACCA TTTGGCTTAT |
| 1621 | ATCATATTCA ACCTAACTCC TTGGAAACAT TATGATGTTC TATTGGGGTG AAGATGTCAC |
| 1681 | TACTAAAAAA AGATCTTATG AGAGGTGTTT TGAAAACTGC CCGAGGTGGT TAAAGGAGAC |
| 1741 | GGACGAGTTA GGACAACTGC CTCTATTAAT GTGTATTAAC CGAGGTAGTT ACCGTAACGT |
| 1801 | GCCTGACTTG ATTAACAGAT TCAACCGTCT CAGTAAAGAC CATGATTAAC CGAAACGGAA |
| 1861 | TCGAGAGTTT TCTCAAGTAG TTAAACTATT TTAAACTGCA CCGAACTTAT AAAAATGGTA |
| 1921 | GAGCTAACAC CAATATTTAT AAAAATAAAT TAGTATCACT AAATACATCA CGAAATCTAT |
| 1981 | TTGGTGTTGT AGAAGTTATC CTTTTCTATA AAATTGATCA AATTTATGAT AACTTAGTTT |
| 2041 | TAGGAATTGA TTTATTTTAG GACAACTAAG GAAGTACATT TTTTAAAGTC ATCCACAAAG |
| 2101 | TAGTGGATCC AATTTATTAC ATTACTCCAC TACTTCAAAC TGAACAAAAG CCTAATCCTG |
| 2161 | GTTATTTTGA GAGTGATTTT TTACAACATC AGCAGTAGTC CAGAAAATGG GAGGACATTA |
| 2221 | ATAAAAGTGA AAAGGAGCAG AAGAAAGATT ACGGTATTTT ATTTGTGCTA TTTGTTTAAC |
| 2281 | TATTGGCAGT TTGGGACCGA AAATAAATAA CTGTTCGTAG CTCTATATTT GTCCATTCGA |
| 2341 | AAGTGTAACG ATGATTATTG TGTTTCAAAA GATAAATAAA GAAGTGCACC AATGATTTGA |
| 2401 | TATCATAGGC TATATAATCC AACATGGTGA AAATGCTTTT CAATCAAGTA ATCTTCGAGC |
| 2461 | GGTTACCAGT TTTAATAGTT GCGAGTCGTC GTTTTTTATG TACCCTAGGA CATATATATA |
| 2521 | TCCGCATGTA GACGATGAGA CTAGCTAGTT TTTTTTTTTT TGAGCAAATA CATAATTATT |
| 2581 | GGATTTGCAG GCCGTGAGAT GATGTGCTAC GAGCCCCCTG CCCCGTCCAC GGGCATCCAC |
| 2641 | CGGATGGTGC TGGTGCTATT CCAGCAGCTT GGCCGTGACA CGGTGTTCGC GGCGCCGTCC |
| 2701 | AGGCGCCACA ACTTCAACAC CCGTGCCTTC GCCCGCCGCT ACAACCTCGG CGCGCCCGTC |
| 2761 | GCCGCCATGT TCTTCAACTG CCAGCGCCAG ACCGGCTCCG GTGGCCCCAG GTTCACCGGG |
| 2821 | CCCTACACCA GCCGCCGTCG TGCGGGCTGA |

(SEQ ID NO:10 Sb06g012260—S. bicolor) or functional fragment or variant, such as a codon optimized variant, thereof having at least 75%, 80%, 85%, 90%, 95%, 96%, 97%, 98%, or 99% sequence identity to SEQ ID NO:10.

The coding sequence, without introns, of the maturity Ma1 gene as it is found in day-neutral S. bicolor can include the nucleic acid sequence:

| | |
|---|---|
| 1 | ATGGCGGCTA ACGATTCCTT GGTTACTGCT CATGTGATAG GAGATGTCTT GGACCCCTTC |
| 61 | TATACAACCG TTGATATGAT GATCCTATTC GATGGTACTC CTATTATCAG CGGCATGGAG |
| 121 | TTGCGTGCTC CGGCGGTTTC TGACAGGCCA AGGGTTGAGA TTGGAGGAGA TGATTATCGA |
| 181 | GTTGCATATA CTCTGGTGAT GGTCGATCCT GATGCTCCTA ACCCAAGCAA CCCAACCTTG |
| 241 | AGGGAGTACT TGCACTGGAT GGTGACTGAC ATCCCAGCAT CAACTGATAA TACATACGGC |
| 301 | CGTGAGATGA TGTGCTACGA GCCCCCTGCC CCGTCCACGG GCATCCACCG GATGGTGCTG |
| 361 | GTGCTATTCC AGCAGCTTGG CCGTGACACG GTGTTCGCGG CGCCGTCCAG GCGCCACAAC |
| 421 | TTCAACACCC GTGCCTTCGC CCGCCGCTAC AACCTCGGCG CGCCCGTCGC CGCCATGTTC |
| 481 | TTCAACTGCC AGCGCCAGAC CGGCTCCGGT GGCCCCAGGT TCACCGGGCC CTACACCAGC |
| 541 | CGCCGTCGTG CGGGCTGA |

(SEQ ID NO:11, Sb06g012260 —S. bicolor), or a variant thereof, for example a codon optimized variant, having at least 75%, 80%, 85%, 90%, 95%, 96%, 97%, 98%, or 99% sequence identity to SEQ ID NO:11.

In this embodiment, the maturity Ma1 protein as it is found in short-day—S. bicolor can include the amino acid sequence SEQ ID NO:8, or a variant thereof having at least 95% sequence identity to SEQ ID NO:8.

In some embodiments, the maturity Ma1 gene (including non-coding sequence) as it is found in day-neutral S. bicolor can include the nucleic acid sequence:

| | |
|---|---|
| 1 | TTCCACCTGG GCAGGGAAAA CGGTTTATTA TGTTCCTCTT TAATTTATCT ATCGTGGTCT |
| 61 | GTTTTCACTA AAACTGTCAT ATTGCTACAC TCCAGTACTA CCAGTACGTC GCCCGCACAT |
| 121 | AGTGGCCAAG GATTTTACTG CTACTGTTGA TTAACATAAG CACTTGCGAC TTTCCCTAAC |
| 181 | ATCTTTTATA AAACAACGGC CGCAATAATA TTGAACTGTT TTTTTCTAGT ACCAAAAATA |
| 241 | GAATTTGATC CCTCACCTCA TTACATCCAT AGTAACATGA CCAGATATAT ATGGACAGGC |
| 301 | CGGGATCACT CGCCAGCAGA TACCCTGAGC GATTCATAAC CAGAATTTTT AATTTTTTCT |
| 361 | AGTGAAGTGG GGTTCTCCTA GTCCTTTAAC ATTCAAAATT TAGTACAAAC TTTCCTTAGT |
| 421 | AAATGTCTTC TAGTAAAGAT TTCCTAGTGT TTTGATTTGG TAGTGTTTTA TTACTAATTA |
| 481 | AAAATATTAG AAGAACTCCA TCATTTTGGT AGTGATTGGT TGTTTGGATT AGTCTTCTCA |
| 541 | CGTTAGACCT ATATATGCAG GACAACTCAA GCCAGCATAA ATATATGAAA TATCTTGGTG |
| 601 | TTTGTTTGTC TGACACAGGC AACCGTGTTT GGTATAAATG TGTTTTCTTG TTTACGTTTT |
| 661 | ACCATCTATA GTCATCTCAA TGTTTATATA GTAGAGACTT CATGTTTGTA GTAGATAAGG |
| 721 | TAGAGAATTG AGAATATTTT ATTTTTGTGC GACCATCAAT TTTATGTAAT CTGCATTGTC |
| 781 | TAATGCTTTA TTTGACATTT GAAACTACTT AATTTGACCG TTATGCAGGT CCGCATGATC |
| 841 | CTATGAAAGC AATTAATTAG TACGGGTACT GCACTACACA AGTTTGCTAG TACTATTCTA |
| 901 | TTAACCGACC TGTCAATATT ACCTTAAGTT ACTGATTTCA ATTAGAATCT AACACATTCA |
| 961 | GGAAAAGAAG TTTTCCTTAT TAGTAGTAAC TTTTTATACT AATTAAGATT CAATAAAAAT |
| 1021 | TCACCATGAC ATCCCCATTG CCAAGAGAAT ATTTCGCCGC CCCTCAAAGC AGCCAAGGCT |
| 1081 | TTACTAAAAA GACTATCCAC GCAGTAGAGA TTTAGTCAAA ATATTCCAAT AGCAATTGTT |
| 1141 | TTCTGCCTGC TTGACCTTCG TCAGCCACTC ACTGTATAAA TATCGCACCA CGCCCTTTGC |
| 1201 | AGGCTTACAG AGCTTGTACT ACGTACTAAC AAGGCACACA CAATACCCTG TGTTCACCGG |
| 1261 | CCCTGCACAA AACTCAAGCA GTTATTACTA ACATGGCGGC TAACGATTCC TTGGTTACTG |
| 1321 | CTCATGTGAT AGGAGATGTC TTGGACCCCT TCTATACAAC CGTTGATATG ATGATCCTAT |
| 1381 | TCGATGGTAC TCCTATTATC AGCGGCATGG AGTTGCGTGC TCCGGCGGTT TCTGACAGGC |
| 1441 | CAAGGGTTGA GATTGGAGGA GATGATTATC GAGTTGCATA TACTCTGGTA AACTCATGTC |
| 1501 | ATGTCAATTA ACTAGTAGTT GAATTTAGAT GCTGGTCGTA TCGTGGATAC ATGAACTATA |
| 1561 | TGTTATGGTT GATACATATT TGTTTAATTG ATCGCAACAC CATTTGTGGT AACTTCAAAT |
| 1621 | AACATTCTTT CAATATATAG GTGATGGTCG ATCCTGATGC TCCTAACCCA AGCAACCCAA |
| 1681 | CCTTGAGGGA GTACTTGCAC TGGTAAGAGA AACCTATAGA CGACAATTAT TGTTGTTGGC |
| 1741 | ATGTTCTGCC CACATATACT TTGCTAGTGT GTGTATATTT GTGCTTATGC TTCTCCATAA |
| 1801 | ATTTTGGTGT ATGTCCCAAG AGAGATAGGT ATAGAGGTTA GCAGTCCTTT AAAAATGGTT |
| 1861 | TAATCCAGTA GTTTTTTTTC GGTCGGCCGG ACTGCTAGTA ACTTTCAATC ATTTCATGTT |
| 1921 | TCGAGCAGGA TGGTGACTGA CATCCCAGCA TCAACTGATA ATACATACGG CCGTGAGATC |
| 1981 | ACCCCTATTC CCATTTTGAG ACAAGTAGAA TGTCTATTTT TATGATCTAG TATGTTCGTG |
| 2041 | ACAATAGGCT AGCTATTTTG AAACTTCGGG AGCATAAAAT AGTACTCGAT TTTGTATAAC |
| 2101 | CATAAACACA GCTAGCCAAT CTCTATTCAT ATTTATTTTA GTTTTATTTG CCGAACCATC |
| 2161 | CTCAACATCA TAGCCACTTG ATCGATCATC TCAATCAGCG TTTGTATCCT TGCCCGCTTT |
| 2221 | GATTATCATC CATGACAGTT CATATTTTTT TTCATTTCTT TCATGCTTGT TATAGTTTTA |
| 2281 | TCTGATGAAT CCGAGATGTT ATTGATCAAT TAGTTCAGAT GAGCAGTAAT GTATGTTGGA |
| 2341 | GGTTTGGTAG TATATATACG TTCAATATTT CACGAAATCG GTAATTACGA AAATCCCAAA |
| 2401 | ATTTTGAATT ACATTAATAA TGCATGTGAC TCATATTTTC TATGATTTCT ATTCTGTTGC |
| 2461 | ATATTCTTGT ACTCAATAGA TATTTAAATC ATGCTAATAT TTTGTTTAGA TCTAAATCTT |
| 2521 | TTAGAAAAAT TATAATTTAT ATTTGGGTTT AACAATTTCG GGCGCGTTTA GTGAGATTGG |
| 2581 | GTAATTTCGG AGCGAGGCGG CCGCCGGCCA CGAAAAATTC TATACACGAC TATATGTGTA |
| 2641 | CATGTACATG CATGGCACCT TGATAGGCTA CCCCGGCCCG CATGGGGAAA AAATTGGAAA |
| 2701 | CGGACCATTC ATACGCAGTC GTGGTGCCGA CTGTGGGCCA CAATAGCAGT GTAAACATAA |
| 2761 | TTACGGTAAT CAAATACCCC GTGGGACCAT ATATATCATC CACAGATCCG TACGGTGCTT |
| 2821 | CCGTGTGGAT GGTCTACCCC AGATCTTTTC CACCCCATAA GGGCAGCAAT GCAGCATCAT |
| 2881 | ATTCATATGC ACTAGTGATG TACCATTTGG CTTATATCAT ATTCAACCTA ACTCCTTGGA |
| 2941 | AACATTATGA TGTTCTATTG GGGTGAAGAT GTCACTACTA AAAAAAGATC TTATGAGAGG |
| 3001 | TGTTTTGAAA ACTGCCCGAG GTGGTTAAAG GAGACGGACG AGTTAGGACA ACTGCCTCTA |
| 3061 | TTAATGTGTA TTAACCGAGG TAGTTACCGT AACGTGCCTG ACTTGATTAA CAGATTCAAC |
| 3121 | CGTCTCAGTA AAGACCATGA TTAACCGAAA CGGAATCGAG AGTTTTCTCA AGTAGTTAAA |
| 3181 | CTATTTTAAA CTGCACCGAA CTTATAAAAA TGGTAGAGCT AACACCAATA TTTATAAAAA |
| 3241 | TAAATTAGTA TCACTAAATA CATCACGAAA TCTATTTGGT GTTGTAGAAG TTATCCTTTT |
| 3301 | CTATAAAATT GATCAAATTT ATGATAACTT AGTTTTAGGA ATTGATTTAT TTTAGGACAA |
| 3361 | CTAAGGAAGT ACATTTTTTA AAGTCATCCA CAAAGTAGTG GATCCAATTT ATTACATTAC |
| 3421 | TCCACTACTT CAAACTGAAC AAAAGCCTAA TCCTGGTTAT TTTGAGAGTG ATTTTTTACA |
| 3481 | ACATCAGCAG TAGTCCAGAA AATGGGAGGA CATTAATAAA AGTGAAAAGG AGCAGAAGAA |
| 3541 | AGATTACGGT ATTTTATTTG TGCTATTTGT TTAACTATTG GCAGTTTGGG ACCGAAAATA |
| 3601 | AATAACTGTT CGTAGCTCTA TATTTGTCCA TTCGAAAGTG TAACGATGAT TATTGTGTTT |
| 3661 | CAAAAGATAA ATAAAGAAGT GCACCAATGA TTTGATATCA TAGGCTATAT AATCCAACAT |
| 3721 | GGTGAAAATG CTTTTCAATC AAGTAATCTT CGAGCGGTTA CCAGTTTTAA TAGTTGCGAG |
| 3781 | TCGTCGTTTT TTATGTACCC TAGGACATAT ATATATCCGC ATGTAGACGA TGAGACTAGC |
| 3841 | TAGTTTTTTT TTTTTTGAGC AAATACATAA TTATTGGATT TGCAGGCCGT GAGATGATGT |
| 3901 | GCTACGAGCC CCCTGCCCCG TCCACGGGCA TCCACCGGAT GGTGCTGGTG CTATTCCAGC |
| 3961 | AGCTTGGCCG TGACACGGTG TTCGCGGCGC CGTCCAGGCG CCACAACTTC AACACCCGTG |
| 4021 | CCTTCGCCCG CCGCTACAAC CTCGGCGCGC CCGTCGCCGC CATGTTCTTC AACTGCCAGC |
| 4081 | GCCAGACCGG CTCCGGTGGC CCCAGGTTCA CCGGGCCCTA CACCAGCCGC CGTCGTGCGG |
| 4141 | GCTGATGACG ACGATCGTCG TTACGTCACG TGTACCGTAC ATATATATGT AAGATATACA |
| 4201 | TGCATGTTCC ATGGTAAGGA TCGGTGACAA AACGTCTAAT AATGTATACA CACATATGCA |
| 4261 | TGGAATGCAT GTAATAAGAG AATATATGTA TAATAAGTAG GGGGGAGCAT GCATATATTG |
| 4321 | TACACGCGTC CGATGCGTAT ATAGCCCTAT ACATTATTGT AGTTGTAATC A |

(SEQ ID NO:12, Sb06g012260 —S. bicolor), or a variant, for example a codon optimized variant, thereof having at least at least 75%, 80%, 85%, 90%, 95%, 96%, 97%, 98%, or 99% sequence identity to SEQ ID NO:12.

The coding sequence of the maturity Ma1 gene of SEQ ID NO:12, including introns, can be:

| | |
|---|---|
| 1 | ATGGCGGCTA ACGATTCCTT GGTTACTGCT CATGTGATAG GAGATGTCTT GGACCCCTTC |
| 61 | TATACAACCG TTGATATGAT GATCCTATTC GATGGTACTC CTATTATCAG CGGCATGGAG |
| 121 | TTGCGTGCTC CGGCGGTTTC TGACAGGCCA AGGGTTGAGA TTGGAGGAGA TGATTATCGA |
| 181 | GTTGCATATA CTCTGGTAAA CTCATGTCAT GTCAATTAAC TAGTAGTTGA ATTTAGATGC |
| 241 | TGGTCGTATC GTGGATACAT GAACTATATG TTATGGTTGA TACATATTTG TTTAATTGAT |
| 301 | CGCAACACCA TTTGTGGTAA CTTCAAATAA CATTCTTTCA ATATATAGGT GATGGTCGAT |
| 361 | CCTGATGCTC CTAACCCAAG CAACCCAACC TTGAGGGAGT ACTTGCACTG GTAAGAGAAA |
| 421 | CCTATAGACG ACAATTATTG TTGTTGGCAT GTTCTGCCCA CATATACTTT GCTAGTGTGT |
| 481 | GTATATTTGT GCTTATGCTT CTCCATAAAT TTTGGTGTAT GTCCCAAGAG AGATAGGTAT |
| 541 | AGAGGTTAGC AGTCCTTTAA AAATGGTTTA ATCCAGTAGT TTTTTTTCGG TCGGCCGGAC |
| 601 | TGCTAGTAAC TTTCAATCAT TTCATGTTTC GAGCAGGATG GTGACTGACA TCCCAGCATC |
| 661 | AACTGATAAT ACATACGGCC GTGAGATCAC CCCTATTCCC ATTTTGAGAC AAGTAGAATG |
| 721 | TCTATTTTTA TGATCTAGTA TGTTCGTGAC AATAGGCTAG CTATTTTGAA ACTTCGGGAG |
| 781 | CATAAAATAG TACTCGATTT TGTATAACCA TAAACACAGC TAGCCAATCT CTATTCATAT |
| 841 | TTATTTTAGT TTTATTTGCC GAACCATCCT CAACATCATA GCCACTTGAT CGATCATCTC |
| 901 | AATCAGCGTT TGTATCCTTG CCCGCTTTGA TTATCATCCA TGACAGTTCA TATTTTTTTT |
| 961 | CATTTCTTTC ATGCTTGTTA TAGTTTTATC TGATGAATCC GAGATGTTAT TGATCAATTA |
| 1021 | GTTCAGATGA GCAGTAATGT ATGTTGGAGG TTTGGTAGTA TATATACGTT CAATATTTCA |
| 1081 | CGAAATCGGT AATTACGAAA ATCCCAAAAT TTTGAATTAC ATTAATAATG CATGTGACTC |
| 1141 | ATATTTTCTA TGATTTCTAT TCTGTTGCAT ATTCTTGTAC TCAATAGATA TTTAAATCAT |
| 1201 | GCTAATATTT TGTTTAGATC TAAATCTTTT AGAAAAATTA TAATTTATAT TTGGGTTTAA |
| 1261 | CAATTTCGGG CGCGTTTAGT GAGATTGGGT AATTTCGGAG CGAGGCGGCC GCCGGCCACG |
| 1321 | AAAAATTCTA TACACGACTA TATGTGTACA TGTACATGCA TGGCACCTTG ATAGGCTACC |
| 1381 | CCGGCCCGCA TGGGGAAAAA ATTGGAAACG GACCATTCAT ACGCAGTCGT GGTGCCGACT |
| 1441 | GTGGGCCACA ATAGCAGTGT AAACATAATT ACGGTAATCA AATACCCCGT GGGACCATAT |
| 1501 | ATATCATCCA CAGATCCGTA CGGTGCTTCC GTGTGGATGG TCTACCCCAG ATCTTTTCCA |
| 1561 | CCCCATAAGG GCAGCAATGC AGCATCATAT TCATATGCAC TAGTGATGTA CCATTTGGCT |
| 1621 | TATATCATAT TCAACCTAAC TCCTTGGAAA CATTATGATG TTCTATTGGG GTGAAGATGT |
| 1681 | CACTACTAAA AAAAGATCTT ATGAGAGGTG TTTTGAAAAC TGCCCGAGGT GGTTAAAGGA |
| 1741 | GACGGACGAG TTAGGACAAC TGCCTCTATT AATGTGTATT AACCGAGGTA GTTACCGTAA |
| 1801 | CGTGCCTGAC TTGATTAACA GATTCAACCG TCTCAGTAAA GACCATGATT AACCGAAACG |
| 1861 | GAATCGAGAG TTTTCTCAAG TAGTTAAACT ATTTTAAACT GCACCGAACT TATAAAAATG |
| 1921 | GTAGAGCTAA CACCAATATT TATAAAAATA AATTAGTATC ACTAAATACA TCACGAAATC |
| 1981 | TATTTGGTGT TGTAGAAGTT ATCCTTTTCT ATAAAATTGA TCAAATTTAT GATAACTTAG |
| 2041 | TTTTAGGAAT TGATTTATTT TAGGACAACT AAGGAAGTAC ATTTTTTAAA GTCATCCACA |
| 2101 | AAGTAGTGGA TCCAATTTAT TACATTACTC CACTACTTCA AACTGAACAA AAGCCTAATC |
| 2161 | CTGGTTATTT TGAGAGTGAT TTTTTACAAC ATCAGCAGTA GTCCAGAAAA TGGGAGGACA |
| 2221 | TTAATAAAAG TGAAAAGGAG CAGAAGAAAG ATTACGGTAT TTTATTTGTG CTATTTGTTT |
| 2281 | AACTATTGGC AGTTTGGGAC CGAAAATAAA TAACTGTTCG TAGCTCTATA TTTGTCCATT |
| 2341 | CGAAAGTGTA ACGATGATTA TTGTGTTTCA AAAGATAAAT AAAGAAGTGC ACCAATGATT |
| 2401 | TGATATCATA GGCTATATAA TCCAACATGG TGAAAATGCT TTTCAATCAA GTAATCTTCG |
| 2461 | AGCGGTTACC AGTTTTAATA GTTGCGAGTC GTCGTTTTTT ATGTACCCTA GGACATATAT |
| 2521 | ATATCCGCAT GTAGACGATG AGACTAGCTA GTTTTTTTTT TTTTGAGCAA ATACATAATT |
| 2581 | ATTGGATTTG CAGGCCGTGA GATGATGTGC TACGAGCCCC CTGCCCCGTC CACGGGCATC |
| 2641 | CACCGGATGG TGCTGGTGCT ATTCCAGCAG CTTGGCCGTG ACACGGTGTT CGCGGCGCCG |
| 2701 | TCCAGGCGCC ACAACTTCAA CACCCGTGCC TTCGCCCGCC GCTACAACCT CGGCGCGCCC |
| 2761 | GTCGCCGCCA TGTTCTTCAA CTGCCAGCGC CAGACCGGCT CCGGTGGCCC CAGGTTCACC |
| 2821 | GGGCCCTACA CCAGCCGCCG TCGTGCGGGC TGA |

(SEQ ID NO:13 Sb06g012260—S. bicolor) or functional fragment or variant, such as a codon optimized variant, thereof having at least 75%, 80%, 85%, 90%, 95%, 96%, 97%, 98%, or 99% sequence identity to SEQ ID NO:13.

In some embodiments, the maturity Ma1 gene (including non-coding sequence) as it is found in day-neutral S. bicolor can include the nucleic acid sequence:

| | |
|---|---|
| 1 | ATGCCCCCAT CAAAGGAAGC CCCAAGTGGC GATGTACATG TCAAACAGCC ATCAAGTCAA |
| 61 | CCATTGACCC TAAAGGATAT CAGAAAGCCA ACGATTGATG ATTATGTCAA TGTCCCCAGT |
| 121 | GACTATGTGC CCGGAAGGCC TATGCTCCAA TGGACGCTGC TTGATAAGAT TCAATGGCCG |
| 181 | ATAAAAAGGT TTCATGACTG GTACATGAGA GCAGTGCATG CTGGCCTCCA TGCAATCAGA |
| 241 | GTTGATATAC CAGCAAACGT GTTTGCTACT GGTAACGAAA AAAGCAAGGC ATTTGTTATC |
| 301 | TTTGAGGACA TGCACTTGTT ATTGAACTAT AGGCGGCTTG ACGTCCAACT CATAACAATC |
| 361 | TGGTGTCTGT AAGTACCACT CATGCACACA CAATTATTAT TAATATGTAG TGTGAAACTC |
| 421 | TAATATGTAG ATGTTGTCTG TAGTTTGCAA GATCACGAGT AGAGGTCATT ATTATCTACC |
| 481 | GGATCAATGG TCGGTTATCT GAGCCCTATC AAGTTACAAG AAAATATGCA CAAATTTGTA |
| 541 | TTATCAAAGG AAGATAGAGC AAAGATAGAG GAAGACAAAA CACCAGAAAA AGTTGCAGAA |
| 601 | GCTATAAAAG AGTTGCAAAG AAAATACGAG GATAATTATG CCCTCTACCT TGGTAGATCA |
| 661 | ATGCTGAGGT ATAAGTATAG GGATTTTATA TTGGCACCTT ACAACTTTAG GTAAGCTTGA |
| 721 | CTTCATATAC GTACTTCAAA TAATTATCGT GTAAACAATA TACATGTGTC GCTCACTCAT |
| 781 | TTATTCATGC AGTGACCATT GGATTGTTTT TTATATTTAT CCCTTCGAAA GGAAGGTGCT |
| 841 | TGTCCTAGAC TCTTTACATG TTCCTCCCGA GAAGTATCAA CCATTCTTGG TTCAATTAGA |
| 901 | AAGGTGAGCC AACATGAAAC CACATGCGTA CTTATATAAA TTAGAGTTTC AAAACAACTT |
| 961 | TAGTGATTTA TATTCGATAT CTACAGGGCA TGGCGGTTTT ATAAGAAACA AAAGGGACCG |
| 1021 | GTCGACGCCG CACGCTCAGA TCCTAGGGTG CCATTGATGA TACAACACCA CTATCCGGTA |
| 1081 | AGTTGTCCGA ACACATTTCA TCATATAAAT AATACATAAA GCATGGCAAA TTTAGAATAA |
| 1141 | TCCGTTGCTC ATTATATAGT GCCACAAGCA ACCATCTGGA TCGGTCTATT GTGGGTACTA |
| 1201 | TGTCTGTGAG TTTATAAGGC AGCGGGGACG TTACGTCACG GACAAAAATA TGGTAAATAA |
| 1261 | TATCTATGTA TGAAGTTTTC TCATTAAAGT TGCAAAATTA TATATTGAAC ATGTGTCAAT |
| 1321 | CATGCTTTTA AACTTTGTTT CCAGCCAAAA AAGCAAAAAA AGGACGTGCC CTTTACACCA |
| 1381 | AAGACTCTGG AAGATATAGT AGCAGACTTG TGTGGTTTTA TTATGAGAGA AATAATTCCA |
| 1441 | AGTGACGGTG CATATTTTGA TCATGAGGGC GATTTAGCAA GTGATAAATT TAGAGTGCTG |
| 1501 | ACAGACATAG CAGGTCTAAA TCTGAAGCGA AATGACATG |

(SEQ ID NO:32—Sb07g008600—S. bicolor) or functional fragment or variant, such as a codon optimized variant, thereof having at least 75%, 80%, 85%, 90%, 95%, 96%, 97%, 98%, or 99% sequence identity to SEQ ID NO:32.

The coding sequence, without introns, of the maturity Ma1 gene according to SEQ ID NO:32 as it is found in day-neutral S. bicolor can include the nucleic acid sequence:

| | |
|---|---|
| 1 | ATGCCCCCAT CAAAGGAAGC CCCAAGTGGC GATGTACATG TCAAACAGCC ATCAAGTCAA |
| 61 | CCATTGACCC TAAAGGATAT CAGAAAGCCA ACGATTGATG ATTATGTCAA TGTCCCCAGT |
| 121 | GACTATGTGC CCGGAAGGCC TATGCTCCAA TGGACGCTGC TTGATAAGAT TCAATGGCCG |
| 181 | ATAAAAAGGT TTCATGACTG GTACATGAGA GCAGTGCATG CTGGCCTCCA TGCAATCAGA |
| 241 | GTTGATATAC CAGCAAACGT GTTTGCTACT GGTAACGAAA AAAGCAAGGC ATTTGTTATC |
| 301 | TTTGAGGACA TGCACTTGTT ATTGAACTAT AGGCGGCTTG ACGTCCAACT CATAACAATC |
| 361 | TGGTGTCTGG ACCATTGGAT TGTTTTTTAT ATTTATCCCT TCGAAAGGAA GGTGCTTGTC |
| 421 | CTAGACTCTT TACATGTTCC TCCCGAGAAG TATCAACCAT TCTTGGTTCA ATTAGAAAGG |
| 481 | GCATGGCGGT TTTATAAGAA ACAAAAGGGA CCGGTCGACG CCGCACGCTC AGATCCTAGG |
| 541 | GTGCCATTGA TGATACAACA CCACTATCCG TGCCACAAGC AACCATCTGG ATCGGTCTAT |
| 601 | TGTGGGTACT ATGTCTGTGA GTTTATAAGG CAGCGGGGAC GTTACGTCAC GGACAAAAAT |
| 661 | ATGCCAAAAA AGCAAAAAAA GGACGTGCCC TTTACACCAA AGACTCTGGA AGATATAGTA |
| 721 | GCAGACTTGT GTGGTTTTAT TATGAGAGAA ATAATTCCAA GTGACGGTGC ATATTTTGAT |
| 781 | CATGAGGGCG ATTTAGCAAG TGATAAATTT AGAGTGCTGA CAGACATAGC AGGTCTAAAT |
| 841 | CTGAAGCGAA ATGACATGTA A |

(SEQ ID NO:33, Sb07g008600—S. bicolor), or a variant thereof, for example a codon optimized variant, having at least 75%, 80%, 85%, 90%, 95%, 96%, 97%, 98%, or 99% sequence identity to SEQ ID NO:33.

Therefore, a maturity Ma1 protein as it is found in short-day S. bicolor can include the amino acid sequence:

MPPSKEAPSGDVHVKQPSSQPLTLKDIRKPTIDDYVNVPSDYVPGRPMLQ
WTLLDKIQWPIKRFHDWYMRAVHAGLHAIRVDIPANVFATGNEKSKAFV
IFEDMHLLLNYRRLDVQLITIWCLDHWIVFYIYPFERKVLVLDSLHVPP
EKYQPFLVQLERAWRFYKKQKGPVDAARSDPRVPLMIQHHYPCHKQ
PSGSVYCGYYVCEFIRQRGRYVTDKNMPKKQKKDVPFTPKTLEDIVA
DLCGFIMREIIPSDGAYFDHEGDLASDKFRVLTDIAGLNLKRNDM

(SEQ ID NO:34, Sb07g008600—S. bicolor) or functional fragment, or variant thereof having at least 75%, 80%, 85%, 90%, 95%, 96%, 97%, 98%, or 99% sequence identity to SEQ ID NO:34.

A polynucleotide is therefore disclosed having the nucleic acid sequence SEQ ID NO: 1, 2, 3, 4, 5, 6, 7, 9, 10, 11, 12, 13, 19, 20, 28, 29, 30, 31, 32, and 33. A polynucleotide having a nucleic acid sequence at least 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% identical to SEQ ID NO: 1, 2, 3, 4, 5, 6, 7, 9, 10, 11, 12, 13, 19, 20, 28, 29, 30, 31, 32, or 33 is also disclosed. A polynucleotide that hybridizes under stringent conditions to a polynucleotide consisting of the nucleic acid sequence SEQ ID NO: 1, 2, 3, 4, 5, 6, 7, 9, 10, 11, 12, 13, 19, 20, 28, 29, 30, 31, 32, or 33 is also disclosed.

A polypeptide is therefore disclosed having the amino acid sequence SEQ ID NO: 8 and 34. A polypeptide having an amino acid sequence at least 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% identical to SEQ ID NO: 8 or 34 is also disclosed.

A polynucleotide that is a fragment of Ma1 gene is also disclosed. Therefore, a polynucleotide having a nucleic acid sequence at least 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% identical to a fragment of SEQ ID NO: 1, 2, 3, 4, 5, 6, 7, 9, 10, 11, 12, 13, 19, 20, 28, 29, 30, 31, 32, and 33 is disclosed. The fragment can be at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 50, 75, 100, 150, 200, 250, 300, 350, 400, 500, or more nucleotides shorter than SEQ ID NO: 1, 2, 3, 4, 5, 6, 7, 9, 10, 11, 12, 13, 19, 20, 28, 29, 30, 31, 32, or 33.

A polypeptide that is a fragment of the Ma1 protein is also disclosed having the amino acid sequence SEQ ID NO: 8 or 34. A polypeptide having an amino acid sequence at least 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% identical to a fragment of SEQ ID NO: 8 or 34 is disclosed. The fragment can be at least 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 amino acids shorter than SEQ ID NO: 8 or 34.

B. Photoperiod Sensitivity Expression Control

1. Photoperiod Sensitivity

The expression control sequences of Ma1 are also provided for use in putting expression of other plant genes under photoperiod control. For example, the expression control sequence of the Ma1 gene in the short-day S. propinquum having a dominant (functional) Ma1 allele can be used to induce photoperiod sensitivity of other plant genes.

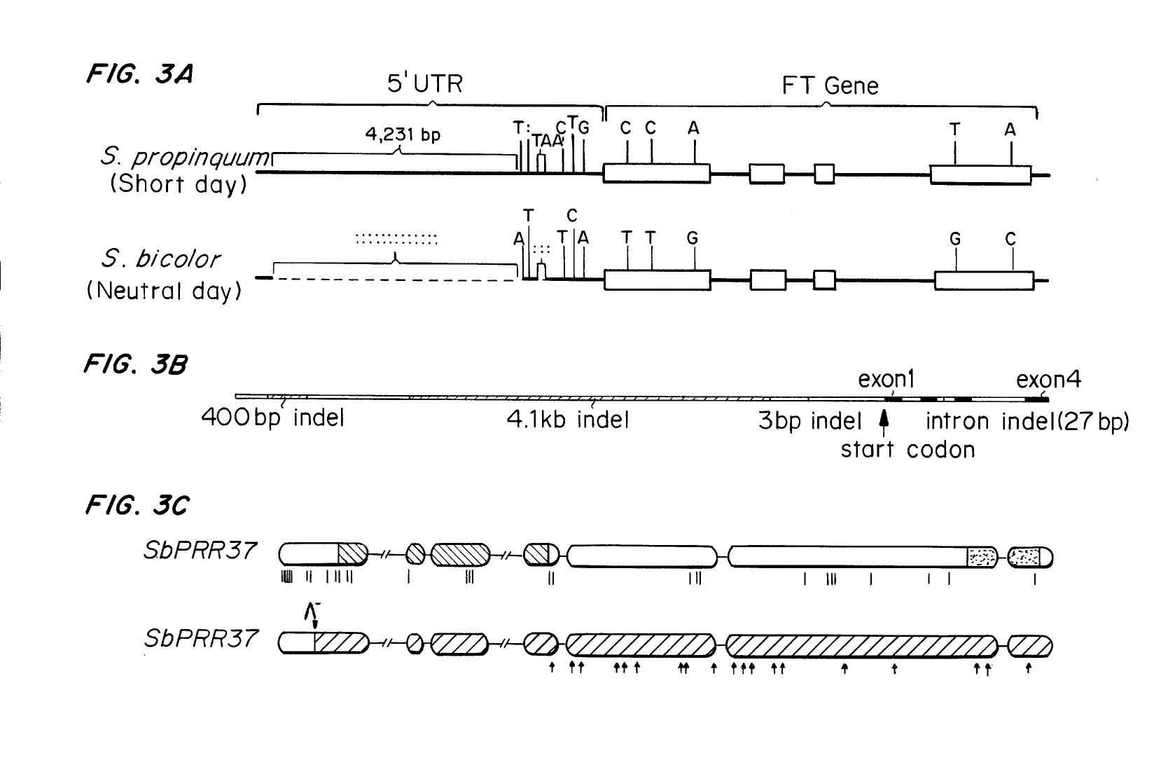

The day-neutral haplotype of S. bicolor is characterized by a number of insertions, deletions and polymorphisms relative to S. propinquum. The mutations in S. bicolor include three deletions in the expression control sequence (5′ UTR) and one deletion in the second intron: (1) a 423 nucleotide deletion beginning with nucleotide 1,132 numbering for the first nucleotide of SEQ ID NO:1 or nucleotide 1597 numbering from the first nucleotide of SEQ ID NO:3; (2) a 4,186 nucleotide deletion beginning with nucleotide 2,465 from SEQ ID NO:1, or 4,231 nucleotide deletion beginning with nucleotide 2,930 numbering from the first nucleotide of SEQ ID NO:3 (3) a 3 nucleotide deletion beginning with nucleotide 6,753 numbering from the first nucleotide of SEQ ID NO:1, or nucleotide 7,263 numbering from the first nucleotide of SEQ ID NO:3 or nucleotide 2,024 numbering from the first nucleotide of SEQ ID NO:5; (4) a 27 nucleotide deletion beginning with nucleotide number 7,563 numbering from the first nucleotide of SEQ ID NO:1, or nucleotide 8,073 numbering from the first nucleotide of SEQ ID NO:3, or nucleotide 2,834 numbering from the first nucleotide of SEQ ID NO:5 (FIG. 3B).

Other insertions, deletions, and polymorphisms in or around S. bicolor Ma1 relative to S. propinquum Ma1, and their association with photoperiod sensitivity can be determined by one of skill in the art using the compositions and methods described herein. For example, additional deletions, insertions, and polymorphisms can be determined by comparing SEQ ID NO: 1, 3, or 5 of S. propinquum Ma1 to SEQ ID NO: 9 or 12 of S. bicolor using global sequence alignment tools. A global alignment shows an end-to-end alignment of two sequences. Tools for preparing global alignments are available in the art, for example, using EMBOSS Needle software available at ebi.ac.uk/Tools/psa/which creates a global alignment of two sequences using the Needleman-Wunsch algorithm.

Accordingly, one or more of the Ma1 expression control sequences in S. propinquum that are mutated or absent from S. bicolor can be operably linked to a plant gene coding sequence to impart photoperiod sensitive (i.e., short-day) control over the plant gene coding sequence.

In some embodiments, a functional Ma1 expression control sequence capable of placing a target gene under photoperiod (short-day) control has the nucleic acid sequence:

| | |
|---|---|
| 1 | AAAAGAAAAG TGAGCACACC ACGACCTGTC ATCAGCTCAT GGTCAGCTCT ACAAACTTAT |
| 61 | AGATTGCATC GAGATCTAAG ACTCAGGTAC AAATCATGTC AACATCTAAT GGTTTAGAAA |
| 121 | ATGAAAAGTT TTGAGTTTCA AAATATGATA CGTGATATTA ACATTTGAAC TTTTAGCAAG |
| 181 | ATCTGAAATA AAAAATTCAA CTAGATCATG TTAACATTGA TATAATCGCT TCCAATCGCC |
| 241 | TCCCATCACT TCCGCTAGAA AACTTTTTTT CTCGATTTAA TTAATGAAAG GGTAATAACA |
| 301 | TCATTGTACA AGATTCTTTC AAACCTCAAC CCCTATCATC GACGGTGACG GCTCCCTATA |
| 361 | ACACGCACTA GTGGACGCCG GGCGGGTGGA ACCCTAAGAA GATTTAAAAA AACTTAAGAA |
| 421 | GAAGATTTTT ATCTAACTAA CTATAGTACT TATATCATAC ACTATACTAT TCAAAATATT |
| 481 | ATTTTCACAA TTATGAATTT ACCCTTTTAC TCTTCATTAA AAAAATACGA AAAAAGAATC |
| 541 | ACCACGTCTC TATTTAGGGT CCTAGTCCCC ATAATTTAAG AGGCGGTGAG AGACGATGTG |
| 601 | ACGTCTATGG ACCACCGACC AAAGACACAC CTATCGTCTC CCATCGCCTT GCTTCCATCG |
| 661 | CCTCTCATCG CTTTTCATAT TCTAGATCCA GCGGCCATAG ACACACCAAT CGTTTCTCAT |
| 721 | CGCCTCTCCA ACCATTGTAA AAATATTTAT AATTTTGATA TAAAATTTGT CTTCACTTGA |
| 781 | GTTCATGCCA AAAAAATTAT ACATATTATT TTCGTGTGAG AATTTACAGA AGTGGACTCT |
| 841 | TAAGATGTCC AAATGTAAAT GACCCTATTT ATTATGAGGC GCGGATCTAT AGGCCTGACT |
| 901 | CTGAAAATGG ATTATGGATT TGAGATAATA AATTTAAGGG CCTATCTTCG CACATAACAT |
| 961 | CTATAGTTCC TAAATTTTTT TTTATTGTAG TAGTAGAACT TTTCTCCCTG TAAACCAAGT |
| 1021 | TGACGCTGGG CTTTATTTTG CGACACAGAA CACCAAATTG GTGGCTATGA ACTCTTCCAC |
| 1081 | CTGGGCAGGG AAAACGGTTT ATTATGTTTC TCTTTAATTT ATCTATCGTG GCACTATAAC |
| 1141 | ACAACATGGC TTTGCCGACA CTTCCAACTA TCGGCAAAGG GTACCTTTAC CGACACTTAA |
| 1201 | CGTCTCACGA AAGGTTTTGC CGACAATTTT CAAACAGTCG CGGTAGAAGC AGTTGGCGAA |
| 1261 | ACTTTTGCCG ACAGTTAAAG GCATCGCCGA CACATTTTCT GTAGTCAAAT GGCATACCTA |
| 1321 | CGCCGACAGT TGAACTTTCA CCGACAGTGA ACCCTTTGCC GACAGTTTGG ACCTACGCCG |
| 1381 | ACAGTTTGGA CCTTTTCCGA CAGTTGGTAT GTTAGCGAAA CCGTTTCTAG GGTGTTTCAT |
| 1441 | AAACCATGCC TTGTCCAACA GTAGAAGTGT CGGCAAAACT ATATTGCTAG GATGTAGATA |
| 1501 | CAATTTAAAT ATTTTAATAA ATACACATCA CATTGATTGA GCAAAATCAC ATGGTCTGTT |
| 1561 | TTCACTAAAA CTGTCAGAGG TACACTCCAG TACTACCAGT ACGTCGCCCG CACAGTGGCC |
| 1621 | AAGGATTTTA CTGCTACTGT TGATTAACAT AAGCACTTGC GACTTTCCCT AAAATCTTTT |
| 1681 | ATAAAACAAC GGCCGCAATA ATATTGAACT ATTTTTTTTC TAGTACCAAA ATTAGAATTT |
| 1741 | GATCCCTCAC CTCATTACAT CCATAGTAAC ATGACCAGAT ATATATGGAC AGGATGGGAT |
| 1801 | CACTCAGCGA GCAGATACAC TGAGCGATTC ATAATCAGAT TTTTTAATTT CTTCTAGTGA |
| 1861 | AGTGGGGTTT TCCTAGTCTT TTAACATTCA AAATTTAGTA CAAACTTTCC CTAGTAAATG |
| 1921 | CCTTCTAGTA AAGATTTCCT AGTATTTTGA CTAGCGATAG TGTTTTATTA CTAATTAAAA |
| 1981 | ACATTAGAAG AACTCCATTT AGTGATTGGT TGTTTGGATT AGTCTTCTCA CGTTAGACCT |
| 2041 | ATATATGCAG GACAACTCAA GCCAGCATAA ATATATGAAA TATCTTGGTG TTTGTTTGTC |
| 2101 | TGACACAGGC AACCGCGTTT GGTATAAATG TGTTTTCTTG TTTACATTTT ACCATCTATA |
| 2161 | GTCATCTCAA TGTTATATAG TAGAGGCTTC ATGTTTGTAG TAGATAAGGT AGAGAATTGA |
| 2221 | GAATATTTTA TTTTTGTGCG ACCATCAATT TTATGTAATC TGCATTGTCT AATGCTTTAT |
| 2281 | TTGACATTTG AAACTACTTA ATTTGACAGT TATGCAGGTC CGCATGATCC TATGAAAGCA |
| 2341 | ATTAATTAGT ACGGGTAAAC TGCACTACAC AAGTTTGCTA GTACTATTCT ATTAACCGAC |
| 2401 | CTGTCAATAT TACCTTAAGT TACTGATTTC AATTAGAATC TAACACATTC AGGAAAAGAA |
| 2461 | GTTTCACTAG TACAAAAATC ATTTTCGTTG GCACGTTGTT TTTTTTTTCA CAGGCAGTTC |
| 2521 | ACAATATCAT GGTGCTAGTA GAAAAATTTC AACGGGCCCA ACAAGAGAAC CGCCAGGCGG |
| 2581 | TCTTCTTAAT TCAACCGCCT GTGTAAACTT TCCATTTACA TAGGCGGCTT ACGATAAAAA |
| 2641 | CCGTGTGTAT AAATACCATT AACACAGGCA GTCGAGTTAC GACAACCGCC TGTGTAAATG |
| 2701 | TGTCTTTTTA CACAGGCGGT TTGTATAGAG GGCCGCCTGT GCTAATATAT TTACACAGGC |
| 2761 | TATGAGCCGC CTGTGTTAAG TCTTCTATAA ATACCCTTCG TCCACCTCCA GACAAGAACA |
| 2821 | GTTACTCCCA TGAGCTCTGC ACACTGGCGG ACCAGACGAT TCCAGTTTCC AAGGGGGGAG |
| 2881 | GTTTTGATTT TCATTTCTTT GGTGAGAAAC TTCCAAAAGG TTAGTTAGTG CCATTGATGC |
| 2941 | TATTTTTTAA GCGATTCTTT GGTTCAATTC TTGTATTGGA GGTGCTCTAG ATCTAGAGTT |
| 3001 | CATCATGCAT TCTTGCTTAG GGTTAGAGTT CATAGGGCAA AAAGAGAGAG ATTTAGCTAA |
| 3061 | ATTTTTATGT AAATTCATAG TAAATTGTAA AAATTAAAAA AAATAAAAAA TAAATACTTT |
| 3121 | TTAGAATTCT TGTGAGTAGA TCTATACAAT AGAGTAATGA TGAGGATATT TTGAAGTTTA |
| 3181 | TAATTTTGAT TCAGTTTTAG CTTTTCTTTT TTCAGATGAA TTAGACTTTA TAAACTCAAA |
| 3241 | CATTAAAATG TTGAAAATCA TAAAATGGCA AATAAATACT TTTTCAAATC TTTGTGCATA |
| 3301 | AATACTTCAT AGAAATCCTT GAATTATTCC TAAATTTTAT ACAATTGTTT CTTATAATTA |
| 3361 | TGAAAATGAG TTTAAACAAT TATTTAAATT CCATAAATTG TAACTCCGTA AGGTGTAGGT |
| 3421 | TTTCATCTCT GTTTAATAGA AGGAGGTTAG TATCTTAGTT AAGTCTGTTT TCGGGGGTTA |
| 3481 | TATTAGTTTT GTTTTTAGAT TGACCTACAT TAATTGTTCT TAACTAATTA CAGCTAAATA |
| 3541 | TGGAGAGGTC ATTATGGATG TACAACTTAT CAAGATTGGA CCTATCATAT GTAGTGCAGG |
| 3601 | TCCAAAAATT TATTGATGTC GCAAAGATAC ATGCTCGCAG AACAAAGGCG AAGCACATAT |
| 3661 | GTTGTCCATG CGCAGACTGC AAAAATATTA TGGTATTTGA CAATGTAGAA GCAATTACTT |
| 3721 | CCCATCTGGT TTGAAGAGGA TTTATGGAGG ACTACTTGAT TTGGACAAAA CATGGTGAGG |
| 3781 | GTAGTTTTGC ACCTTATATG CGGACAACTG ACAACACTGC AACTAACATC AATGTGGAGG |
| 3841 | GTCCAATGCC ACCTCTCAAT GAATTTCATG CTATGCCAGA TGTTAATGAA ACTCATACGT |
| 3901 | CTGATGTCAA TGAAACTCAG CATGCTAACA CAGATGTTGT TGAAGATGCA GATTTCTTAG |
| 3961 | AGGCAATAAT GAACCGTTGT GCGGATCCAT CAATATTCTT CATGAAGGGA ATGAAAGCAT |
| 4021 | TGAAGAAGGC AGCAGAGGAC ACTTTGTACG ACGAGTCAAA AGGTTGTACC AAACAATGGT |
| 4081 | CGACATTATG TGTTGTTCTT CAGTTTTTGA CGATGAAGGC TAGACATGGT TGGTCCGATG |
| 4141 | CTAGCTTCAA TGATTTCTTG CGTGTACTTG GAGACCTTCT TCCTAAGGAG AACAAAGTGC |
| 4201 | CTGCTAACAC ATACTATGCA AAGAAGCTAG TCAGTCCACT TACGATAGGT GTTGAGAAGA |
| 4261 | TCCACGCATG TAGAAATCAT TGTATTCTAT ATCGAGGTGA TCAATATAAA GACTTAGACA |
| 4321 | GTTGTCCAAA CTGTGGTGCC AGTAGGTACA AGACAAACAA AGATTTTCGG GAGGAAGAGA |
| 4381 | ATCTAGCCTC TGTTTCTACA GGGAGGAAGC GAAAGAAGAC CCAAACAAAG ACTCAACAAG |
| 4441 | ACAAGCGCTC AAAGCCTAGT AGCAATGAAG AAGTGGACTA TTATGCATTG AGAAGAGTCT |
| 4501 | CCCTATGAGC CAAAAAAGGG GACAGCAGCA GGCACAACTC TCTTTCTGAA AGGACTTGGA |
| 4561 | AAGCAGCGGA CGGCACGGCT CATTGAGCTC GAACCGTCAC AGAAAAAGGA AGCCACCGCC |
| 4621 | CAGTCAATAG AAGCCATGCC CCCATCAAAG GAAGCCCCAA GTGGCGATGT ACATATTGAA |
| 4681 | CAGCCATCAA GTCAACCATT GACCCTAAAG GATATCAGAA AGCCAACGAT TGATGATTAT |
| 4741 | GTCAATGTCC CTAGTGACTA TGTGCCCGGA AGGCCTATGC TCCAATGGAC GCTGCTCGAT |
| 4801 | TAGATTCAAT GGCTGATAAA AAGGTTTCAT GACTGGTACA TGAGAGCAGT GCATGCTAGC |
| 4861 | CTCCATGGAA TCAGAGTTGA TATACCAACA GACATGTTTG CTACTGGTAA CAAAAAAAGC |
| 4921 | AAGACATTTG TTACCTTTGA GGACATGCAC TTGTTATTGA ACTATAGGCG GCTTGACGTC |
| 4981 | CAACTCATAA CAATCTGGTG CCTGTAAGTA TCACTCATGC ACACACAATT ATTATATATT |
| 5041 | AATATGTAGT GTGAAACTCT AATATGTAGA TGTTGTCTGT AGTTTGCAAG ATCACGAGCA |
| 5101 | GATGTCATTA TTATCTGCCG GATCGATGGT CGGTTATCTG AGCCCTATCA AGTTACAAGA |
| 5161 | AAATATGAAC AAATTCGTAT TATCAAAGGA AGATAGAGCA AAGATAGAGG AAGACAAAAC |
| 5221 | ACCAGGATAA TTATGCCATC TATCTTGGTA GATCAATGCT GAGGTATAAA TATAGGGATT |
| 5281 | TTATATTGGC ACCATACAAC ATTAGGTAAG CTTGACTTCA TATACGTATT TCAAATTATC |
| 5341 | GTGTAAACAA TATACATGTG TCGCTCACTC ATTTATTCAT GCAGTGACCA TTGGATTGTT |
| 5401 | TTTTATATTT ATCCCTTCGA AGGGAAGGTG CTTGTCCTAG ACTCTTTACA TGTTCCTCCC |
| 5461 | GAGAAGTATC AACCATTCTT GGTTCAATTA GAAAGGTGAG CCAACATGAA ACCACATGCG |
| 5521 | TACTTATATA AATTAGAGTT TCAAAATAAC TTTAGTGATT TAGGTTCGAT ATCTACGGGG |
| 5581 | CATGGCGGTT TTATAAGAAA CAAAAGGGAC CTGTCGACGC TGCACGCTCA GATCCTAGGA |
| 5641 | TCCCATTGAT GATACAACAC CACTATCCGG TAAGTTTTCT GAACACATTT CATCATATAA |
| 5701 | ATAATACATA AAGCATGGCA AATTTAGAAT AATCCGTTGC TCATTATATA GTGCCACAAG |
| 5761 | CAACCACCTG GATCGGTCTA TTGTGGGTAC TATGTCTGTG AGTTTATAAG GCAGCGGGGA |
| 5821 | CGTTACGTCA AGGACAAAAA TATGGTAAAT AATATCTATG TATGAAAGTT TTCTCATTAA |
| 5881 | AGCTGCAAAA TTATATATTG AACATGTGTC AATCATGCTT TTAAACTTTA TTTTCAGCCG |
| 5941 | AAAAAGCAAG GAAAAGACGT GCCCTTTACA CCAAAGACTC TGGAAGATAT AGTAGCATAC |
| 6001 | TTGTGTGGTT TTATTATGAG AGAAATAATT TCAAGTGACA GTGCATATTT TGATCATGAG |
| 6061 | GGCGATTTAG CAAGTGATAA ATTTAGAGTG CTGACAGACA TAGCAGGTCT AAATCTGAAG |
| 6121 | CGAAACGACA TGTAAACATT GTATGGTTGT GCGGATAACA TGCATTGACG TGTATATATA |
| 6181 | TAATTTTATG GTTGATGTTT GATTTGTTTA CAATTCTATA ATATATATAT GTGGTGTATG |
| 6241 | TATGATGTTG TGTGTGTATA TATATATATA TATATATATA TATATATATA TATATATATA |
| 6301 | TATATATATA TATATAATGT TTAGCACTGT GTTTGGTGGG AAAAATTAAA ATTTGAAATA |
| 6361 | TATATAAAAA ATTATTTACA CAGACAGTGT ACGTGTCGAG CGTCGTCCTG TGCTATACAA |
| 6421 | ATACATTCTA ACAGGCGGCT CGCCTTGTCC ACCGGTCGGT TAAAAATACA TTTCCACACN |
| 6481 | GGCCTGGCTG GGAGAGCCGC CTGTGAAAAC ATAATTTTCA CAGGCGGCTC GCACAGCCCC |
| 6541 | GCCTGTACTG TGGTCCATTT TGTACTGACC CCTGGTACAG GCGGTGGGCT TGGCCGCCTG |
| 6601 | TGAAGATGCT TTTAGCACCG CCTGTAAAAA TGTTTTTTGT AGCAGTGTTT TTCTTATTAG |
| 6661 | TAGTATCTTT TATACTAATT AAGATTCAAT AAAAATTCAC CATGACATCC CCATTGCCAA |
| 6721 | GAGAATATTT CGCCGCCCCT CAAAGCAGCC AATAAGGCTT TACTAAAAAG ACTATCCACG |
| 6781 | CAGTAGAGAT TTAGTCAAAA TATTCCAATA GCAATTGTTT CCTGCCTGCT TGACCTTCGT |
| 6841 | CAGCCACTCA CTGTATAAAT ATCGCACCAC GCCCTTTGCA GGCTTACAGA GCTTGTATTA |
| 6901 | CGTACTAACA AGGCACACAC AGTACCCTGT GTTCACCGGC CCTGCACAAA ACTCAAGCAG |
| 6961 | TTATTACTAA C |

(SEQ ID NO:14) or a functional fragment or variant thereof having 75, 80, 85, 90, 95, 96, 97, 98, or 99% sequence identity to SEQ ID NO:14.

In some embodiments, a functional Ma1 expression control sequence capable of placing a target gene under photoperiod (short-day) control has the nucleic acid sequence:

| | |
|---|---|
| 1 | CCCTGACCCT TGTTGGGCAA CATTTAGAGT CGTTAGCTTT GCAATTCTTT GGTTCCAATG |
| 61 | GATGGTTATC ATTTAGACAT ATTGGTCATG CTTAGTCAAA ACTTTATTGT TCGGCTATAA |
| 121 | ACTTTTCAGT ACTTTGTAAT AATTGGCTCG ATAGATGAAG CCGGGTATAA CATATCCTTT |
| 181 | ATCTAAAAAA ATTAGTTAAC ATGAACTTCA TATTCAATTC TTCATATCTC ACTAGCATCT |
| 241 | TTATTGTCTA GTTAGTTTTG TAGCATTGCA AAAAGCATGC AACTATATAC AATGAAACGG |
| 301 | AATAAAATTT CAGCTCTATT AATTTATATT TCAAATATAG GCCACTATAG CCATATTTCG |
| 361 | TGCTCAAGGC CACAAAATCT TGCGTACTTC CCTGTTGGTA CCAAAGAGAA GACGTTATTT |
| 421 | AACTTTGTTT GACTCTTCAA TATGGTTTGA ATCAGAAAAT TAGTTAAAAG AAAAGTGAGC |
| 481 | ACACCACGAC CTGTCATCAG CTCATGGTCA GCTCTACAAA CTTATAGATT GCATCGAGAT |
| 541 | CTAAGACTCA GGTACAAATC ATGTCAACAT CTAATGGTTT AGAAAATGAA AAGTTTTGAG |
| 601 | TTTCAAAATA TGATACGTGA TATTAACATT TGAACTTTTA GCAAGATCTG AAATAAAAAA |
| 661 | TTCAACTAGA TCATGTTAAC ATTGATATAA TCGCTTCCAA TCGCCTCCCA TCACTTCCGC |
| 721 | TAGAAAACTT TTTTTCTCGA TTTAATTAAT GAAAGGGTAA TAACATCATT GTACAAGATT |
| 781 | CTTTCAAACC TCAACCCCTA TCATCGACGG TGACGGCTCC CTATAACACG CACTAGTGGA |
| 841 | CGCCGGGCGG GTGGAACCCT AAGAAGATTT AAAAAAACTT AAGAAGAAGA TTTTTATCTA |
| 901 | ACTAACTATA GTACTTATAT CATACACTAT ACTATTCAAA ATATTATTTT CACAATTATG |
| 961 | AATTTACCCT TTTACTCTTC ATTAAAAAAA TACGAAAAAA GAATCACCAC GTCTCTATTT |
| 1021 | AGGGTCCTAG TCCCCATAAT TTAAGAGGCG GTGAGAGACG ATGTGACGTC TATGGACCAC |
| 1081 | CGACCAAAGA CACACCTATC GTCTCCCATC GCCTTGCTTC CATCGCCTCT CATCGCTTTT |
| 1141 | CATATTCTAG ATCCAGCGGC CATAGACACA CCAATCGTTT CTCATCGCCT CTCCAACCAT |
| 1201 | TGTAAAAATA TTTATAATTT TGATATAAAA TTTGTCTTCA CTTGAGTTCA TGCCAAAAAA |
| 1261 | ATTATACATA TTATTTTCGT GTGAGAATTT ACAGAAGTGG ACTCTTAAGA TGTCCAAATG |
| 1321 | TAAATGACCC TATTTATTAT GAGGCGCGGA TCTATAGGCC TGACTCTGAA AATGGATTAT |
| 1381 | GGATTTGAGA TAATAAATTT AAGGGCCTAT CTTCGCACAT AACATCTATA GTTCCTAAAT |
| 1441 | TTTTTTTTAT TGTAGTAGTA GAACTTTTCT CCCTGTAAAC CAAGTTGACG CTGGGCTTTA |
| 1501 | TTTTGCGACA CAGAACACCA AATTGGTGGC TATGAACTCT TCCACCTGGG CAGGGAAAAC |
| 1561 | GGTTTATTAT GTTTCTCTTT AATTTATCTA TCGTGGCACT ATAACACAAC ATGGCTTTGC |
| 1621 | CGACACTTCC AACTATCGGC AAAGGGTACC TTTACCGACA CTTAACGTCT CACGAAAGGT |
| 1681 | TTTGCCGACA ATTTTCAAAC AGTCGCGGTA GAAGCAGTTG GCGAAACTTT TGCCGACAGT |
| 1741 | TAAAGGCATC GCCGACACAT TTTCTGTAGT CAAATGGCAT ACCTACGCCG ACAGTTGAAC |
| 1801 | TTTCACCGAC AGTGAACCCT TTGCCGACAG TTTGGACCTA CGCCGACAGT TTGGACCTTT |
| 1861 | TCCGACAGTT GGTATGTTAG CGAAACCGTT TCTAGGGTGT TTCATAAACC ATGCCTTGTC |
| 1921 | CAACAGTAGA AGTGTCGGCA AAACTATATT GCTAGGATGT AGATACAATT TAAATATTTT |
| 1981 | AATAAATACA CATCACATTG ATTGAGCAAA ATCACATGGT CTGTTTTCAC TAAAACTGTC |
| 2041 | AGAGGTACAC TCCAGTACTA CCAGTACGTC GCCCGCACAG TGGCCAAGGA TTTTACTGCT |
| 2101 | ACTGTTGATT AACATAAGCA CTTGCGACTT TCCCTAAAAT CTTTTATAAA ACAACGGCCG |
| 2161 | CAATAATATT GAACTATTTT TTTTCTAGTA CCAAAATTAG AATTTGATCC CTCACCTCAT |
| 2221 | TACATCCATA GTAACATGAC CAGATATATA TGGACAGGAT GGGATCACTC AGCGAGCAGA |
| 2281 | TACACTGAGC GATTCATAAT CAGATTTTTT AATTTCTTCT AGTGAAGTGG GGTTTTCCTA |
| 2341 | GTCTTTTAAC ATTCAAAATT TAGTACAAAC TTTCCCTAGT AAATGCCTTC TAGTAAAGAT |
| 2401 | TTCCTAGTAT TTTGACTAGC GATAGTGTTT TATTACTAAT TAAAAACATT AGAAGAACTC |
| 2461 | CATTTAGTGA TTGGTTGTTT GGATTAGTCT TCTCACGTTA GACCTATATA TGCAGGACAA |
| 2521 | CTCAAGCCAG CATAAATATA TGAAATATCT TGGTGTTTGT TTGTCTGACA CAGGCAACCG |
| 2581 | CGTTTGGTAT AAATGTGTTT TCTTGTTTAC ATTTTACCAT CTATAGTCAT CTCAATGTTA |
| 2641 | TATAGTAGAG GCTTCATGTT TGTAGTAGAT AAGGTAGAGA ATTGAGAATA TTTTATTTTT |
| 2701 | GTGCGACCAT CAATTTTATG TAATCTGCAT TGTCTAATGC TTTATTTGAC ATTTGAAACT |
| 2761 | ACTTAATTTG ACAGTTATGC AGGTCCGCAT GATCCTATGA AAGCAATTAA TTAGTACGGG |
| 2821 | TAAACTGCAC TACACAAGTT TGCTAGTACT ATTCTATTAA CCGACCTGTC AATATTACCT |
| 2881 | TAAGTTACTG ATTTCAATTA GAATCTAACA CATTCAGGAA AAGAAGTTTC ACTAGTACAA |
| 2941 | AAATCATTTT CGTTGGCACG TTGTTTTTTT TTTCACAGGC AGTTCACAAT ATCATGGTGC |
| 3001 | TAGTAGAAAA ATTTCAACGG GCCCAACAAG AGAACCGCCA GGCGGTCTTC TTAATTCAAC |
| 3061 | CGCCTGTGTA AACTTTCCAT TTACATAGGC GGCTTACGAT AAAAACCGTG TGTATAAATA |
| 3121 | CCATTAACAC AGGCAGTCGA GTTACGACAA CCGCCTGTGT AAATGTGTCT TTTTACACAG |
| 3181 | GCGGTTTGTA TAGAGGGCCG CCTGTGCTAA TATATTTACA CAGGCTATGA GCCGCCTGTG |
| 3241 | TTAAGTCTTC TATAAATACC CTTCGTCCAC CTCCAGACAA GAACAGTTAC TCCCATGAGC |
| 3301 | TCTGCACACT GGCGGACCAG ACGATTCCAG TTTCCAAGGG GGGAGGTTTT GATTTTCATT |
| 3361 | TCTTTGGTGA GAAACTTCCA AAAGGTTAGT TAGTGCCATT GATGCTATTT TTTAAGCGAT |
| 3421 | TCTTTGGTTC AATTCTTGTA TTGGAGGTGC TCTAGATCTA GAGTTCATCA TGCATTCTTG |
| 3481 | CTTAGGGTTA GAGTTCATAG GGCAAAAAGA GAGAGATTTA GCTAAATTTT TATGTAAATT |
| 3541 | CATAGTAAAT TGTAAAAATT AAAAAAAATA AAAAATAAAT ACTTTTTAGA ATTCTTGTGA |
| 3601 | GTAGATCTAT ACAATAGAGT AATGATGAGG ATATTTTGAA GTTTATAATT TTGATTCAGT |
| 3661 | TTTAGCTTTT CTTTTTTCAG ATGAATTAGA CTTTATAAAC TCAAACATTA AAATGTTGAA |
| 3721 | AATCATAAAA TGGCAAATAA ATACTTTTTC AAATCTTTGT GCATAAATAC TTCATAGAAA |
| 3781 | TCCTTGAATT ATTCCTAAAT TTTATACAAT TGTTTCTTAT AATTATGAAA ATGAGTTTAA |
| 3841 | ACAATTATTT AAATTCCATA AATTGTAACT CCGTAAGGTG TAGGTTTTCA TCTCTGTTTA |
| 3901 | ATAGAAGGAG GTTAGTATCT TAGTTAAGTC TGTTTTCGGG GGTTATATTA GTTTTGTTTT |
| 3961 | TAGATTGACC TACATTAATT GTTCTTAACT AATTACAGCT AAATATGGAG AGGTCATTAT |
| 4021 | GGATGTACAA CTTATCAAGA TTGGACCTAT CATATGTAGT GCAGGTCCAA AAATTTATTG |
| 4081 | ATGTCGCAAA GATACATGCT CGCAGAACAA AGGCGAAGCA CATATGTTGT CCATGCGCAG |
| 4141 | ACTGCAAAAA TATTATGGTA TTTGACAATG TAGAAGCAAT TACTTCCCAT CTGGTTTGAA |
| 4201 | GAGGATTTAT GGAGGACTAC TTGATTTGGA CAAAACATGG TGAGGGTAGT TTTGCACCTT |
| 4261 | ATATGCGGAC AACTGACAAC ACTGCAACTA ACATCAATGT GGAGGGTCCA ATGCCACCTC |
| 4321 | TCAATGAATT TCATGCTATG CCAGATGTTA ATGAAACTCA TACGTCTGAT GTCAATGAAA |
| 4381 | CTCAGCATGC TAACACAGAT GTTGTTGAAG ATGCAGATTT CTTAGAGGCA ATAATGAACC |
| 4441 | GTTGTGCGGA TCCATCAATA TTCTTCATGA AGGGAATGAA AGCATTGAAG AAGGCAGCAG |
| 4501 | AGGACACTTT GTACGACGAG TCAAAAGGTT GTACCAAACA ATGGTCGACA TTATGTGTTG |
| 4561 | TTCTTCAGTT TTTGACGATG AAGGCTAGAC ATGGTTGGTC CGATGCTAGC TTCAATGATT |
| 4621 | TCTTGCGTGT ACTTGGAGAC CTTCTTCCTA AGGAGAACAA AGTGCCTGCT AACACATACT |
| 4681 | ATGCAAAGAA GCTAGTCAGT CCACTTACGA TAGGTGTTGA GAAGATCCAC GCATGTAGAA |
| 4741 | ATCATTGTAT TCTATATCGA GGTGATCAAT ATAAAGACTT AGACAGTTGT CCAAACTGTG |
| 4801 | GTGCCAGTAG GTACAAGACA AACAAAGATT TTCGGGAGGA AGAGAATCTA GCCTCTGTTT |
| 4861 | CTACAGGGAG GAAGCGAAAG AAGACCCAAA CAAAGACTCA ACAAGACAAG CGCTCAAAGC |
| 4921 | CTAGTAGCAA TGAAGAAGTG GACTATTATG CATTGAGAAG AGTCTCCCTA TGAGCCAAAA |
| 4981 | AAGGGGACAG CAGCAGGCAC AACTCTCTTT CTGAAAGGAC TTGGAAAGCA GCGGACGGCA |
| 5041 | CGGCTCATTG AGCTCGAACC GTCACAGAAA AAGGAAGCCA CCGCCCAGTC AATAGAAGCC |
| 5101 | ATGCCCCCAT CAAAGGAAGC CCCAAGTGGC GATGTACATA TTGAACAGCC ATCAAGTCAA |
| 5161 | CCATTGACCC TAAAGGATAT CAGAAAGCCA ACGATTGATG ATTATGTCAA TGTCCCTAGT |
| 5221 | GACTATGTGC CCGGAAGGCC TATGCTCCAA TGGACGCTGC TCGATTAGAT TCAATGGCTG |
| 5281 | ATAAAAAGGT TTCATGACTG GTACATGAGA GCAGTGCATG CTAGCCTCCA TGGAATCAGA |
| 5341 | GTTGATATAC CAACAGACAT GTTTGCTACT GGTAACAAAA AAAGCAAGAC ATTTGTTACC |
| 5401 | TTTGAGGACA TGCACTTGTT ATTGAACTAT AGGCGGCTTG ACGTCCAACT CATAACAATC |
| 5461 | TGGTGCCTGT AAGTATCACT CATGCACACA CAATTATTAT ATATTAATAT GTAGTGTGAA |
| 5521 | ACTCTAATAT GTAGATGTTG TCTGTAGTTT GCAAGATCAC GAGCAGATGT CATTATTATC |
| 5581 | TGCCGGATCG ATGGTCGGTT ATCTGAGCCC TATCAAGTTA CAAGAAAATA TGAACAAATT |
| 5641 | CGTATTATCA AAGGAAGATA GAGCAAAGAT AGAGGAAGAC AAAACACCAG GATAATTATG |
| 5701 | CCATCTATCT TGGTAGATCA ATGCTGAGGT ATAAATATAG GGATTTTATA TTGGCACCAT |
| 5761 | ACAACATTAG GTAAGCTTGA CTTCATATAC GTATTTCAAA TTATCGTGTA AACAATATAC |
| 5821 | ATGTGTCGCT CACTCATTTA TTCATGCAGT GACCATTGGA TTGTTTTTTA TATTTATCCC |
| 5881 | TTCGAAGGGA AGGTGCTTGT CCTAGACTCT TTACATGTTC CTCCCGAGAA GTATCAACCA |
| 5941 | TTCTTGGTTC AATTAGAAAG GTGAGCCAAC ATGAAACCAC ATGCGTACTT ATATAAATTA |
| 6001 | GAGTTTCAAA ATAACTTTAG TGATTTAGGT TCGATATCTA CGGGGCATGG CGGTTTTATA |
| 6061 | AGAAACAAAA GGGACCTGTC GACGCTGCAC GCTCAGATCC TAGGATCCCA TTGATGATAC |
| 6121 | AACACCACTA TCCGGTAAGT TTTCTGAACA CATTTCATCA TATAAATAAT ACATAAAGCA |
| 6181 | TGGCAAATTT AGAATAATCC GTTGCTCATT ATATAGTGCC ACAAGCAACC ACCTGGATCG |
| 6241 | GTCTATTGTG GGTACTATGT CTGTGAGTTT ATAAGGCAGC GGGGACGTTA CGTCAAGGAC |
| 6301 | AAAAATATGG TAAATAATAT CTATGTATGA AGTTTTCTCA TTAAAGCTGC AAAATTATAT |
| 6361 | ATTGAACATG TGTCAATCAT GCTTTTAAAC TTTATTTTCA GCCGAAAAAG CAAGGAAAAG |
| 6421 | ACGTGCCCTT TACACCAAAG ACTCTGGAAG ATATAGTAGC ATACTTGTGT GGTTTTATTA |
| 6481 | TGAGAGAAAT AATTTCAAGT GACAGTGCAT ATTTTGATCA TGAGGGCGAT TTAGCAAGTG |
| 6541 | ATAAATTTAG AGTGCTGACA GACATAGCAG GTCTAAATCT GAAGCGAAAC GACATGTAAA |
| 6601 | CATTGTATGG TTGTGCGGAT AACATGCATT GACGTGTATA TATATAATTT TATGGTTGAT |
| 6661 | GTTTGATTTG TTTACAATTC TATAATATAT ATATGTGGTG TATGTATGAT GTTGTGTGTG |
| 6721 | TATATATATA TATATATATA TATATATATA TATATATATA TATATATATA TATATATATA |
| 6781 | ATGTTTAGCA CTGTGTTTGG TGGGAAAAAT TAAAATTTGA AATATATATA AAAAATTATT |
| 6841 | TACACAGACA GTGTAGTGTG AGCTGCCTGT GTAAAAATAC ATTTATACAG GCGGCTCACC |
| 6901 | TTGTCNNNNC AGGCGGTGCT AAAAGCATCT TCACAGGCGG CCAAGCCCAC CGCCTGTACC |
| 6961 | AGGGGTCAGT ACAAAATGGA CCACAGTACA GGCGGGGCTG TGCGAGCCGC CTGTGAAAAC |
| 7021 | ATAATTTTCA CAGGCGGCTC GCACAGCCCC GCCTGTACTG TGGTCCATTT TGTACTGACC |
| 7081 | CCTGGTACAG GCGGTGGGCT TGGCCGCCTG TGAAGATGCT TTTAGCACCG CCTGTAAAAA |
| 7141 | TGTTTTTTGT AGCAGTGTTT TTCTTATTAG TAGTATCTTT TATACTAATT AAGATTCAAT |
| 7201 | AAAAATTCAC CATGACATCC CCATTGCCAA GAGAATATTT CGCCGCCCCT CAAAGCAGCC |
| 7261 | AATAAGGCTT TACTAAAAAG ACTATCCACG CAGTAGAGAT TTAGTCAAAA TATTCCAATA |
| 7321 | GCAATTGTTT CCTGCCTGCT TGACCTTCGT CAGCCACTCA CTGTATAAAT ATCGCACCAC |
| 7381 | GCCCTTTGCA GGCTTACAGA GCTTGTATTA CGTACTAACA AGGCACACAC AGTACCCTGT |
| 7441 | GTTCACCGGC CCTGCACAAA ACTCAAGCAG TTATTACTAA C |

(SEQ ID NO:15) or a functional fragment or variant thereof having 75, 80, 85, 90, 95, 96, 97, 98, or 99% sequence identity to SEQ ID NO:15. Each N=can be any nucleotide or combination of any 2, 3, 4, or 5 nucleotides.

In some embodiments, a functional Ma1 expression control sequence capable of placing a target gene under photoperiod (short-day) control has the nucleic acid sequence:

| | |
|---|---|
| 1 | CTATGCTCCA ATGGACGCTG CTCGATTAGA TTCAATGGCT GATAAAAAGG TTTCATGACT |
| 61 | GGTACATGAG AGCAGTGCAT GCTAGCCTCC ATGGAATCAG AGTTGATATA CCAACAGACA |
| 121 | TGTTTGCTAC TGGTAACAAA AAAAGCAAGA CATTTGTTAC CTTTGAGGAC ATGCACTTGT |
| 181 | TATTGAACTA TAGGCGGCTT GACGTCCAAC TCATAACAAT CTGGTGCCTG TAAGTATCAC |
| 241 | TCATGCACAC ACAATTATTA TATATTAATA TGTAGTGTGA AACTCTAATA TGTAGATGTT |
| 301 | GTCTGTAGTT TGCAAGATCA CGAGCAGATG TCATTATTAT CTGCCGGATC GATGGTCGGT |
| 361 | TATCTGAGCC CTATCAAGTT ACAAGAAAAT ATGAACAAAT TCGTATTATC AAAGGAAGAT |
| 421 | AGAGCAAAGA TAGAGGAAGA CAAAACACCA GGATAATTAT GCCATCTATC TTGGTAGATC |
| 481 | AATGCTGAGG TATAAATATA GGGATTTTAT ATTGGCACCA TACAACATTA GGTAAGCTTG |
| 541 | ACTTCATATA CGTATTTCAA ATTATCGTGT AAACAATATA CATGTGTCGC TCACTCATTT |
| 601 | ATTCATGCAG TGACCATTGG ATTGTTTTTT ATATTTATCC CTTCGAAGGG AAGGTGCTTG |
| 661 | TCCTAGACTC TTTACATGTT CCTCCCGAGA AGTATCAACC ATTCTTGGTT CAATTAGAAA |
| 721 | GGTGAGCCAA CATGAAACCA CATGCGTACT TAT ATAAATT AGAGTTTCAA AATAACTTTA |
| 781 | GTGATTTAGG TTCGATATCT ACGGGGCATG GCGGTTTTAT AAGAAACAAA AGGGACCTGT |
| 841 | CGACGCTGCA CGCTCAGATC CTAGGATCCC ATTGATGATA CAACACCACT ATCCGGTAAG |
| 901 | TTTTCTGAAC ACATTTCATC ATATAAATAA TACATAAAGC ATGGCAAATT TAGAATAATC |
| 961 | CGTTGCTCAT TATATAGTGC CACAAGCAAC CACCTGGATC GGTCTATTGT GGGTACTATG |
| 1021 | TCTGTGAGTT TATAAGGCAG CGGGGACGTT ACGTCAAGGA CAAAAATATG GTAAATAATA |
| 1081 | TCTATGTATG AAGTTTTCTC ATTAAAGCTG CAAAATTATA TATTGAACAT GTGTCAATCA |
| 1141 | TGCTTTTAAA CTTTATTTTC AGCCGAAAAA GCAAGGAAAA GACGTGCCCT TTACACCAAA |
| 1201 | GACTCTGGAA GAT ATAGTAG CATACTTGTGTGGTTTTATT ATGAGAGAAA TAATTTCAAG |
| 1261 | TGACAGTGCA TATTTTGATC ATGAGGGCGA TTTAGCAAGT GATAAATTTA GAGTGCTGAC |
| 1321 | AGACATAGCA GGTCTAAATC TGAAGCGAAA CGACATGTAA ACATTGTATG GTTGTGCGGA |
| 1381 | TAACATGCAT TGACGTGTAT ATATATAATT TTATGGTTGA TGTTTGATTT GTTTACAATT |
| 1441 | CTATAATATA TATATGTGGT GTATGTATGA TGTTGTGTGT GTATATATAT ATATATATAT |
| 1501 | ATATATATAT ATATATATAT ATATATATAT ATATATATAT AATGTTTAGC ACTGTGTTTG |
| 1561 | GTGGGAAAAA TTAAAATTTG AAATATATAT AAAAAATTAT TTACACAGAC AGTGTAGTGT |
| 1621 | GAGCTGCCTG TGTAAAAATA CATTTATACA GGCGGCTCAC CTTGTNNNNN CAGGCGGTGC |
| 1681 | TAAAAGCATC TTCACAGGCG GCCAAGCCCA CCGCCTGTAC CAGGGGTCAG TACAAAATGG |
| 1741 | ACCACAGTAC AGGCGGGGCT GTGCGAGCCG CCTGTGAAAA CATAATTTTC ACAGGCGGCT |
| 1801 | CGCACAGCCC CGCCTGTACT GTGGTCCATT TTGTACTGAC CCCTGGTACA GGCGGTGGGC |
| 1861 | TTGGCCGCCT GTGAAGATGC TTTTAGCACC GCCTGTAAAA ATGTTTTTTG TAGCAGTGTT |
| 1921 | TTTCTTATTA GTAGTATCTT TTATACTAAT TAAGATTCAA TAAAAATTCA CCATGACATC |
| 1981 | CCCATTGCCA AGAGAATATT TCGCCGCCCC TCAAAGCAGC CAATAAGGCT TTACTAAAAA |
| 2041 | GACTATCCAC GCAGTAGAGA TTTAGTCAAA ATATTCCAAT AGCAATTGTT TCCTGCCTGC |
| 2101 | TTGACCTTCG TCAGCCACTC ACTGTATAAA TATCGCACCA CGCCCTTTGC AGGCTTACAG |
| 2161 | AGCTTGTATT ACGTACTAAC AAGGCACACA CAGTACCCTG TGTTCACCGG CCCTGCACAA |
| 2221 | AACTCAAGCA GTTATTACTA AC |

(SEQ ID NO:16) or a functional fragment or variant thereof having 75, 80, 85, 90, 95, 96, 97, 98, or 99% sequence identity to SEQ ID NO:16. Each N=can be any nucleotide or combination of any 2, 3, 4, or 5 nucleotides. In some embodiments, a functional Ma1 expression control sequence capable of placing a target gene under photoperiod (short-day) control has the nucleic acid sequence:

| | |
|---|---|
| 1 | CACTATAACA CAACATGGCT TTGCCGACAC TTCCAACTAT CGGCAAAGGG TACCTTTACC |
| 61 | GACACTTAAC GTCTCACGAA AGGTTTTGCC GACAATTTTC AAACAGTCGC GGTAGAAGCA |
| 121 | GTTGGCGAAA CTTTTGCCGA CAGTTAAAGG CATCGCCGAC ACATTTTCTG TAGTCAAATG |
| 181 | GCATACCTAC GCCGACAGTT GAACTTTCAC CGACAGTGAA CCCTTTGCCG ACAGTTTGGA |
| 241 | CCTACGCCGA CAGTTTGGAC CTTTTCCGAC AGTTGGTATG TTAGCGAAAC CGTTTCTAGG |
| 301 | GTGTTTCATA AACCATGCCT TGTCCAACAG TAGAAGTGTC GGCAAAACTA TATTGCTAGG |
| 361 | ATGTAGATAC AATTTAAATA TTTTAATAAA TACACATCAC ATTGATTGAG CAAAATCACA |
| 421 | TGGTCTGTTT TCACTAAAAC TGTCAGAGGT ACACTCCAGT ACTACCAGTA CGTCGCCCGC |
| 481 | ACAGTGGCCA AGGATTTTAC TGCTACTGTT GATTAACATA AGCACTTGCG ACTTTCCCTA |
| 541 | AAATCTTTTA TAAAACAACG GCCGCAATAA TATTGAACTA TTTTTTTTCT AGTACCAAAA |
| 601 | TTAGAATTTG ATCCCTCACC TCATTACATC CATAGTAACA TGACCAGATA TATATGGACA |
| 661 | GGATGGGATC ACTCAGCGAG CAGATACACT GAGCGATTCA TAATCAGATT TTTTAATTTC |
| 721 | TTCTAGTGAA GTGGGGTTTT CCTAGTCTTT TAACATTCAA AATTTAGTAC AAACTTTCCC |
| 781 | TAGTAAATGC CTTCTAGTAA AGATTTCCTA GTATTTTGAC TAGCGATAGT GTTTTATTAC |
| 841 | TAATTAAAAA CATTAGAAGA ACTCCATTTA GTGATTGGTT GTTTGGATTA GTCTTCTCAC |
| 901 | GTTAGACCTA TATATGCAGG ACAACTCAAG CCAGCATAAA TATATGAAAT ATCTTGGTGT |
| 961 | TTGTTTGTCT GACACAGGCA ACCGCGTTTG GTATAAATGT GTTTTCTTGT TTACATTTTA |
| 1021 | CCATCTATAG TCATCTCAAT GTTATATAGT AGAGGCTTCA TGTTTGTAGT AGATAAGGTA |
| 1081 | GAGAATTGAG AATATTTTAT TTTTGTGCGA CCATCAATTT TATGTAATCT GCATTGTCTA |
| 1141 | ATGCTTTATT TGACATTTGA AACTACTTAA TTTGACAGTT ATGCAGGTCC GCATGATCCT |
| 1201 | ATGAAAGCAA TTAATTAGTA CGGGTAAACT GCACTACACA AGTTTGCTAG TACTATTCTA |
| 1261 | TTAACCGACC TGTCAATATT ACCTTAAGTT ACTGATTTCA ATTAGAATCT AACACATTCA |
| 1321 | GGAAAAGAAG TTTCACTAGT ACAAAAATCA TTTTCGTTGG CACGTTGTTT TTTTTTTCAC |
| 1381 | AGGCAGTTCA CAATATCATG GTGCTAGTAG AAAAATTTCA ACGGGCCCAA CAAGAGAACC |
| 1441 | GCCAGGCGGT CTTCTTAATT CAACCGCCTG TGTAAACTTT CCATTTACAT AGGCGGCTTA |
| 1501 | CGATAAAAAC CGTGTGTATA AATACCATTA ACACAGGCAG TCGAGTTACG ACAACCGCCT |
| 1561 | GTGTAAATGT GTCTTTTTAC ACAGGCGGTT TGT ATAGAGG GCCGCCTGTG CTAATATATT |
| 1621 | TACACAGGCT ATGAGCCGCC TGTGTTAAGT CTTCTATAAA TACCCTTCGT CCACCTCCAG |
| 1681 | ACAAGAACAG TTACTCCCAT GAGCTCTGCA CACTGGCGGA CCAGACGATT CCAGTTTCCA |
| 1741 | AGGGGGGAGG TTTTGATTTT CATTTCTTTG GTGAGAAACT TCCAAAAGGT TAGTTAGTGC |
| 1801 | CATTGATGCT ATTTTTTAAG CGATTCTTTG GTTCAATTCT TGTATTGGAG GTGCTCTAGA |
| 1861 | TCTAGAGTTC ATCATGCATT CTTGCTTAGG GTTAGAGTTC ATAGGGCAAA AAGAGAGAGA |
| 1921 | TTTAGCTAAA TTTTTATGTA AATTCATAGT AAATTGTAAA AATTAAAAAA AATAAAAAAT |
| 1981 | AAATACTTTT TAGAATTCTT GTGAGTAGAT CTATACAATA GAGTAATGAT GAGGATATTT |
| 2041 | TGAAGTTTAT AATTTTGATT CAGTTTTAGC TTTTCTTTTT TCAGATGAAT TAGACTTTAT |
| 2101 | AAACTCAAAC ATTAAAATGT TGAAAATCAT AAAATGGCAA ATAAATACTT TTTCAAATCT |
| 2161 | TTGTGCATAA ATACTTCATA GAAATCCTTG AATTATTCCT AAATTTTATA CAATTGTTTC |
| 2221 | TTATAATTAT GAAAATGAGT TTAAACAATT ATTTAAATTC CATAAATTGT AACTCCGTAA |
| 2281 | GGTGTAGGTT TTCATCTCTG TTTAATAGAA GGAGGTTAGT ATCTTAGTTA AGTCTGTTTT |
| 2341 | CGGGGGTTAT ATTAGTTTTG TTTTTAGATT GACCTACATT AATTGTTCTT AACTAATTAC |
| 2401 | AGCTAAATAT GGAGAGGTCA TTATGGATGT ACAACTTATC AAGATTGGAC CTATCATATG |
| 2461 | TAGTGCAGGT CCAAAAATTT ATTGATGTCG CAAAGATACA TGCTCGCAGA ACAAAGGCGA |
| 2521 | AGCACATATG TTGTCCATGC GCAGACTGCA AAAATATTAT GGTATTTGAC AATGTAGAAG |
| 2581 | CAATTACTTC CCATCTGGTT TGAAGAGGAT TTATGGAGGA CTACTTGATT TGGACAAAAC |
| 2641 | ATGGTGAGGG TAGTTTTGCA CCTTATATGC GGACAACTGA CAACACTGCA ACTAACATCA |
| 2701 | ATGTGGAGGG TCCAATGCCA CCTCTCAATG AATTTCATGC TATGCCAGAT GTTAATGAAA |
| 2761 | CTCATACGTC TGATGTCAAT GAAACTCAGC ATGCTAACAC AGATGTTGTT GAAGATGCAG |
| 2821 | ATTTCTTAGA GGCAATAATG AACCGTTGTG CGGATCCATC AATATTCTTC ATGAAGGGAA |
| 2881 | TGAAAGCATT GAAGAAGGCA GCAGAGGACA CTTTGTACGA CGAGTCAAAA GGTTGTACCA |
| 2941 | AACAATGGTC GACATTATGT GTTGTTCTTC AGTTTTTGAC GATGAAGGCT AGACATGGTT |
| 3001 | GGTCCGATGC TAGCTTCAAT GATTTCTTGC GTGTACTTGG AGACCTTCTT CCTAAGGAGA |
| 3061 | ACAAAGTGCC TGCTAACACA TACTATGCAA AGAAGCTAGT CAGTCCACTT ACGATAGGTG |
| 3121 | TTGAGAAGAT CCACGCATGT AGAAATCATT GTATTCTATA TCGAGGTGAT CAATATAAAG |
| 3181 | ACTTAGACAG TTGTCCAAAC TGTGGTGCCA GTAGGTACAA GACAAACAAA GATTTTCGGG |
| 3241 | AGGAAGAGAA TCTAGCCTCT GTTTCTACAG GGAGGAAGCG AAAGAAGACC CAAACAAAGA |
| 3301 | CTCAACAAGA CAAGCGCTCA AAGCCTAGTA GCAATGAAGA AGTGGACTAT TATGCATTGA |
| 3361 | GAAGAGTCTC CCTATGAGCC AAAAAAGGGG ACAGCAGCAG GCACAACTCT CTTTCTGAAA |
| 3421 | GGACTTGGAA AGCAGCGGAC GGCACGGCTC ATTGAGCTCG AACCGTCACA GAAAAAGGAA |
| 3481 | GCCACCGCCC AGTCAATAGA AGCCATGCCC CCATCAAAGG AAGCCCCAAG TGGCGATGTA |
| 3541 | CATATTGAAC AGCCATCAAG TCAACCATTG ACCCTAAAGG ATATCAGAAA GCCAACGATT |
| 3601 | GATGATTATG TCAATGTCCC TAGTGACTAT GTGCCCGGAA GGCCTATGCT CCAATGGACG |
| 3661 | CTGCTCGATT AGATTCAATG GCTGATAAAA AGGTTTCATG ACTGGTACAT GAGAGCAGTG |
| 3721 | CATGCTAGCC TCCATGGAAT CAGAGTTGAT ATACCAACAG ACATGTTTGC TACTGGTAAC |
| 3781 | AAAAAAAGCA AGACATTTGT TACCTTTGAG GACATGCACT TGTTATTGAA CTATAGGCGG |
| 3841 | CTTGACGTCC AACTCATAAC AATCTGGTGC CTGTAAGTAT CACTCATGCA CACACAATTA |
| 3901 | TTATATATTA ATATGTAGTG TGAAACTCTA ATATGTAGAT GTTGTCTGTA GTTTGCAAGA |
| 3961 | TCACGAGCAG ATGTCATTAT TATCTGCCGG ATCGATGGTC GGTTATCTGA GCCCTATCAA |
| 4021 | GTTACAAGAA AATATGAACA AATTCGTATT ATCAAAGGAA GATAGAGCAA AGATAGAGGA |
| 4081 | AGACAAAACA CCAGGATAAT TATGCCATCT ATCTTGGTAG ATCAATGCTG AGGTATAAAT |
| 4141 | ATAGGGATTT TATATTGGCA CCATACAACA TTAGGTAAGC TTGACTTCAT ATACGTATTT |
| 4201 | CAAATTATCG TGTAAACAAT ATACATGTGT CGCTCACTCA TTTATTCATG CAGTGACCAT |
| 4261 | TGGATTGTTT TTTATATTTA TCCCTTCGAA GGGAAGGTGC TTGTCCTAGA CTCTTTACAT |
| 4321 | GTTCCTCCCG AGAAGTATCA ACCATTCTTG GTTCAATTAG AAAGGTGAGC CAACATGAAA |
| 4381 | CCACATGCGT ACTTATATAA ATTAGAGTTT CAAAATAACT TTAGTGATTT AGGTTCGATA |
| 4441 | TCTACGGGGC ATGGCGGTTT TATAAGAAAC AAAAGGGACC TGTCGACGCT GCACGCTCAG |
| 4501 | ATCCTAGGAT CCCATTGATG ATACAACACC ACTATCCGGT AAGTTTTCTG AACACATTTC |
| 4561 | ATCATATAAA TAATACATAA AGCATGGCAA ATTTAGAATA ATCCGTTGCT CATTATATAG |
| 4621 | TGCCACAAGC AACCACCTGG ATCGGTCTAT TGTGGGTACT ATGTCTGTGA GTTTATAAGG |
| 4681 | CAGCGGGGAC GTTACGTCAA GGACAAAAAT ATGGTAAATA ATATCTATGT ATGAAAGTTT |
| 4741 | TCTCATTAAA GCTGCAAAAT TATATATTGA ACATGTGTCA ATCATGCTTT TAAACTTTAT |
| 4801 | TTTCAGCCGA AAAAGCAAGG AAAAGACGTG CCCTTTACAC CAAAGACTCT GGAAGATATA |
| 4861 | GTAGCATACT TGTGTGGTTT TATTATGAGA GAAATAATTT CAAGTGACAG TGCATATTTT |
| 4921 | GATCATGAGG GCGATTTAGC AAGTGATAAA TTTAGAGTGC TGACAGACAT AGCAGGTCTA |
| 4981 | AATCTGAAGC GAAACGACAT GTAAACATTG TATGGTTGTG CGGATAACAT GCATTGACGT |
| 5041 | GTATATATAT AATTTTATGG TTGATGTTTG ATTTGTTTAC AATTCTATAA TATATATATG |
| 5101 | TGGTGTATGT ATGATGTTGT GTGTGTATAT ATATATATAT ATATATATAT ATATATATAT |
| 5161 | ATATATATAT ATATATATAT ATATAATGTT TAGCACTGTG TTTGGTGGGA AAAATTAAAA |
| 5221 | TTTGAAATAT ATATAAAAAA TTATTTACAC AGACAGTGTA CGTGTCGAGC GTCGTCCTGT |
| 5281 | GCTATACAAA TACATTCTAA CAGGCGGCTC GCCTTGTCCA CCGGTCGGTT AAAAATACAT |
| 5341 | TTCCACACNG GCCTGGCTGG GAGAGCCGCC TGTGAAAACA TAATTTTCAC AGGCGGCTCG |
| 5401 | CACAGCCCCG CCTGTACTGT GGTCCATTTT GTACTGACCC CTGGTACAGG CGGTGGGCTT |
| 5461 | GGCCGCCTGT GAAGATGCTT TTAGCACCGC CTGTAAAAAT GTTTTTTGTA GCAGTGTTTT |
| 5521 | TCTTATTAGT AGTATCTTTT ATACTAATTA AGATTCAATA AAAATTCACC ATGACATCCC |
| 5581 | CATTGCCAAG AGAATATTTC GCCGCCCCTC AAAGCAGCCA AT |

(SEQ ID NO:17) or a functional fragment or variant thereof having 75, 80, 85, 90, 95, 96, 97, 98, or 99% sequence identity to SEQ ID NO:17.

In some embodiments, a functional Ma1 expression control sequence capable of placing a target gene under photoperiod (short-day) control has the nucleic acid sequence:

| | |
|---|---|
| 1 | CACTATAACA CAACATGGCT TTGCCGACAC TTCCAACTAT CGGCAAAGGG TACCTTTACC |
| 61 | GACACTTAAC GTCTCACGAA AGGTTTTGCC GACAATTTTC AAACAGTCGC GGTAGAAGCA |
| 121 | GTTGGCGAAA CTTTTGCCGA CAGTTAAAGG CATCGCCGAC ACATTTTCTG TAGTCAAATG |
| 181 | GCATACCTAC GCCGACAGTT GAACTTTCAC CGACAGTGAA CCCTTTGCCG ACAGTTTGGA |
| 241 | CCTACGCCGA CAGTTTGGAC CTTTTCCGAC AGTTGGTATG TTAGCGAAAC CGTTTCTAGG |
| 301 | GTGTTTCATA AACCATGCCT TGTCCAACAG TAGAAGTGTC GGCAAAACTA TATTGCTAGG |
| 361 | ATGTAGATAC AATTTAAATA TTTTAATAAA TACACATCAC ATTGATTGAG CAAAATCACA |
| 421 | TGGTCTGTTT TCACTAAAAC TGTCAGAGGT ACACTCCAGT ACTACCAGTA CGTCGCCCGC |
| 481 | ACAGTGGCCA AGGATTTTAC TGCTACTGTT GATTAACATA AGCACTTGCG ACTTTCCCTA |
| 541 | AAATCTTTTA TAAAACAACG GCCGCAATAA TATTGAACTA TTTTTTTTCT AGTACCAAAA |
| 601 | TTAGAATTTG ATCCCTCACC TCATTACATC CATAGTAACA TGACCAGATA TATATGGACA |
| 661 | GGATGGGATC ACTCAGCGAG CAGATACACT GAGCGATTCA TAATCAGATT TTTTAATTTC |
| 721 | TTCTAGTGAA GTGGGGTTTT CCTAGTCTTT TAACATTCAA AATTTAGTAC AAACTTTCCC |
| 781 | TAGTAAATGC CTTCTAGTAA AGATTTCCTA GTATTTTGAC TAGCGATAGT GTTTTATTAC |
| 841 | TAATTAAAAA CATTAGAAGA ACTCCATTTA GTGATTGGTT GTTTGGATTA GTCTTCTCAC |
| 901 | GTTAGACCTA TATATGCAGG ACAACTCAAG CCAGCATAAA TATATGAAAT ATCTTGGTGT |
| 961 | TTGTTTGTCT GACACAGGCA ACCGCGTTTG GTATAAATGT GTTTTCTTGT TTACATTTTA |
| 1021 | CCATCTATAG TCATCTCAAT GTTATATAGT AGAGGCTTCA TGTTTGTAGT AGATAAGGTA |
| 1081 | GAGAATTGAG AATATTTTAT TTTTGTGCGA CCATCAATTT TATGTAATCT GCATTGTCTA |
| 1141 | ATGCTTTATT TGACATTTGA AACTACTTAA TTTGACAGTT ATGCAGGTCC GCATGATCCT |
| 1201 | ATGAAAGCAA TTAATTAGTA CGGGTAAACT GCACTACACA AGTTTGCTAG TACTATTCTA |
| 1261 | TTAACCGACC TGTCAATATT ACCTTAAGTT ACTGATTTCA ATTAGAATCT AACACATTCA |
| 1321 | GGAAAAGAAG TTTCACTAGT ACAAAAATCA TTTTCGTTGG CACGTTGTTT TTTTTTTCAC |
| 1381 | AGGCAGTTCA CAATATCATG GTGCTAGTAG AAAAATTTCA ACGGGCCCAA CAAGAGAACC |
| 1441 | GCCAGGCGGT CTTCTTAATT CAACCGCCTG TGTAAACTTT CCATTTACAT AGGCGGCTTA |
| 1501 | CGATAAAAAC CGTGTGTATA AATACCATTA ACACAGGCAG TCGAGTTACG ACAACCGCCT |
| 1561 | GTGTAAATGT GTCTTTTTAC ACAGGCGGTT TGT ATAGAGG GCCGCCTGTG CTAATATATT |
| 1621 | TACACAGGCT ATGAGCCGCC TGTGTTAAGT CTTCTATAAA TACCCTTCGT CCACCTCCAG |
| 1681 | ACAAGAACAG TTACTCCCAT GAGCTCTGCA CACTGGCGGA CCAGACGATT CCAGTTTCCA |
| 1741 | AGGGGGGAGG TTTTGATTTT CATTTCTTTG GTGAGAAACT TCCAAAAGGT TAGTTAGTGC |
| 1801 | CATTGATGCT ATTTTTTAAG CGATTCTTTG GTTCAATTCT TGTATTGGAG GTGCTCTAGA |
| 1861 | TCTAGAGTTC ATCATGCATT CTTGCTTAGG GTTAGAGTTC ATAGGGCAAA AAGAGAGAGA |
| 1921 | TTTAGCTAAA TTTTTATGTA AATTCATAGT AAATTGTAAA AATTAAAAAA AATAAAAAAT |
| 1981 | AAATACTTTT TAGAATTCTT GTGAGTAGAT CTATACAATA GAGTAATGAT GAGGATATTT |
| 2041 | TGAAGTTTAT AATTTTGATT CAGTTTTAGC TTTTCTTTTT TCAGATGAAT TAGACTTTAT |
| 2101 | AAACTCAAAC ATTAAAATGT TGAAAATCAT AAAATGGCAA ATAAATACTT TTTCAAATCT |
| 2161 | TTGTGCATAA ATACTTCATA GAAATCCTTG AATTATTCCT AAATTTTATA CAATTGTTTC |
| 2221 | TTATAATTAT GAAAATGAGT TTAAACAATT ATTTAAATTC CATAAATTGT AACTCCGTAA |
| 2281 | GGTGTAGGTT TTCATCTCTG TTTAATAGAA GGAGGTTAGT ATCTTAGTTA AGTCTGTTTT |
| 2341 | CGGGGGTTAT ATTAGTTTTG TTTTTAGATT GACCTACATT AATTGTTCTT AACTAATTAC |
| 2401 | AGCTAAATAT GGAGAGGTCA TTATGGATGT ACAACTTATC AAGATTGGAC CTATCATATG |
| 2461 | TAGTGCAGGT CCAAAAATTT ATTGATGTCG CAAAGATACA TGCTCGCAGA ACAAAGGCGA |
| 2521 | AGCACATATG TTGTCCATGC GCAGACTGCA AAAATATTAT GGTATTTGAC AATGTAGAAG |
| 2581 | CAATTACTTC CCATCTGGTT TGAAGAGGAT TTATGGAGGA CTACTTGATT TGGACAAAAC |
| 2641 | ATGGTGAGGG TAGTTTTGCA CCTTATATGC GGACAACTGA CAACACTGCA ACTAACATCA |
| 2701 | ATGTGGAGGG TCCAATGCCA CCTCTCAATG AATTTCATGC TATGCCAGAT GTTAATGAAA |
| 2761 | CTCATACGTC TGATGTCAAT GAAACTCAGC ATGCTAACAC AGATGTTGTT GAAGATGCAG |
| 2821 | ATTTCTTAGA GGCAATAATG AACCGTTGTG CGGATCCATC AATATTCTTC ATGAAGGGAA |
| 2881 | TGAAAGCATT GAAGAAGGCA GCAGAGGACA CTTTGTACGA CGAGTCAAAA GGTTGTACCA |
| 2941 | AACAATGGTC GACATTATGT GTTGTTCTTC AGTTTTTGAC GATGAAGGCT AGACATGGTT |
| 3001 | GGTCCGATGC TAGCTTCAAT GATTTCTTGC GTGTACTTGG AGACCTTCTT CCTAAGGAGA |
| 3061 | ACAAAGTGCC TGCTAACACA TACTATGCAA AGAAGCTAGT CAGTCCACTT ACGATAGGTG |
| 3121 | TTGAGAAGAT CCACGCATGT AGAAATCATT GTATTCTATA TCGAGGTGAT CAATATAAAG |
| 3181 | ACTTAGACAG TTGTCCAAAC TGTGGTGCCA GTAGGTACAA GACAAACAAA GATTTTCGGG |
| 3241 | AGGAAGAGAA TCTAGCCTCT GTTTCTACAG GGAGGAAGCG AAAGAAGACC CAAACAAAGA |
| 3301 | CTCAACAAGA CAAGCGCTCA AAGCCTAGTA GCAATGAAGA AGTGGACTAT TATGCATTGA |
| 3361 | GAAGAGTCTC CCTATGAGCC AAAAAAGGGG ACAGCAGCAG GCACAACTCT CTTTCTGAAA |
| 3421 | GGACTTGGAA AGCAGCGGAC GGCACGGCTC ATTGAGCTCG AACCGTCACA GAAAAAGGAA |
| 3481 | GCCACCGCCC AGTCAATAGA AGCCATGCCC CCATCAAAGG AAGCCCCAAG TGGCGATGTA |
| 3541 | CATATTGAAC AGCCATCAAG TCAACCATTG ACCCTAAAGG ATATCAGAAA GCCAACGATT |
| 3601 | GATGATTATG TCAATGTCCC TAGTGACTAT GTGCCCGGAA GGCCTATGCT CCAATGGACG |
| 3661 | CTGCTCGATT AGATTCAATG GCTGATAAAA AGGTTTCATG ACTGGTACAT GAGAGCAGTG |
| 3721 | CATGCTAGCC TCCATGGAAT CAGAGTTGAT ATACCAACAG ACATGTTTGC TACTGGTAAC |
| 3781 | AAAAAAAGCA AGACATTTGT TACCTTTGAG GACATGCACT TGTTATTGAA CTATAGGCGG |
| 3841 | CTTGACGTCC AACTCATAAC AATCTGGTGC CTGTAAGTAT CACTCATGCA CACACAATTA |
| 3901 | TTATATATTA ATATGTAGTG TGAAACTCTA ATATGTAGAT GTTGTCTGTA GTTTGCAAGA |
| 3961 | TCACGAGCAG ATGTCATTAT TATCTGCCGG ATCGATGGTC GGTTATCTGA GCCCTATCAA |
| 4021 | GTTACAAGAA AATATGAACA AATTCGTATT ATCAAAGGAA GATAGAGCAA AGATAGAGGA |
| 4081 | AGACAAAACA CCAGGATAAT TATGCCATCT ATCTTGGTAG ATCAATGCTG AGGTATAAAT |
| 4141 | ATAGGGATTT TATATTGGCA CCATACAACA TTAGGTAAGC TTGACTTCAT ATACGTATTT |
| 4201 | CAAATTATCG TGTAAACAAT ATACATGTGT CGCTCACTCA TTTATTCATG CAGTGACCAT |
| 4261 | TGGATTGTTT TTTATATTTA TCCCTTCGAA GGGAAGGTGC TTGTCCTAGA CTCTTTACAT |
| 4321 | GTTCCTCCCG AGAAGTATCA ACCATTCTTG GTTCAATTAG AAAGGTGAGC CAACATGAAA |
| 4381 | CCACATGCGT ACTTATATAA ATTAGAGTTT CAAAATAACT TTAGTGATTT AGGTTCGATA |
| 4441 | TCTACGGGGC ATGGCGGTTT TATAAGAAAC AAAAGGGACC TGTCGACGCT GCACGCTCAG |
| 4501 | ATCCTAGGAT CCCATTGATG ATACAACACC ACTATCCGGT AAGTTTTCTG AACACATTTC |
| 4561 | ATCATATAAA TAATACATAA AGCATGGCAA ATTTAGAATA ATCCGTTGCT CATTATATAG |
| 4621 | TGCCACAAGC AACCACCTGG ATCGGTCTAT TGTGGGTACT ATGTCTGTGA GTTTATAAGG |
| 4681 | CAGCGGGGAC GTTACGTCAA GGACAAAAAT ATGGTAAATA ATATCTATGT ATGAAGTTTT |
| 4741 | CTCATTAAAG CTGCAAAATT ATATATTGAA CATGTGTCAA TCATGCTTTT AAACTTTATT |
| 4801 | TTCAGCCGAA AAAGCAAGGA AAAGACGTGC CCTTTACACC AAAGACTCTG GAAGATATAG |
| 4861 | TAGCATACTT GTGTGGTTTT ATTATGAGAG AAATAATTTC AAGTGACAGT GCATATTTTG |
| 4921 | ATCATGAGGG CGATTTAGCA AGTGATAAAT TTAGAGTGCT GACAGACATA GCAGGTCTAA |
| 4981 | ATCTGAAGCG AAACGACATG TAAACATTGT ATGGTTGTGC GGATAACATG CATTGACGTG |
| 5041 | TATATATATA ATTTTATGGT TGATGTTTGA TTTGTTTACA ATTCTATAAT ATATATATGT |
| 5101 | GGTGTATGTA TGATGTTGTG TGTGTATATA TATATATATA TATATATATA TATATATATA |
| 5161 | TATATATATA TATATATATA TATAATGTTT AGCACTGTGT TTGGTGGGAA AAATTAAAAT |
| 5221 | TTGAAATATA TATAAAAAAT TATTTACACA GACAGTGTAG TGTGAGCTGC CTGTGTAAAA |
| 5281 | ATACATTTAT ACAGGCGGCT CACCTTGTCN NNNCAGGCGG TGCTAAAAGC ATCTTCACAG |
| 5241 | GCGGCCAAGC CCACCGCCTG TACCAGGGGT CAGTACAAAA TGGACCACAG TACAGGCGGG |
| 5401 | GCTGTGCGAG CCGCCTGTGA AAACATAATT TTCACAGGCG GCTCGCACAG CCCCGCCTGT |
| 5461 | ACTGTGGTCC ATTTTGTACT GACCCCTGGT ACAGGCGGTG GGCTTGGCCG CCTGTGAAGA |
| 5521 | TGCTTTTAGC ACCGCCTGTA AAAATGTTTT TTGTAGCAGT GTTTTTCTTA TTAGTAGTAT |
| 5581 | CTTTTATACT AATTAAGATT CAATAAAAAT TCACCATGAC ATCCCCATTG CCAAGAGAAT |
| 5641 | ATTTCGCCGC CCCTCAAAGC AGCCAAT |

(SEQ ID NO:18) or a functional fragment or variant thereof having 75, 80, 85, 90, 95, 96, 97, 98, or 99% sequence identity to SEQ ID NO:18. Each N=can be any nucleotide or combination of any 2, 3, 4, or 5 nucleotides.

In some embodiments, a functional Ma1 expression control sequence capable of placing a target gene under photoperiod (short-day) control has the nucleic acid sequence:

| | |
|---|---|
| 1 | CACTAGTACA AAAATCATTT TCGTTGGCAC GTTGTTTTTT TTTTCACAGG CAGTTCACAA |
| 61 | TATCATGGTG CTAGTAGAAA AATTTCAACG GGCCCAACAA GAGAACCGCC AGGCGGTCTT |
| 121 | CTTAATTCAA CCGCCTGTGT AAACTTTCCA TTTACATAGG CGGCTTACGA TAAAAACCGT |
| 181 | GTGTATAAAT ACCATTAACA CAGGCAGTCG AGTTACGACA ACCGCCTGTG TAAATGTGTC |
| 241 | TTTTTACACA GGCGGTTTGT ATAGAGGGCC GCCTGTGCTA ATATATTTAC ACAGGCTATG |
| 301 | AGCCGCCTGT GTTAAGTCTT CTATAAATAC CCTTCGTCCA CCTCCAGACA AGAACAGTTA |
| 361 | CTCCCATGAG CTCTGCACAC TGGCGGACCA GACGATTCCA GTTTCCAAGG GGGGAGGTTT |
| 421 | TGATTTTCAT TTCTTTGGTG AGAAACTTCC AAAAGGTTAG TTAGTGCCAT TGATGCTATT |
| 481 | TTTTAAGCGA TTCTTTGGTT CAATTCTTGT ATTGGAGGTG CTCTAGATCT AGAGTTCATC |
| 541 | ATGCATTCTT GCTTAGGGTT AGAGTTCATA GGGCAAAAAG AGAGAGATTT AGCTAAATTT |
| 601 | TTATGTAAAT TCATAGTAAA TTGTAAAAAT TAAAAAAAT AAAAAATAAA TACTTTTTAG |
| 661 | AATTCTTGTG AGTAGATCTA TACAATAGAG TAATGATGAG GATATTTTGA AGTTTATAAT |
| 721 | TTTGATTCAG TTTTAGCTTT TCTTTTTTCA GATGAATTAG ACTTTATAAA CTCAAACATT |
| 781 | AAAATGTTGA AAATCATAAA ATGGCAAATA AATACTTTTT CAAATCTTTG TGCATAAATA |
| 841 | CTTCATAGAA ATCCTTGAAT TATTCCTAAA TTTTATACAA TTGTTTCTTA TAATTATGAA |
| 901 | AATGAGTTTA AACAATTATT TAAATTCCAT AAATTGTAAC TCCGTAAGGT GTAGGTTTTC |
| 961 | ATCTCTGTTT AATAGAAGGA GGTTAGTATC TTAGTTAAGT CTGTTTTCGG GGGTTATATT |
| 1021 | AGTTTTGTTT TTAGATTGAC CTACATTAAT TGTTCTTAAC TAATTACAGC TAAATATGGA |
| 1081 | GAGGTCATTA TGGATGTACA ACTTATCAAG ATTGGACCTA TCATATGTAG TGCAGGTCCA |
| 1141 | AAAATTTATT GATGTCGCAA AGATACATGC TCGCAGAACA AAGGCGAAGC ACATATGTTG |
| 1201 | TCCATGCGCA GACTGCAAAA ATATTATGGT ATTTGACAAT GTAGAAGCAA TTACTTCCCA |
| 1261 | TCTGGTTTGA AGAGGATTTA TGGAGGACTA CTTGATTTGG ACAAAACATG GTGAGGGTAG |
| 1321 | TTTTGCACCT TATATGCGGA CAACTGACAA CACTGCAACT AACATCAATG TGGAGGGTCC |
| 1381 | AATGCCACCT CTCAATGAAT TTCATGCTAT GCCAGATGTT AATGAAACTC ATACGTCTGA |
| 1441 | TGTCAATGAA ACTCAGCATG CTAACACAGA TGTTGTTGAA GATGCAGATT TCTTAGAGGC |
| 1501 | AATAATGAAC CGTTGTGCGG ATCCATCAAT ATTCTTCATG AAGGGAATGA AAGCATTGAA |
| 1561 | GAAGGCAGCA GAGGACACTT TGTACGACGA GTCAAAAGGT TGTACCAAAC AATGGTCGAC |
| 1621 | ATTATGTGTT GTTCTTCAGT TTTTGACGAT GAAGGCTAGA CATGGTTGGT CCGATGCTAG |
| 1681 | CTTCAATGAT TTCTTGCGTG TACTTGGAGA CCTTCTTCCT AAGGAGAACA AAGTGCCTGC |
| 1741 | TAACACATAC TATGCAAAGA AGCTAGTCAG TCCACTTACG ATAGGTGTTG AGAAGATCCA |
| 1801 | CGCATGTAGA AATCATTGTA TTCTATATCG AGGTGATCAA TATAAAGACT TAGACAGTTG |
| 1861 | TCCAAACTGT GGTGCCAGTA GGTACAAGAC AAACAAAGAT TTTCGGGAGG AAGAGAATCT |
| 1921 | AGCCTCTGTT TCTACAGGGA GGAAGCGAAA GAAGACCCAA ACAAAGACTC AACAAGACAA |
| 1981 | GCGCTCAAAG CCTAGTAGCA ATGAAGAAGT GGACTATTAT GCATTGAGAA GAGTCTCCCT |
| 2041 | ATGAGCCAAA AAAGGGGACA GCAGCAGGCA CAACTCTCTT TCTGAAAGGA CTTGGAAAGC |
| 2101 | AGCGGACGGC ACGGCTCATT GAGCTCGAAC CGTCACAGAA AAAGGAAGCC ACCGCCCAGT |
| 2161 | CAATAGAAGC CATGCCCCCA TCAAAGGAAG CCCCAAGTGG CGATGTACAT ATTGAACAGC |
| 2221 | CATCAAGTCA ACCATTGACC CTAAAGGATA TCAGAAAGCC AACGATTGAT GATTATGTCA |
| 2281 | ATGTCCCTAG TGACTATGTG CCCGGAAGGC CTATGCTCCA ATGGACGCTG CTCGATTAGA |
| 2341 | TTCAATGGCT GATAAAAAGG TTTCATGACT GGTACATGAG AGCAGTGCAT GCTAGCCTCC |
| 2401 | ATGGAATCAG AGTTGATATA CCAACAGACA TGTTTGCTAC TGGTAACAAA AAAAGCAAGA |
| 2461 | CATTTGTTAC CTTTGAGGAC ATGCACTTGT TATTGAACTA TAGGCGGCTT GACGTCCAAC |
| 2521 | TCATAACAAT CTGGTGCCTG TAAGTATCAC TCATGCACAC ACAATTATTA TATATTAATA |
| 2581 | TGTAGTGTGA AACTCTAATA TGTAGATGTT GTCTGTAGTT TGCAAGATCA CGAGCAGATG |
| 2641 | TCATTATTAT CTGCCGGATC GATGGTCGGT TATCTGAGCC CTATCAAGTT ACAAGAAAAT |
| 2701 | ATGAACAAAT TCGTATTATC AAAGGAAGAT AGAGCAAAGA TAGAGGAAGA CAAAACACCA |
| 2761 | GGATAATTAT GCCATCTATC TTGGTAGATC AATGCTGAGG TATAAATATA GGGATTTTAT |
| 2821 | ATTGGCACCA TACAACATTA GGTAAGCTTG ACTTCATATA CGTATTTCAA ATTATCGTGT |
| 2881 | AAACAATATA CATGTGTCGC TCACTCATTT ATTCATGCAG TGACCATTGG ATTGTTTTTT |
| 2941 | ATATTTATCC CTTCGAAGGG AAGGTGCTTG TCCTAGACTC TTTACATGTT CCTCCCGAGA |
| 3001 | AGTATCAACC ATTCTTGGTT CAATTAGAAA GGTGAGCCAA CATGAAACCA CATGCGTACT |
| 3061 | TAT ATAAATT AGAGTTTCAA AATAACTTTA GTGATTTAGG TTCGATATCT ACGGGGCATG |
| 3121 | GCGGTTTTAT AAGAAACAAA AGGGACCTGT CGACGCTGCA CGCTCAGATC CTAGGATCCC |
| 3181 | ATTGATGATA CAACACCACT ATCCGGTAAG TTTTCTGAAC ACATTTCATC ATATAAATAA |
| 3241 | TACATAAAGC ATGGCAAATT TAGAATAATC CGTTGCTCAT TAT ATAGTGC CACAAGCAAC |
| 3301 | CACCTGGATC GGTCTATTGT GGGTACTATG TCTGTGAGTT TATAAGGCAG CGGGGACGTT |
| 3361 | ACGTCAAGGA CAAAAATATG GTAAATAATA TCTATGTATG AAAGTTTTCT CATTAAAGCT |
| 3421 | GCAAAATTAT ATATTGAACA TGTGTCAATC ATGCTTTTAA ACTTTATTTT CAGCCGAAAA |
| 3481 | AGCAAGGAAA AGACGTGCCC TTTACACCAA AGACTCTGGA AGATATAGTA GCATACTTGT |
| 3541 | GTGGTTTTAT TATGAGAGAA ATAATTTCAA GTGACAGTGC ATATTTTGAT CATGAGGGCG |
| 3601 | ATTTAGCAAG TGATAAATTT AGAGTGCTGA CAGACATAGC AGGTCTAAAT CTGAAGCGAA |
| 3661 | ACGACATGTA AACATTGTAT GGTTGTGCGG ATAACATGCA TTGACGTGTA TATATATAAT |
| 3721 | TTTATGGTTG ATGTTTGATT TGTTTACAAT TCTATAATAT ATATATGTGG TGTATGTATG |
| 3781 | ATGTTGTGTG TGTATATATA TATATATATA TATATATATA TATATATATA TATATATATA |
| 3841 | TATATATATA TAATGTTTAG CACTGTGTTT GGTGGGAAAA ATTAAAATTT GAAATATATA |
| 3901 | TAAAAAATTA TTTACACAGA CAGTGTACGT GTCGAGCGTC GTCCTGTGCT ATACAAATAC |
| 3961 | ATTCTAACAG GCGGCTCGCC TTGTCCACCG GTCGGTTAAA AATACATTTC CACACNGGCC |
| 4021 | TGGCTGGGAG AGCCGCCTGT GAAAACATAA TTTTCACAGG CGGCTCGCAC AGCCCCGCCT |
| 4081 | GTACTGTGGT CCATTTTGTA CTGACCCCTG GTACAGGCGG TGGGCTTGGC CGCCTGTGAA |
| 4141 | GATGCTTTTA GCACCGCCTG TAAAAATGTT TTTTGTAGCA GTGTTT |

(SEQ ID NO:19) or a functional fragment or variant thereof having 75%, 80%, 85%, 90%, 95%, 96%, 97%, 98%, or 99% sequence identity to SEQ ID NO:19.

In some embodiments, a functional Ma1 expression control sequence capable of placing a target gene under photoperiod (short-day) control has the nucleic acid sequence:

| | |
|---|---|
| 1 | CACTAGTACA AAAATCATTT TCGTTGGCAC GTTGTTTTTT TTTTCACAGG CAGTTCACAA |
| 61 | TATCATGGTG CTAGTAGAAA AATTTCAACG GGCCCAACAA GAGAACCGCC AGGCGGTCTT |
| 121 | CTTAATTCAA CCGCCTGTGT AAACTTTCCA TTTACATAGG CGGCTTACGA TAAAAACCGT |
| 181 | GTGTATAAAT ACCATTAACA CAGGCAGTCG AGTTACGACA ACCGCCTGTG TAAATGTGTC |
| 241 | TTTTTACACA GGCGGTTTGT ATAGAGGGCC GCCTGTGCTA ATATATTTAC ACAGGCTATG |
| 301 | AGCCGCCTGT GTTAAGTCTT CTATAAATAC CCTTCGTCCA CCAACAGACA AGAACAGTTA |
| 361 | CTCCCATGAG CTCTGCACAC TGGCGGACCA GACGATTCCA GTTTCCAAGG GGGGAGGTTT |
| 421 | TGATTTTCAT TTCTTTGGTG AGAAACTTCC AAAAGGTTAG TTAGTGCCAT TGATGCTATT |
| 481 | TTTTAAGCGA TTCTTTGGTT CAATTCTTGT ATTGGAGGTG CTCTAGATCT AGAGTTCATC |
| 541 | ATGCATTCTT GCTTAGGGTT AGAGTTCATA GGGCAAAAAG AGAGAGATTT AGCTAAATTT |
| 601 | TTATGTAAAT TCATAGTAAA TTGTAAAAAT TAAAAAAAAT AAAAAATAAA TACTTTTTAG |
| 661 | AATTCTTGTG AGTAGATCTA TACAATAGAG TAATGATGAG GATATTTTGA AGTTTATAAT |
| 721 | TTTGATTCAG TTTTAGCTTT TCTTTTTTCA GATGAATTAG ACTTTATAAA CTCAAACATT |
| 781 | AAAATGTTGA AAATCATAAA ATGGCAAATA AATACTTTTT CAAATCTTTG TGCATAAATA |
| 841 | CTTCATAGAA ATCCTTGAAT TATTCCTAAA TTTTATACAA TTGTTTCTTA TAATTATGAA |
| 901 | AATGAGTTTA AACAATTATT TAAATTCCAT AAATTGTAAC TCCGTAAGGT GTAGGTTTTC |
| 961 | ATCTCTGTTT AATAGAAGGA GGTTAGTATC TTAGTTAAGT CTGTTTTCGG GGGTTATATT |
| 1021 | AGTTTTGTTT TTAGATTGAC CTACATTAAT TGTTCTTAAC TAATTACAGC TAAATATGGA |
| 1081 | GAGGTCATTA TGGATGTACA ACTTATCAAG ATTGGACCTA TCATATGTAG TGCAGGTCCA |
| 1141 | AAAATTTATT GATGTCGCAA AGATACATGC TCGCAGAACA AAGGCGAAGC ACATATGTTG |
| 1201 | TCCATGCGCA GACTGCAAAA ATATTATGGT ATTTGACAAT GTAGAAGCAA TTACTTCCCA |
| 1261 | TCTGGTTTGA AGAGGATTTA TGGAGGACTA CTTGATTTGG ACAAAACATG GTGAGGGTAG |
| 1321 | TTTTGCACCT TATATGCGGA CAACTGACAA CACTGCAACT AACATCAATG TGGAGGGTCC |
| 1381 | AATGCCACCT CTCAATGAAT TTCATGCTAT GCCAGATGTT AATGAAACTC ATACGTCTGA |
| 1441 | TGTCAATGAA ACTCAGCATG CTAACACAGA TGTTGTTGAA GATGCAGATT TCTTAGAGGC |
| 1501 | AATAATGAAC CGTTGTGCGG ATCCATCAAT ATTCTTCATG AAGGGAATGA AAGCATTGAA |
| 1561 | GAAGGCAGCA GAGGACACTT TGTACGACGA GTCAAAAGGT TGTACCAAAC AATGGTCGAC |
| 1621 | ATTATGTGTT GTTCTTCAGT TTTTGACGAT GAAGGCTAGA CATGGTTGGT CCGATGCTAG |
| 1681 | CTTCAATGAT TTCTTGCGTG TACTTGGAGA CCTTCTTCCT AAGGAGAACA AAGTGCCTGC |
| 1741 | TAACACATAC TATGCAAAGA AGCTAGTCAG TCCACTTACG ATAGGTGTTG AGAAGATCCA |
| 1801 | CGCATGTAGA AATCATTGTA TTCTATATCG AGGTGATCAA TATAAAGACT TAGACAGTTG |
| 1861 | TCCAAACTGT GGTGCCAGTA GGTACAAGAC AAACAAAGAT TTTCGGGAGG AAGAGAATCT |
| 1921 | AGCCTCTGTT TCTACAGGGA GGAAGCGAAA GAAGACCCAA ACAAAGACTC AACAAGACAA |
| 1981 | GCGCTCAAAG CCTAGTAGCA ATGAAGAAGT GGACTATTAT GCATTGAGAA GAGTCTCCCT |
| 2041 | ATGAGCCAAA AAAGGGGACA GCAGCAGGCA CAACTCTCTT TCTGAAAGGA CTTGGAAAGC |
| 2101 | AGCGGACGGC ACGGCTCATT GAGCTCGAAC CGTCACAGAA AAAGGAAGCC ACCGCCCAGT |
| 2161 | CAATAGAAGC CATGCCCCCA TCAAAGGAAG CCCCAAGTGG CGATGTACAT ATTGAACAGC |
| 2221 | CATCAAGTCA ACCATTGACC CTAAAGGATA TCAGAAAGCC AACGATTGAT GATTATGTCA |
| 2281 | ATGTCCCTAG TGACTATGTG CCCGGAAGGC CTATGCTCCA ATGGACGCTG CTCGATTAGA |
| 2341 | TTCAATGGCT GATAAAAAGG TTTCATGACT GGTACATGAG AGCAGTGCAT GCTAGCCTCC |
| 2401 | ATGGAATCAG AGTTGATATA CCAACAGACA TGTTTGCTAC TGGTAACAAA AAAAGCAAGA |
| 2461 | CATTTGTTAC CTTTGAGGAC ATGCACTTGT TATTGAACTA TAGGCGGCTT GACGTCCAAC |
| 2521 | TCATAACAAT CTGGTGCCTG TAAGTATCAC TCATGCACAC ACAATTATTA TATATTAATA |
| 2581 | TGTAGTGTGA AACTCTAATA TGTAGATGTT GTCTGTAGTT TGCAAGATCA CGAGCAGATG |
| 2641 | TCATTATTAT CTGCCGGATC GATGGTCGGT TATCTGAGCC CTATCAAGTT ACAAGAAAAT |
| 2701 | ATGAACAAAT TCGTATTATC AAAGGAAGAT AGAGCAAAGA TAGAGGAAGA CAAAACACCA |
| 2761 | GGATAATTAT GCCATCTATC TTGGTAGATC AATGCTGAGG TATAAATATA GGGATTTTAT |
| 2821 | ATTGGCACCA TACAACATTA GGTAAGCTTG ACTTCATATA CGTATTTCAA ATTATCGTGT |
| 2881 | AAACAATATA CATGTGTCGC TCACTCATTT ATTCATGCAG TGACCATTGG ATTGTTTTTT |
| 2941 | ATATTTATCC CTTCGAAGGG AAGGTGCTTG TCCTAGACTC TTTACATGTT CCTCCCGAGA |
| 3001 | AGTATCAACC ATTCTTGGTT CAATTAGAAA GGTGAGCCAA CATGAAACCA CATGCGTACT |
| 3061 | TATATAAATT AGAGTTTCAA AATAACTTTA GTGATTTAGG TTCGATATCT ACGGGGCATG |
| 3121 | GCGGTTTTAT AAGAAACAAA AGGGACCTGT CGACGCTGCA CGCTCAGATC CTAGGATCCC |
| 3181 | ATTGATGATA CAACACCACT ATCCGGTAAG TTTTCTGAAC ACATTTCATC ATATAAATAA |
| 3241 | TACATAAAGC ATGGCAAATT TAGAATAATC CGTTGCTCAT TATATAGTGC CACAAGCAAC |
| 3301 | CACCTGGATC GGTCTATTGT GGGTACTATG TCTGTGAGTT TATAAGGCAG CGGGGACGTT |
| 3361 | ACGTCAAGGA CAAAAATATG GTAAATAATA TCTATGTATG AAGTTTTCTC ATTAAAGCTG |
| 3421 | CAAAATTATA TATTGAACAT GTGTCAATCA TGCTTTTAAA CTTTATTTTC AGCCGAAAAA |
| 3481 | GCAAGGAAAA GACGTGCCCT TTACACCAAA GACTCTGGAA GATATAGTAG CATACTTGTG |
| 3541 | TGGTTTTATT ATGAGAGAAA TAATTTCAAG TGACAGTGCA TATTTTGATC ATGAGGGCGA |
| 3601 | TTTAGCAAGT GATAAATTTA GAGTGCTGAC AGACATAGCA GGTCTAAATC TGAAGCGAAA |
| 3661 | CGACATGTAA ACATTGTATG GTTGTGCGGA TAACATGCAT TGACGTGTAT ATATATAATT |
| 3721 | TTATGGTTGA TGTTTGATTT GTTTACAATT CTATAATATA TATATGTGGT GTATGTATGA |
| 3781 | TGTTGTGTGT GTATATATAT ATATATATAT ATATATATAT ATATATATAT ATATATATAT |
| 3841 | ATATATATAT AATGTTTAGC ACTGTGTTTG GTGGGAAAAA TTAAAATTTG AAATATATAT |
| 3901 | AAAAAATTAT TTACACAGAC AGTGTAGTGT GAGCTGCCTG TGTAAAAATA CATTTATACA |
| 3961 | GGCGGCTCAC CTTGTCNNNN CAGGCGGTGC TAAAAGCATC TTCACAGGCG GCCAAGCCCA |
| 4021 | CCGCCTGTAC CAGGGGTCAG TACAAAATGG ACCACAGTAC AGGCGGGGCT GTGCGAGCCG |
| 4081 | CCTGTGAAAA CATAATTTTC ACAGGCGGCT CGCACAGCCC CGCCTGTACT GTGGTCCATT |
| 4141 | TTGTACTGAC CCCTGGTACA GGCGGTGGGC TTGGCCGCCT GTGAAGATGC TTTTAGCACC |
| 4201 | GCCTGTAAAA ATGTTTTTTG TAGCAGTGTT T |

(SEQ ID NO:20) or a functional fragment or variant thereof having 75%, 80%, 85%, 90%, 95%, 96%, 97%, 98%, or 99% sequence identity to SEQ ID NO:20. Each N=can be any nucleotide or combination of any 2, 3, 4, or 5 nucleotides.

CACTA elements have been implicated as a mechanism of movement of genes and gene fragments in sorghum (Paterson A H et al. Nature, 457(7229):551-56 (2009)). In some embodiments, a functional Ma1 expression control sequence capable of placing a target gene under photoperiod (short-day) control includes the CACTA element of SEQ ID NO:1 or a functional fragment or variant thereof. For example, in some embodiments, a functional Ma1 expression control sequence capable of placing a target gene under photoperiod (short-day) control includes the nucleic acid sequence:

| | |
|---|---|
| 1 | CACTATAACA CAACATGGCT TTGCCGACAC TTCCAACTAT CGGCAAAGGG TACCTTTACC |
| 61 | GACACTTAAC GTCTCACGAA AGGTTTTGCC GACAATTTTC AAACAGTCGC GGTAGAAGCA |
| 121 | GTTGGCGAAA CTTTTGCCGA CAGTTAAAGG CATCGCCGAC ACATTTTCTG TAGTCAAATG |
| 181 | GCATACCTAC GCCGACAGTT GAACTTTCAC CGACAGTGAA CCCTTTGCCG ACAGTTTGGA |
| 241 | CCTACGCCGA CAGTTTGGAC CTTTTCCGAC AGTTGGTATG TTAGCGAAAC CGTTTCTAGG |
| 301 | GTGTTTCATA AACCATGCCT TGTCCAACAG TAGAAGTGTC GGCAAAACTA TATTGCTAGG |
| 361 | ATGTAGATAC AATTTAAATA TTTTAATAAA TACACATCAC ATTGATTGAG CAAAATCACA |
| 421 | TGG |

(SEQ ID NO:21) or a functional fragment or variant thereof having 75, 80, 85, 90, 95, 96, 97, 98, or 99% sequence identity to SEQ ID NO:21.

In some embodiments, a functional Ma1 expression control sequence capable of placing a target gene under photoperiod (short-day) control includes the nucleic acid sequence:

| | |
|---|---|
| 1 | TTCCACCTGG GCAGGGAAAA CGGTTTATTA TGTTCCTCTT TAATTTATCT ATCGTGGCAC |
| 61 | TATAACACAA CATGGCTTTG CCGACACTTC CAACTATCGG CAAAGGGTAC CTTTGCCGAC |
| 121 | ACTTAACGTC TCACGAAAGG TTTTGCCGAC AATTTTCAAA CAGTCGCGGT AGAAGCAGTC |
| 181 | GGCGAAACTT TTGCCGACAG TTAAAGGAGG ACACATTTTC TGTAGTCAAA TGGGCATGCC |
| 241 | TCCCGCGTTG ACTTTCACCG ACAGTGAACC CTTTGCCGAC AGTTTGGACC TACGCCGACA |
| 301 | GTTTGGATCT TTTCCGACAG TTGGTATGTT AGCGAAACCG TTTCTAGGGT GTTTCATAAA |
| 361 | CCATGCCTTG TCCAACAGTA GAAGTGTCGG CAAAACTATA TTGCAGATAG TAGGGTGTAG |
| 421 | ATACAATTTA AATATTTTAA TAAATACACA TCACATTGAT CGAGCAAAAT CACATGGTCT |
| 481 | GTTTTCACTA AAACTGTCAT AGGTACACTC CAGTACTACC AGTACGTCGC CCGCACATAG |
| 541 | TGGCCAAGGA TTTTACTGCT ACTGTTGATT AACATAAGCA CTTGCGACTT TCCCTAAAAT |
| 601 | CTTTTATAAA ACAACGGCCG CAATAATATT GAACTATTTT TGTTCTAGTA CCAAAATTAG |
| 661 | AATTTGATCC CTCACCTCAT TACATCCATA G |

(SEQ ID NO:22) or a functional fragment or variant thereof having 75, 80, 85, 90, 95, 96, 97, 98, or 99% sequence identity to SEQ ID NO:22.

In some embodiments, a functional Ma1 expression control sequence capable of placing a target gene under photoperiod (short-day) control includes the nucleic acid sequence:

| | |
|---|---|
| 1 | TGGCACTATA ACACAACATG GCTTTGCCGA CACTTCCAAC TATCGGCAAA GGGTACCTTT |
| 61 | GCCGACACTT AACGTCTCAC GAAAGGTTTT GCCGACAATT TTCAAACAGT CGCGGTAGAA |
| 121 | GCAGTCGGCG AAACTTTTGC CGACAGTTAA AGGAGGACAC ATTTTCTGTA GTCAAATGGG |
| 181 | CATGCCTCCC GCGTTGACTT TCACCGACAG TGAACCCTTT GCCGACAGTT TGGACCTACG |
| 241 | CCGACAGTTT GGATCTTTTC CGACAGTTGG TATGTTAGCG AAACCGTTTC TAGGGTGTTT |
| 301 | CATAAACCAT GCCTTGTCCA ACAGTAGAAG TGTCGGCAAA ACTATATTGC AGATAGTAGG |
| 361 | GTGTAGATAC AATTTAAATA TTTTAATAAA TACACATCAC ATTGATCGAG CAAAATCACA |
| 421 | TGG |

(SEQ ID NO:23) or a functional fragment or variant thereof having 75, 80, 85, 90, 95, 96, 97, 98, or 99% sequence identity to SEQ ID NO:23.

In some embodiments, a functional Ma1 expression control sequence capable of placing a target gene under photoperiod (short-day) control includes a functional CAAT box, for example the CAAT box of SEQ ID NO:12 or a functional fragment or variant thereof. In some embodiments, a functional Ma1 expression control sequence capable of placing a target gene under photoperiod control includes the nucleic acid sequence: GCCAAT (SEQ ID NO:24) or a variant thereof, for example a consensus CAAT Box sequence such as GGCCAATCT (SEQ ID NO:25). The CAAT box of a functional Ma1 expression control sequence capable of placing a target gene under photoperiod (short-day) control is typically between 50 and 250 bases upstream of the initial transcription site.

In some embodiments, a functional Ma1 expression control sequence capable of placing a target gene under photoperiod (short-day) control includes the nucleic acid sequence:

| | |
|---|---|
| 1 | TTCTTATTAG TAGTATCTTT TATACTAATT AAGATTCAAT AAAAATTCAC CATGACATCC |
| 61 | CCATTGCCAA GAGAATATTT CGCCGCCCCT CAAAGCAGCC AATAAGGCTT TACTAAAAAG |
| 121 | ACTATCCACG CAGTAGAGAT TTAGTCAAAA TATTCCAATA GCAATTGTTT CCTGCCTGCT |
| 181 | TGACCTTCGT CAGCCACTCA CTGTATAAAT ATCGCACCAC GCCCTTTGCA GGCTTACAGA |
| 241 | GCTTGTATTA CGTACTAACA AGGCACACAC AGTACCCTGT GTTCACCGGC CCTGCACAAA |
| 301 | ACTCAAGCAG TTATTACTAA C |

(SEQ ID NO:26) or a functional fragment or variant thereof having 75, 80, 85, 90, 95, 96, 97, 98, or 99% sequence identity to SEQ ID NO:26.

A polynucleotide having a nucleic acid sequence at least 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% identical to a fragment of SEQ ID NO: 14, 15, 16, 17, 18, 19, 20, 21, 22, 23, 24, 25, and 26 is also disclosed. The Ma1 gene in the day-neutral S. bicolor has a recessive (loss of function) Ma1 allele characterized by one or more mutations or deletions in the 5′UTR relative to the 5′UTR of S. propinquum that results in loss of photoperiod sensitivity. Therefore, the nucleic acids in SEQ ID NO: 14, 15, 16, 17, 18, 19, 20, 21, 22, 23, 24, 25, or 26 can be present in short-day expression control sequences. Therefore, in some embodiments, the photoperiod sensitive Ma1 expression control sequence has 20, 30, 40, 50, 60, 70, 80, 90, 100, 110, 120, 130, 140, 150, 160, 170, 180, 190, 200, 250, 300 or more of the nucleic acids in SEQ ID NO: 14, 15, 16, 17, 18, 19, 20, 21, 22, 23, 24, 25, or 26, and is capable of inducing short-day expression of a target gene.

2. Photoperiod Insensitivity

The expression control sequence of the Ma1 gene in the day-neutral S. bicolor having a recessive (functional) Ma1 allele can be used to induce photoperiod insensitivity of other plant genes. Accordingly, the Ma1 expression control sequences from S. bicolor can be operably linked to a plant gene coding sequence to impart photo-insensitive (i.e., day-neutral) control over the plant gene coding sequence.

In some embodiments, a functional Ma1 expression control sequence capable of placing a target gene under photo-insensitive (day neutral) control has the nucleic acid sequence:

| | |
|---|---|
| 1 | AAAAGAAAAG TGAGCACACC ACGACCTATC ATCAGCTCAT GGTCAGCTCT ACAAACTTAT |
| 61 | AGATTGCATC GAGATCTAAG ACTCAGGTAC AAATCATGTC AACATCTAAT GGTTTAGAAA |
| 121 | ATGAAAAAAG TTTTGAGTTT CAAAATATGA TACTTGAAAT TAACATTTGA ACTTTTTAGC |
| 181 | AAGATCTGAA AATAAAAAAT TCAACTAAAA AATTTATAGA TCATGTTAAC ATTGATATAA |
| 241 | TCGCTTCCAA TCGCCTCCCA TCGCTTCAGC TAGAAAACTT TTTTTCTCGA TTTAATTAAT |
| 301 | GAAATAGTAA TAACGTCATT GTACAAGATT CTTTCAAACC CCAACCCCTA TCATCGACGG |
| 361 | TGAGGGCTCC TATAATATGC ACTAGTGGAC GCCGGGTGGG TGGAACCTAA GAAGATTTTA |
| 421 | AAAAAAAAAT TAAGAAGAAG ATTTTTATCT AACTAACTAT ATATAGTACT TATATCATAC |
| 481 | ACTATACTAT TCAAAATATT ATTTTCACAA TTATGAATTT ACCCTTTTAC TCTTTATTAA |
| 541 | AAAAATATGA ATAAAGAATT ATCACGCCTC TATTTAGGGT CCTAATCCCC ATAATTTAAG |
| 601 | AGGCGATGAG AGGCGATGTG ACATCTATGG CCCACCGACC AAAGACACAA CTATCGCCTC |
| 661 | CCATCACCTT GCTTCTATCG CCTCTCATAG CTTTTCATAT TCTAGGTCCA CCGGCCATAG |
| 721 | ACACACCAAT CGCTTATCAT CGCCTTTTCC AACCATTGTA AAAATATTCA TAATTTTGAT |
| 781 | ATAAAATTTG TCTTCACTTG AGTATGGGAA AAAAATTATA CATAATGTTT TCGTGTGAGA |
| 841 | ATTTACAGGA ATGAACCCTT AAGATGTCCA AATGTAAATG ACCCTATTTA TTAAGAGGAG |
| 901 | CGGATCTATA GGCCTGGCTC TGAAAATGGA TTATGGATTG GAGATACTAA ATTTAAGGGC |
| 961 | CTATCTTCGC ACATAACATC TATAGTTCCT AAATAATTTT TTATTGTAGT AGTAGAACTT |
| 1021 | TTCTCCCTGT AAACCATAAA CCAAGTTGAC GCTGGGCTTT ATTTTGCGAC ACAGAACACC |
| 1081 | AAATTGGTGG CTATGAACTC TTCCACCTGG GCAGGGAAAA CGGTTTATTA TGTTCCTCTT |
| 1141 | TAATTTATCT ATCGTGGTCT GTTTTCACTA AAACTGTCAT ATTGCTACAC TCCAGTACTA |
| 1201 | CCAGTACGTC GCCCGCACAT AGTGGCCAAG GATTTTACTG CTACTGTTGA TTAACATAAG |
| 1261 | CACTTGCGAC TTTCCCTAAC ATCTTTTATA AAACAACGGC CGCAATAATA TTGAACTGTT |
| 1321 | TTTTTCTAGT ACCAAAAATA GAATTTGATC CCTCACCTCA TTACATCCAT AGTAACATGA |
| 1381 | CCAGATATAT ATGGACAGGC CGGGATCACT CGCCAGCAGA TACCCTGAGC GATTCATAAC |
| 1441 | CAGAATTTTT AATTTTTTCT AGTGAAGTGG GGTTCTCCTA GTCCTTTAAC ATTCAAAATT |
| 1501 | TAGTACAAAC TTTCCTTAGT AAATGTCTTC TAGTAAAGAT TTCCTAGTGT TTTGATTTGG |
| 1561 | TAGTGTTTTA TTACTAATTA AAAATATTAG AAGAACTCCA TCATTTTGGT AGTGATTGGT |
| 1621 | TGTTTGGATT AGTCTTCTCA CGTTAGACCT ATATATGCAG GACAACTCAA GCCAGCATAA |
| 1681 | ATATATGAAA TATCTTGGTG TTTGTTTGTC TGACACAGGC AACCGTGTTT GGTATAAATG |
| 1741 | TGTTTTCTTG TTTACGTTTT ACCATCTATA GTCATCTCAA TGTTTATATA GTAGAGACTT |
| 1801 | CATGTTTGTA GTAGATAAGG TAGAGAATTG AGAATATTTT ATTTTTGTGC GACCATCAAT |
| 1861 | TTTATGTAAT CTGCATTGTC TAATGCTTTA TTTGACATTT GAAACTACTT AATTTGACCG |
| 1921 | TTATGCAGGT CCGCATGATC CTATGAAAGC AATTAATTAG TACGGGTACT GCACTACACA |
| 1981 | AGTTTGCTAG TACTATTCTA TTAACCGACC TGTCAATATT ACCTTAAGTT ACTGATTTCA |
| 2041 | ATTAGAATCT AACACATTCA GGAAAAGAAG TTTTCCTTAT TAGTAGTAAC TTTTTATACT |
| 2101 | AATTAAGATT CAATAAAAAT TCACCATGAC ATCCCCATTG CCAAGAGAAT ATTTCGCCGC |
| 2161 | CCCTCAAAGC AGCCAAGGCT TTACTAAAAA GACTATCCAC GCAGTAGAGA TTTAGTCAAA |
| 2221 | ATATTCCAAT AGCAATTGTT TTCTGCCTGC TTGACCTTCG TCAGCCACTC ACTGTATAAA |
| 2281 | TATCGCACCA CGCCCTTTGC AGGCTTACAG AGCTTGTACT ACGTACTAAC AAGGCACACA |
| 2341 | CAATACCCTG TGTTCACCGG CCCTGCACAA AACTCAAGCA GTTATTACTA AC |

(SEQ ID NO:27) or a functional fragment or variant thereof having 75, 80, 85, 90, 95, 96, 97, 98, or 99% sequence identity to SEQ ID NO:27.

In some embodiments, a functional Ma1 expression control sequence capable of placing a target gene under photo-insensitive (day neutral) control has the nucleic acid sequence:

| | |
|---|---|
| 1 | TCCTTATTAG TAGTAACTTT TTATACTAAT TAAGATTCAA TAAAAATTCA CCATGACATC |
| 61 | CCCATTGCCA AGAGAATATT TCGCCGCCCC TCAAAGCAGC CAAGGCTTTA CTAAAAAGAC |
| 121 | TATCCACGCA GTAGAGATTT AGTCAAAATA TTCCAATAGC AATTGTTTTC TGCCTGCTTG |
| 181 | ACCTTCGTCA GCCACTCACT GTATAAATAT CGCACCACGC CCTTTGCAGG CTTACAGAGC |
| 241 | TTGTACTACG TACTAACAAG GCACACACAA TACCCTGTGT TCACCGGCCC TGCACAAAAC |
| 301 | TCAAGCAGTT ATTACTAAC |

(SEQ ID NO:35) or a functional fragment or variant thereof having 75, 80, 85, 90, 95, 96, 97, 98, or 99% sequence identity to SEQ ID NO:35.

A polynucleotide having a nucleic acid sequence at least 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% identical to a fragment of SEQ ID NO:27 or 35 is also disclosed. The Ma1 gene in the day-neutral S. bicolor has a recessive (loss of function) Ma1 allele characterized by one or more mutations or deletions in the 5′UTR relative to the 5′UTR of S. propinquum that results in loss of photoperiod sensitivity. Therefore, in some embodiments, the photo-insensitive Ma1 expression control sequence has 20, 30, 40, 50, 60, 70, 80, 90, 100, 110, 120, 130, 140, 150, 160, 170, 180, 190, 200, 250, 300 or more of the nucleic acids in SEQ ID NO: 27 or 35, and is capable of controlling day-neutral expression of the target gene.

### III. Methods of Modulating Photoperiod Sensitivity

Methods of modulating photoperiod sensitivity and flowering time in sorghum are disclosed. The methods can be used, for example, to increase high biomass production, by extending the growing period.

Methods are also disclosed for modulating photoperiod sensitivity involving operably linking the expression control sequence of a Ma1 gene from a photoperiod sensitive Sorghum variety or cultivar to the endogenous maturity gene in the plant. Methods are disclosed for imposing photoperiod sensitivity on other genes that are not normally controlled by photoperiod by operably linking the expression control sequence of a Ma1 gene from a photoperiod sensitive Sorghum variety or cultivar to the endogenous gene in the plant. Similarly, methods are also disclosed for imposing photoperiod s insensitivity on other genes that are normally controlled by photoperiod by operably linking the expression control sequence of a Ma1 gene from a photoperiod insensitive Sorghum variety or cultivar to the endogenous gene in the plant.

The disclosed method can involve modulating the expression or activity of a Ma1 gene in a plant. Activities of a gene include transcriptional activation of the gene and activities of the resulting encoded protein. The method can involve modulating the activity of a protein encoded by the Maturity gene. Activities of a protein include, for example, transcription, translation, intracellular translocation, secretion, phosphorylation by kinases, cleavage by proteases, homophilic and heterophilic binding to other proteins, ubiquitination.

In some embodiments, the method involves increasing photoperiod sensitivity in a plant. For example, in some embodiments, the method involves introducing to a plant a nucleic acid sequence that promotes photoperiod dependent expression of a functional Ma1 maturity gene. As a result of this method, the transgenic plant preferably has higher photoperiod sensitivity to flowering compared to control (e.g., wild-type) plant of the same species.

In some embodiments, the method involves inhibiting photoperiod sensitivity in a plant. In some embodiments, the method involves engineering a transgenic plant to express the Ma1 under the control of photoperiod insensitive control sequence of Ma1. As a result of this method, the transgenic plant preferably has reduced photoperiod sensitivity to flowering compared to control (e.g., wild-type) plant of the same species.

In some embodiments, the method involves engineering a transgenic plant to inhibit gene expression of the Ma1 gene or translation of the Ma1 protein. In other embodiments, the method involves introducing to the plant a composition that silences gene expression. For example, the composition can include an antisense, RNAi, dsRNA, miRNA, or siRNA that targets the maturity gene in the plant and inhibits translation of the encoded protein. In still other embodiments, the method involves introducing to the plant a composition that binds to the protein encoded by the maturity gene and inhibits one or more of the protein's activities.

In some embodiments, the method involves introducing to the plant or plant cell a nucleic acid sequence that silences expression of the maturity gene in the plant. Preferably, the nucleic acid is operably linked to an expression control sequence. The expression control sequence can be a heterologous control sequence. Selection of this control sequence can be used to select the amount of gene-silencing nucleic acid expressed and therefore control photoperiod sensitivity in the plant. As a result of this method, the transgenic plant preferably has lower photoperiod sensitivity compared to control (e.g., wild-type) plant of the same species. In some embodiments, the nucleic acid can silence a polynucleotide having the nucleic acid sequence SEQ ID NO: 1, 2, 3, 4, 5, 6, 7, 9, 10, 11, 12, 13, 14, 15, 16, 17, 18, 19, 20, 21, 22, 23, 24, 25, 26, 27, 28, 29, 30, 31, 32, 33, 35 or a nucleic acid encoding the polypeptide of SEQ ID NO: 8 or 34, for fragments or variants thereof.

In some embodiments, photoperiod sensitivity can be modulated by elements within the nucleic acid sequence. For instance, as discussed above, wild type short day flowering sorghum contains at least four additional non-coding segments not found in day-neutral sorghum: a segment of about 400 base pairs in the 5′ UTR, a segment of about 4.2 kb in the 5′ UTR, a segment of 3 base pairs in the 5′ UTR, and a segment of 27 base pairs in the second intron of the coding sequence.

Methods of interfering with the non-coding segments can be used to modulate the photoperiod sensitivity of short day plants. Deleting or altering some or all of the non-coding segments or inserting additional nucleotides into the non-coding segments can be effective. Deleting, mutating, or inserting nucleotides in one or more of the Ma1 expression control sequences disclosed herein can decrease the photoperiod sensitivity of a gene or polynucleotide of interest. Therefore, in some embodiments deleting or mutating nucleotides in one or more of these regions of the Ma1 expression control sequence with shift the plant from short-day flowering to day-neutral flowering. For example, in some embodiments insertions, mutations, or deletions are introduced into a polynucleotide having SEQ ID NO: 14, 15, 16, 17, 18, 19, 20, 21, 22, 23, 24, 25, 26, 27, 28, 29, 30, 31, 32, 33, 35 or a functional fragment, variant, or complement thereof to reduce the photoperiod sensitivity of the expression control sequence. In a preferred embodiment, mutations or deletions are introduced into a CAAT box, for example a polynucleotide having the sequence of SEQ ID NO: 23, 24, or 25 or a functional fragment, variant, or complement thereof. The insertions, mutations or deletions can shift the plant from short-day flowering to day-neutral flowering, or make the plant less photoperiod sensitive.

Inhibiting the regulatory function of the non-coding segments can also be used to modulate photoperiod sensitivity. For instance, inhibiting or preventing the interaction of one or more of the non-coding segments with another nucleic acid sequence or protein.

The additional nucleotides can be dependent or independent on a functional copy of the flowering gene. In some forms, one or more of the non-coding segments is insufficient to produce the short day trait alone. However, the combination of one or more of the non-coding segments and a functional copy of the flowering gene can result in a short day flowering plant. The non-coding segments can interact with the gene it resides within. The interaction can be non-linear. This interaction can be based on one or more of the non-coding segments containing a gene regulatory feature that confers the short day sensing mechanism.

In some embodiments, the photoperiod sensitivity of expression control sequences disclosed herein is increased. Deleting, mutating, or inserting nucleotides in one or more of these regions of the Ma1 expression control sequences disclosed herein can increase the photoperiod sensitivity of a gene or polynucleotide of interest. For example, in some embodiments deleting, mutating, or inserting nucleotides in one or more of these regions of the Ma1 expression control sequence with shift the plant from day-neutral flowering to short-day flowering. For example, in some embodiments insertions, mutations or deletions are introduced into a polynucleotide having SEQ ID NO: 14, 15, 16, 17, 18, 19, 20, 21, 22, 23, 24, 25, 26, 27 28, 29, 30, 31, 32, 33, 35 or a functional fragment, variant, or complement thereof to increase the photoperiod sensitivity of the control sequence. In a preferred embodiment, an insertion includes multiple copies of a CAAT box, for example a polynucleotide having the sequence of SEQ ID NO: 23, 24, or 25 or a functional fragment, variant, or complement thereof. In some embodiments the additional CAAT boxes, include, but not limited to one or more copies of SEQ ID NO:23, 24, or 25. The inserted sequences can be added sequentially to the promoter region of the gene or polynucleotide of interest. For example, in some embodiments, one or more CAAT boxes are added beginning between about 50 and 250 nucleotides upstream of the “ATG” start site of a plant gene such as Ma1. The insertions, mutations or deletions can shift the plant from day-neutral flowering to short-day flowering plants, or increase the photoperiod sensitivity of the plant.

In some embodiments, photoperiod sensitivity can be modulated by using the Ma1 control sequences of S. bicolor. For example, in some embodiments, the control sequences of S. bicolor, including by not limited to SEQ ID NO:27 or 35, are inserted upstream of a coding sequence of a gene of interest and cause photoperiod insensitive, or day neutral expression of the gene of interest. In some embodiments the gene of interest is Ma1.

Methods of modifying the photoperiod sensitivity of Ma1 by replacing or supplementing the endogenous control sequences of Ma1 with heterologous control sequences are also disclosed. The expression control sequences of Ma1 can be altered or replaced with an expression control sequence that reduces photosensitivity, but wherein expression of Ma1 is still photoperiod sensitive relative to Ma1 expression in S. bicolor. The expression control sequences of Ma1 can also be altered or replaced with an expression control sequence that increases photosensitivity of Ma1 expression relative to Ma1 expression in S. propinquum. For example, in some embodiments, the expression control sequence of Ma1 is replaced with an expression control sequence from another photoperiod sensitive gene. Cis-regulatory elements in the promoter of photoperiod-responsive genes, coordinated motifs integrating hormones and stresses to photoperiod responses, and photo-responsive genes and their promoters are known in art, and can be used to alter the photosensitivity Ma1, see for example, Mongkolsiriwatana C, Katsetsart J. (Nat. Sci.) 43: 164-177 (2009).

A. Recombinant Plant Gene Expression

Compositions and methods are therefore provided for operably linking plant genes to a Ma1 expression control sequence. Therefore, methods of imposing photoperiod sensitivity or insensitivity on a plant process are disclosed. The methods can involve producing a recombinant nucleic acid molecule that contains a plant gene responsible for the plant process operably linked to an Ma1 expression control sequence, for example a polynucleotide having the sequence of SEQ ID NO: 14, 15, 16, 17, 18, 19, 20, 21, 22, 23, 24, 25, 26, or a functional fragment or variant thereof. The plant process can be naturally photoperiod sensitive, or photoperiod insensitive. In some embodiments a photoperiod sensitive control sequence of Ma1, for example SEQ ID NO: 14, 15, 16, 17, 18, 19, 20, 21, 22, 23, 24, 25, 26, or a functional fragment or variant thereof is operably linked to a plant gene to impart photoperiod sensitive control over the gene. In some embodiments a photoperiod insensitive control sequence of Ma1, for example SEQ ID NO: 27, or a functional fragment or variant thereof is operably linked to a plant gene or coding sequence thereof to impart photoperiod sensitive control over the polypeptide encoded by the gene.

A transgenic plant or transgenic plant cell is also disclosed that has a photoperiod sensitive or insensitive plant process. These plants can contain a plant gene controlling the plant process that is operably linked to a Ma1 expression control sequence, for example a polynucleotide having the sequence of SEQ ID NO: 14, 15, 16, 17, 18, 19, 20, 21, 22, 23, 24, 25, 26, or a functional fragment or variant thereof, as described above.

Nucleic acid vectors are also disclosed that include the Ma1 expression control sequence, for example a polynucleotide having the sequence of SEQ ID NO: 14, 15, 16, 17, 18, 19, 20, 21, 22, 23, 24, 25, 26, or a functional fragment or variant thereof. In some embodiments, the vectors also include an insertion site, such as a multiple cloning site, for insertion of a plant gene of interest. The insertion site can include, for example, one or more restriction enzyme digestion sites for operably linking a gene to the expression control sequence.

Methods of modifying a plant gene to be under photoperiod control are also disclosed. The method generally involves operably linking the plant gene to a functional Ma1 expression control sequence. The Ma1 sequence can in some embodiments be from any Sorghum plant variety or cultivar that is photoperiod sensitive. Likewise, the optimum conditions for photoperiod selectivity can be selected for the plant gene by selecting a Ma1 expression control sequence from a Sorghum variety or cultivar that flowers under the desired photoperiod conditions. Therefore, Sorghum varieties having undesirable photoperiod sensitivity can be optimized by modifying or replacing the expression control sequence of the endogenous Ma1 gene according to the disclosed method.

As an example, SEQ ID NOs: 14, 15, 16, 17, 18, 19, 20, 21, 22, 23, 24, 25, and 26 contain Ma1 expression control sequences from a short-day cultivar of S. propinquum, i.e., flowers when the days are short. This expression control sequence can in some embodiments be used to impose short-day photoperiodic control on other valuable plant processes.

B. Constructs and Vectors

1. Recombinant Expression of Ma1

Vectors and constructs containing a Ma1 gene, or coding sequence, operably linked to an endogenous or heterologous expression control sequence are also disclosed. The constructs can include an expression cassette containing an Ma1 gene or a Ma1 coding, for example SEQ ID NO: 1, 2, 3, 4, 5, 6, 7, 9, 10, 11, 12, 13, 28, 29, 30, 31, 32, 33, or a nucleic acid encoding the amino acid sequence of SEQ ID NO:8 or 34. The expression sequences can be used to cause flowering in plants as described in more detail below.

2. Genes of Interest

Methods of modifying a plant gene, polynucleotide, or coding sequence to be photoperiod sensitive or insensitive are also disclosed. The method generally involves operably linking the polynucleotide to a Ma1 photoperiod sensitive or insensitive expression control sequence to polynucleotide or interest. The polynucleotide of interest can be a coding sequence for example a sequence encoding a polypeptide (with or without introns), or non-coding sequence such as an antisense or inhibitory nucleic acid. In some embodiments the polynucleotide includes a cDNA of a polypeptide of interest. Plant genes and coding sequences that can be engineered to be photoperiod sensitive or insensitive are known in the art, and including, but are not limited to, those gene and coding sequences that influence traits such as germination, flowering, ripening, senescence, and combinations thereof. For example, in some embodiments it is desirable to make more or less photoperiod sensitive, genes or coding sequences that regulate or contribute to remobilization of plant constituents from vegetative tissues to harvested organs; to underground parts such as roots; rhizomes to sustain future regrowth; or combinations thereof.

3. Antisense

Ma1 antisense oligonucleotides are also disclosed. Ma1 antisense oligonucleotides can be used to delay, inhibit, or prevent expression of Ma1 in plants. Antisense molecules are designed to interact with a target nucleic acid molecule through either canonical or non-canonical base pairing. The interaction of the antisense molecule and the target molecule is designed to promote the destruction of the target molecule through, for example, RNAseH mediated RNA-DNA hybrid degradation. Alternatively the antisense molecule is designed to interrupt a processing function that normally would take place on the target molecule, such as transcription or replication. Antisense molecules can be designed based on the sequence of the target molecule, for example Ma1 coding sequences including, but not limited to SEQ ID NO: 1, 2, 3, 4, 5, 6, 7, 9, 10, 11, 12, 13, 28, 29, 30, 31, 32, 33, 35 or a nucleic acid encoding the amino acid sequence of SEQ ID NO:8 or 34. Antisense molecules are known in the art include, but are not limited to, RNA interference (RNAi) and siRNA. Methods of designing antisense molecules directed to a target sequence, for example SEQ ID NO: 1, 2, 3, 4, 5, 6, 7, 9, 10, 11, 12, 13, 28, 29, 30, 31, 32, 33, 35 or a nucleic acid encoding the amino acid sequence of SEQ ID NO:8 or 34 are well also well known in the art. See for example, Elbashir, et al., Methods, 26:199-213 (2002).

The production of siRNA from a vector is more commonly done through the transcription of a short hairpin RNAs (shRNAs). Accordingly, vectors and constructs containing a nucleic acid sequence that silences Ma1 gene expression (e.g., siRNA, RNAi, shRNA) operably linked to a heterologous expression control sequence are also disclosed.

4. Transformation Constructs

Transformation constructs can be engineered such that transformation of the nuclear genome and expression of transgenes from the nuclear genome occurs. Alternatively, transformation constructs can be engineered such that transformation of the plastid genome and expression of the plastid genome occurs.

An exemplary construct contains a nucleic acid sequence containing an Ma1 gene operatively linked in the 5′ to 3′ direction to a promoter that directs transcription of the nucleic acid sequence, and a 3′ polyadenylation signal sequence. Typically, the construct will increase the amount of Ma1 in the plant by at least about 10, 20, 30, 40, 50, 60, 70, 80, 90, or 100 percent.

Another exemplary construct contains a nucleic acid sequence that silences Ma1 gene expression operatively linked in the 5′ to 3′ direction to a promoter that directs transcription of the nucleic acid sequence, and a 3′ polyadenylation signal sequence. Typically, the transcribed nucleic acid sequence can result in at least about 10, 20, 30, 40, 50, 60, 70, 80, 90, or 100 percent inhibition of the Ma1 gene.

Another exemplary construct contains a nucleic acid sequence containing a polynucleotide of interest operatively linked in the 5′ to 3′ direction to a Ma1 expression control sequence that directs transcription of the polynucleotide, and a 3′ polyadenylation signal sequence. The Ma1 expression control sequence can impart photoperiod sensitivity or photoperiod insensitivity to the polynucleotide of interest.

Generally, nucleic acid sequences containing an Ma1 gene, a Ma1 coding sequence, or a nucleic acid sequence that silences an Ma1 gene, are first assembled in expression cassettes behind a suitable promoter expressible in plants. The expression cassettes may also include any further sequences required or selected for the expression of the transgene. Such sequences include, but are not restricted to, transcription terminators, extraneous sequences to enhance expression such as introns, vital sequences, and sequences intended for the targeting of the gene product to specific organelles and cell compartments. In some embodiments the expression cassettes includes a Ma1 expression control sequence discussed above. These expression cassettes can then be easily transferred to the plant transformation vectors. Representative plant transformation vectors are described in plant transformation vector options available (Gene Transfer to Plants (1995), Potrykus, I. and Spangenberg, G. eds. Springer-Verlag Berlin Heidelberg New York; “Transgenic Plants: A Production System for Industrial and Pharmaceutical Proteins” (1996), Owen, M. R. L. and Pen, J. eds. John Wiley & Sons Ltd. England and Methods in Plant Molecular biology-a laboratory course manual (1995), Maliga, P., Klessig, D. F., Cashmore, A. R., Gruissem, W. and Varner, J. E. eds. Cold Spring Laboratory Press, New York).

An additional approach is to use a vector to specifically transform the plant plastid chromosome by homologous recombination (U.S. Pat. No. 5,545,818 to McBride, et al.), in which case it is possible to take advantage of the prokaryotic nature of the plastid genome and insert a number of transgenes as an operon.

The following is a description of various components of typical expression cassettes.

1. Promoters

Plant promoters can be selected to control the expression of the transgene in different plant tissues or organelles, for all of which methods are known to those skilled in the art (Gasser & Fraley, Science 244:1293-99 (1989)). In a preferred embodiment, promoters are selected from those of plant or prokaryotic origin that are known to yield high expression in plastids. In certain embodiments the promoters are inducible. Inducible plant promoters are known in the art.

The transgenes can be inserted into an existing transcription unit (such as, but not limited to, psbA) to generate an operon. However, other insertion sites can be used to add additional expression units as well, such as existing transcription units and existing operons (e.g., atpE, accD). Such methods are described in, for example, U.S. Pat. App. Pub. 2004/0137631, which is incorporated herein by reference in its entirety. For an overview of other insertion sites used for integration of transgenes into the tobacco plastome, see Staub (Staub, J. M., “Expression of Recombinant Proteins via the Plastid Genome,” in: Vinci V A, Parekh S R (eds.) Handbook of Industrial Cell Culture: Mammalian, and Plant Cells, pp. 259-278, Humana Press Inc., Totowa, N.J. (2002)).

In general, the promoter can be from any class I, II or III gene. For example, any of the following plastidial promoters and/or transcription regulation elements can be used for expression in plastids. Sequences can be derived from the same species as that used for transformation. Alternatively, sequences can be derived from other species to decrease homology and to prevent homologous recombination with endogenous sequences.

For instance, the following plastidial promoters can be used for expression in plastids.

PrbcL promoter (Allison L A, Simon L D, Maliga P, EMBO J. 15:2802-2809 (1996); Shiina T, Allison L, Maliga P, Plant Cell 10:1713-1722 (1998));

PpsbA promoter (Agrawal G K, Kato H, Asayama M, Shirai M, Nucleic Acids Research 29:1835-1843 (2001));

Pan 16 promoter (Svab Z, Maliga P, Proc. Natl. Acad. Sci. USA 90:913-917 (1993); Allison L A, Simon L D, Maliga P, EMBO J. 15:2802-2809 (1996));

PaccD promoter (Hajdukiewicz P T J, Allison L A, Maliga P, EMBO J. 16:4041-4048 (1997); WO 97/06250);

PclpP promoter (Hajdukiewicz P T J, Allison L A, Maliga P, EMBO J. 16:4041-4048 (1997); WO 99/46394);

PatpB, Patpl, PpsbB promoters (Hajdukiewicz P T J, Allison L A, Maliga P, EMBO J. 16:4041-4048 (1997));

PrpoB promoter (Liere K, Maliga P, EMBO J. 18:249-257 (1999));

PatpB/E promoter (Kapoor S, Suzuki J Y, Sugiura M, Plant J. 11:327-337 (1997)).

In addition, prokaryotic promoters (such as those from, e.g., E. coli or Synechocystis) or synthetic promoters can also be used.

Promoters vary in their strength, i.e., ability to promote transcription. Depending upon the host cell system utilized, any one of a number of suitable promoters known in the art may be used. For example, for constitutive expression, the CaMV 35S promoter, the rice actin promoter, or the ubiquitin promoter may be used. For example, for regulatable expression, the chemically inducible PR-1 promoter from tobacco or Arabidopsis may be used (see, e.g., U.S. Pat. No. 5,689,044 to Ryals, et al.).

A suitable category of promoters is that which is wound inducible. Numerous promoters have been described which are expressed at wound sites. Preferred promoters of this kind include those described by Stanford et al. Mol. Gen. Genet. 215: 200-208 (1989), Xu et al. Plant Molec. Biol. 22: 573-588 (1993), Logemann et al. Plant Cell 1: 151-158 (1989), Rohrmeier & Lehle, Plant Molec. Biol. 22: 783-792 (1993), Firek et al. Plant Molec. Biol. 22: 129-142 (1993), and Warner et al. Plant J. 3: 191-201 (1993).

Suitable tissue specific expression patterns include green tissue specific, root specific, stem specific, and flower specific. Promoters suitable for expression in green tissue include many which regulate genes involved in photosynthesis, and many of these have been cloned from both monocotyledons and dicotyledons. A suitable promoter is the maize PEPC promoter from the phosphoenol carboxylase gene (Hudspeth & Grula, Plant Molec. Biol. 12: 579-589 (1989)). A suitable promoter for root specific expression is that described by de Framond FEBS 290: 103-106 (1991); EP 0 452 269 to de Framond and a root-specific promoter is that from the T-1 gene. A suitable stem specific promoter is that described in U.S. Pat. No. 5,625,136 and which drives expression of the maize trpA gene.

The promoter can be a relatively weak plant expressible promoter. Thus, the promoter can in some embodiments initiate and control transcription of the operably linked nucleic acids about 10 to about 100 times less efficient that an optimal CaMV35S promoter. Relatively weak plant expressible promoters include the promoters or promoter regions from the opine synthase genes of Agrobacterium spp. such as the promoter or promoter region of the nopaline synthase, the promoter or promoter region of the octopine synthase, the promoter or promoter region of the mannopine synthase, the promoter or promoter region of the agropine synthase and any plant expressible promoter with comparably activity in transcription initation. Other relatively weak plant expressible promoters may be dehiscence zone selective promoters, or promoters expressed predominantly or selectively in dehiscence zone and/or valve margins of fruits, such as the promoters described in WO97/13865.

Cis-regulatory elements from the promoter of photoperiod-responsive genes, coordinated motifs integrating hormones and stresses to photoperiod responses, and the promoters of photo-responsive genes such as those described in Mongkolsiriwatana C, Katsetsart J. (Nat. Sci.) 43: 164-177 (2009), can also be used.

2. Transcriptional Terminators

A variety of transcriptional terminators are available for use in expression cassettes. These are responsible for the termination of transcription beyond the transgene and its correct polyadenylation. Appropriate transcriptional terminators are those that are known to function in plants and include the CaMV 35S terminator, the tm1 terminator, the nopaline synthase terminator and the pea rbcS E9 terminator. These are used in both monocotyledonous and dicotyledonous plants.

At the extreme 3′ end of the transcript, a polyadenylation signal can be engineered. A polyadenylation signal refers to any sequence that can result in polyadenylation of the mRNA in the nucleus prior to export of the mRNA to the cytosol, such as the 3′ region of nopaline synthase (Bevan, M., et al., Nucleic Acids Res., 11, 369-385 (1983)).

3. Sequences for Expression Enhancement or Regulation

Numerous sequences have been found to enhance gene expression from within the transcriptional unit and these sequences can be used in conjunction with the genes to increase their expression in transgenic plants. For example, various intron sequences such as introns of the maize Adhl gene have been shown to enhance expression, particularly in monocotyledonous cells. In addition, a number of non-translated leader sequences derived from viruses are also known to enhance expression, and these are particularly effective in dicotyledonous cells.

4. Coding Sequence Optimization

The coding sequence of the selected gene may be genetically engineered by altering the coding sequence for optimal expression (also referred to herein as “codon optimized”) in the crop species of interest. Methods for modifying coding sequences to achieve optimal expression in a particular crop species are well known (see, e.g. Perlak et al., Proc. Natl. Acad. Sci. USA 88: 3324 (1991); and Koziel et al, Biotechnol. 11: 194 (1993)). Therefore, in some embodiments, the disclosed nucleic acids sequences, or fragments or variants thereof, are genetically engineered for optimal expression in the crop species of interest.

5. Selectable Markers

Genetic constructs may encode a selectable marker to enable selection of plastid transformation events. There are many methods that have been described for the selection of transformed plants [for review see (Miki et al., Journal of Biotechnology, 2004, 107, 193-232) and references incorporated within]. Selectable marker genes that have been used extensively in plants include the neomycin phosphotransferase gene nptII (U.S. Pat. No. 5,034,322, U.S. Pat. No. 5,530,196), hygromycin resistance gene (U.S. Pat. No. 5,668,298), the bar gene encoding resistance to phosphinothricin (U.S. Pat. No. 5,276,268), the expression of aminoglycoside 3″-adenyltransferase (aadA) to confer spectinomycin resistance (U.S. Pat. No. 5,073,675), the use of inhibition resistant 5-enolpyruvyl-3-phosphoshikimate synthetase (U.S. Pat. No. 4,535,060) and methods for producing glyphosate tolerant plants (U.S. Pat. No. 5,463,175; U.S. Pat. No. 7,045,684). Methods of plant selection that do not use antibiotics or herbicides as a selective agent have been previously described and include expression of glucosamine-6-phosphate deaminase to inactive glucosamine in plant selection medium (U.S. Pat. No. 6,444,878) and a positive/negative system that utilizes D-amino acids (Erikson et al., Nat Biotechnol, 2004, 22, 455-8). European Patent Publication No. EP 0 530 129 A1 describes a positive selection system which enables the transformed plants to outgrow the non-transformed lines by expressing a transgene encoding an enzyme that activates an inactive compound added to the growth media. U.S. Pat. No. 5,767,378 describes the use of mannose or xylose for the positive selection of transgenic plants. Methods for positive selection using sorbitol dehydrogenase to convert sorbitol to fructose for plant growth have also been described (WO 2010/102293). Screenable marker genes include the beta-glucuronidase gene (Jefferson et al., 1987, EMBO J. 6: 3901-3907; U.S. Pat. No. 5,268,463) and native or modified green fluorescent protein gene (Cubitt et al., 1995, Trends Biochem. Sci. 20: 448-455; Pan et al., 1996, Plant Physiol. 112: 893-900).

Transformation events can also be selected through visualization of fluorescent proteins such as the fluorescent proteins from the nonbioluminescent Anthozoa species which include DsRed, a red fluorescent protein from the Discosoma genus of coral (Matz et al. (1999), Nat Biotechnol 17: 969-73). An improved version of the DsRed protein has been developed (Bevis and Glick (2002), Nat Biotech 20: 83-87) for reducing aggregation of the protein. Visual selection can also be performed with the yellow fluorescent proteins (YFP) including the variant with accelerated maturation of the signal (Nagai, T. et al. (2002), Nat Biotech 20: 87-90), the blue fluorescent protein, the cyan fluorescent protein, and the green fluorescent protein (Sheen et al. (1995), Plant J 8: 777-84; Davis and Vierstra (1998), Plant Molecular Biology 36: 521-528). A summary of fluorescent proteins can be found in Tzfira et al. (Tzfira et al. (2005), Plant Molecular Biology 57: 503-516) and Verkhusha and Lukyanov (Verkhusha, V. V. and K. A. Lukyanov (2004), Nat Biotech 22: 289-296) whose references are incorporated in entirety. Improved versions of many of the fluorescent proteins have been made for various applications. Use of the improved versions of these proteins or the use of combinations of these proteins for selection of transformants will be obvious to those skilled in the art. It is also practical to simply analyze progeny from transformation events for the presence of the PHB thereby avoiding the use of any selectable marker.

For plastid transformation constructs, a preferred selectable marker is the spectinomycin-resistant allele of the plastid 16S ribosomal RNA gene (Staub J M, Maliga P, Plant Cell 4: 39-45 (1992); Svab Z, Hajdukiewicz P, Maliga P, Proc. Natl. Acad. Sci. USA 87: 8526-8530 (1990)). Selectable markers that have since been successfully used in plastid transformation include the bacterial aadA gene that encodes aminoglycoside 3′-adenyltransferase (AadA) conferring spectinomycin and streptomycin resistance (Svab et al., Proc. Natl. Acad. Sci. USA, 1993, 90, 913-917), nptII that encodes aminoglycoside phosphotransferase for selection on kanamycin (Caner H, Hockenberry T N, Svab Z, Maliga P., Mol. Gen. Genet. 241: 49-56 (1993); Lutz K A, et al., Plant J. 37: 906-913 (2004); Lutz K A, et al., Plant Physiol. 145: 1201-1210 (2007)), aphA6, another aminoglycoside phosphotransferase (Huang F-C, et al, Mol. Genet. Genomics 268: 19-27 (2002)), and chloramphenicol acetyltransferase (Li, W., et al. (2010), Plant Mol Biol, DOI 10.1007/s11103-010-9678-4). Another selection scheme has been reported that uses a chimeric betaine aldehyde dehydrogenase gene (BADH) capable of converting toxic betaine aldehyde to nontoxic glycine betaine (Daniell H, et al., Curr. Genet. 39: 109-116 (2001)).

5. Targeting Sequences

The disclosed vectors and constructs may further include, within the region that encodes the protein to be expressed, one or more nucleotide sequences encoding a targeting sequence. A “targeting” sequence is a nucleotide sequence that encodes an amino acid sequence or motif that directs the encoded protein to a particular cellular compartment, resulting in localization or compartmentalization of the protein. Presence of a targeting amino acid sequence in a protein typically results in translocation of all or part of the targeted protein across an organelle membrane and into the organelle interior. Alternatively, the targeting peptide may direct the targeted protein to remain embedded in the organelle membrane. The “targeting” sequence or region of a targeted protein may contain a string of contiguous amino acids or a group of noncontiguous amino acids. The targeting sequence can be selected to direct the targeted protein to a plant organelle such as a nucleus, a microbody (e.g., a peroxisome, or a specialized version thereof, such as a glyoxysome) an endoplasmic reticulum, an endosome, a vacuole, a plasma membrane, a cell wall, a mitochondria, a chloroplast or a plastid. A chloroplast targeting sequence is any peptide sequence that can target a protein to the chloroplasts or plastids, such as the transit peptide of the small subunit of the alfalfa ribulose-biphosphate carboxylase (Khoudi, et al., Gene, 197:343-351 (1997)). A peroxisomal targeting sequence refers to any peptide sequence, either N-terminal, internal, or C-terminal, that can target a protein to the peroxisomes, such as the plant C-terminal targeting tripeptide SKL (Banjoko, A. & Trelease, R. N. Plant Physiol., 107:1201-1208 (1995); T. P. Wallace et al., “Plant Organellular Targeting Sequences,” in Plant Molecular Biology, Ed. R. Croy, BIOS Scientific Publishers Limited (1993) pp. 287-288, and peroxisomal targeting in plant is shown in M. Volokita, The Plant J., 361-366 (1991)).

Plastid targeting sequences are known in the art and include the chloroplast small subunit of ribulose-1,5-bisphosphate carboxylase (Rubisco) (de Castro Silva Filho et al. Plant Mol. Biol. 30:769-780 (1996); Schnell et al. J. Biol. Chem. 266(5):3335-3342 (1991)); 5-(enolpyruvyl)shikimate-3-phosphate synthase (EPSPS) (Archer et al. J. Bioenerg. Biomemb. 22(6):789-810 (1990)); tryptophan synthase (Zhao et al. J. Biol. Chem. 270(11):6081-6087 (1995)); plastocyanin (Lawrence et al. J. Biol. Chem. 272(33):20357-20363 (1997)); chorismate synthase (Schmidt et al. J. Biol. Chem. 268(36):27447-27457 (1993)); and the light harvesting chlorophyll a/b binding protein (LHBP) (Lamppa et al. J. Biol. Chem. 263:14996-14999 (1988)). See also Von Heijne et al. Plant Mol. Biol. Rep. 9:104-126 (1991); Clark et al. J. Biol. Chem. 264:17544-17550 (1989); Della-Cioppa et al. Plant Physiol. 84:965-968 (1987); Romer et al. Biochem. Biophys. Res. Commun. 196:1414-1421 (1993); and Shah et al. Science 233:478-481 (1986). Alternative plastid targeting signals have also been described in the following: US 2008/0263728; Miras, S. et al. (2002), J Biol Chem 277(49): 47770-8; Miras, S. et al. (2007), J Biol Chem 282: 29482-29492.

6. Plants and Tissues for Transfection

Both dicotyledons (“dicots”) and monocotyledons (“monocots”) can be used in the disclosed positive selection system. Monocot seedlings typically have one cotyledon (seed-leaf), in contrast to the two cotyledons typical of dicots. Eudicots are dicots whose pollen has three apertures (i.e. triaperturate pollen), through one of which the pollen tube emerges during pollination. Eudicots contrast with the so-called ‘primitive’ dicots, such as the magnolia family, which have uniaperturate pollen (i.e. with a single aperture).

Monocots include one of the large divisions of Angiosperm plants (flowering plants with seeds protected within a vessel). They are herbaceous plants with parallel veined leaves and have an embryo with a single cotyledon, as opposed to dicot plants (dicotyledonous), which have an embryo with two cotyledons. Most of the important staple crops of the world, the so-called cereals, such as wheat, barley, rice, maize, sorghum, oats, rye and millet, are monocots. Thus, the plant can be a grass, such as wheat, barley, rice, maize, sorghum, oats, rye and millet.

The plant can therefore be a cereal crop such as wheat, oat, barley, or rice; a forage such as bahiagrass, dallisgrass, kleingrass, guineagrass, reed canarygrass, orchardgrass, ricegrass, foxtail, or vetch; a legume such as soybean, lentil, or chickpea; an oilseed such as canola; a vegetable such as onion or carrot; or a specialty crop such as caraway, hemp, or sesame.

In some embodiments, the plant is a sorghum. For example, the plant can be of the species Sorghum almum, Sorghum amplum, Sorghum angustum, Sorghum arundinaceum, Sorghum bicolor, Sorghum brachypodum, Sorghum bulbosum, Sorghum burmahicum, Sorghum controversum, Sorghum drummondii, Sorghum ecarinatum, Sorghum exstans, Sorghum grande, Sorghum halepense, Sorghum interjectum, Sorghum intrans, Sorghum laxiflorum, Sorghum leiocladum, Sorghum macrospermum, Sorghum matarankense, Sorghum miliaceum, Sorghum nigrum, Sorghum nitidum, Sorghum plumosum, Sorghum propinquum, Sorghum purpureosericeum, Sorghum stipoideum, Sorghum timorense, Sorghum trichocladum, Sorghum versicolor, Sorghum virgatum, or Sorghum vulgare

In some embodiments, the plant is a miscanthus. Thus, the plant can be of the species Miscanthus floridulus, Miscanthus x. giganteus, Miscanthus sacchariflorus (Amur silver-grass), Miscanthus sinensis, Miscanthus tinctorius, or Miscanthus transmorrisonensis.

Additional representative plants useful in the compositions and methods disclosed herein include the Brassica family including sp. napus, rapa, oleracea, nigra, carinata and juncea; industrial oilseeds such as Camelina sativa, Crambe, Jatropha, castor; Arabidopsis thaliana; soybean; cottonseed; sunflower; palm; coconut; rice; safflower; peanut; mustards including Sinapis alba; sugarcane and flax.

Crops harvested as biomass, such as silage corn, alfalfa, switchgrass, or tobacco, also are useful with the methods disclosed herein. Representative tissues for transformation using these vectors include protoplasts, cells, callus tissue, leaf discs, pollen, and meristems.

### IV. Methods of Making Transgenic Plants

A. Plant Transformation Techniques

The transformation of suitable agronomic plant hosts using vectors expressing transgenes can be accomplished with a variety of methods and plant tissues. Representative transformation procedures include Agrobacterium-mediated transformation, biolistics, microinjection, electroporation, polyethylene glycol-mediated protoplast transformation, liposome-mediated transformation, and silicon fiber-mediated transformation (U.S. Pat. No. 5,464,765 to Coffee, et al.; “Gene Transfer to Plants” (Potrykus, et al., eds.) Springer-Verlag Berlin Heidelberg New York (1995); “Transgenic Plants: A Production System for Industrial and Pharmaceutical Proteins” (Owen, et al., eds.) John Wiley & Sons Ltd. England (1996); and “Methods in Plant Molecular Biology: A Laboratory Course Manual” (Maliga et al. eds.) Cold Spring Laboratory Press, New York (1995)).

Plants can be transformed by a number of reported procedures (U.S. Pat. No. 5,015,580 to Christou, et al.; U.S. Pat. No. 5,015,944 to Bubash; U.S. Pat. No. 5,024,944 to Collins, et al.; U.S. Pat. No. 5,322,783 to Tomes et al.; U.S. Pat. No. 5,416,011 to Hinchee et al.; U.S. Pat. No. 5,169,770 to Chee et al.). A number of transformation procedures have been reported for the production of transgenic maize plants including pollen transformation (U.S. Pat. No. 5,629,183 to Saunders et al.), silicon fiber-mediated transformation (U.S. Pat. No. 5,464,765 to Coffee et al.), electroporation of protoplasts (U.S. Pat. No. 5,231,019 Paszkowski et al.; U.S. Pat. No. 5,472,869 to Krzyzek et al.; U.S. Pat. No. 5,384,253 to Krzyzek et al.), gene gun (U.S. Pat. No. 5,538,877 to Lundquist et al. and U.S. Pat. No. 5,538,880 to Lundquist et al.), and Agrobacterium-mediated transformation (EP 0 604 662 A1 and WO 94/00977 both to Hiei Yukou et al.). The Agrobacterium-mediated procedure is particularly preferred as single integration events of the transgene constructs are more readily obtained using this procedure which greatly facilitates subsequent plant breeding. Cotton can be transformed by particle bombardment (U.S. Pat. No. 5,004,863 to Umbeck and U.S. Pat. No. 5,159,135 to Umbeck). Sunflower can be transformed using a combination of particle bombardment and Agrobacterium infection (EP 0 486 233 A2 to Bidney, Dennis; U.S. Pat. No. 5,030,572 to Power et al.). Flax can be transformed by either particle bombardment or Agrobacterium-mediated transformation. Switchgrass can be transformed using either biolistic or Agrobacterium mediated methods (Richards et al. Plant Cell Rep. 20: 48-54 (2001); Somleva et al. Crop Science 42: 2080-2087 (2002)). Methods for sugarcane transformation have also been described (Franks & Birch Aust. J. Plant Physiol. 18, 471-480 (1991); WO 2002/037951 to Elliott, Adrian, Ross et al.).

Recombinase technologies which are useful in practicing the current invention include the cre-lox, FLP/FRT and Gin systems. Methods by which these technologies can be used for the purpose described herein are described for example in (U.S. Pat. No. 5,527,695 to Hodges et al.; Dale and Ow, Proc. Natl. Acad. Sci. USA, 88:10558-10562 (1991); Medberry et al., Nucleic Acids Res., 23: 485-490 (1995)).

Engineered minichromosomes can also be used to express one or more genes in plant cells. Cloned telomeric repeats introduced into cells may truncate the distal portion of a chromosome by the formation of a new telomere at the integration site. Using this method, a vector for gene transfer can be prepared by trimming off the arms of a natural plant chromosome and adding an insertion site for large inserts (Yu et al., Proc Natl Acad Sci USA, 103:17331-6 (2006); Yu et al., Proc Natl Acad Sci USA, 104:8924-9 (2007)). The utility of engineered minichromosome platforms has been shown using Cre/lox and FRT/FLP site-specific recombination systems on a maize minichromosome where the ability to undergo recombination was demonstrated (Yu et al., Proc Natl Acad Sci USA, 103:17331-6 (2006); Yu et al., Proc Natl Acad Sci U S A, 104:8924-9 (2007)). Such technologies could be applied to minichromosomes, for example, to add genes to an engineered plant. Site specific recombination systems have also been demonstrated to be valuable tools for marker gene removal (Kerbach, S. et al., Theor. Appl. Genet. 111:1608-1616 (2005)), gene targeting (Chawla, R. et al., Plant Biotechnol. J, 4:209-218 (2006); Choi, S. et al., Nucleic Acids Res., 28, E19 (2000); Srivastava V & Ow D W, Plant Mol. Biol. 46:561-566 (2001); Lyznik L A et al., Nucleic Acids Res., 21: 969-975 (1993)) and gene conversion (Djukanovic V et al., Plant Biotechnol J., 4:345-357 (2006).

An alternative approach to chromosome engineering in plants involves in vivo assembly of autonomous plant minichromosomes (Carlson et al., PLoS Genet., 3:1965-74 (2007). Plant cells can be transformed with centromeric sequences and screened for plants that have assembled autonomous chromosomes de novo. Useful constructs combine a selectable marker gene with genomic DNA fragments containing centromeric satellite and retroelement sequences and/or other repeats.

Another approach useful to the described invention is Engineered Trait Loci (“ETL”) technology (U.S. Pat. No. 6,077,697; US Patent Application 2006/0143732). This system targets DNA to a heterochromatic region of plant chromosomes, such as the pericentric heterochromatin, in the short arm of acrocentric chromosomes. Targeting sequences may include ribosomal DNA (rDNA) or lambda phage DNA. The pericentric rDNA region supports stable insertion, low recombination, and high levels of gene expression. This technology is also useful for stacking of multiple traits in a plant (US Patent Application 2006/0246586).

Zinc-finger nucleases (ZFNs) are also useful for practicing the invention in that they allow double strand DNA cleavage at specific sites in plant chromosomes such that targeted gene insertion or deletion can be performed (Shukla et al., Nature, (2009); Townsend et al., Nature, (2009).

Following transformation by any one of the methods described above, the following procedures can, for example, be used to obtain a transformed plant expressing the transgenes: select the plant cells that have been transformed on a selective medium, regenerate the plant cells that have been transformed to produce differentiated plants, select transformed plants expressing the transgene producing the desired level of desired polypeptide(s) in the desired tissue and cellular location.

Transformation techniques for dicotyledons are well known in the art and include Agrobacterium-based techniques and techniques that do not require Agrobacterium. Non-Agrobacterium techniques involve the uptake of heterologous genetic material directly by protoplasts or cells. This is accomplished by PEG or electroporation mediated uptake, particle bombardment-mediated delivery, or microinjection. In each case the transformed cells may be regenerated to whole plants using standard techniques known in the art.

Transformation of most monocotyledon species has now become somewhat routine. Preferred techniques include direct gene transfer into protoplasts using PEG or electroporation techniques, particle bombardment into callus tissue or organized structures, as well as Agrobacterium-mediated transformation.

Plants from transformation events are grown, propagated and bred to yield progeny with the desired trait, and seeds are obtained with the desired trait, using processes well known in the art.

B. Plastid Transformation

In another embodiment the transgene is directly transformed into the plastid genome. Plastid transformation technology is extensively described in U.S. Pat. No. 5,451,513 to Maliga et al., U.S. Pat. No. 5,545,817 to McBride et al., and U.S. Pat. No. 5,545,818 to McBride et al., in PCT application no. WO 95/16783 to McBride et al., and in McBride et al. Proc. Natl. Acad. Sci. USA 91, 7301-7305 (1994). The basic technique for chloroplast transformation involves introducing regions of cloned plastid DNA flanking a selectable marker together with the gene of interest into a suitable target tissue, e.g., using biolistics or protoplast transformation (e.g., calcium chloride or PEG mediated transformation). The 1 to 1.5 kb flanking regions, termed targeting sequences, facilitate homologous recombination with the plastid genome and thus allow the replacement or modification of specific regions of the plastome. Suitable plastids that can be transfected include, but are not limited to, chloroplasts, etioplasts, chromoplasts, leucoplasts, amyloplasts, proplastids, statoliths, elaioplasts, proteinoplasts and combinations thereof

C. Methods for Reproducing Transgenic Plants

Following transformation by any one of the methods described above, the following procedures can be used to obtain a transformed plant expressing the transgenes: select the plant cells that have been transformed on a selective medium; regenerate the plant cells that have been transformed to produce differentiated plants; select transformed plants expressing the transgene producing the desired level of desired polypeptide(s) in the desired tissue and cellular location.

In plastid transformation procedures, further rounds of regeneration of plants from explants of a transformed plant or tissue can be performed to increase the number of transgenic plastids such that the transformed plant reaches a state of homoplasmy (all plastids contain uniform plastomes containing transgene insert).

The cells that have been transformed may be grown into plants in accordance with conventional techniques. See, for example, McCormick et al. Plant Cell Reports 5:81-84 (1986). These plants may then be grown, and either pollinated with the same transformed variety or different varieties, and the resulting hybrid having constitutive expression of the desired phenotypic characteristic identified. Two or more generations may be grown to ensure that constitutive expression of the desired phenotypic characteristic is stably maintained and inherited and then seeds harvested to ensure constitutive expression of the desired phenotypic characteristic has been achieved.

In some scenarios, it may be advantageous to insert a multi-gene pathway into the plant by crossing of lines containing portions of the pathway to produce hybrid plants in which the entire pathway has been reconstructed. This is especially the case when high levels of product in a seed compromises the ability of the seed to germinate or the resulting seedling to survive under normal soil growth conditions. Hybrid lines can be created by crossing a line containing one or more PHB genes with a line containing the other gene(s) needed to complete the PHB biosynthetic pathway. Use of lines that possess cytoplasmic male sterility (Esser, K. et al., 2006, Progress in Botany, Springer Berlin Heidelberg. 67, 31-52) with the appropriate maintainer and restorer lines allows these hybrid lines to be produced efficiently. Cytoplasmic male sterility systems are already available for some Brassicaceae species (Esser, K. et al., 2006, Progress in Botany, Springer Berlin Heidelberg. 67, 31-52). These Brassicaceae species can be used as gene sources to produce cytoplasmic male sterility systems for other oilseeds of interest such as Camelina.

### V. Screening Methods

Methods are also provided for identifying treatments, such as chemical treatments, that can modify photoperiod sensitivity in a plant.

In some embodiments, the method involves administering a candidate agent to a transgenic plant disclosed herein and comparing the effect of the administration on photoperiod sensitivity in the plant to a control. For example, the purpose of the method can be to identify an agent that causes the transgenic plant to delay or prevent flowering.

In some embodiments, the method involves contacting cells expressing an Ma1 gene disclosed herein with a candidate agent, monitoring the effect of the candidate agent on Ma1 gene expression, and comparing the effect of the candidate agent on Ma1 gene expression to a control. For example, the purpose of the method can be to identify an agent that promotes Ma1 gene expression. In these embodiments, an increase in Ma1 gene expression would identify an agent that could be used to increase photoperiod sensitivity. Likewise, the purpose of the method can be to identify an agent that inhibits Ma1 gene expression. In these embodiments, a decrease in Ma1 gene expression would identify an agent that could be used to reduce photoperiod sensitivity.

Ma1 gene expression can be detected using routine methods, such as immunodetection methods. The methods can be cell-based or cell-free assays. The steps of various useful immunodetection methods have been described in the scientific literature, such as, e.g., Maggio et al., Enzyme-Immunoassay, (1987) and Nakamura, et al., Enzyme Immunoassays: Heterogeneous and Homogeneous Systems, Handbook of Experimental Immunology, Vol. 1: Immunochemistry, 27.1-27.20 (1986), each of which is incorporated herein by reference in its entirety and specifically for its teaching regarding immunodetection methods. Immunoassays, in their most simple and direct sense, are binding assays involving binding between antibodies and antigen. Many types and formats of immunoassays are known and all are suitable for detecting the disclosed biomarkers. Examples of immunoassays are enzyme linked immunosorbent assays (ELISAs), radioimmunoassays (RIA), radioimmune precipitation assays (RIPA), immunobead capture assays, Western blotting, dot blotting, gel-shift assays, Flow cytometry, protein arrays, multiplexed bead arrays, magnetic capture, in vivo imaging, fluorescence resonance energy transfer (FRET), and fluorescence recovery/localization after photobleaching (FRAP/FLAP).

In some embodiments, a reporter construct, such as a fluorochrome or enzyme, is operably linked to an Ma1 expression control sequence. In these embodiments, the purpose of the method can be to identify an agent that modulates activation of the Ma1 expression control sequence by detecting the affect of a candidate agent on reporter expression.

In general, candidate agents can be identified from large libraries of natural products or synthetic (or semi-synthetic) extracts or chemical libraries according to methods known in the art. Those skilled in the field of drug discovery and development will understand that the precise source of test extracts or compounds is not critical to the disclosed screening procedure. Accordingly, virtually any number of chemical extracts or compounds can be screened using the exemplary methods described herein. Examples of such extracts or compounds include, but are not limited to, plant-, fungal-, prokaryotic- or animal-based extracts, fermentation broths, and synthetic compounds, as well as modification of existing compounds. Numerous methods are also available for generating random or directed synthesis (e.g., semi-synthesis or total synthesis) of any number of chemical compounds.

Synthetic compound libraries are commercially available, e.g., from Brandon Associates (Merrimack, N.H.) and Aldrich Chemical (Milwaukee, Wis.). Alternatively, libraries of natural compounds in the form of bacterial, fungal, plant, and animal extracts are commercially available from a number of sources, including Biotics (Sussex, UK), Xenova (Slough, UK), Harbor Branch Oceangraphics Institute (Ft. Pierce, Fla.), and PharmaMar, U.S.A. (Cambridge, Mass.). In addition, natural and synthetically produced libraries are produced, if desired, according to methods known in the art, e.g., by standard extraction and fractionation methods. Furthermore, if desired, any library or compound is readily modified using standard chemical, physical, or biochemical methods.

When a crude extract is found to have a desired activity, further fractionation of the positive lead can be used to isolate chemical constituents responsible for the observed effect. Thus, the goal of the extraction, fractionation, and purification process is the careful characterization and identification of a chemical entity within the crude extract having the activity. The same assays described herein for the detection of activities in mixtures of compounds can be used to purify the active component and to test derivatives thereof. Methods of fractionation and purification of such heterogenous extracts are known in the art. If desired, compounds shown to be useful agents for treatment are chemically modified according to methods known in the art. Compounds identified as being of therapeutic value may be subsequently analyzed using animal models for diseases or conditions, such as those disclosed herein.

Candidate agents encompass numerous chemical classes, but are most often organic molecules, e.g., small organic compounds having a molecular weight of more than 100 and less than about 2,500 daltons. Candidate agents comprise functional groups necessary for structural interaction with proteins, particularly hydrogen bonding, and typically include at least an amine, carbonyl, hydroxyl or carboxyl group, for example, at least two of the functional chemical groups. The candidate agents often comprise cyclical carbon or heterocyclic structures and/or aromatic or polyaromatic structures substituted with one or more of the above functional groups. Candidate agents are also found among biomolecules including peptides, saccharides, fatty acids, steroids, purines, pyrimidines, derivatives, structural analogs or combinations thereof. In a further embodiment, candidate agents are peptides.

### VI. Methods of Identifying Photoperiod Sensitivity Genes in Related Plants

Methods are also provided for identifying genes that control photoperiod sensitivity in other plants. Therefore, methods for identifying maturity gene orthologues in plants are provided. The methods generally involve using the gene sequences for Ma1 in S. bicolor or S. propinquum disclosed herein.

In preferred embodiments, the plant is closely related to Sorghum bicolor. Thus, in some embodiments, the plant is a Sorghum, Miscanthus, or Saccharum. In some embodiments, the method involves scanning the genetic sequences of a plant for genes that are orthologous to Ma1.

In some embodiments, the method involves conducting a BLAST search of plant genomes for genes having the highest nucleic acid sequence identity to that of Ma1 in S. bicolor or S. propinquum. For example, the orthologous gene can have 10%, 15%, 20%, 25%, 30%, 35%, 40%, 45%, 50%, 55%, 60%, 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to the nucleic acid sequence SEQ ID NO: 1, 2, 3, 4, 5, 6, 7, 9, 10, 11, 12, 13, 28, 29, 30, 31, 32, 33, or a nucleic acid encoding the amino acid sequence of SEQ ID NO:8 or 34, or a fragment or variant thereof.

### VII. Methods of Genotyping Photoperiod Sensitive Flowering

A. Haplotypes

The sequences disclosed herein can be used to screen for photoperiod sensitive flowering in plants. For example, the genotype of one or more insertions, deletions, and polymorphisms in or around S. bicolor Ma1 relative to S. propinquum Ma1, and can be used to phenotype a plant as photoperiod sensitive (i.e., having the S. propinquum genotype) or photoperiod insensitive (i.e., having the S. bicolor). For example, deletions, insertions, and polymorphisms can be determined by comparing SEQ ID NO: 1, 3, or 5 of S. propinquum Ma1 to SEQ ID NO: 9 or 12 of S. bicolor using global sequence alignment tools, and include, but are not limited to the insertions, deletions, and polymorphisms specifically disclosed above and in FIG. 3A below.

For example, the exons of short-day S. propinquum and day neutral S. bicolor differ by five synonymous mutations: C->T at position 47; C->T at position 126; A->G at position 159; T->G at position 351; and A->C at position 543 of SEQ ID NO:7 (S. propinquum) relative to SEQ ID NO:11 (S. bicolor). These single nucleotide polymorphisms (SNPs) within the Ma1 gene locus can serve as a haplotype for photoperiod sensitivity. As used herein, the term “haplotype” refers to the allelic pattern of a group of (usually contiguous) DNA markers or other polymorphic loci along an individual chromosome or double helical DNA segment.

Having three, four or five of the S. propinquum SNPs can be diagnostic of a photoperiod sensitive plant (i.e., short day flowering), while having three, four or five of the S. bicolor SNPs can be diagnostic of a photoperiod insensitive plant (i.e., day-neutral flowering). A plant is photoperiod sensitive plant (i.e., short day flowering) when it has all five S. propinquum SNPs. A plant is photoperiod insensitive plant (i.e., day-neutral flowering), when it has all five S. bicolor SNPs. For example, C:C:A:T:C relative to positions 47:126:159:351:543 of SEQ ID NO:7 is indicative of a photoperiod sensitive (short day flowering) plant, while T:T:G:G:C relative to positions 47:126:159:351:543 of SEQ ID NO:11 is indicative of a photoperiod insensitive (day-neutral flowering) plant.

In some embodiments, there is a correlation between the number of S. propinquum SNPs and level of photoperiod sensitivity. For example, an increasing number of S. propinquum SNPs relative to S. bicolor SNPs is correlated with increasing photoperiod sensitivity.

As described in more detail below, it is understood that genomic DNA will typically be used for determining the SNP genotype of a plant of interest. Methods of aligning sequences are known in the art, and described herein. One of skill in the art can readily identify the positions of the above-disclosed SNPs within genomic sequences, including but not limited to those disclosed herein, such as SEQ ID NO: 1, 2, 3, 4, 5, 6, 9, 10, 12, 13, or a nucleic acid encoding the amino acid sequence of SEQ ID NO:8 or 34, or variants, fragments, homologs, or orthologs thereof, by aligning the sequence of SEQ ID NO:7 or 11 to the genomic sequence.

Increased height naturally confers a competitive advantage in light interception. As discussed in the Examples below, favorable alleles at different genes that conferred both optimal height and flowering time to the same progeny by virtue of the suppressed recombination in this genomic region, might have become fixed more quickly than independently-segregating alleles. Accordingly, the S. propinquum haplotype of C:C:A:T:C at positions 47:126:159:351:543 of SEQ ID NO:7 is diagnostic of increased height relative to the S. bicolor haplotype of T:T:G:G:C at positions 47:126:159:351:543 of SEQ ID NO:11.

B. Methods for Detecting SNPs and Haplotypes

The process of determining which specific nucleotide (i.e., allele) is present at each of one or more SNP positions, such as a disclosed SNP position in the Ma1 gene locus, is referred to as SNP genotyping. Methods for SNP genotyping are generally known in the art (Chen et al., Pharmacogenomics J., 3(2):77-96 (2003); Kwok, et al., Curr. Issues Mol. Biol., 5(2):43-60 (2003); Shi, Am. J. Pharmacogenomics, 2(3):197-205 (2002); and Kwok, Annu. Rev. Genomics Hum. Genet., 2:235-58 (2001)).

SNP genotyping can include the steps of collecting a biological sample from a plant, isolating genomic DNA from the cells of the sample, contacting the nucleic acids with one or more primers which specifically hybridize to a region of the isolated nucleic acid containing a target SNP under conditions such that hybridization and amplification of the target nucleic acid region occurs, and determining the nucleotide present at the SNP position of interest, or, in some assays, detecting the presence or absence of an amplification product (assays can be designed so that hybridization and/or amplification will only occur if a particular SNP allele is present or absent). In some assays, the size of the amplification product is detected and compared to the length of a control sample; for example, deletions and insertions can be detected by a change in size of the amplified product compared to a normal genotype.

The neighboring sequence can be used to design SNP detection reagents such as oligonucleotide probes and primers. In some embodiment probe or primers are designed based on the cDNA of S. propinquum (SEQ ID NO:7), or S. bicolor (SEQ ID NO:11), In some embodiments, it may desirable for the probe or primer to bind non-coding regions of the Ma1 gene. Accordingly, one of skill in the art can map the above disclosed haplotype to the genomic sequence of Ma1, such as SEQ ID NO:1, 2, 3, 4, 5, or 6 of S. propinquum, or SEQ ID NO: 9, 10, 12, or 13 of S. bicolor for the purpose of designing the SNP probes or primers.

Common SNP genotyping methods include, but are not limited to, TaqMan assays, molecular beacon assays, nucleic acid arrays, allele-specific primer extension, allele-specific PCR, arrayed primer extension, homogeneous primer extension assays, primer extension with detection by mass spectrometry, pyrosequencing, multiplex primer extension sorted on genetic arrays, ligation with rolling circle amplification, homogeneous ligation, multiplex ligation reaction sorted on genetic arrays, restriction-fragment length polymorphism, single base extension-tag assays, and the Invader assay. Such methods may be used in combination with detection mechanisms such as, for example, luminescence or chemiluminescence detection, fluorescence detection, time-resolved fluorescence detection, fluorescence resonance energy transfer, fluorescence polarization, mass spectrometry, and electrical detection.

SNPs can be scored by direct DNA sequencing. A variety of automated sequencing procedures can be utilized, including sequencing by mass spectrometry. Methods for amplifying DNA fragments and sequencing them are well known in the art.

Other suitable methods for detecting polymorphisms include methods in which protection from cleavage agents is used to detect mismatched bases in RNA/RNA or RNA/DNA duplexes (Myers et al., Science, 230:1242 (1985); Cotton, et al., PNAS, 85:4397 (1988); and Saleeba, et al., Meth. Enzymol., 217:286-295 (1992)), comparison of the electrophoretic mobility of variant and wild type nucleic acid molecules (Orita et al., PNAS, 86:2766 (1989); Cotton, et al, Mutat. Res., 285:125-144 (1993); and Hayashi, et al., Genet. Anal. Tech. Appl., 9:73-79 (1992)), and assaying the movement of polymorphic or wild-type fragments in polyacrylamide gels containing a gradient of denaturant using denaturing gradient gel electrophoresis (DGGE) (Myers et al., Nature, 313:495 (1985)). Sequence variations at specific locations can also be assessed by nuclease protection assays such as RNase and S1 protection or chemical cleavage methods.

In one embodiment, SNP genotyping is performed using the TaqMan® assay, which is also known as the 5′ nuclease assay. The TaqMan® assay detects the accumulation of a specific amplified product during PCR. The TaqMan® assay utilizes an oligonucleotide probe labeled with a fluorescent reporter dye and a quencher dye. The reporter dye is excited by irradiation at an appropriate wavelength, it transfers energy to the quencher dye in the same probe via a process called fluorescence resonance energy transfer (FRET). When attached to the probe, the excited reporter dye does not emit a signal. The proximity of the quencher dye to the reporter dye in the intact probe maintains a reduced fluorescence for the reporter. The reporter dye and quencher dye may be at the 5′-most and the 3′-most ends, respectively, or vice versa. Alternatively, the reporter dye may be at the 5′- or 3′-most end while the quencher dye is attached to an internal nucleotide, or vice versa. In yet another embodiment, both the reporter and the quencher may be attached to internal nucleotides at a distance from each other such that fluorescence of the reporter is reduced.

During PCR, the 5′ nuclease activity of DNA polymerase cleaves the probe, thereby separating the reporter dye and the quencher dye and resulting in increased fluorescence of the reporter. Accumulation of PCR product is detected directly by monitoring the increase in fluorescence of the reporter dye. The DNA polymerase cleaves the probe between the reporter dye and the quencher dye only if the probe hybridizes to the target SNP-containing template which is amplified during PCR, and the probe is designed to hybridize to the target SNP site only if a particular SNP allele is present.

Another method for genotyping SNPs is the use of two oligonucleotide probes in an OLA (U.S. Pat. No. 4,988,617). In this method, one probe hybridizes to a segment of a target nucleic acid with its 3′-most end aligned with the SNP site. A second probe hybridizes to an adjacent segment of the target nucleic acid molecule directly 3′ to the first probe. The two juxtaposed probes hybridize to the target nucleic acid molecule, and are ligated in the presence of a linking agent such as a ligase if there is perfect complementarity between the 3′ most nucleotide of the first probe with the SNP site. If there is a mismatch, ligation would not occur. After the reaction, the ligated probes are separated from the target nucleic acid molecule, and detected as indicators of the presence of a SNP.

Another method for SNP genotyping is based on mass spectrometry. Mass spectrometry takes advantage of the unique mass of each of the four nucleotides of DNA. SNPs can be unambiguously genotyped by mass spectrometry by measuring the differences in the mass of nucleic acids having alternative SNP alleles. MALDI-TOF (Matrix Assisted Laser Desorption Ionization—Time of Flight) mass spectrometry technology is useful for extremely precise determinations of molecular mass, such as SNPs. Numerous approaches to SNP analysis have been developed based on mass spectrometry. Exemplary mass spectrometry-based methods of SNP genotyping include primer extension assays, which can also be utilized in combination with other approaches, such as traditional gel-based formats and microarrays.

Typically, the primer extension assay involves designing and annealing a primer to a template PCR amplicon upstream (5′) from a target SNP position. A mix of dideoxynucleotide triphosphates (ddNTPs) and/or deoxynucleotide triphosphates (dNTPs) are added to a reaction mixture containing template (e.g., a SNP-containing nucleic acid molecule which has typically been amplified, such as by PCR), primer, and DNA polymerase. Extension of the primer terminates at the first position in the template where a nucleotide complementary to one of the ddNTPs in the mix occurs. The primer can be either immediately adjacent (i.e., the nucleotide at the 3′ end of the primer hybridizes to the nucleotide next to the target SNP site) or two or more nucleotides removed from the SNP position. If the primer is several nucleotides removed from the target SNP position, the only limitation is that the template sequence between the 3′ end of the primer and the SNP position cannot contain a nucleotide of the same type as the one to be detected, or this will cause premature termination of the extension primer. Alternatively, if all four ddNTPs alone, with no dNTPs, are added to the reaction mixture, the primer will always be extended by only one nucleotide, corresponding to the target SNP position. In this instance, primers are designed to bind one nucleotide upstream from the SNP position (i.e., the nucleotide at the 3′ end of the primer hybridizes to the nucleotide that is immediately adjacent to the target SNP site on the 5′ side of the target SNP site). Extension by only one nucleotide is preferable, as it minimizes the overall mass of the extended primer, thereby increasing the resolution of mass differences between alternative SNP nucleotides. Furthermore, mass-tagged ddNTPs can be employed in the primer extension reactions in place of unmodified ddNTPs. This increases the mass difference between primers extended with these ddNTPs, thereby providing increased sensitivity and accuracy, and is particularly useful for typing heterozygous base positions. Mass-tagging also alleviates the need for intensive sample-preparation procedures and decreases the necessary resolving power of the mass spectrometer. The extended primers can then be purified and analyzed by MALDI-TOF mass spectrometry to determine the identity of the nucleotide present at the target SNP position.

Other methods that can be used to genotype the SNPs include single-strand conformational polymorphism (SSCP), and denaturing gradient gel electrophoresis (DGGE). SSCP identifies base differences by alteration in electrophoretic migration of single stranded PCR products. Single-stranded PCR products can be generated by heating or otherwise denaturing double stranded PCR products. Single-stranded nucleic acids may refold or form secondary structures that are partially dependent on the base sequence. The different electrophoretic mobilities of single-stranded amplification products are related to base-sequence differences at SNP positions. DGGE differentiates SNP alleles based on the different sequence-dependent stabilities and melting properties inherent in polymorphic DNA and the corresponding differences in electrophoretic migration patterns in a denaturing gradient gel.

Sequence-specific ribozymes (U.S. Pat. No. 5,498,531) can also be used to score SNPs based on the development or loss of a ribozyme cleavage site. Perfectly matched sequences can be distinguished from mismatched sequences by nuclease cleavage digestion assays or by differences in melting temperature. If the SNP affects a restriction enzyme cleavage site, the SNP can be identified by alterations in restriction enzyme digestion patterns, and the corresponding changes in nucleic acid fragment lengths determined by gel electrophoresis.

C. SNP Detection Kits

Detection reagents can be developed and used to assay the disclosed SNPs individually or in combination, and such detection reagents can be readily incorporated into a kit or system format. The terms “kits” and “systems”, as used herein in the context of SNP detection reagents, are intended to refer to such things as combinations of multiple SNP detection reagents, or one or more SNP detection reagents in combination with one or more other types of elements or components (e.g., other types of biochemical reagents, containers, packages such as packaging intended for commercial sale, substrates to which SNP detection reagents are attached, electronic hardware components, etc.). SNP detection kits and systems, including but not limited to, packaged probe and primer sets (e.g., TaqMan probe/primer sets), arrays/microarrays of nucleic acid molecules, and beads that contain one or more probes, primers, or other detection reagents for detecting one or more of the disclosed SNPs are provided. The kits/systems can optionally include various electronic hardware components; for example, arrays (“DNA chips”) and microfluidic systems (“lab-on-a-chip” systems) provided by various manufacturers typically comprise hardware components. Other kits/systems (e.g., probe/primer sets) may not include electronic hardware components, but may be comprised of, for example, one or more SNP detection reagents (along with, optionally, other biochemical reagents) packaged in one or more containers.

In some embodiments, a SNP detection kit typically contains one or more detection reagents and other components (e.g., a buffer, enzymes such as DNA polymerases or ligases, chain extension nucleotides such as deoxynucleotide triphosphates, and in the case of Sanger-type DNA sequencing reactions, chain terminating nucleotides, positive control sequences, negative control sequences, and the like) necessary to carry out an assay or reaction, such as amplification and/or detection of a SNP-containing nucleic acid molecule. A kit may further contain means for determining the amount of a target nucleic acid, and means for comparing the amount with a standard, and can comprise instructions for using the kit to detect the SNP-containing nucleic acid molecule of interest. In one embodiment, kits are provided which contain the necessary reagents to carry out one or more assays to detect one or more of the disclosed SNPs. In an exemplary embodiment, SNP detection kits/systems are in the form of nucleic acid arrays, or compartmentalized kits, including microfluidic/lab-on-a-chip systems.

SNP detection kits may contain, for example, one or more probes, or pairs of probes, that hybridize to a nucleic acid molecule at or near each target SNP position. Multiple pairs of allele-specific probes may be included in the kit/system to simultaneously assay large numbers of SNPs. In some kits, the allele-specific probes are immobilized to a substrate such as an array or bead.

The terms “arrays”, “microarrays”, and “DNA chips” are used herein interchangeably to refer to an array of distinct polynucleotides affixed to a substrate, such as glass, plastic, paper, nylon or other type of membrane, filter, chip, or any other suitable solid support. The polynucleotides can be synthesized directly on the substrate, or synthesized separate from the substrate and then affixed to the substrate.

Any number of probes, such as allele-specific probes, may be implemented in an array, and each probe or pair of probes can hybridize to a different SNP position. In the case of polynucleotide probes, they can be synthesized at designated areas (or synthesized separately and then affixed to designated areas) on a substrate using a light-directed chemical process. Each DNA chip can contain, for example, thousands to millions of individual synthetic polynucleotide probes arranged in a grid-like pattern and miniaturized. Probes can be attached to a solid support in an ordered, addressable array.

A microarray can be composed of a large number of unique, single-stranded polynucleotides, usually either synthetic antisense polynucleotides or fragments of cDNAs, fixed to a solid support. Typical polynucleotides are about 6-60 nucleotides in length, or about 15-30 nucleotides in length, or about 18-25 nucleotides in length. For certain types of microarrays or other detection kits/systems, it may be preferable to use oligonucleotides that are only about 7-20 nucleotides in length. In other types of arrays, such as arrays used in conjunction with chemiluminescent detection technology, exemplary probe lengths can be, for example, about 15-80 nucleotides in length, or about 50-70 nucleotides in length, or about 55-65 nucleotides in length, or about 60 nucleotides in length. The microarray or detection kit can contain polynucleotides that cover the known 5′ or 3′ sequence of a gene/transcript or target SNP site, sequential polynucleotides that cover the full-length sequence of a gene/transcript; or unique polynucleotides selected from particular are as along the length of a target gene/transcript sequence. Polynucleotides used in the microarray or detection kit can be specific to a SNP or SNPs of interest (e.g., specific to a particular SNP allele at a target SNP site, or specific to particular SNP alleles at multiple different SNP sites).

Hybridization assays based on polynucleotide arrays rely on the differences in hybridization stability of the probes to perfectly matched and mismatched target sequence variants. For SNP genotyping, it is generally preferable that stringency conditions used in hybridization assays are high enough such that nucleic acid molecules that differ from one another at as little as a single SNP position can be differentiated. Such high stringency conditions may be preferable when using, for example, nucleic acid arrays of allele-specific probes for SNP detection. In some embodiments, the arrays are used in conjunction with chemiluminescent detection technology.

A polynucleotide probe can be synthesized on the surface of the substrate by using a chemical coupling procedure and an inkjet application apparatus, as described in PCT Publication No. WO 95/251116. In another aspect, a “gridded” array analogous to a dot (or slot) blot may be used to arrange and link cDNA fragments or oligonucleotides to the surface of a substrate using a vacuum system, thermal, UV, mechanical or chemical bonding procedures.

Methods for using such arrays or other kits/systems, to identify SNPs and haplotypes disclosed herein in a test sample are provided. Such methods typically involve incubating a test sample of nucleic acids with an array comprising one or more probes corresponding to at least one SNP position of the present invention, and assaying for binding of a nucleic acid from the test sample with one or more of the probes. Conditions for incubating a SNP detection reagent (or a kit/system that employs one or more such SNP detection reagents) with a test sample vary. Incubation conditions depend on such factors as the format employed in the assay, the detection methods employed, and the type and nature of the detection reagents used in the assay.

A SNP detection kit/system can include components that are used to prepare nucleic acids from a test sample for the subsequent amplification and/or detection of a SNP-containing nucleic acid molecule. Such sample preparation components can be used to produce nucleic acid extracts (including DNA and/or RNA), proteins or membrane extracts from any bodily fluids (such as blood, serum, plasma, urine, saliva, phlegm, gastric juices, semen, tears, sweat, etc.), skin, hair, cells (especially nucleated cells), biopsies, buccal swabs or tissue specimens.

Another form of kit is a compartmentalized kit. A compartmentalized kit includes any kit in which reagents are contained in separate containers. Such containers include, for example, small glass containers, plastic containers, strips of plastic, glass or paper, or arraying material such as silica. Such containers allow one to efficiently transfer reagents from one compartment to another compartment such that the test samples and reagents are not cross-contaminated, or from one container to another vessel not included in the kit, and the agents or solutions of each container can be added in a quantitative fashion from one compartment to another or to another vessel. Such containers may include, for example, one or more containers which will accept the test sample, one or more containers which contain at least one probe or other SNP detection reagent for detecting one or more of the disclosed SNPs, one or more containers which contain wash reagents (such as phosphate buffered saline, Tris-buffers, etc.), and one or more containers which contain the reagents used to reveal the presence of the bound probe or other SNP detection reagents. The kit can optionally further include compartments and/or reagents for, for example, nucleic acid amplification or other enzymatic reactions such as primer extension reactions, hybridization, ligation, electrophoresis (e.g., capillary electrophoresis), mass spectrometry, and/or laser-induced fluorescent detection. The kit may also include instructions for using the kit.

Microfluidic devices may also be used for analyzing SNPs. Such systems miniaturize and compartmentalize processes such as probe/target hybridization, nucleic acid amplification, and capillary electrophoresis reactions in a single functional device. Such microfluidic devices typically utilize detection reagents in at least one aspect of the system, and such detection reagents may be used to detect one or more of the disclosed SNPs. For genotyping SNPs, an exemplary microfluidic system may integrate, for example, nucleic acid amplification, primer extension, capillary electrophoresis, and a detection method such as laser induced fluorescence detection.

### EXAMPLES

### Example 1

### Genetic Mapping of Ma1

Materials and Methods

The methods for genetic mapping are published in Lin, et al., Genetics, 141:391-411 (1995).

Association genetics used a 384-member worldwide sorghum diversity panel from ICRISAT, previously characterized with 41 SSR markers Hash, et al., In 2008 Annual Research Meeting Generation Challege Programme, Bangkok, Thailand; 2008), evaluated in 2007 under short-day conditions (11.8-12.15 hrs light) and high humidity, under which short-day sorghums are expected to initiate flowering promptly. A 2008 planting was characterized by a transition from long to short-day (13.1 to 11.0 hr) photoperiod and dry conditions, and short-day sorghums would be expected to delay flowering. Flowering time was the number of days required for 50% of the plants in a single row to flower (DFL50%). Photoperiod Response Index (PRI) was defined as the mean difference in DFL50% between the two planting seasons (i.e. PRI=DFL50%₂₀₀₈−DFL50%₂₀₀₇).

Resequencing used BigDye terminator chemistry, and sequences were manually checked and aligned for single nucleotide polymorphism (SNP) identification with Sequencher 4.1.

Results

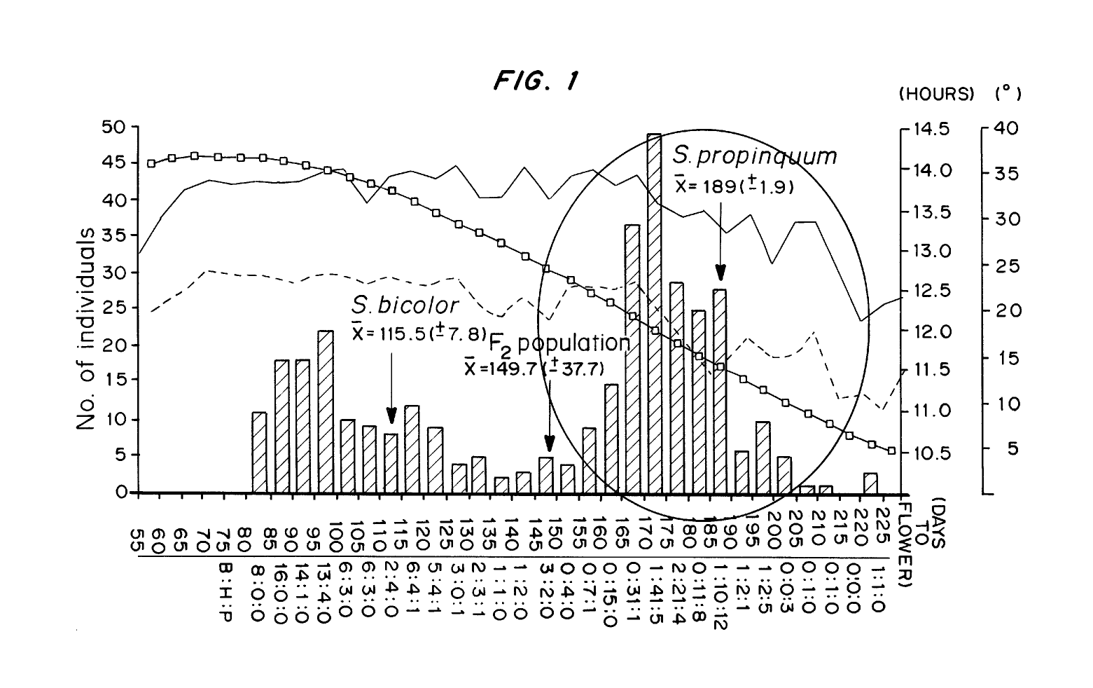

S. propinquum containing the Ma1 locus flowers later than cultivars of S. bicolor used in the U.S.A. Segregation for S. bicolor BTx623 versus S. propinquum alleles at the Ma1 locus imparts dichotomous phenotype when grown in a temperate environment (Lin, et al., Genetics, 141:391-411 (1995)). Interval mapping (Lander, et al., Genetics, 121:185-199 (1989)) was used to analyze an F₂ population of S. bicolor BTx623, a temperate cultivated sorghum, crossed with S. propinquum, a wild tropical sorghum. As shown in FIG. 1, the F₂ population of S. bicolor×S. propinquum demonstrated bimodal distribution of flowering time frequency when grown in a temperate environment. Specifically, S. propinquum (189±1.9 days) and most F₂s flowered later than S. bicolor (115.4±7.8 days) when photoperiod was less than 12.5 hours. The Ma1 locus alone accounts for 85.7% of phenotypic variation in flowering time (Lin, et al., Genetics, 141:391-411 (1995)) and mapped to chromosome 6, as later corroborated by independent work in different germplasm (Klein, et al., The Plant Genome, 1:S12-S26 (2008)).

To conduct interval mapping of flowering time in sorghum, an F2 population of Sorghum bicolor, BTx623 [S. bicolor (L.) Moench.], (S. propinquum was analyzed using 78 RFLP loci spanning 935 cM with an average distance of 14 cM between markers (Paterson, et al., Science, 269:1714-1718 (1995) Lin, et al., Genetics, 141:391-411 (1995). Ma1 was placed in the 21 cM interval between DNA markers pSB095 and pSB428a.

To more finely map the photoperiodic gene, 34 plants were selected that were putatively recombinant in the interval containing Ma1 based on flanking RFLP markers. An additional 27 DNA markers were applied to pooled DNA from 50 to 150 selfed F3 progenies that were also grown in the field near College Station, Texas. Four of the 34 F3 families, #10, 187, 191, and 211, were excluded because the DNA marker genotypes of F2 and pooled F3 tissue were not consistent (#211), or because the Ma1 genotype of their F2 parents predicted from the phenotype segregation in F3 progenies was contradicted by both flanking markers, as well as by virtually all other markers on the chromosome (all others). In each case, the inconsistency would have required a double recombination event, and three such events among 34 progeny is highly improbable. A modest number of such incongruous plants were also observed in the F2, and were an important example of the need for progeny testing—since flowering can be influenced by other genetic effects, temperature, and other factors such as some diseases (Quinby, Sorghum Improvement and the Genetics of Growth. College Station: Texas A&M University Press: 1974).

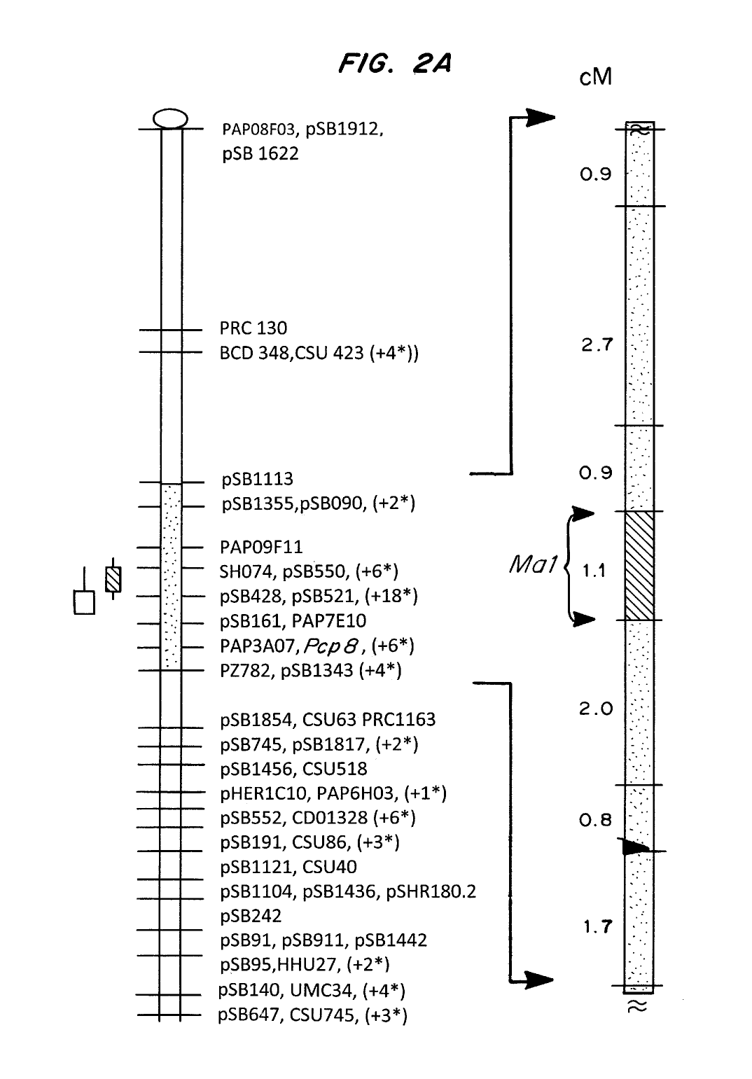

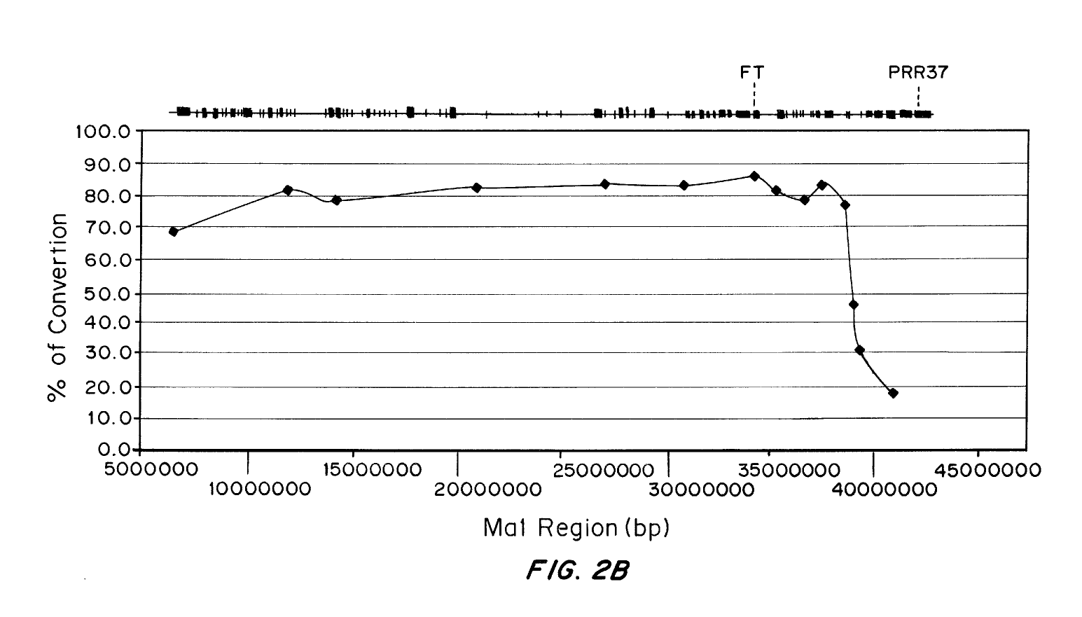

By testing F3 progeny of recombinants in the region, Ma1 was placed between markers pSB1113 and CDSR084, DNA markers estimated to be separated in the range from 0.3 to 1.1 cM in two different progeny arrays studied (FIG. 2A). While BAC clones were identified containing each of these DNA markers and others nearby, efforts to ‘chromosome walk’ in this region failed. The 1.1 cM region containing Ma1 is, physically, among the largest in the genome, with 60-fold less recombination than the genome-wide average of 0.7 mbp/cM. Spanning 34 million base-pairs (mbp), this region alone contains about 5% of sorghum genomic DNA and 1.3% (˜400) of genes. QTLs for many additional traits (beyond flowering) are also closely associated with Ma1, including a major dwarfing gene (Lin, et al., Genetics, 141:391-411 (1995)). Classical literature has defined these loci as Ma1 and Dw2.

Exotic-converted sorghum pairs were compared in the Ma1 region to access recombinational information resulting from the independent conversion(s) of about 90 sorghum genotypes. “Conversion” takes 12 generations and 4 years (Stephens, et al. Crop Sci., 7:396 (1967)), with one backcross followed by two generations of selfing (lacking DNA markers, this was necessary to phenotypically distinguish heterozygotes from homozygotes for the recessive photoperiod-insensitive allele). Across the sorghum gene pool, Ma1 has a singularly large role in the genetic determination of flowering. Among nine diverse exotic-converted sorghum pairs, all nine are ‘converted’ (introgressed with chromatin from the photoperiod-insensitive donor line) in the Ma1 region (Lin, et al. Genetics, 141:391-411 (1995)).

In the Ma1 region and any other regions that remain heterozygous, an exotic-converted pair offers about 3-4× the recombinational information than could be obtained from a single F2 or recombinant inbred genotype (estimated using standard formulas: (Allard, Hilgardia, 24:235-278 (1956)). A set of 90 exotic-converted pairs that broadly sample sorghum diversity and BTx406, the donor of day-neutral flowering, were genotyped with 9 SSR loci distributed through the region containing Ma1, with a peak introgression frequency of 84%. Haplotypes were determined and are illustrated in FIG. 2C, with the dark line indicating the span of converted regions. In the region of greatest conversion, additional genes and DNA markers were characterized, with a peak conversion frequency of 87% for the 400 bp indel that occurs upstream (5′) of the Sb06g012260 gene (FIG. 2B; Sb06g012260 itself was not characterized in this study). Frequencies of conversion at the DNA marker loci are plotted along the sorghum genome sequence, with approximate locations of genes in the sequence shown as cross-hatches along the axis. While the terminal regions that these data exclude from consideration are physically small, they contain the majority of genes.

PRR37, a candidate gene with expression patterns correlated with short-day flowering (Murphy, et al., Proceedings of the National Academy of Science of the United States of America, 108:16469-16474 (2011)), maps outside of this region, with less than 20% conversion (FIG. 2B), indicating that it does not account for short-day flowering in most if any exotic sorghums. Further, in short-day S. propinquum, PRR37 is non-functional with a 2 nt insertion causing 19 nonsense mutations, effectively ruling out that it could confer a dominant phenotype in crosses with S. bicolor. PRR37 is, however, very near the reported genetically-mapped location of Ma6 (Brady, Sorghum Ma5 and Ma6 Maturity Genes. Texas A&M University, 2006) a gene with a smaller effect on flowering. Thus, while PRR37 is not Ma1, it may be Ma6 and play a different role in the regulation of flowering.

Genes in the genomic region experiencing high frequencies of ‘conversion’ (introgression of day-neutral flowering) were re-sequenced in a diversity panel of 384 (Hash, et al., In 2008 Annual Research Meeting Generation Challege Programme, Bangkok, Thailand; 2008) accessions (87% landraces, 6% wild types, 6% breeding materials and 1% advanced cultivars) phenotyped for flowering under both short-day and long-day conditions, permitting calculation of a “Photoperiod Index” (PRI) reflecting the flowering behavior of each accession (see Methods for Association Genetics). Prior data on 41 SSR markers permitted investigation of population structure and genetic diversity of the panel, providing the relatedness information needed for formal testing of associations between specific alleles and PRI (Remington, et al., Proceedings of the National Academy of Science of the United States of America, 98:11479-11484 (2001); Thornsberry, et al., Nature Genetics, 28:286-289 (2001); Yu, et al., Nature Genetics, 38:203-208 (2005)).

Sb06g012260 was a gene discovered to be near the peak frequency of conversion. Sb06g012260 is a gene containing an ‘FT’ functional domain associated with regulation of flowering in Arabidopsis (Kardailsky, et al., Science, 286:1962-1965 (1999) and Oryza (Kojima, et al., Plant and Cell Physiology, 43:1096-1105 (2002)). Candidate alleles of Sb06g012260 were resequenced in a diversity panel of 384 individuals for which flowering time was known (see Example 3).

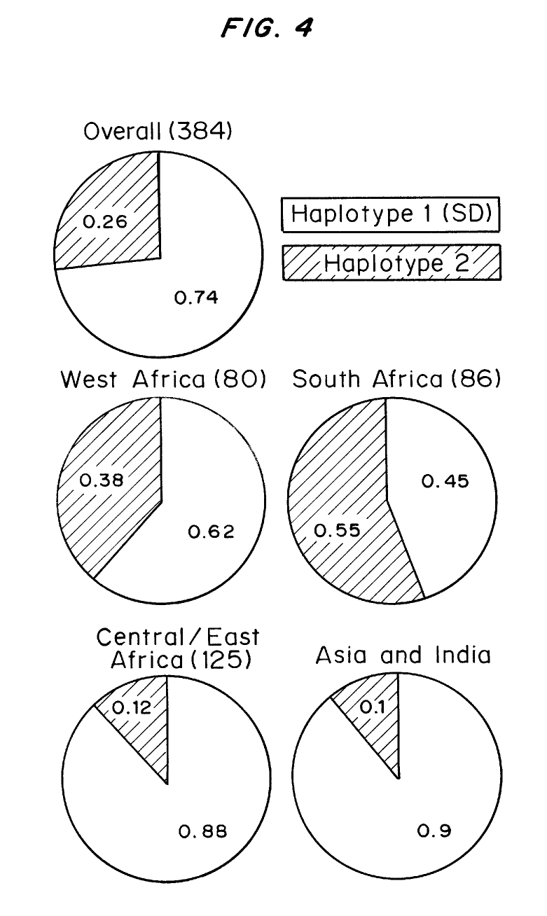

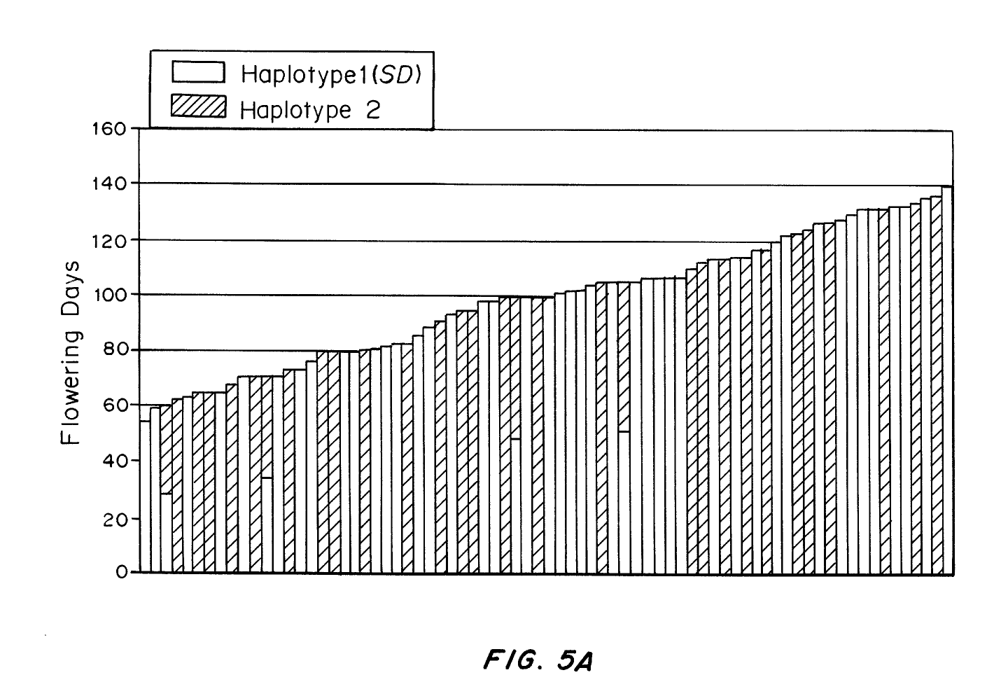

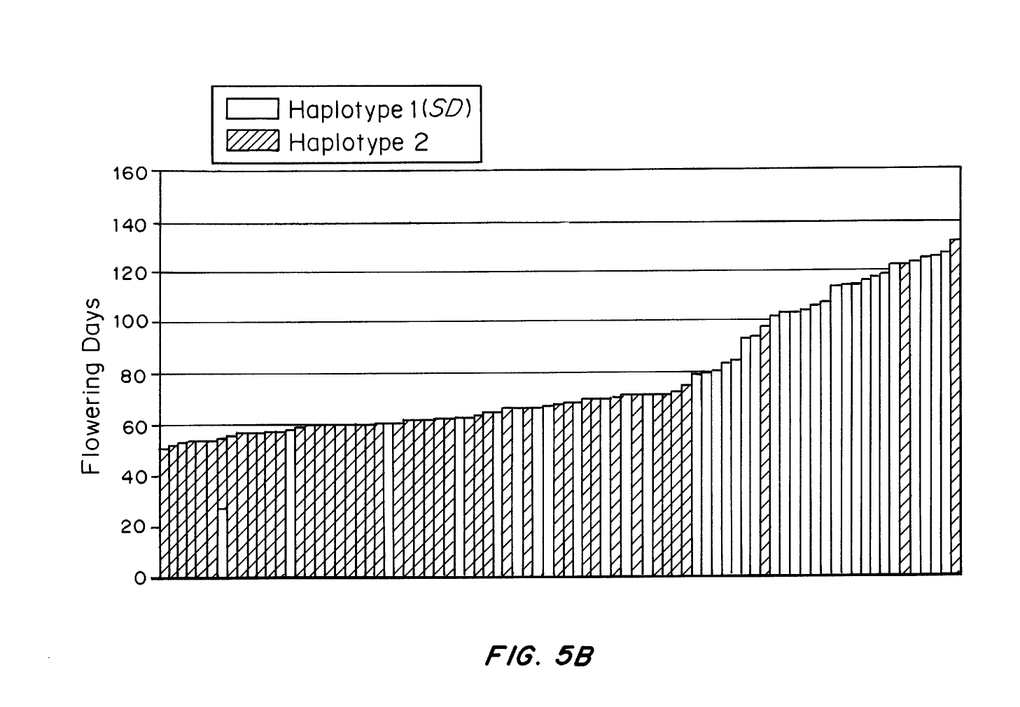

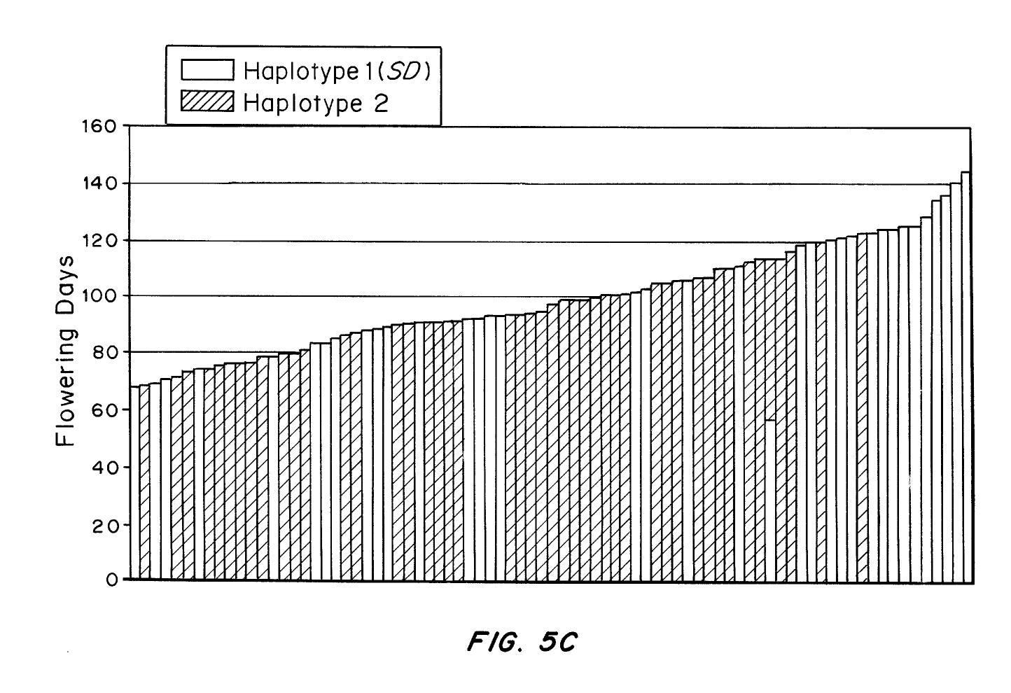

Analysis of this resequencing data identified two major haplotypes (each with two rare variants), one closely resembling the allele found in the short-day flowering accession of S. propinquum (FIG. 3A), and the other showing greatest abundance in sorghums from South Africa, the most temperate part of the pre-Columbian range (FIG. 4). Statistically-significant association of these haplotypes with PRI were found in subpopulations in which the two haplotypes each occur at similar frequencies (FIG. 4 and FIGS. 5A, 5B, and 5C). FIGS. 5A, 5B, 5C are based on SNPs from the coding sequence of the Ma1 gene. The Figures show independent analysis of each subpopulation. TASSEL association analysis including all the subpopulations and the covariance by the population structure is discussed in more detail below and shown in Tables 1 and 2.

FIGS. 5A, 5B, and 5C showing flowering (days) for individuals having a short-day haplotype or a day neutral haplotype for the gene Sb06g012260 in West Africa (FIG. 5A, 2008, p=0.005; R²=0.13) and South Africa (FIG. 5B (2008), p=3.84 E-08; R²=0.33; and FIG. 5C (2007), p=0.0346; R²=0.08). These data also show a statistically-significant association of the haplotypes with flowering in subpopulations in which the two haplotypes each occur at similar frequencies. The most informative subpopulation, sorghums originating in the South Africa region, is the subpopulation in which the day-neutral allele (haplotype) occurs at highest frequency.

The day-neutral haplotype included four deletions: (1) a 423 base pair deletion in the 5′ UTR of the Sb06g012260 (2) a ˜4.2 kb deletion in the 5′ UTR of the Sb06g012260, (3) a three base pair deletion starting about 221 base pairs upstream of the Sb06g012260 transcription-start site, and (4) a 27 base pair deletion in the second intron; and five synonymous single nucleotide polymorphism mutations (SNPs) in the coding sequence (FIG. 3B). Among the four deletions of the day-neutral haplotype, the 3-bp deletion is particularly damaging, removing from the Sb06g012260 promoter a CAAT box, an invariant DNA sequence in many eukaryotic promoters required for sufficient transcription (Berg, et al., Biochemistry, 5^{th} Ed. (2002)).

Other elements of the haplotype appear likely to be associated with the phenotype by linkage drag. For example, the 423 bp insertion appears to be a CACTA transposon. CACTA elements have been implicated as a mechanism of movement of genes and gene fragments in sorghum (Paterson, et al., Nature, 457:551-556 (2009)). The element present in the short-day haplotype has a close match in the day-neutral S. bicolor BTx623 genome sequence, presumably its ‘parent’ element, since that hit is to an autonomous element, while the insertion into S. propinquum has lost ability to transpose. Sequence divergence between the putative ‘parent’ element and the insertion is 94%—using published approaches to ‘date’ transposon insertions (SanMiguel, et al., Nature Genetics, 20:43-45 (1998)) suggests an ‘age’ of about 2 million years for the element. This suggests that the insertion may have only occurred in the S. bicolor/S. propinquum lineage, since this is much more recent than its divergence from Saccharum and other near relatives.

The approximately 4.2 kb element present in the short-day haplotype contains an inferred open reading frame found on a different chromosome of day-neutral S. bicolor BTx623 (chr. 7, Sb07g008600). Further, this element does not correspond discernibly to any gene of known function, and shows only limited similarity to two other sorghum genes, both also “putative uncharacterized proteins” (Sb03g005850, Sb08g011060). While a role in short-day flowering cannot yet be ruled out, its presence in day-neutral sorghum argues against a direct role in short-day flowering, and its mobility since the S. bicolor/S. propinquum divergence implies (as for the CACTA element) that it is likely to be an as-yet unrecognized transposon.

The remaining deletion is in the second intron.

Additional indels of 2 and 7 nt (5,451 and 5,025 nt upstream), and three synonymous mutations in exon 1 and two in exon 2 were not analyzed in depth.

### Example 2

### Association Analysis Among Ma1 Region Markers

Materials and Methods

A public sorghum reference germplasm set that substantially represents the spectrum of diversity in S. bicolor has been characterized with a genome-wide panel of SSRs, and phenotyped for flowering time across a number of diverse environments including some in photoperiods long enough to delay flowering of daylength-sensitive types. These data are freely available, and provide the information needed for formal testing of associations between specific alleles and phenotypes. Because it is predominantly self-pollinating with linkage disequilibrium extending over ˜15 kb, sorghum is an attractive system in which to employ association genetics to link DNA sequences to their phenotypic consequences.

The diversity panel was evaluated during two different planting seasons representing different day length conditions. The first planting (2007) represented short-day conditions (11.8-12.15 hrs light) and high humidity conditions, conditions under which short-day sorghums (i.e. photoperiod sensitive) are expected to initiate flowering promptly or similar to neutral day (i.e. photoperiod insensitive). The second (2008) planting was characterized by a transition from long to short-day (13.1-11.0 hrs) photoperiod and dry conditions, and short-day sorghums would be expected to delay flowering under these conditions.

Flowering time was recorded as the number of days required for 50% of the plants in a single row to flower (DFL50%). Photoperiod Index (PRI) of each accession was defined as the mean difference in DFL50% between the two planting seasons (i.e. PRI=DFL50%2008−DFL50%2007). Photoperiod sensitive accessions showed positive PRI values, while negative values identified photoperiod insensitive materials.

The quantity and frequency of haplotypes, and linkage disequilibrium were determined by Haplotyper 1.0, and TASSEL 2.1, respectively. TASSEL was used to perform tests of association, employing population structure covariates and a kinship matrix for the GCP/ICRISAT germplasm panel based on published SSRs (Hash, et al., In 2008 Annual Research Meeting Generation Challege Programme, Bangkok, Thailand; 2008).

Results

TASSEL (Bradbury, et al., Bioinformatics, 23:2633-2635 (2007)) was used to perform both linkage disequilibrium analysis and tests of association, the latter employing population structure covariates and a kinship matrix determined for the germplasm panel based on the 80 SSRs.

10 genes distributed across the target region were resequenced, in most members of the diversity panel (excepting those for which reactions failed, etc). TASSEL has been used to perform both linkage disequilibrium analysis and tests of association, the latter employing population structure covariates and a kinship matrix determined for the germplasm panel based on the existing SSRs.

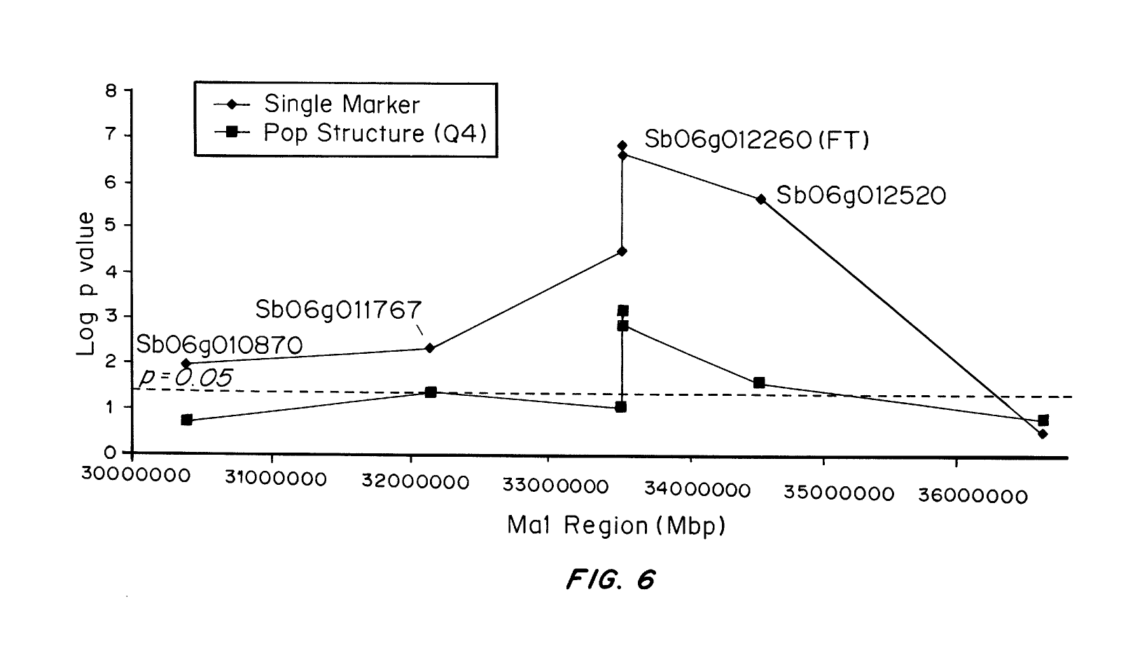

The results of the resequencing is presented in Tables 1-2, and FIG. 6. In partial summary, these data delimit the target region to the interval between genes Sb06g0111767 and Sb06g012520, which is 1.3 Mb with 20 annotated genes. The strongest evidence is found at the ˜4.2 kb indel in the Sb06g012260 gene.

TABLE 1
Association analysis among Ma1 region markers and the photoperiod by single marker analysis

| Data | Gene Marker | df | F | pF | df | df Error | MS Error | Rsq Model | Rsq Marker |
|---|---|---|---|---|---|---|---|---|---|
| FLOW_2008_2007 | SSR7 | 2 | 6.0672 | 0.0026 | 2 | 347 | 472.206 | 0.0338 | 0.0338 |
| FLOW_2008_2007 | SSR8 | 2 | 8.5317 | 2.42E−04 | 2 | 345 | 449.7492 | 0.0471 | 0.0471 |
| FLOW_2008_2007 | Sb06g010870 | 2 | 4.52 | 0.0116 | 2 | 320 | 483.4132 | 0.0275 | 0.0275 |
| FLOW_2008_2007 | Sb06g011767 | 1 | 8.0108 | 0.0049 | 1 | 322 | 450.9787 | 0.0243 | 0.0243 |
| FLOW_2008_2007 | 400bpINDEL | 2 | 10.8095 | 2.94E−05 | 2 | 296 | 454.8433 | 0.0681 | 0.0681 |
| FLOW_2008_2007 | 4kbINDEL | 2 | 16.5587 | 1.37E−07 | 2 | 340 | 429.1795 | 0.0888 | 0.0888 |
| FLOW_2008_2007 | Sb06g012260 | 2 | 16.2049 | 1.83E−07 | 2 | 358 | 437.37 | 0.083 | 0.083 |
| | (FT) | | | | | | | | |
| FLOW_2008_2007 | Sb06g012520 | 2 | 13.7554 | 1.88E−06 | 2 | 313 | 439.8079 | 0.0808 | 0.0808 |
| FLOW_2008_2007 | Sb06g013230 | 2 | 1.3111 | 0.271 | 2 | 315 | 473.4048 | 0.0083 | 0.0083 |
| FLOW_2008_2007 | Sb06g013810 | 2 | 1.4601 | 0.2337 | 2 | 338 | 474.4558 | 0.0086 | 0.0086 |

TABLE 2
Association analysis among Ma1 region markers and the photoperiod
with the correction of population structure (Q)

| Data | Gene Marker | df | F | pF | df | df Error | MS Error | Rsq Model | Rsq Marker |
|---|---|---|---|---|---|---|---|---|---|
| FLOW_2008_2007 | SSR7 | 2 | 0.0216 | 0.9786 | 6 | 343 | 398.859 | 0.1933 | 1.02E−04 |
| FLOW_2008_2007 | SSR8 | 2 | 12.522 | 5.65E−06 | 6 | 341 | 358.8457 | 0.2485 | 0.0552 |
| FLOW_2008_2007 | Sb06g010870 | 2 | 1.529 | 0.2183 | 6 | 316 | 405.8768 | 0.1937 | 0.0078 |
| FLOW_2008_2007 | Sb06g011767 | 1 | 4.0098 | 0.0461 | 5 | 318 | 386.0947 | 0.175 | 0.0104 |
| FLOW_2008_2007 | 400bpINDEL | 2 | 2.4506 | 0.088 | 6 | 292 | 377.6165 | 0.2368 | 0.0128 |
| FLOW_2008_2007 | 4kbINDEL | 2 | 7.4975 | 6.52E−04 | 6 | 336 | 362.9691 | 0.2384 | 0.034 |
| FLOW_2008_2007 | Sb06g012260 | 2 | 6.7615 | 0.0013 | 6 | 354 | 375.5924 | 0.2213 | 0.0297 |
| | (FT) | | | | | | | | |
| FLOW_2008_2007 | Sb06g012520 | 2 | 3.6981 | 0.0259 | 6 | 309 | 376.269 | 0.2236 | 0.0186 |
| FLOW_2008_2007 | Sb06g013230 | 2 | 1.9067 | 0.1503 | 6 | 311 | 375.0981 | 0.2242 | 0.0095 |
| FLOW_2008_2007 | Sb06g013810 | 2 | 6.5932 | 0.0016 | 6 | 334 | 376.9455 | 0.2216 | 0.0307 |

### Example 3

### PRR37 is not Ma1

As noted above, PRR37, a candidate gene with expression patterns correlated with short-day flowering (Murphy, et al., Proceedings of the National Academy of Science of the United States of America, 108:16469-16474 (2011)), maps outside of this region, with less than 20% conversion (FIG. 3B), indicating that it does not account for short-day flowering in most if any exotic sorghums.

Several additional lines of evidence also show that PRR37 cannot be Ma1. The sorghum genotype 100M, used to discern expression patterns correlating the PRR37 candidate allele to short-day flowering (Murphy et al., Proceedings of the National Academy of Science of the United States of America, 108:16469-16474 (2011)), also contains the short-day haplotype for Sb06g012260, which was confirmed by comparison to the short-day genotype PI209217.

Accordingly, differences in expression patterns between 100M and its near-isogenic line SM100 could be attributable either to PRR37, Sb06g012260, or other intervening genes on the introgressed segment. Indeed, in short-day S. propinquum, PRR37 contains a frameshift mutation that renders the PRR domain and much of the protein nonsensical and also causes premature termination (FIG. 3C). While PRR37 cannot account for short-day flowering in most sorghums, prior work by members of the PRR37 team showed it to be in the approximate location of Ma6 (Brady, Texas A&M University, (2006)), a gene with a much smaller effect on flowering.

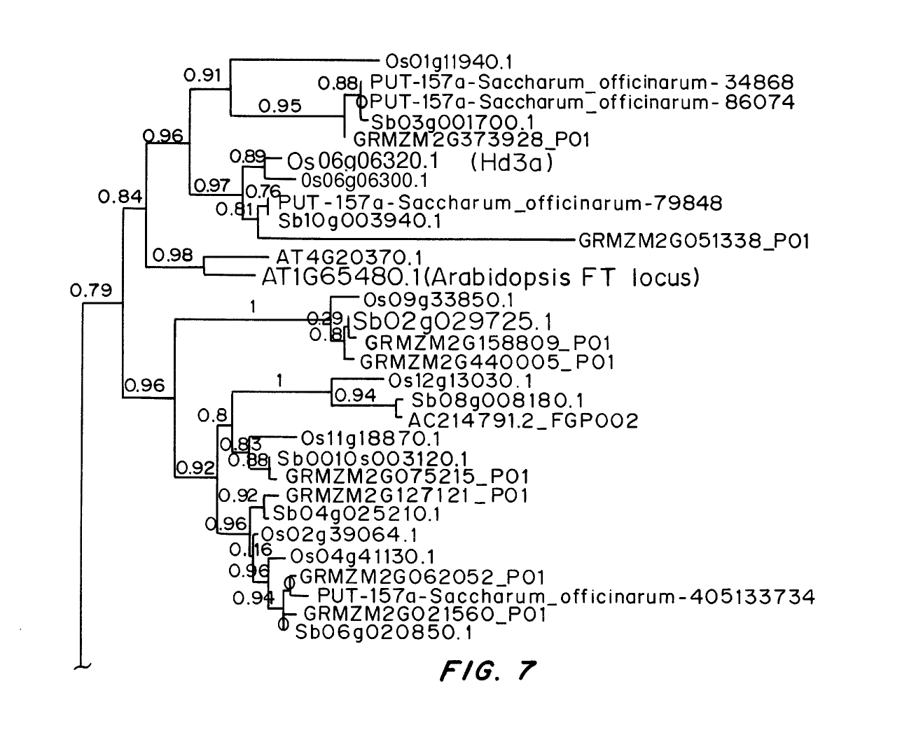

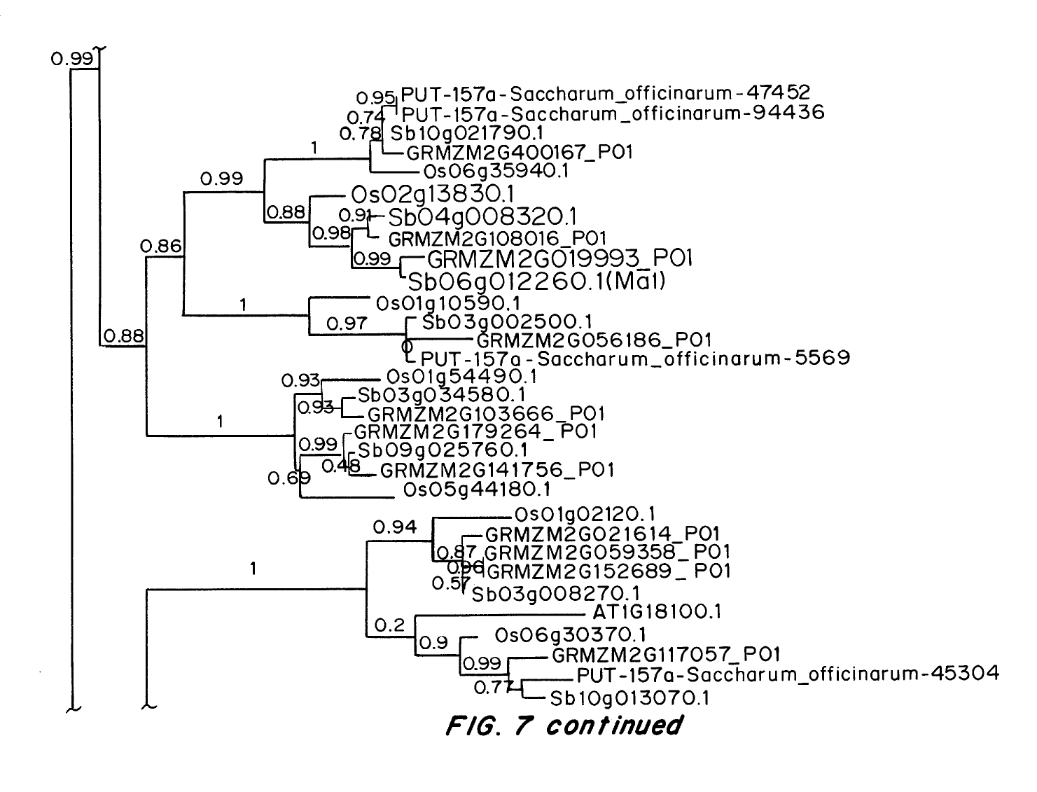

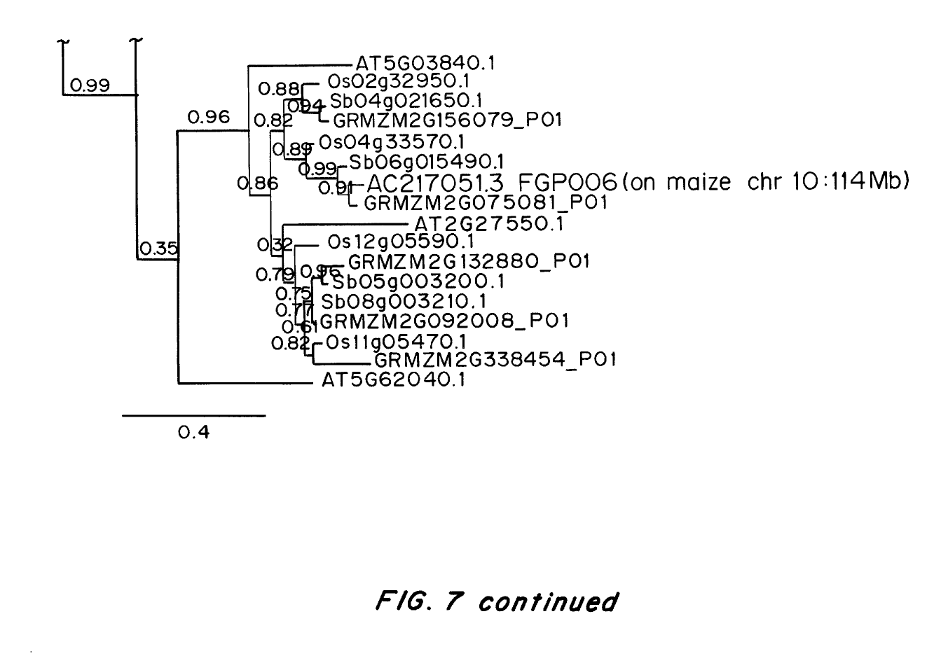

Homologs of the Ma1 candidate gene Sb06g012260 in sorghum (Paterson, et al., Nature, 457:551-56 (2009)), rice (Matsumoto, et al., Nature, 436:793-800 (2005)), and Arabidopsis (The Arabidopsis Genome Initiative. Nature, 408:796-815 (2000)) genomes, maize and sugarcane ESTs were identified by BLAST. The sugarcane ESTs were then translated to protein sequences. In total, 6 homologs were found in Arabidopsis (including the FT gene (Kardailsky, et al., Science, 286(5446):1962-1965 (1999)), 19 in rice (including Hd3a (Kojima, et al., Plant and Cell Physiology, 43(10):1096-1105 (2002)) and sorghum, 26 in maize and 8 in sugarcane (FIG. 7).

The candidate gene Sb06g012260 appears to have evolved as a single-gene duplication. Based on a synonymous substitution rate (Ks) of 0.43 from Sb04g008320, currently-used cereal molecular clocks suggest that this duplication occurred ˜40Mya (Gaut, et al., Proc Nat Acad Sci USA 93(19):10274-10279 (1996)). This date is more recent than the estimated divergence of rice and the sorghum/sugarcane/maize lineage, consistent with the finding that a positional ortholog was not discerned in rice. Sb04g008320 does have a rice ortholog (Os02g13830.1) of unknown function. Other members of the sorghum gene family do have rice orthologs, and several of the sorghum family members are much more similar to rice Hd3a (Os06g06320.1) than is the Ma1 candidate gene (Sb06g012260).

For Sb06g012260, a single maize ortholog, GRMZM2G019993, was identified on maize chromosome 2. Since maize has experienced a genome duplication since the divergence of the sorghum and maize lineages, the apparent presence of only one ortholog in the maize genome implies that a second duplicated copy was lost in maize. The missing homeolog would, if still present, be located on maize chr10, at approximately 105 Mb. Independent research has suggested the possibility of a major flowering time quantitative trait locus on maize chromosome 10 (Ducrocq, et al., Genetics, 183:1555-1563 (2009); Coles, et al., Genetics, 184:799-812 (2010)) and the presence of numerous candidate genes including an FT homolog (ZCN19; (Chardon, et al., Genetics, 168(4):2169-85 (2004); (Danielevskaya, et al., Plant Physiology, 146:250-64 (2008)). In the present maize genome sequence (Schnable, et al., Science, 326(5956):1112-15 (2009)), there are 4 maize FT genes on chromosome 10, but none at 105 Mb (GRMZM2G338454 chr10:5 Mb; AC214791.2_FG002 chr10:45 Mb; AC217051.3_FG006 chr10:114 Mb; GRMZM2G062052 chr10:127 Mb). The one of these closest to the target position (AC217051.3_FG006 chr10:114 Mb) is highly divergent in sequence from Sb06g012260, suggesting that it is not likely to be the ortholog.

### Example 4

### S. Halepense has a Mutation in the Sb06g012260 Promoter

The invasive plant Sorghum halepense, or ‘Johnson Grass’, has adapted to day-neutral photoperiod independently of, and perhaps even more rapidly than, breeder-improved sorghum. Sorghum halepense is a tetraploid derived from a naturally-occurring cross between wild forms of S. bicolor and S. propinquum (Celarier, Bull Torrey Bot Club, 85:49-62 (1958); Paterson, et al., Proceedings of the National Academy of Sciences of the United States of America, 92:6127-6131 (1995)). Being largely inbreeding, its wild progenitors would have each been expected to be homozygous for the short-day flowering Ma1 allele, with tetraploid S. halepense (also inbreeding) receiving 4 copies of the allele. Among the limited sampling available in the US National Plant Germplasm collection, two Old World accessions PI209217 from South Africa and PI271616 from India were confirmed to be short-day flowering—these were also both homozygous for the short-day haplotype. However, many or all U.S. populations of S. halepense are believed to include many members that flower in the long days of the temperate summer.

In S. halepense naturalized in the U.S., the central portion of the short-day flowering haplotype has been largely replaced with a segment that includes a different mutation in the Sb06g012260 promoter. The results of a sampling of 480 plants is summarized in Table 4.

TABLE 4
Presence or Absence of 4 mutations in S. halepense
(% among unambiguous genotypes)

| | 400 bp | 4.2 kb | 3 bp | intron |
|---|---|---|---|---|
| Non-ambiguous genotypes | % among non-ambiguous genotypes | | | |
| B: Day-neutral S. bicolor | 0.47% | 5.71% | 0.00% | 0.22% |
| genotype; | | | | |
| P: Short-day S. propinquum | 81.63% | 1.14% | 10.37% | 88.16% |
| genotype | | | | |
| H: S. halepense genotype | 0.00% | 0.00% | 1.67% | 0.00% |
| BP | 17.91% | 93.15% | 3.68% | 11.62% |
| BH (at least one B and one H | 0.00% | 0.00% | 0.67% | 0.00% |
| allele) | | | | |
| PH | 0.00% | 0.00% | 72.24% | 0.00% |
| BPH (at least one allele each of | 0.00% | 0.00% | 10.70% | 0.00% |
| B, P, and H) | | | | |
| Ambiguous genotypes | % among total sample | | | |
| PH-like (closely resembles PH) | 0.00% | 0.00% | 29.43% | 0.00% |
| Other | 11.63% | 9.59% | 24.03% | 5.26% |

Among 480 plants sampled equally from each of five S. halepense populations from GA, TX (2), NE, and NJ, USA (Morrell, et al., Molecular Ecology, 14:2143-2154 (2005)), 81.6% and 88.2% of plants scorable (i.e. excluding amplification failures or ambiguous migration patterns) were homozygous for the short-day haplotype at both terminal loci (423 bp, intron indels), but only 1.1 and 10.4% at the two internal loci (4,186 and 3 nt indels) (Table 4). Only 39 bp upstream from the site of the CAAT box deletion in day-neutral S. bicolor, 85.3% of the tetraploid S. halepense plants have at least one copy (with 1.7% being homozygous for all four copies, but noting that 1, 2, or 3 copies cannot be distinguished in this tetraploid) of a 4 nt insertion (i.e. not found in either progenitor) that disrupts a TC-rich repeat, a cis-acting element involved in defense and stress response (bioinformatics.psb.ugent.be/webtools/plantcare/html/). TC-rich repeats are enriched in the promoters of photoperiod-responsive genes, and photoperiod-responsiveness is thought to integrate multiple light-, hormone-, and stress-responsive elements (Mongkolsiriwatana, et al., Nat. Sci., 43:164-177 (2009)). Further, 98.9% also have at least one copy of the day-neutral (deletion) allele at the 4,186 nt indel, 5.7% being homozygous for the deletion. Finally, 15.7% of plants also carry one or more copies of the 3 nt deletion.

The adaptation of S. halepense to the temperate climate of the continental U.S.A. may predate the scientific breeding of day-neutral sorghums. Selection of day-neutral Ma1 alleles occurred during the first 40 years of the 20^{th} century (Quinby, Texas A&M University Press (1974); Smith, et al., John Wiley and Sons, (2000)) while S. halepense was well-established in the U.S.A. by 1847 and of sufficient importance in 1900 to be the subject of the first federal appropriation for weed control (McWhorter, Weed Science, 19:496 (1971)).

Sb06g012260 appears to have evolved as a single-gene duplication (FIG. 7), shortly after the oryzoid (rice)—panicoid (sorghum/sugarcane/maize) divergence. Based on a Ks of 0.43 from its nearest homolog, Sb04g008320, this duplication is an estimated 40 million years old (Gaut, et al., Proceedings of the National Academy of Sciences of the United States of America, 93:10274-10279 (1996)), consistent with the lack of a rice ortholog. Sb04g008320 does have a rice ortholog (Os02g13830.1), although of unknown function.

Sb06g012260 is extensively diverged from other known floral regulators—indeed, no members of its Glade have empirically-demonstrated functions (FIG. 7). Other sorghum family members do have rice orthologs, and some resemble a rice flowering time QTL Hd3a (Os06g06320.1)(Kojima, et al., Plant and Cell Physiology, 43:1096-1105 (2002)). However, Hd3a is well over 100 million years distant from Sb06g012260, even more than are the nearest Arabidopsis genes.

One family member, Sb02g029725, locates near the likelihood peak of a second sorghum flowering QTL with a small phenotypic effect (FlrAvgB1: Lin et al 1995). Resequencing of this gene in the 384-member diversity panel used above (Hash, In 2008 Annual Research Meeting Generation Challege Programme. Bangkok, Thailand; 2008). revealed two abundant haplotypes (resembling S. propinquum and BTx623 respectively), which showed highly significant association with PRI (p=1.53×10-6). Thus, at least two members of the FT gene family are implicated in the modulation of flowering in sorghum, reminiscent of sunflower domestication in which five FT paralogs experienced selective sweeps (Blackman, Genetics, 187:271-287 (2011)).

Sb06g012260 has a single maize ortholog, GRMZM2G019993, on chromosome 2. Since the maize genome duplicated after its divergence with the sorghum lineage, the presence of only one maize ortholog implies that a second one was lost, from chromosome 10 at ˜105 Mb. Maize chromosome 10 contains a major flowering time QTL (Ducrocq, et al., Genetics, 183:1555-1563 (2009); Coles, et al., Genetics, 184 (2010)) and four FT homologs (Schnable, et al., Science, 326:1112-1115 (2009)), but the nearest to 105 Mb (AC217051.3_FG006 chr10: 114 Mb) is so divergent in sequence from Sb06g012260 that it is not considered orthologous (FIG. 7).

The importance of Ma1 to fecundity, via flowering, may have contributed to the evolution of a ‘coadapted gene complex’ (Lande, Genetical Research, 26:221-235 (1975)) with cis-linkage of alleles at different loci that collectively confer an adaptive phenotype, perhaps facilitated by the recalcitrance of the region to recombination. The Ma1 region also holds dw2, the gene of largest effect on sorghum stature (height) (Lin, et al., Genetics, 141:391-411 (1995)), but which can be separated from Ma1 by infrequent recombination (Quinby, Texas A&M University Press (1974); Lin, Texas A&M University (1998)). Quinby indicated that Ma1 and Dw2 were different closely-linked genes, with ca. 8% crossing over (Quinby J R: Sorghum Improvement and the Genetics of Growth. College Station: Texas A&M University Press; 1974), but only 47 families were evaluated (based on phenotype).

Based on the observation that the late-flowering phenotype can occasionally be a result of factors other than allelic status at the Ma1 locus and that progeny testing is necessary to validate it, such a small study must be considered tenuous. Among the 30 validated F₃ families in the study, three showed different segregation patterns for flowering time and plant height. Since these 30 individuals comprised all confirmed recombinants in the region from a population of 370 individuals, this suggests a 0.5 cM linkage distance between Ma1 and Dw2 (Lin, Genetic analysis and progress in chromosome walking to the sorghum photoperiodic gene, Ma1. Texas A&M, Soil and Crop Science; 1998).

Increased height naturally confers a competitive advantage in light interception. Favorable alleles at different genes that conferred both optimal height and flowering time to the same progeny by virtue of the suppressed recombination in this genomic region, might have become fixed more quickly than independently-segregating alleles. Flowering time and plant height were correlated in the diversity panel (r=0.53 in 2007, 0.73 in 2008, each significant at 0.001). While the strongest statistical association found with plant height was at Sb06g012260 itself (p=0.007), there was also an association at Sb06g007330 (p=0.023), a putative cation efflux family protein. A putatively intervening gene, Sb06g010870, showed no association but could have recently formed alleles or be at an incorrect location, noting that this recombinationally-recalcitrant region is among the most repetitive in the sorghum genome and therefore one of the most difficult in which to assemble whole-genome shotgun sequence (Paterson A H et al. Nature, 457(7229):551-56 (2009)).

### Example 5

### Transformation of Short Day S. Propinquum Sb06g012260 into Day-Neutral Tx430 Delayed Flowering of F2 Progeny

Materials and Methods

Two constructs containing short-day S. propinquum Sb06g012260 alleles were transformed into day-neutral Tx430 (Howe, Plant Cell Reports, 25:784-791 (2006)). Widely used for sorghum transformation because of its high efficiency, Tx430 has a rare Ma1 mutation, containing the short-day haplotype except for deletion of 7 amino acids in the 4th exon. Independent TO transformants were selfed to produce T1 segregating progenies, then 15-24 plants from each T1 family were evaluated in the greenhouse under ambient long day conditions (at 33.95o N latitude), recording the number of days from planting on 17 May to flower emergence. Plants were genotyped by PCR to determine allele state for the transgene.

Transformation used published methods (Howe, Plant Cell Rep., 25:784-791 (2006)). Independent TO transformants were selfed to produce T1 segregating progenies, then 15-24 plants from each T1 family were evaluated in the greenhouse under ambient long day conditions (at 33.95° N latitude), recording the number of days from planting on 17 May to flower emergence. Plants were genotyped by PCR to determine allele state for the transgene.

Results

Transformation events involving two constructs containing short-day S. propinquum Sb06g012260 alleles transformed into day-neutral Tx430 each delayed flowering of transgenic F2 progeny in long days, although generally by less than the 24.6 (+3.5) day delay between the Ma1-containing reference genetic stock 100M (Murphy, et al., PNAS, 108:16469-16474 (2011) and Tx430, under the conditions used in this transformation. Among 13 transformation events carrying a transgene limited to Sb06g012260 and its immediate upstream elements, two conferred statistically significant delays averaging 13.1 (p=0.03) and 24.8 days (p=0.09), and one unexpected line showed accelerated flowering (14.1 days, p=0.05).

Shorter flowering delays than the Ma1 reference genotype100M relative to putatively near-isogenic SM100 [18] may indicate that some distant regulatory elements are missing from the construct and/or that its native heterochromatic chromatin environment is important to its natural function. However, among 10 independent events harboring a ˜10 kb construct spanning the entire haplotype (from Sb06g012260 through the 4,186 nt element), transgenic F2 progeny of only three showed significantly altered flowering, with delays of 4.1 (p=0.002), 4.2 (p=0.07) and 5.2 (p=0.008) days, suggesting that any such element(s) are still more distant.

The predominant day-neutral Sb06g012260 haplotype includes one mutation likely to cripple the gene. The 3-bp deletion located 219 nt upstream of Sb06g012260 removed a CAAT box, an invariant DNA sequence in many eukaryotic promoters required for sufficient transcription [26]. Other elements of the haplotype appear innocuous. The 423 bp deletion removes a non-autonomous CACTA transposon; and the 4,186 nt deletion removes an open reading frame also found on chr. 7 of day-neutral sorghum (Sb07g008600), with limited similarity only to two “putative uncharacterized proteins” (Sb03g005850, Sb08g011060) and with a ‘stop’ codon in its first exon.

The near-isogenic lines 100M and SM100 that differ in PRR37 expression patterns (Murphy, et al., PNAS, 108:16469-16474 (2011)) also contain different Sb06g012260 alleles, hence phenotypic differences between these lines could be explained by either of these two genes or interactions between them. The genotype 100M is introgressed with not only a putatively short-day PRR37 allele but also with the short-day Sb06g012260 haplotype, based on genotyping at both the 423 and 4,186 nt indels that are on the distal side of the gene relative to PRR37. A proposed functional pathway for PRR37 (Murphy, et al., PNAS, 108:16469-16474 (2011)) indicates that it influences flowering by regulation of FT—thus a loss of function in an FT homolog such as Sb06g012260 could supercede the effects of PRR37.

Several independent lines of evidence including fine mapping, association genetics, mutant complementation, and evolutionary analysis all implicate a single gene, Sb06g012260, as the cause of the Ma1 short-day flowering trait in sorghum. This new evidence also explains the reasons for a prior, erroneous, conclusion that another nearby gene was Ma1.

Potential applications of Ma1 are numerous. For example, in some embodiments, engineered genotypes that silence Ma1 may render obsolete the need to laboriously ‘convert’ tropical grasses to day-neutral flowering by twelve generations of breeding, potentially dramatically accelerating methods of cross-utilization of sorghum, sugarcane, and other crop germplasm between temperate and tropical regions. In some embodiments, compositions and methods of suppressing flowering by targeted selection or engineering of strong Ma1 alleles in biomass crops may confer consistent high yields, and can be used in broad ranging methods, for example, improving the economics of cellulosic biofuel production.

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize, or be able to ascertain using no more than routine experimentation, many equivalents to the specific embodiments of the invention described herein. Such equivalents are intended to be encompassed by the following claims.

We claim:

1. A method of delaying flowering in a plant, comprising introducing to the plant a nucleic acid sequence that silences expression of a polynucleotide having the nucleic acid sequence SEQ ID NO: 1, 2, 3, 4, 5, 6, 7, 9, 10, 11, 12, 13, 28, 29, 30, 31, 32, 33 or a complement thereof.

2. The method of claim 1, wherein the plant is a dicotyledon.

3. The method of claim 1, wherein the plant is a monocotyledon.

4. The method of claim 1, wherein the plant has lower photoperiod sensitivity compared to a control plant of the same species.

5. A method of delaying flowering in plant comprising altering the sequence of SEQ ID NO: 14, 15, 16, 17, 18, 19, 20, 21, 22, 23, 24, 25, 26 or variants thereof in the plant.

6. The method of claim 5, wherein the altering comprises introducing one or more nucleic acid substitutions, additions, deletions or a combination thereof in SEQ ID NO: 14, 15, 16, 17, 18, 19, 20, 21, 22, 23, 24, 25, 26 or variants thereof.

7. A method of increasing or accelerating flowering in a plant, comprising introducing to the plant a nucleic acid sequence comprising a nucleic acid sequence at least 90% identical to SEQ ID NO: 1, 2, 3, 4, 5, 6, 7, 9, 10, 11, 12, 13, 28, 29, 30, 31, 32, 33 or a complement thereof.
